(19) United States
(12) Patent Application Publication
(10) Pub. No.: US 2024/0110245 A1
(43) Pub. Date: Apr. 4, 2024

(54) COMPOSITIONS AND METHODS FOR DETECTING GYNECOLOGICAL CANCER

(71) Applicant: Mayo Foundation for Medical Education and Research, Rochester, MN (US)

(72) Inventors: William R. Taylor, Lake City, MN (US); John B. Kisiel, Rochester, MN (US); Douglas W. Mahoney, Rochester, MN (US)

(21) Appl. No.: 18/241,411

(22) Filed: Sep. 1, 2023

Related U.S. Application Data
(60) Provisional application No. 63/374,415, filed on Sep. 2, 2022.

(51) Int. Cl. C12Q 1/6886

(52) U.S. Cl. CPC C12Q 1/6886; C12Q 2600/112; C12Q 2600/154

(57) ABSTRACT

The present disclosure relates to detecting one or more types of gynecological cancer in a biological sample from a subject. In particular, the present disclosure provides compositions and methods for detecting the presence or absence of one or more types of gynecological cancer (e.g., cervical cancer, ovarian cancer, endometrial cancer) in a biological sample from a subject having or suspected of having a gynecological cancer.

### CROSS REFERENCE TO RELATED APPLICATIONS

This application claims priority to and the benefit of U.S. Provisional Patent Application No. 63/374,415 filed Sep. 2, 2022, which is incorporated herein by reference in its entirety and for all purposes.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 57,580 Byte ASCII (Text) file named “40960-202 SEQUENCE LISTING” created on Sep. 1, 2023.

### FIELD

The present disclosure relates to detecting one or more types of gynecological cancer in a biological sample from a subject. In particular, the present disclosure provides compositions and methods for detecting the presence or absence of one or more types of gynecological cancer (e.g., cervical cancer, ovarian cancer, endometrial cancer) in a biological sample from a subject having or suspected of having a gynecological cancer.

### BACKGROUND

Compared to other types of cancer (e.g., breast or colon cancer), gynecological cancers are not as common, occurring in about 100,000 women in the United States each year. However, all women are at risk for developing gynecological cancers, and the risk increases with age. The five main types of gynecological cancer include cervical, ovarian, uterine, vaginal, and vulvar. A sixth type of gynecological cancer is the very rare fallopian tube cancer. Among gynecological cancers, clinically relevant screening tests are currently available only for cervical cancer, despite evidence indicating that early detection of gynecological cancers is particularly important for improving survival rates. Since there is no simple and reliable way to screen for multiple gynecological cancers, it is especially important to recognize warning signs to reduce risk. Additionally, although gynecological cancer screening programs (e.g., HPV test, Pap test) are intended to increase survival rates through early detection, these tests are typically only available to a subset of the population (e.g., those at highest risk) and are limited to a small number of cancers (e.g., cervical cancer). Therefore, healthcare professionals are often only able to make accurate cancer diagnoses after symptoms have developed, which can be too late for effective treatment. As such, there is an urgent need for improved diagnostic tools for detecting multiple types or subtypes of gynecological cancers in a single biological sample to not only provide earlier detection, but also more accurate patient stratification and greater insight into therapeutic strategies.

### SUMMARY

Embodiments of the present disclosure provide methods, compositions, and systems for screening multiple types of gynecological cancer from a biological sample. In accordance with these embodiments, the present disclosure includes, but is not limited to, methods and compositions for detecting the presence of multiple types or subtypes of gynecological cancer from a biological sample. In some embodiments, the biological sample is a tissue sample, a blood sample, a plasma sample, a serum sample, a whole blood sample, a secretion sample, an organ secretion sample, a cerebrospinal fluid (CSF) sample, a saliva sample, a urine sample, and/or a stool sample. In some embodiments, the tissue sample is a gynecological tissue sample comprising one or more of vaginal tissue, vaginal cells, cervical tissue, cervical cells, endometrial tissue, endometrial cells, ovarian tissue, and ovarian cells. In some embodiments, the tissue sample is an ovarian tissue sample, an endometrial tissue sample, or a cervical tissue sample. In some embodiments, the secretion sample is a gynecological secretion sample. In some embodiments, the subject is a human.

As described further herein, embodiments of the present disclosure include novel differentially methylated regions (DMRs), each individually capable of distinguishing a specific type of gynecological cancer (i.e., endometrial cancer (EC), or ovarian cancer (OC), or cervical cancer (CC)) from a benign gynecological tissue sample. In accordance with these embodiments, these novel DMRs comprise one or more CpG sites in ADAM8, ADHFE1, AES, AGBL2, AIM1, AK5, ALKBH3, ARAP1, ARHGAP20, ASCL2, BCAT1, BEGAIN, BEND4_3696, BMP6, C12orf68, C13orf18, C14orf169_7694, C14orf169_8382, C18orf18, C1orf61, C20orf195, C4orf31, C5orf52, C6orf147, C7orf51, CD14, CELF2, CHCHD5, CHMP2A, CHST10, CLIC6, CLIP4, COL13A1, COL19A1, COL6A2, COPZ2, CREB3L1, CXCL2, CXXC5, CYTH2, DAB2IP, DGKZ, DLGAP3, DNASE2, DSCAML1, EBF1, EDARADD, EGR2, EIF5A2, ELMO1, ELMOD1, ELOVL4, EME2, EML6, EPSTI1, FADS2, FAM109B, FAM126A, FAM174B, FGF18, FKBP11, FLI1, FLOT1, FOXD3, FYN, GAL3ST2, GALR3, GAS7, GATA2_5878, GLT25D2, GNB2, HDAC7, HIC1, HLA-F, HNRNPF, HPDL, HS3ST4, HSPA1A, IDUA, IGSF9B, IL12RB2, IRAK3, IRF7, IRF8, ITPKA, KCNA2, KCNC3_6487, KCNC3_7105, KCNC4, KCNH8, KDM2B, LBX2, LCMT2, LOC100129726, LOC100287216, LOC255130, LOC339290, LOC729678, LPPR3, LRRC41, LRRC8D_8856, LTBP2, LYPLAL1, MAST4, MAX.chr1.2152, HIVEP3, GRAMD1B, MAX.chr11.0394, MAX.chr11.3750, FAT3, SLC16A7, MTUS2, LINC02323, MAX.chr14.7696, MCTP2, LOC107984974, TRIM80P, MAX.chr19.5552, ZNF433-AS1, ZNF254, MAX.chr19.0548, B3GALT1, MAX.chr2.8918, MAX.chr2.4778, MAX.chr20.3853, MAX.chr20.2903, MAX.chr21.5011, DSCR9, MAX.chr22.5665, MAX.chr3.6408, LINC02028, LINC02084, MAX.chr5.3588, CTD-2532K18.1, HS3ST5, ARHGAP18, GRM4, LINC01004, MAX.chr8.5938, MAX.chr9.4007, MAX.chr9.2025, TRPM3, MED12L, MIAT, MLH1_4513, MLH1_5193, MMP16, MRPS21, MSI1, MT1E, MX1, MYC, MYH10, MY015B, N4BP2L1, NBR1, NDRG2, NEGR1, NEU1, NOL3, NR3C1_2223, NR3C1_4614, NRP2, NTN1, NTNG1, PAPL, PAQR9, PDE10A, PDE3B, PDE4A, PDXK, PER1, PISD, PLEC, PLIN2, PLXND1, PPM1E, PPP1R9A, PPP2R5C, PRDM5, PTP4A3, PYCARD, RAB3C, RAI1, RARG, RASA3, RPRM, RREB1, S100A6, SAMD5, SBNO2, SDC2, SDK2, SELM, SERP2, SFMBT2_2029, SHF, SHE, SLC16A11, SLC16A5, SLC25A22, SLCO3A1, SMTN, SPDYA, SPINK2, SPOCK2, SPON1, SQSTM1_4156, ST8SIA1, TAF4B, TAF7, TEAD3, TERC, TIAM1, TLE4, TMEM101, TMEM106A, TRIM9, TRPC3, TSC22D4, TSPAN2, TSPAN5, TTC14, UBB_4001, UBB_4646, UST, VAMP5, VIM, VSTM2B, ZBTB7B, ZEB2, ZFP3, ZFP36L2, ZIC2, ZMIZ1, ZNF14, ZNF211, ZNF280B, ZNF302, ZNF382, ZNF480, ZNF483, ZNF491, ZNF569, ZNF610, ZNF702P, ZNF709, ZNF773, ZNF845, ZNF91, CDH4, LRRC34, MAX.chr10.4460, NBPF24, OBSCN, SEPT9, ZNF323, ZNF506, and/or ZNF90 (Table 1), including any combinations thereof. In some embodiments, the novel DMR(s) is from any gene or region selected from Table 1, including any combinations thereof. Each novel DMR alone is capable of distinguishing one or more gynaecological cancers from a control sample, and combining two or more of the novel DMRs can provide increased sensitivity. Therefore, combinations of two or more novel DMRs selected from Table 1 are provided.

Embodiments of the present disclosure also include novel differentially methylated regions (DMRs), each individually capable of distinguishing gynecological cancer from a benign gynecological tissue sample; these DMRs are universally present in all three types of gynecological cancer (i.e., endometrial cancer (EC), ovarian cancer (OC), and cervical cancer (CC)). In accordance with these embodiments, these novel DMRs comprise one or more CpG sites in ACSF2, AJAP1, ARL10, ARL5C, ASCL4, ATP6V1B1, BARHL1, BEND4_2963, C17orf64, C1QL3, C2orf55, C4orf48, CA3, CDO1, CELF2, CLEC14A, CSDAP1, CYTH2_4197, DLGAP1, DSCR6, EPS8L1_2819, EPS8L1_8496, FAIM2, FGF12, GATA2, HIST1H2BE, IRF4, IRX4, ITGA5, KCNA1, LECT1, LHX1, LOC440925, LPHN1, LINC02767, MAX.chr1.2533, SOX1-OT, MAX.chr13.3357, MAX.chr14.2093, MAX.chr17.2455, MAX.chr18.4390, MAX.chr19.2732, MAX.chr19.4467, PANTR1, MAX.chr2.0490, MAX.chr2.8148, MAX.chr2.3137, RIPOR3, SCRG1, MAX.chr4.4210, HMX1, CTC-359M8.1, MAX.chr5.0931, MAX.chr5.9924, LIN28B, MAX.chr6.9522, TTLL2, RNA5SP243, DLGAP2, MEX3B, MNX1, NEFL, NETO1, PAX2, PDX1, psiTPTE22, RASGEF1A, SALL3_9136, SALL3_0615, SEZ6L2, SHANK2, SHANK3, SKI, SLC35D3, SORCS3_0305, SORCS3_1038, SOX1, SQSTM1, TBXT, TCERG1L, TERT, TNFSF11, TUBB6, ULBP1, VAC14, VWC2, WDR69, ZBTB16, ZNF132, ZSCAN12, ZSCAN23, KRT86, CYP26C1, GYPC, DIDO1, EEF1A2, EMX2OS, GDF7, JSRP1, SMPD5, MDFI, MPZ, and/or VILL (Table 2), including any combinations thereof. In some embodiments, the novel DMR(s) is from any gene or region selected from Table 2, including any combinations thereof. Each novel DMR alone is capable of distinguishing one or more gynaecological cancers from a control sample, and combining two or more of the novel DMRs can provide increased sensitivity. Therefore, combinations of two or more novel DMRs selected from Table 2 are provided.

Embodiments of the present disclosure also include novel differentially methylated regions (DMRs), each individually capable of distinguishing a specific subtype of a gynecological cancer (i.e., serous ovarian cancer, clear cell ovarian cancer, endometrioid ovarian cancer, mucinous ovarian cancer, adenocarcinoma cervical cancer, squamous cervical cancer, or endometrioid endometrial cancer) from a benign gynecological tissue sample. In accordance with these embodiments, these novel DMRs comprise one or more CpG sites in AIM1, AK5, c18orf18, CDO1, DLGAP1, ELMOD1, FKBP11, FLOT1, GAL3ST2, LRRC41, LYPLAL1, MAX.chr11.3750, MLH1_4513, NR3C1_2223, PISD. RABC3, RAI1, TERC, TRPC3, ZIC2, ZMIZ1, ZNF480, ZNF491, ZNF610, and/or ZNF91 (Table 3), including any combinations thereof. In some embodiments, the novel DMR(s) is from any gene or region selected from Table 3, including any combinations thereof. Each novel DMR alone is capable of distinguishing one or more gynaecological cancers from a control sample, and combining two or more of the novel DMRs can provide increased sensitivity. Therefore, combinations of two or more novel DMRs selected from Table 3 are provided.

Embodiments of the present disclosure also include novel differentially methylated regions (DMRs), each individually capable of distinguishing a specific subtype of a gynecological cancer (i.e., serous ovarian cancer, clear cell ovarian cancer, endometrioid ovarian cancer, mucinous ovarian cancer, adenocarcinoma cervical cancer, squamous cervical cancer, or endometrioid endometrial cancer) from a benign gynecological tissue sample. In accordance with these embodiments, these novel DMRs comprise one or more CpG sites in LBX2, SPDYA, TERC, ZSCAN12, CYP26C1, and/or GYPC (Table 4), including any combinations thereof. In some embodiments, the novel DMR(s) is from any gene or region selected from Table 4, including any combinations thereof. Each novel DMR alone is capable of distinguishing one or more gynaecological cancers from a control sample, and combining two or more of the novel DMRs can provide increased sensitivity. Therefore, combinations of two or more novel DMRs selected from Table 4 are provided.

Embodiments of the present disclosure also include novel differentially methylated regions (DMRs), each individually capable of distinguishing a specific subtype of a gynecological cancer (i.e., serous ovarian cancer, clear cell ovarian cancer, endometrioid ovarian cancer, mucinous ovarian cancer, adenocarcinoma cervical cancer, squamous cervical cancer, or endometrioid endometrial cancer) from a benign gynecological tissue sample. In accordance with these embodiments, these novel DMRs comprise one or more CpG sites in KRT86, CDH4, c17orf64, EMX2OS, NBPF24, SFMBT2_0970, JSRP1, DIDO1, MAX.chr10.4460, MPZ, ZNF506, GATA2_6370, VILL, LINC02323, CYTH2_4043, LRRC8D_8831, LYPLAL1, SMPD5, SQSTM1_3864, ZNF323, OBSCN, ZNF90, LRRC34, GDF7, MDFI, EEF1A2, LRRC41, and/or SEPT9 (Table 8), including any combinations thereof. In some embodiments, the novel DMR(s) is from any gene or region selected from Table 8, including any combinations thereof. Each novel DMR alone is capable of distinguishing one or more gynaecological cancers from a control sample, and combining two or more of the novel DMRs can provide increased sensitivity. Therefore, combinations of two or more novel DMRs selected from Table 8 are provided.

Embodiments of the present disclosure include a method of characterizing a biological sample. In accordance with these embodiments, the method includes determining a methylation profile in at least one differentially methylated region (DMR) of a DNA sample obtained from a subject having or suspected of having a gynecological cancer by treating the sample with a reagent that modifies DNA in a methylation-specific manner.

In some embodiments, the methylation profile in the at least one DMR indicates the subject has or is suspected of having at least one of ovarian cancer (OC), cervical cancer (CC), and endometrial cancer (EC).

In some embodiments, the at least one DMR comprises one or more CpG sites in ADAM8, ADHFE1, AES, AGBL2, AIM1, AK5, ALKBH3, ARAP1, ARHGAP20, ASCL2, BCAT1, BEGAIN, BEND4_3696, BMP6, C12orf68, C13orf18, C14orf169_7694, C14orf169_8382, C18orf18, C1orf61, C20orf195, C4orf31, C5orf52, C6orf147, C7orf51, CD14, CELF2, CHCHD5, CHMP2A, CHST10, CLIC6, CLIP4, COL13A1, COL19A1, COL6A2, COPZ2, CREB3L1, CXCL2, CXXC5, CYTH2, DAB2IP, DGKZ, DLGAP3, DNASE2, DSCAML1, EBF1, EDARADD, EGR2, EIF5A2, ELMO1, ELMOD1, ELOVL4, EME2, EML6, EPSTI1, FADS2, FAM109B, FAM126A, FAM174B, FGF18, FKBP11, FLI1, FLOT1, FOXD3, FYN, GAL3ST2, GALR3, GAS7, GATA2_5878, GLT25D2, GNB2, HDAC7, HIC1, HLA-F, HNRNPF, HPDL, HS3ST4, HSPA1A, IDUA, IGSF9B, IL12RB2, IRAK3, IRF7, IRF8, ITPKA, KCNA2, KCNC3_6487, KCNC3_7105, KCNC4, KCNH8, KDM2B, LBX2, LCMT2, LOC100129726, LOC100287216, LOC255130, LOC339290, LOC729678, LPPR3, LRRC41, LRRC8D_8856, LTBP2, LYPLAL1, MAST4, MAX.chr1.2152, HIVEP3, GRAMD1B, MAX.chr11.0394, MAX.chr11.3750, FAT3, SLC16A7, MTUS2, LINC02323, MAX.chr14.7696, MCTP2, LOC107984974, TRIM80P, MAX.chr19.5552, ZNF433-AS1, ZNF254, MAX.chr19.0548, B3GALT1, MAX.chr2.8918, MAX.chr2.4778, MAX.chr20.3853, MAX.chr20.2903, MAX.chr21.5011, DSCR9, MAX.chr22.5665, MAX.chr3.6408, LINC02028, LINC02084, MAX.chr5.3588, CTD-2532K18.1, HS3ST5, ARHGAP18, GRM4, LINC01004, MAX.chr8.5938, MAX.chr9.4007, MAX.chr9.2025, TRPM3, MED12L, MIAT, MLH1_4513, MLH1_5193, MMP16, MRPS21, MSI1, MT1E, MX1, MYC, MYH10, MYO15B, N4BP2L1, NBR1, NDRG2, NEGR1, NEU1, NOL3, NR3C1_2223, NR3C1_4614, NRP2, NTN1, NTNG1, PAPL, PAQR9, PDE10A, PDE3B, PDE4A, PDXK, PER1, PISD, PLEC, PLIN2, PLXND1, PPM1E, PPP1R9A, PPP2R5C, PRDM5, PTP4A3, PYCARD, RAB3C, RAI1, RARG, RASA3, RPRM, RREB1, S100A6, SAMD5, SBNO2, SDC2, SDK2, SELM, SERP2, SFMBT2_2029, SHF, SHH, SLC16A11, SLC16A5, SLC25A22, SLCO3A1, SMTN, SPDYA, SPINK2, SPOCK2, SPON1, SQSTM1_4156, ST8SIA1, TAF4B, TAF7, TEAD3, TERC, TIAM1, TLE4, TMEM101, TMEM106A, TRIM9, TRPC3, TSC22D4, TSPAN2, TSPAN5, TTC14, UBB_4001, UBB_4646, UST, VAMP5, VIM, VSTM2B, ZBTB7B, ZEB2, ZFP3, ZFP36L2, ZIC2, ZMIZ1, ZNF14, ZNF211, ZNF280B, ZNF302, ZNF382, ZNF480, ZNF483, ZNF491, ZNF569, ZNF610, ZNF702P, ZNF709, ZNF773, ZNF845, ZNF91, CDH4, LRRC34, MAX.chr10.4460, NBPF24, OBSCN, SEPT9, ZNF323, ZNF506, and/or ZNF90.

In some embodiments, the at least one DMR comprises one or more CpG sites in ACSF2, AJAP1, ARL10, ARL5C, ASCL4, ATP6V1B1, BARHL1, BEND4_2963, C17orf64, C1QL3, C2orf55, C4orf48, CA3, CDO1, CELF2, CLEC14A, CSDAP1, CYTH2_4197, DLGAP1, DSCR6, EPS8L1_2819, EPS8L1_8496, FAIM2, FGF12, GATA2, HIST1H2BE, IRF4, IRX4, ITGA5, KCNA1, LECT1, LHX1, LOC440925, LPHN1, LINC02767, MAX.chr1.2533, SOX1-OT, MAX.chr13.3357, MAX.chr14.2093, MAX.chr17.2455, MAX.chr18.4390, MAX.chr19.2732, MAX.chr19.4467, PANTR1, MAX.chr2.0490, MAX.chr2.8148, MAX.chr2.3137, RIPOR3, SCRG1, MAX.chr4.4210, HMX1, CTC-359M8.1, MAX.chr5.0931, MAX.chr5.9924, LIN28B, MAX.chr6.9522, TTLL2, RNA5SP243, DLGAP2, MEX3B, MNX1, NEFL, NETO1, PAX2, PDX1, psiTPTE22, RASGEF1A, SALL3_9136, SALL3_0615, SEZ6L2, SHANK2, SHANK3, SKI, SLC35D3, SORCS3_0305, SORCS3_1038, SOX1, SQSTM1, TBXT, TCERG1L, TERT, TNFSF11, TUBB6, ULBP1, VAC14, VWC2, WDR69, ZBTB16, ZNF132, ZSCAN12, ZSCAN23, KRT86, CYP26C1, GYPC, DIDO1, EEF1A2, EMX2OS, GDF7, JSRP1, SMPD5, MDFI, MPZ, and/or VILL.

In some embodiments, the at least one DMR comprises one or more CpG sites in FLOT1, GAL3ST2, LRRC41, LYPLAL1, MAX.chr11.3750, PISD, RAI1, ZIC2, ZMIZ1, CDH4, ZNF506, ZNF323, OBSCN, ZNF90, and/or SEPT9; and the subject has or is suspected of having OC. In some embodiments, the at least one DMR comprises one or more CpG sites in AIM1, FLOT1, GAL3ST2, and LYPLAL1, and/or OBSCN; and the subject has or is suspected of having serous OC. In some embodiments, the at least one DMR comprises one or more CpG sites in LRRC41, PISD, ZIC2, OBSCN, and/or SEPT9; and the subject has or is suspected of having clear cell OC. In some embodiments, the at least one DMR comprises one or more CpG sites in MAX.chr11.3750; and the subject has or is suspected of having endometroid OC. In some embodiments, the at least one DMR comprises one or more CpG sites in RAH and/or ZMIZ1; and the subject has or is suspected of having mucinous OC. In some embodiments, determining the methylation profile of one or more CpG sites in AIM1, FLOT1, GAL3ST2, LRRC41, LYPLAL1, MAX.chr11.3750, PISD, RAI1, ZIC2, ZMIZ1, CDH4, ZNF506, ZNF323, OBSCN, ZNF90, and/or SEPT9 comprises comparing the methylation profile to a corresponding region from a control DNA sample obtained from a subject that does not have OC.

In some embodiments, the at least one DMR comprises one or more CpG sites in AK5, ELMOD1, RABC3, TRPC3, ZNF480, ZNF491, ZNF610, ZNF91, and/or NBPF24; and the subject has or is suspected of having CC. In some embodiments, the at least one DMR comprises one or more CpG sites in AK5, ELMOD1, TRPC3, and/or ZNF480; and the subject has or is suspected of having adenocarcinoma CC. In some embodiments, the at least one DMR comprises one or more CpG sites in ZNF491, ZNF610, ZNF91, and/or NBPF24; and the subject has or is suspected of having squamous cell CC. In some embodiments, determining the methylation profile of one or more CpG sites in AK5, ELMOD1, RABC3, TRPC3, ZNF480, ZNF491, ZNF610, and/or ZNF91 comprises comparing the methylation profile to a corresponding region from a control DNA sample obtained from a subject that does not have CC.

In some embodiments, the at least one DMR comprises one or more CpG sites in c18orf18, FKBP11, MLH1, NR3C1 and/or TERC; and the subject has or is suspected of having EC. In some embodiments, the at least one DMR comprises one or more CpG sites in MLH1 and/or SEPT9; and the subject has or is suspected of having clear cell EC. In some embodiments, the at least one DMR comprises one or more CpG sites in NR3C1; and the subject has or is suspected of having endometrioid EC.

In some embodiments, determining the methylation profile of one or more CpG sites in c18orf18, FKBP11, MLH1, NR3C1, and/or TERC comprises comparing the methylation profile to a corresponding region from a control DNA sample obtained from a subject that does not have EC.

In some embodiments, the at least one DMR comprises one or more CpG sites in CDO1 and/or DLGAP1; and wherein the subject has or is suspected of having CC, OC, or EC. In some embodiments, determining the methylation profile of one or more CpG sites in CDO1 and/or DLGAP1 comprises comparing the methylation profile to a corresponding region from a control DNA sample obtained from a subject that does not have CC, OC, or EC.

In some embodiments, the method further comprises determining the methylation profile of one or more CpG sites in AIM1, FLOT1, GAL3ST2, LRRC41, LYPLAL1, MAX.chr11.3750, PISD, RAI1, ZIC2, and/or ZMIZ1. In some embodiments, the method further comprises determining the methylation profile of one or more CpG sites in AK5, ELMOD1, RABC3, TRPC3, ZNF480, ZNF491, ZNF610, and/or ZNF91. In some embodiments, the method further comprises determining the methylation profile of one or more CpG sites in c18orf18, FKBP11, MLH1, NR3C1, and/in TERC.

In some embodiments, the at least one DMR comprises one or more CpG sites in NBPF24, and wherein the subject has or is suspected of having CC. In some embodiments, determining the methylation profile of one or more CpG sites in NBPF24 comprises comparing the methylation profile to a corresponding region from a control DNA sample obtained from a subject that does not have CC.

In some embodiments, the at least one DMR comprises one or more CpG sites in CDH4, NBPF24, MAX.chr10.4460, ZNF506, ZNF323, OBSCN, ZNF90, LRRC34, SFMBT2, LINC02323, CYTH2, LRRC8D, LYPLAL1, LRRC41, and/or SEPT9, and wherein the subject has or is suspected of having EC. In some embodiments, determining the methylation profile of one or more CpG sites in CDH4, NBPF24, MAX.chr10.4460, ZNF506, ZNF323, OBSCN, ZNF90, LRRC34, SFMBT2, LINC02323, CYTH2, LRRC8D, LYPLAL1, LRRC41, and/or SEPT9 comprises comparing the methylation profile to a corresponding region from a control DNA sample obtained from a subject that does not have EC.

In some embodiments, the at least one DMR comprises one or more CpG sites in CDH4, ZNF506, ZNF323, OBSCN, ZNF90, SFMBT2, LINC02323, CYTH2, LRRC8D, LYPLAL1, LRRC41, and/or SEPT9, and wherein the subject has or is suspected of having OC. In some embodiments, determining the methylation profile of one or more CpG sites in CDH4, ZNF506, ZNF323, OBSCN, ZNF90, SFMBT2, LINC02323, CYTH2, LRRC8D, LYPLAL1, LRRC41, and/or SEPT9 comprises comparing the methylation profile to a corresponding region from a control DNA sample obtained from a subject that does not have OC.

In some embodiments, the at least one DMR comprises one or more CpG sites in KRT86, EMX2OS, JSRP1, DIDO1, MPZ, VILL, SMPD5, GDF7, MDFI, c17orf64, GATA2, SQSTM1, and/or EEF1A2; and wherein the subject has or is suspected of having CC, OC, or EC. In some embodiments, determining the methylation profile of one or more CpG sites in KRT86, EMX2OS, JSRP1, DIDO1, MPZ, VILL, SMPD5, GDF7, MDFI, c17orf64, GATA2, SQSTM1, and/or EEF1A2 comprises comparing the methylation profile to a corresponding region from a control DNA sample obtained from a subject that does not have CC, OC, or EC.

In some embodiments, the at least one DMR is associated with an area under a ROC curve (AUC) greater than or equal to 0.8, and the ROC curve discriminates between a subject having or suspected of having OC, CC, or EC and a control sample.

In some embodiments, the biological sample is selected from a tissue sample, a blood sample, a plasma sample, a serum sample, a whole blood sample, a secretion sample, an organ secretion sample, a cerebrospinal fluid (CSF) sample, a saliva sample, a urine sample, and a stool sample. In some embodiments, the tissue sample is a gynecological tissue sample. In some embodiments, the gynecological tissue sample comprises one or more of vaginal tissue, vaginal cells, cervical tissue, cervical cells, endometrial tissue, endometrial cells, ovarian tissue, and ovarian cells. In some embodiments, the tissue sample is an ovarian tissue sample, an endometrial tissue sample, or a cervical tissue sample. In some embodiments, the secretion sample is a gynecological secretion sample. In some embodiments, the subject is a human.

In some embodiments, the biological sample is obtained from the subject, and the method further comprises extracting the DNA sample from the biological sample. In some embodiments, the biological sample is collected with a collection device having an absorbing member capable of collecting the biological sample upon contact. In some embodiments, the absorbing member is a sponge configured for insertion into an orifice. In some embodiments, the collection device is selected from a tampon, a lavage that releases liquid into the vagina and re-collects fluid, a cervical brush, a Fournier cervical self-sampling device, and a swab.

In some embodiments, the reagent that modifies DNA in a methylation-specific manner is a borane reducing agent. In some embodiments, the reagent that modifies DNA in a methylation-specific manner comprises one or more of a methylation-sensitive restriction enzyme, a methylation-dependent restriction enzyme, and a bisulfite reagent.

In some embodiments, determining the methylation profile of at least one DMR comprises amplifying at least a portion of the DMR using a set of primers.

In some embodiments, determining the methylation profile of at least one DMR comprises performing at least one of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR.

In some embodiments, determining the methylation profile of at least one DMR comprises determining the presence or absence of methylation at a CpG site.

Embodiments of the present disclosure also include a method of identifying a gynecological cancer. In accordance with these embodiments, the method includes determining a methylation profile in at least one differentially methylated region (DMR) of a DNA sample obtained from a subject having or suspected of having a gynecological cancer by treating the sample with a reagent that modifies DNA in a methylation-specific manner. In some embodiments, the at least one DMR comprises one or more CpG sites in AIM1, FLOT1, GAL3ST2, LRRC41, LYPLAL1, MAX.chr11.3750, PISD, RAI1, ZIC2, and/or ZMIZ1; and the methylation profile indicates that the subject has ovarian cancer. In some embodiments, the method further includes treating the subject with an anti-cancer therapy.

Embodiments of the present disclosure also include a method of identifying a gynecological cancer. In accordance with these embodiments, the method includes determining a methylation profile in at least one differentially methylated region (DMR) of a DNA sample obtained from a subject having or suspected of having a gynecological cancer by treating the sample with a reagent that modifies DNA in a methylation-specific manner. In some embodiments, the at least one DMR comprises one or more CpG sites in AK5, ELMOD1, RABC3, TRPC3, ZNF480, ZNF491, ZNF610, and/or ZNF91; and the methylation profile indicates that the subject has cervical cancer. In some embodiments, the method further includes treating the subject with an anti-cancer therapy.

Embodiments of the present disclosure also include a method of identifying a gynecological cancer. In accordance with these embodiments, the method includes determining a methylation profile in at least one differentially methylated region (DMR) of a DNA sample obtained from a subject having or suspected of having a gynecological cancer by treating the sample with a reagent that modifies DNA in a methylation-specific manner. In some embodiments, the at least one DMR comprises one or more CpG sites in c18orf18, FKBP11, MLH1, NR3C1, and/or TERC; and the methylation profile indicates that the subject has endometrial cancer. In some embodiments, the method further includes treating the subject with an anti-cancer therapy.

Embodiments of the present disclosure also include a method of identifying a gynecological cancer. In accordance with these embodiments, the method includes determining a methylation profile in at least one differentially methylated region (DMR) of a DNA sample obtained from a subject having or suspected of having a gynecological cancer by treating the sample with a reagent that modifies DNA in a methylation-specific manner. In some embodiments, the at least one DMR comprises one or more CpG sites in CDO1 and/or DLGAP1; and the methylation profile indicates that the subject has ovarian cancer, cervical cancer, or endometrial cancer. In some embodiments, the method further includes treating the subject with an anti-cancer therapy.

Embodiments of the present disclosure also include a method of identifying a gynecological cancer. In accordance with these embodiments, the method includes determining a methylation profile in at least one differentially methylated region (DMR) of a DNA sample obtained from a subject having or suspected of having a gynecological cancer by treating the sample with a reagent that modifies DNA in a methylation-specific manner. In some embodiments, the at least one DMR comprises one or more CpG sites in NBPF24; and wherein the methylation profile indicates that the subject has cervical cancer. In some embodiments, the method further includes treating the subject with an anti-cancer therapy.

Embodiments of the present disclosure also include a method of identifying a gynecological cancer. In accordance with these embodiments, the method includes determining a methylation profile in at least one differentially methylated region (DMR) of a DNA sample obtained from a subject having or suspected of having a gynecological cancer by treating the sample with a reagent that modifies DNA in a methylation-specific manner. In some embodiments, the at least one DMR comprises one or more CpG sites in CDH4, NBPF24, MAX.chr10.4460, ZNF506, ZNF323, OBSCN, ZNF90, LRRC34, SFMBT2, LINC02323, CYTH2, LRRC8D, LYPLAL1, LRRC41, and/or SEPT9; and wherein the methylation profile indicates that the subject has endometrial cancer. In some embodiments, the method further includes treating the subject with an anti-cancer therapy.

Embodiments of the present disclosure also include a method of identifying a gynecological cancer. In accordance with these embodiments, the method includes determining a methylation profile in at least one differentially methylated region (DMR) of a DNA sample obtained from a subject having or suspected of having a gynecological cancer by treating the sample with a reagent that modifies DNA in a methylation-specific manner. In some embodiments, the at least one DMR comprises one or more CpG sites in CDH4, ZNF506, ZNF323, OBSCN, ZNF90, SFMBT2, LINC02323, CYTH2, LRRC8D, LYPLAL1, LRRC41, and/or SEPT9; and wherein the methylation profile indicates that the subject has ovarian cancer. In some embodiments, the method further includes treating the subject with an anti-cancer therapy.

Embodiments of the present disclosure also include a method of identifying a gynecological cancer. In accordance with these embodiments, the method includes determining a methylation profile in at least one differentially methylated region (DMR) of a DNA sample obtained from a subject having or suspected of having a gynecological cancer by treating the sample with a reagent that modifies DNA in a methylation-specific manner. In some embodiments, the at least one DMR comprises one or more CpG sites in KRT86, EMX2OS, JSRP1, DIDO1, MPZ, VILL, SMPD5, GDF7, MDFI, c17orf64, GATA2, SQSTM1, and/or EEF1A2; and wherein the methylation profile indicates that the subject has ovarian cancer, cervical cancer, or endometrial cancer. In some embodiments, the method further includes treating the subject with an anti-cancer therapy.

### BRIEF DESCRIPTION OF THE DRAWINGS

FIG. 1: Representative heatmap illustrating the ability of candidate methylated DNA markers to distinguish among gynecological cancers and cancer subtypes (see also Table 3).

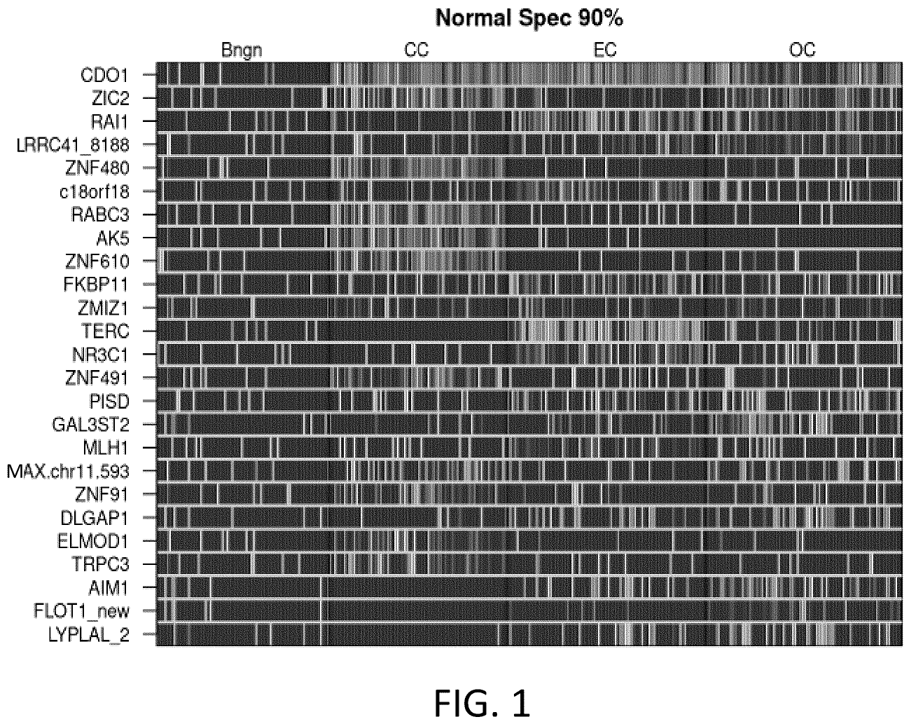

FIGS. 2A-2C: Representative data corresponding to DNA methylation marker LRRC41, including a calibration plot based on ACTB normalization (FIG. 2A), and adjusted boxplots demonstrating epigenetic relationships among the three main gynecological cancers (FIG. 2B), among gynecological cancer subtypes (FIG. 2C), and controls (FIGS. 2A and 2B).

FIGS. 3A-3C: Representative data corresponding to DNA methylation marker CDO1, including a calibration plot based on ACTB normalization (FIG. 3A), and adjusted boxplots demonstrating epigenetic relationships among the three main gynecological cancers (FIG. 3B), among gynecological cancer subtypes (FIG. 3C), and controls (FIGS. 3A and 3B).

FIGS. 4A-4C: Representative data corresponding to DNA methylation marker ZMIZ1, including a calibration plot based on ACTB normalization (FIG. 4A), and adjusted boxplots demonstrating epigenetic relationships among the three main gynecological cancers (FIG. 4B), among gynecological cancer subtypes (FIG. 4C), and controls (FIGS. 4A and 4B).

FIGS. 5A-5C: Representative data corresponding to DNA methylation marker PISD, including a calibration plot based on ACTB normalization (FIG. 5A), and adjusted boxplots demonstrating epigenetic relationships among the three main gynecological cancers (FIG. 5B), among gynecological cancer subtypes (FIG. 5C), and controls (FIGS. 5A and 5B).

FIGS. 6A-6C: Representative data corresponding to DNA methylation marker AIM1, including a calibration plot based on ACTB normalization (FIG. 6A), and adjusted boxplots demonstrating epigenetic relationships among the three main gynecological cancers (FIG. 6B), among gynecological cancer subtypes (FIG. 6C), and controls (FIGS. 6A and 6B).

FIGS. 7A-7C: Representative data corresponding to DNA methylation marker AK5, including a calibration plot based on ACTB normalization (FIG. 7A), and adjusted boxplots demonstrating epigenetic relationships among the three main gynecological cancers (FIG. 7B), among gynecological cancer subtypes (FIG. 7C), and controls (FIGS. 7A and 7B).

FIGS. 8A-8C: Representative data corresponding to DNA methylation marker c18orf18, including a calibration plot based on ACTB normalization (FIG. 8A), and adjusted boxplots demonstrating epigenetic relationships among the three main gynecological cancers (FIG. 8B), among gynecological cancer subtypes (FIG. 8C), and controls (FIGS. 8A and 8B).

FIGS. 9A-9C: Representative data corresponding to DNA methylation marker ELMOD1, including a calibration plot based on ACTB normalization (FIG. 9A), and adjusted boxplots demonstrating epigenetic relationships among the three main gynecological cancers (FIG. 9B), among gynecological cancer subtypes (FIG. 9C), and controls (FIGS. 9A and 9B).

FIGS. 10A-10C: Representative data corresponding to DNA methylation marker FKBP11, including a calibration plot based on ACTB normalization (FIG. 10A), and adjusted boxplots demonstrating epigenetic relationships among the three main gynecological cancers (FIG. 10B), among gynecological cancer subtypes (FIG. 10C), and controls (FIGS. 10A and 10B).

FIGS. 11A-11C: Representative data corresponding to DNA methylation marker FLOT1, including a calibration plot based on ACTB normalization (FIG. 11A), and adjusted boxplots demonstrating epigenetic relationships among the three main gynecological cancers (FIG. 11B), among gynecological cancer subtypes (FIG. 11C), and controls (FIGS. 11A and 11B).

FIGS. 12A-12C: Representative data corresponding to DNA methylation marker GAL3ST2, including a calibration plot based on ACTB normalization (FIG. 12A), and adjusted boxplots demonstrating epigenetic relationships among the three main gynecological cancers (FIG. 12B), among gynecological cancer subtypes (FIG. 12C), and controls (FIGS. 12A and 12B).

FIGS. 13A-13C: Representative data corresponding to DNA methylation marker MAX.chr11.593, including a calibration plot based on ACTB normalization (FIG. 13A), and adjusted boxplots demonstrating epigenetic relationships among the three main gynecological cancers (FIG. 13B), among gynecological cancer subtypes (FIG. 13C), and controls (FIGS. 13A and 13B).

FIGS. 14A-14C: Representative data corresponding to DNA methylation marker MLH1, including a calibration plot based on ACTB normalization (FIG. 14A), and adjusted boxplots demonstrating epigenetic relationships among the three main gynecological cancers (FIG. 14B), among gynecological cancer subtypes (FIG. 14C), and controls (FIGS. 14A and 14B).

FIGS. 15A-15C: Representative data corresponding to DNA methylation marker NR3C1, including a calibration plot based on ACTB normalization (FIG. 15A), and adjusted boxplots demonstrating epigenetic relationships among the three main gynecological cancers (FIG. 15B), among gynecological cancer subtypes (FIG. 15C), and controls (FIGS. 15A and 15B).

FIGS. 16A-16C: Representative data corresponding to DNA methylation marker RABC3, including a calibration plot based on ACTB normalization (FIG. 16A), and adjusted boxplots demonstrating epigenetic relationships among the three main gynecological cancers (FIG. 16B), among gynecological cancer subtypes (FIG. 16C), and controls (FIGS. 16A and 16B).

FIGS. 17A-17C: Representative data corresponding to DNA methylation marker RAI1, including a calibration plot based on ACTB normalization (FIG. 17A), and adjusted boxplots demonstrating epigenetic relationships among the three main gynecological cancers (FIG. 17B), among gynecological cancer subtypes (FIG. 17C), and controls (FIGS. 17A and 17B).

FIGS. 18A-18C: Representative data corresponding to DNA methylation marker TERC, including a calibration plot based on ACTB normalization (FIG. 18A), and adjusted boxplots demonstrating epigenetic relationships among the three main gynecological cancers (FIG. 18B), among gynecological cancer subtypes (FIG. 18C), and controls (FIGS. 18A and 18B).

FIGS. 19A-19C: Representative data corresponding to DNA methylation marker TRPC3, including a calibration plot based on ACTB normalization (FIG. 19A), and adjusted boxplots demonstrating epigenetic relationships among the three main gynecological cancers (FIG. 19B), among gynecological cancer subtypes (FIG. 19C), and controls (FIGS. 19A and 19B).

FIGS. 20A-20C: Representative data corresponding to DNA methylation marker ZIC2, including a calibration plot based on ACTB normalization (FIG. 20A), and adjusted boxplots demonstrating epigenetic relationships among the three main gynecological cancers (FIG. 20B), among gynecological cancer subtypes (FIG. 20C), and controls (FIGS. 20A and 20B).

FIGS. 21A-21C: Representative data corresponding to DNA methylation marker ZNF480, including a calibration plot based on ACTB normalization (FIG. 21A), and adjusted boxplots demonstrating epigenetic relationships among the three main gynecological cancers (FIG. 21B), among gynecological cancer subtypes (FIG. 21C), and controls (FIGS. 21A and 21B).

FIGS. 22A-22C: Representative data corresponding to DNA methylation marker ZNF491, including a calibration plot based on ACTB normalization (FIG. 22A), and adjusted boxplots demonstrating epigenetic relationships among the three main gynecological cancers (FIG. 22B), among gynecological cancer subtypes (FIG. 22C), and controls (FIGS. 22A and 22B).

FIGS. 23A-23C: Representative data corresponding to DNA methylation marker ZNF610, including a calibration plot based on ACTB normalization (FIG. 23A), and adjusted boxplots demonstrating epigenetic relationships among the three main gynecological cancers (FIG. 23B), among gynecological cancer subtypes (FIG. 23C), and controls (FIGS. 23A and 23B).

FIGS. 24A-24C: Representative data corresponding to DNA methylation marker ZNF91, including a calibration plot based on ACTB normalization (FIG. 24A), and adjusted boxplots demonstrating epigenetic relationships among the three main gynecological cancers (FIG. 24B), among gynecological cancer subtypes (FIG. 24C), and controls (FIGS. 24A and 24B).

FIGS. 25A-25C: Representative data corresponding to DNA methylation marker DLGAP1, including a calibration plot based on ACTB normalization (FIG. 25A), and adjusted boxplots demonstrating epigenetic relationships among the three main gynecological cancers (FIG. 25B), among gynecological cancer subtypes (FIG. 25C), and controls (FIGS. 25A and 25B).

FIGS. 26A-26C: Representative data corresponding to DNA methylation marker LYPLAP_2, including a calibration plot based on ACTB normalization (FIG. 26A), and adjusted boxplots demonstrating epigenetic relationships among the three main gynecological cancers (FIG. 26B), among gynecological cancer subtypes (FIG. 26C), and controls (FIGS. 26A and 26B).

### DETAILED DESCRIPTION

The present disclosure relates to detecting one or more types of gynecological cancer in a biological sample from a subject. In particular, the present disclosure provides compositions and methods for detecting the presence or absence of one or more types of gynecological cancer (e.g., cervical cancer, ovarian cancer, endometrial cancer) in a biological sample from a subject having or suspected of having a gynecological cancer.

Section headings as used in this section and the entire disclosure herein are merely for organizational purposes and are not intended to be limiting.

### 1. DEFINITIONS

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The transitional phrase “consisting essentially of” as used in claims in the present application limits the scope of a claim to the specified materials or steps “and those that do not materially affect the basic and novel characteristic(s)” of the claimed invention, as discussed in In re Herz, 537 F.2d 549, 551-52, 190 USPQ 461, 463 (CCPA 1976). For example, a composition “consisting essentially of” recited elements may contain an unrecited contaminant at a level such that, though present, the contaminant does not alter the function of the recited composition as compared to a pure composition, i.e., a composition “consisting of” the recited components.

The term “one or more”, as used herein, refers to a number higher than one. For example, the term “one or more” encompasses any of the following: two or more, three or more, four or more, five or more, six or more, seven or more, eight or more, nine or more, ten or more, twelve or more, thirteen or more, fourteen or more, fifteen or more, twenty or more, fifty or more, 100 or more, or an even greater number.

The term “one or more but less than a higher number,” “two or more but less than a higher number,” “three or more but less than a higher number,” “four or more but less than a higher number,” “five or more but less than a higher number,” “six or more but less than a higher number,” “seven or more but less than a higher number,” “eight or more but less than a higher number,” “nine or more but less than a higher number,” “ten or more but less than a higher number,” “eleven or more but less than a higher number,” “twelve or more but less than a higher number,” “thirteen or more but less than a higher number,” “fourteen or more but less than a higher number,” or “fifteen or more but less than a higher number” is not limited to a higher number. For example, the higher number can be 10,000, 1,000, 100, 50, etc. For example, the higher number can be approximately 50 (e.g., 50, 49, 48, 47, 46, 45, 44, 43, 42, 41, 40, 39, 38, 37, 36, 35, 34, 33, 32, 31, 32, 30, 29, 28, 27, 26, 25, 24, 23, 22, 21, 20, 19, 18, 17, 16, 15, 14, 13, 12, 11, 10, 9, 8, 7, 6, 5, 4, 3 or 2).

The term “one or more methylated markers” or “one or more DMRs” or “one or more genes” or “one or more markers” or “a plurality of methylated markers” or “a plurality of markers” or “a plurality of genes” or “a plurality of DMRs” is similarly not limited to a particular numerical combination. Indeed, any numerical combination of methylated markers is contemplated (e.g., 1-2 methylated markers, 1-3, 1-4, 1-5. 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, 1-30, 1-31, 1-32, 1-33, 1-34, 1-35, 1-36, 1-37, 1-38) (e.g., 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38) (e.g., 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38) (e.g., 4-5, 4-6, 4-7, 4-8, 4-9, 4-10, 4-11, 4-12, 4-13, 4-14, 4-15, 4-16, 4-17, 4-18, 4-19, 4-20, 4-21, 4-22, 4-23, 4-24, 4-25, 4-26, 4-27, 4-28, 4-29, 4-30, 4-31, 4-32, 4-33, 4-34, 4-35, 4-36, 4-37, 4-38) (e.g., 5-6, 5-7, 5-8, 5-9, 5-10, 5-11, 5-12, 5-13, 5-14, 5-15, 5-16, 5-17, 5-18, 5-19, 5-20, 5-21, 5-22, 5-23, 5-24, 5-25, 5-26, 5-27, 5-28, 5-29, 5-30, 5-31, 5-32, 5-33, 5-34, 5-35, 5-36, 5-37, 5-38) (e.g., 6-7, 6-8, 6-9, 6-10, 6-11, 6-12, 6-13, 6-14, 6-15, 6-16, 6-17, 6-18, 6-19, 6-20, 6-21, 6-22, 6-23, 6-24, 6-25, 6-26, 6-27, 6-28, 6-29, 6-30, 6-31, 6-32, 6-33, 6-34, 6-35, 6-36, 6-37, 6-38) (e.g., 7-8, 7-9, 7-10, 7-11, 7-12, 7-13, 7-14, 7-15, 7-16, 7-17, 7-18, 7-19, 7-20, 7-21, 7-22, 7-23, 7-24, 7-25, 7-26, 7-27, 7-28, 7-29, 7-30, 7-31, 7-32, 7-33, 7-34, 7-35, 7-36, 7-37, 7-38) (e.g., 8-9, 8-10, 8-11, 8-12, 8-13, 8-14, 8-15, 8-16, 8-17, 8-18, 8-19, 8-20, 8-21, 8-22, 8-23, 8-24, 8-25, 8-26, 8-27, 8-28, 8-29, 8-30, 8-31, 8-32, 8-33, 8-34, 8-35, 8-36, 8-37, 8-38) (e.g., 9-10, 9-11, 9-12, 9-13, 9-14, 9-15, 9-16, 9-17, 9-18, 9-19, 9-20, 9-21, 9-22, 9-23, 9-24, 9-25, 9-26, 9-27, 9-28, 9-29, 9-30, 9-31, 9-32, 9-33, 9-34, 9-35, 9-36, 9-37, 9-38) (e.g., 10-11, 10-12, 10-13, 10-14, 10-15, 10-16, 10-17, 10-18, 10-19, 10-20, 10-21, 10-22, 10-23, 10-24, 10-25, 10-26, 10-27, 10-28, 10-29, 10-30, 10-31, 10-32, 10-33, 10-34, 10-35, 10-36, 10-37, 10-38) (e.g., 11-12, 11-13, 11-14, 11-15, 11-16, 11-17, 11-18, 11-19, 11-20, 11-21, 11-22, 11-23, 11-24, 11-25, 11-26, 11-27, 11-28, 11-29, 11-30, 11-31, 11-32, 11-33, 11-34, 11-35, 11-36, 11-37, 11-38) (e.g., 12-13, 12-14, 12-15, 12-16, 12-17, 12-18, 12-19, 12-20, 12-21, 12-22, 12-23, 12-24, 12-25, 12-26, 12-27, 12-28, 12-29, 12-30, 12-31, 12-32, 12-33, 12-34, 12-35, 12-36, 12-37, 12-38) (e.g., 13-14, 13-15, 13-16, 13-17, 13-18, 13-19, 13-20, 13-21, 13-22, 13-23, 13-24, 13-25, 13-26, 13-27, 13-28, 13-29, 13-30, 13-31, 13-32, 13-33, 13-34, 13-35, 13-36, 13-37, 13-38) (e.g., 14-15, 14-16, 14-17, 14-18, 14-19, 14-20, 14-21, 14-22, 14-23, 14-24, 14-25, 14-26, 14-27, 14-28, 14-29, 14-30, 14-31, 14-32, 14-33, 14-34, 14-35, 14-36, 14-37, 14-38) (e.g., 15-16, 15-17, 15-18, 15-19, 15-20, 15-21, 15-22, 15-23, 15-24, 15-25, 15-26, 15-27, 15-28, 15-29, 15-30, 15-31, 15-32, 15-33, 15-34, 15-35, 15-36, 15-37, 15-38) (e.g., 16-17, 16-18, 16-19, 16-20, 16-21, 16-22, 16-23, 16-24, 16-25, 16-26, 16-27, 16-28, 16-29, 16-30, 16-31, 16-32, 16-33, 16-34, 16-35, 16-36, 16-37, 16-38) (e.g., 17-18, 17-19, 17-20, 17-21, 17-22, 17-23, 17-24, 17-25, 17-26, 17-27, 17-28, 17-29, 17-30, 17-31, 17-32, 17-33, 17-34, 17-35, 17-36, 17-37, 17-38) (e.g., 18-19, 18-20, 18-21, 18-22, 18-23, 18-24, 18-25, 18-26, 18-27, 18-28, 18-29, 18-30, 18-31, 18-32, 18-33, 18-34, 18-35, 18-36, 18-37, 18-38) (e.g., 19-20, 19-21, 19-22, 19-23, 19-24, 19-25, 19-26, 19-27, 19-28, 19-29, 19-30, 19-31, 19-32, 19-33, 19-34, 19-35, 19-36, 19-37, 19-38) (e.g., 20-21, 20-22, 20-23, 20-24, 20-25, 20-26, 20-27, 20-28, 20-29, 20-30, 20-31, 20-32, 20-33, 20-34, 20-35, 20-36, 20-37, 20-38) (e.g., 21-22, 21-23, 21-24, 21-25, 21-26, 21-27, 21-28, 21-29, 21-30, 21-31, 21-32, 21-33, 21-34, 21-35, 21-36, 21-37, 21-38) (e.g., 22-23, 22-24, 22-25, 22-26, 22-27, 22-28, 22-29, 22-30, 22-31, 22-32, 22-33, 22-34, 22-35, 22-36, 22-37, 22-38) (e.g., 23-24, 23-25, 23-26, 23-27, 23-28, 23-29, 23-30, 23-31, 23-32, 23-33, 23-34, 23-35, 23-36, 23-37, 23-38) (e.g., 24-25, 24-26, 24-27, 24-28, 24-29, 24-30, 24-31, 24-32, 24-33, 24-34, 24-35, 24-36, 24-37, 24-38) (e.g., 25-26, 25-27, 25-28, 25-29, 25-30, 25-31, 25-32, 25-33, 25-34, 25-35, 25-36, 25-37, 25-38) (e.g., 26-27, 26-28, 26-29, 26-30, 26-31, 26-32, 26-33, 26-34, 26-35, 26-36, 26-37, 26-38) (e.g., 27-28, 27-29, 27-30, 27-31, 27-32, 27-33, 27-34, 27-35, 27-36, 27-37, 27-38) (e.g., 28-29, 28-30, 28-31, 28-32, 28-33, 28-34, 28-35, 28-36, 28-37, 28-38) (e.g., 29-30, 29-31, 29-32, 29-33, 29-34, 29-35, 29-36, 29-37, 29-38) (e.g., 30-31, 30-32, 30-33, 30-34, 30-35, 30-36, 30-37, 30-38) (e.g., 31-32, 31-33, 31-34, 31-35, 31-36, 31-37, 31-38) (e.g., 32-33, 32-34, 32-35, 32-36, 32-37, 32-38) (e.g., 33-34, 33-35, 33-36, 33-37, 33-38) (e.g., 34-35, 34-36, 34-37, 34-38) (e.g., 35-36, 35-37, 35-38) (e.g., 36-37, 36-38) (e.g., 37-38) (e.g., 38 or fewer; 37 or fewer; 36 or fewer; 35 or fewer; 34 or fewer; 33 or fewer; 32 or fewer; 31 or fewer; 30 or fewer; 29 or fewer; 28 or fewer; 27 or fewer; 26 or fewer; 25 or fewer; 24 or fewer; 23 or fewer; 22 or fewer; 21 or fewer; 20 or fewer; 19 or fewer; 18 or fewer; 17 or fewer; 16 or fewer; 15 or fewer; 14 or fewer; 13 or fewer; 12 or fewer; 11 or fewer; 10 or fewer; 9 or fewer; 8 or fewer; 7 or fewer; 6 or fewer; 5 or fewer; 4 or fewer; 3 or fewer; 2 or 1).

The term “multiple types of cancer” or “one or more types of cancer” or “one or more subtypes of cancer” or “a plurality of different types or subtypes of cancer” is similarly not limited to a particular numerical combination. Any numerical combination of types or subtypes of gynecological cancers can be identified using the DNA methylation markers of the present disclosure, including, but not limited to, ovarian cancer, serous ovarian cancer, clear cell ovarian cancer, endometrioid ovarian cancer, mucinous ovarian cancer, cervical cancer, adenocarcinoma cervical cancer, squamous cervical cancer, endometrial cancer, and endometrioid endometrial cancer.

As used herein, a “nucleic acid” or “nucleic acid molecule” generally refers to any ribonucleic acid or deoxyribonucleic acid, which may be unmodified or modified DNA or RNA. “Nucleic acids” include, without limitation, single- and double-stranded nucleic acids. As used herein, the term “nucleic acid” also includes DNA as described above that contains one or more modified bases. Thus, DNA with a backbone modified for stability or for other reasons is a “nucleic acid”. The term “nucleic acid” as it is used herein embraces such chemically, enzymatically, or metabolically modified forms of nucleic acids, as well as the chemical forms of DNA characteristic of viruses and cells, including for example, simple and complex cells.

The terms “oligonucleotide” or “polynucleotide” or “nucleotide” or “nucleic acid” refer to a molecule having two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof. Typical deoxyribonucleotides for DNA are thymine, adenine, cytosine, and guanine. Typical ribonucleotides for RNA are uracil, adenine, cytosine, and guanine.

As used herein, the terms “locus” or “region” of a nucleic acid refer to a subregion of a nucleic acid, e.g., a gene on a chromosome, a single nucleotide, a CpG island, etc.

The terms “complementary” and “complementarity” refer to nucleotides (e.g., 1 nucleotide) or polynucleotides (e.g., a sequence of nucleotides) related by the base-pairing rules. For example, the sequence 5′-A-G-T-3′ is complementary to the sequence 3′-T-C-A-5′. Complementarity may be “partial,” in which only some of the nucleic acids' bases are matched according to the base pairing rules. Or, there may be “complete” or “total” complementarity between the nucleic acids. The degree of complementarity between nucleic acid strands affects the efficiency and strength of hybridization between nucleic acid strands. This is of particular importance in amplification reactions and in detection methods that depend upon binding between nucleic acids.

The term “gene” refers to a nucleic acid (e.g., DNA or RNA) sequence that comprises coding sequences necessary for the production of an RNA, or of a polypeptide or its precursor. A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” encompasses the coding regions of a structural gene and the sequences located adjacent to the coding region on both the 5′ and 3′ ends, such that the gene corresponds to the length of the full-length mRNA (e.g., comprising coding, regulatory, structural and other sequences). The sequences that are located 5′ of the coding region and that are present on the mRNA are referred to as 5′ non-translated or untranslated sequences. The sequences that are located 3′ or downstream of the coding region and that are present on the mRNA are referred to as 3′ non-translated or 3′ untranslated sequences. The term “gene” encompasses both cDNA and genomic forms of a gene. In some organisms (e.g., eukaryotes), a genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. As would be understood by one of ordinary skill in the art based on the present disclosure, one or more CpG sites in a DMR can be located in a coding region of a gene, a non-coding regulator region of a gene, or a non-coding region that is not known to be associated with a particular gene, such as a region comprising a long non-coding RNA (lncRNA). In some embodiments, sequences corresponding to these regions can be obtained using an accession number (see, e.g., Tables 1 and 2) corresponding to a genomic database (e.g., GenBank, NCBI, UniProt, etc.). In some embodiments, one or more CpG sites in a DMR can be located in a genomic region that is unannotated. As provided further herein, unannotated genomic regions comprising one or more CpG sites in a DMR can be described using SEQ ID NOs (see, e.g., Tables 1 and 2; SEQ ID NOs: 1-32).

As would be recognized by one of ordinary skill in the art based on the present disclosure, the location of one or more CpG sites within a gene or region (e.g., CpG island) and its relevance to a disease or condition can be determined using a variety of techniques, including but not limited to, those disclosed in Chen et al., “Methods for identifying differentially methylated regions for sequence- and array-based data,” Briefings in Functional Genomics, Volume 15, Issue 6, November 2016, Pages 485-490, which is herein incorporated by reference in its entirety and for all purposes.

In addition to containing introns, genomic forms of a gene may also include sequences located on both the 5′ and 3′ ends of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage, and polyadenylation. These flanking regions may be non-coding, and thus may be absent from the mRNA transcript.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “wild-type” when made in reference to a protein refers to a protein that has the characteristics of a naturally occurring protein. The term “naturally-occurring” as applied to an object refers to the fact that an object can be found in nature. For example, a polypeptide or polynucleotide sequence that is present in an organism (including viruses) that can be isolated from a source in nature, and which has not been intentionally modified by the hand of a person in the laboratory is naturally-occurring. A wild-type gene is often that gene or allele that is most frequently observed in a population and is thus arbitrarily designated the “normal” or “wild-type” form of the gene. In contrast, the term “modified” or “mutant” when made in reference to a gene or to a gene product refers, respectively, to a gene or to a gene product that displays modifications in sequence and/or functional properties (e.g., altered characteristics) when compared to the wild-type gene or gene product. It is noted that naturally-occurring mutants can be isolated; these are identified by the fact that they have altered characteristics when compared to the wild-type gene or gene product.

The term “allele” refers to a variation of a gene; the variations include but are not limited to variants and mutants, polymorphic loci, and single nucleotide polymorphic loci, frameshift, and splice mutations. An allele may occur naturally in a population, or it might arise during the lifetime of any particular individual of the population.

Thus, the terms “variant” and “mutant” when used in reference to a nucleotide sequence refer to a nucleic acid sequence that differs by one or more nucleotides from another, usually related, nucleotide acid sequence. A “variation” is a difference between two different nucleotide sequences; typically, one sequence is a reference sequence.

The term “primer” refers to an oligonucleotide, whether occurring naturally as, e.g., a nucleic acid fragment from a restriction digest, or produced synthetically, that is capable of acting as a point of initiation of synthesis when placed under conditions in which synthesis of a primer extension product that is complementary to a nucleic acid template strand is induced, (e.g., in the presence of nucleotides and an inducing agent such as a DNA polymerase, and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, and the use of the method. In some embodiments, the primer pair is specific for a specific differentially methylated region (e.g., DMRs in Tables 1 and 2) and specifically binds at least a portion of a genetic region comprising the DMR.

The term “probe” refers to an oligonucleotide (e.g., a sequence of nucleotides), whether occurring naturally as in a purified restriction digest or produced synthetically, recombinantly, or by PCR amplification, which is capable of hybridizing to another oligonucleotide of interest. A probe may be single-stranded or double-stranded. Probes are useful in the detection, identification, and isolation of particular gene sequences (e.g., a “capture probe”). It is contemplated that any probe used in the embodiments of the present disclosure may, in some embodiments, be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the various embodiment of the present disclosure be limited to any particular detection system or label.

The term “target,” as used herein refers to a nucleic acid sought to be sorted out from other nucleic acids, e.g., by probe binding, amplification, isolation, capture, etc. For example, when used in reference to the polymerase chain reaction, “target” refers to the region of nucleic acid bounded by the primers used for polymerase chain reaction, while when used in an assay in which target DNA is not amplified, e.g., in some embodiments of an invasive cleavage assay, a target comprises the site at which a probe and invasive oligonucleotides (e.g., INVADER oligonucleotide) bind to form an invasive cleavage structure, such that the presence of the target nucleic acid can be detected. A “segment” is defined as a region of nucleic acid within the target sequence.

Accordingly, as used herein, “non-target”, e.g., as it is used to describe a nucleic acid such as a DNA, refers to nucleic acid that may be present in a reaction, but that is not the subject of detection or characterization by the reaction. In some embodiments, non-target nucleic acid may refer to nucleic acid present in a sample that does not, e.g., contain a target sequence, while in some embodiments, non-target may refer to exogenous nucleic acid, i.e., nucleic acid that does not originate from a sample containing or suspected of containing a target nucleic acid, and that is added to a reaction, e.g., to normalize the activity of an enzyme (e.g., polymerase) to reduce variability in the performance of the enzyme in the reaction.

As used herein, “methylation” refers to cytosine methylation at positions C5 or N4 of cytosine, the N6 position of adenine, or other types of nucleic acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

As used herein, the term “amplification reagents” refers to those reagents (deoxyribonucleoside triphosphates, buffer, etc.), needed for amplification except for primers, nucleic acid template, and the amplification enzyme. Typically, amplification reagents along with other reaction components are placed and contained in a reaction vessel.

As used herein, the term “control” when used in reference to nucleic acid detection or analysis refers to a nucleic acid having known features (e.g., known sequence, known copy-number per cell), for use in comparison to an experimental target (e.g., a nucleic acid of unknown concentration). A control may be an endogenous, preferably invariant gene against which a test or target nucleic acid in an assay can be normalized. Such normalizing controls for sample-to-sample variations that may occur in, for example, sample processing, assay efficiency, etc., and allows accurate sample-to-sample data comparison. Genes that find use for normalizing nucleic acid detection assays on human samples include, e.g., b-actin, ZDHHC1, and B3GALT6 (see, e.g., U.S. patent application Ser. Nos 14/966,617 and 62/364,082, each incorporated herein by reference). As used herein “ZDHHC1” refers to a gene encoding a protein characterized as a zinc finger, DHHC-type containing 1, located in human DNA on Chr 16 (16q22.1) and belonging to the DHHC palmitoyltransferase family. In some embodiments, reference genes include, but are not limited to, FNBP1, NCOR2, and S1PR4 (see Table 4).

Controls may also be external. For example, in quantitative assays such as qPCR, QuARTS, etc., a “calibrator” or “calibration control” is a nucleic acid of known sequence, e.g., having the same sequence as a portion of an experimental target nucleic acid, and a known concentration or series of concentrations (e.g., a serially diluted control target for generation of calibration curved in quantitative PCR). Typically, calibration controls are analyzed using the same reagents and reaction conditions as are used on an experimental DNA. In certain embodiments, the measurement of the calibrators is done at the same time, e.g., in the same thermal cycler, as the experimental assay. In preferred embodiments, multiple calibrators may be included in a single plasmid, such that the different calibrator sequences are easily provided in equimolar amounts. In particularly preferred embodiments, plasmid calibrators are digested, e.g., with one or more restriction enzymes, to release calibrator portion from the plasmid vector. See, e.g., WO 2015/066695, which is included herein by reference.

As used herein a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring; however, for purposes herein, thymine is not considered a methylated nucleotide when present in DNA since thymine is a typical nucleotide base of DNA.

As used herein, a “methylated nucleic acid molecule” refers to a nucleic acid molecule that contains one or more methylated nucleotides.

As used herein, a “methylation state”, “methylation profile”, and “methylation status” of a nucleic acid molecule refers to the presence or absence of one or more methylated nucleotide bases in the nucleic acid molecule. For example, a nucleic acid molecule containing a methylated cytosine is considered methylated (e.g., the methylation state of the nucleic acid molecule is methylated). A nucleic acid molecule that does not contain any methylated nucleotides is considered unmethylated.

As used herein, the term “methylation level” as applied to a methylation marker refers to the amount of methylation within a particular methylation marker. Methylation level may also refer to the amount of methylation within a particular methylation marker in comparison with an established norm or control. Methylation level may also refer to whether one or more cytosine residues present in a CpG context have or do not have a methylation group. Methylation level may also refer to the fraction of cells in a sample that do or do not have a methylation group on such cytosines. Methylation level may also alternatively describe whether a single CpG di-nucleotide is methylated.

The methylation state of a particular nucleic acid sequence (e.g., a gene marker or DNA region as described herein) can indicate the methylation state of every base in the sequence or can indicate the methylation state of a subset of the bases (e.g., of one or more cytosines) within the sequence, or can indicate information regarding regional methylation density within the sequence with or without providing precise information of the locations within the sequence the methylation occurs.

The methylation state of a nucleotide locus in a nucleic acid molecule refers to the presence or absence of a methylated nucleotide at a particular locus in the nucleic acid molecule. For example, the methylation state of a cytosine at the 7th nucleotide in a nucleic acid molecule is methylated when the nucleotide present at the 7th nucleotide in the nucleic acid molecule is 5-methylcytosine. Similarly, the methylation state of a cytosine at the 7th nucleotide in a nucleic acid molecule is unmethylated when the nucleotide present at the 7th nucleotide in the nucleic acid molecule is cytosine (and not 5-methylcytosine).

The methylation status can optionally be represented or indicated by a “methylation value” (e.g., representing a methylation frequency, fraction, ratio, percent, etc.). A methylation value can be generated, for example, by quantifying the amount of intact nucleic acid present following restriction digestion with a methylation dependent restriction enzyme or by comparing amplification profiles after bisulfite reaction or by comparing sequences of bisulfite-treated and untreated nucleic acids or by comparing TET-treated and untreated nucleic acids. Accordingly, a value, e.g., a methylation value, represents the methylation status and can thus be used as a quantitative indicator of methylation status across multiple copies of a locus. This is of particular use when it is desirable to compare the methylation status of a sequence in a sample to a threshold or reference value.

As used herein, “methylation frequency” or “methylation percent (%)” refer to the number of instances in which a molecule or locus is methylated relative to the number of instances the molecule or locus is unmethylated.

The term “methylation score” as used herein is a score indicative of detected methylation events in a marker or panel of markers in comparison with median methylation events for the marker or panel of markers from a random population of mammals (e.g., a random population of 10, 20, 30, 40, 50, 100, or 500 mammals) that do not have a specific neoplasm of interest. An elevated methylation score in a marker or panel of markers can be any score provided that the score is greater than a corresponding reference score. For example, an elevated score of methylation in a marker or panel of markers can be 0.5, 1, 2, 3, 4, 5, 6, 7, 8, 9, 10, or more fold greater than the reference methylation score.

As such, the methylation state describes the state of methylation of a nucleic acid (e.g., a genomic sequence). In addition, the methylation state refers to the characteristics of a nucleic acid segment at a particular genomic locus relevant to methylation. Such characteristics include, but are not limited to, whether any of the cytosine (C) residues within this DNA sequence are methylated, the location of methylated C residue(s), the frequency or percentage of methylated C throughout any particular region of a nucleic acid, and allelic differences in methylation due to, e.g., difference in the origin of the alleles. The terms “methylation state”, “methylation profile”, and “methylation status” also refer to the relative concentration, absolute concentration, or pattern of methylated C or unmethylated C throughout any particular region of a nucleic acid in a biological sample. For example, if the cytosine (C) residue(s) within a nucleic acid sequence are methylated it may be referred to as “hypermethylated” or having “increased methylation”, whereas if the cytosine (C) residue(s) within a DNA sequence are not methylated it may be referred to as “hypomethylated” or having “decreased methylation”. Likewise, if the cytosine (C) residue(s) within a nucleic acid sequence are methylated as compared to another nucleic acid sequence (e.g., from a different region or from a different individual, etc.) that sequence is considered hypermethylated or having increased methylation compared to the other nucleic acid sequence. Alternatively, if the cytosine (C) residue(s) within a DNA sequence are not methylated as compared to another nucleic acid sequence (e.g., from a different region or from a different individual, etc.) that sequence is considered hypomethylated or having decreased methylation compared to the other nucleic acid sequence. Additionally, the term “methylation pattern” as used herein refers to the collective sites of methylated and unmethylated nucleotides over a region of a nucleic acid. Two nucleic acids may have the same or similar methylation frequency or methylation percent but have different methylation patterns when the number of methylated and unmethylated nucleotides are the same or similar throughout the region but the locations of methylated and unmethylated nucleotides are different. Sequences are said to be “differentially methylated” or as having a “difference in methylation” or having a “different methylation state” when they differ in the extent (e.g., one has increased or decreased methylation relative to the other), frequency, or pattern of methylation. The term “differential methylation” refers to a difference in the level or pattern of nucleic acid methylation in a cancer positive sample as compared with the level or pattern of nucleic acid methylation in a cancer negative sample. It may also refer to the difference in levels or patterns between patients that have recurrence of cancer after surgery versus patients who do not have recurrence. Differential methylation and specific levels or patterns of DNA methylation are prognostic and predictive biomarkers (e.g., once the correct cut-off or predictive characteristics have been defined). DMRs can be located within any region of a gene. In some embodiments, a DMR comprises, is from, or is located within, one or more regions of a gene, including but not limited to, coding regions, non-coding regions, regulatory regions, introns, exons, promoters, enhancers, termination sequences, 3′UTRs, and 5′UTRs. In some embodiments, one or more CpG sites in a DMR can be located in non-coding regions, such as regions corresponding to long non-coding RNAs (lncRNAs).

Methylation state frequency can be used to describe a population of individuals or a sample from a single individual. For example, a nucleotide locus having a methylation state frequency of 50% is methylated in 50% of instances and unmethylated in 50% of instances. Such a frequency can be used, for example, to describe the degree to which a nucleotide locus or nucleic acid region is methylated in a population of individuals or a collection of nucleic acids. Thus, when methylation in a first population or pool of nucleic acid molecules is different from methylation in a second population or pool of nucleic acid molecules, the methylation state frequency of the first population or pool will be different from the methylation state frequency of the second population or pool. Such a frequency also can be used, for example, to describe the degree to which a nucleotide locus or nucleic acid region is methylated in a single individual. For example, such a frequency can be used to describe the degree to which a group of cells from a tissue sample are methylated or unmethylated at a nucleotide locus or nucleic acid region.

Typically, methylation of human DNA occurs on a dinucleotide sequence including an adjacent guanine and cytosine where the cytosine is located 5′ of the guanine (also termed CpG dinucleotide sequences). Most cytosines within the CpG dinucleotides are methylated in the human genome, however some remain unmethylated in specific CpG dinucleotide rich genomic regions, known as CpG islands (see, e.g., Antequera et al. (1990) Cell 62: 503-514).

As used herein, a “CpG island” or “cytosine-phosphate-guanine island”) refers to a G:C-rich region of genomic DNA containing an increased number of CpG dinucleotides relative to total genomic DNA. A CpG island can be at least 100, 200, or more base pairs in length, where the G:C content of the region is at least 50% and the ratio of observed CpG frequency over expected frequency is 0.6; in some instances, a CpG island can be at least 500 base pairs in length, where the G:C content of the region is at least 55%) and the ratio of observed CpG frequency over expected frequency is 0.65. The observed CpG frequency over expected frequency can be calculated according to the method provided in Gardiner-Garden et al (1987) J. Mol. Biol. 196: 261-281. For example, the observed CpG frequency over expected frequency can be calculated according to the formula R=(A×B)/(C×D), where R is the ratio of observed CpG frequency over expected frequency, A is the number of CpG dinucleotides in an analyzed sequence, B is the total number of nucleotides in the analyzed sequence, C is the total number of C nucleotides in the analyzed sequence, and D is the total number of G nucleotides in the analyzed sequence. Methylation state is typically determined in CpG islands, e.g., at promoter regions. It will be appreciated though that other sequences in the human genome are prone to DNA methylation such as CpA and CpT (see Ramsahoye (2000) Proc. Natl. Acad. Sci. USA 97: 5237-5242; Salmon and Kaye (1970) Biochim. Biophys. Acta. 204: 340-351; Grafstrom (1985) Nucleic Acids Res. 13: 2827-2842; Nyce (1986) Nucleic Acids Res. 14: 4353-4367; Woodcock (1987) Biochem. Biophys. Res. Commun. 145: 888-894).

As used herein, a “methylation-specific reagent” refers to a reagent that modifies a nucleotide of the nucleic acid molecule as a function of the methylation state of the nucleic acid molecule, or a methylation-specific reagent, refers to a compound or composition or other agent that can change the nucleotide sequence of a nucleic acid molecule in a manner that reflects the methylation state of the nucleic acid molecule. Methods of treating a nucleic acid molecule with such a reagent can include contacting the nucleic acid molecule with the reagent, coupled with additional steps, if desired, to accomplish the desired change of nucleotide sequence. Such methods can be applied in a manner in which unmethylated nucleotides (e.g., each unmethylated cytosine) is modified to a different nucleotide. For example, in some embodiments, such a reagent can deaminate unmethylated cytosine nucleotides to produce deoxy uracil residues. Examples of such reagents include, but are not limited to, a methylation-sensitive restriction enzyme, a methylation-dependent restriction enzyme, a bisulfite reagent, a TET enzyme, and a borane reducing agent.

A change in the nucleic acid nucleotide sequence by a methylation—specific reagent can also result in a nucleic acid molecule in which each methylated nucleotide is modified to a different nucleotide.

The term “methylation assay” refers to any assay for determining the methylation state of one or more CpG dinucleotide sequences within a sequence of a nucleic acid.

The term “MS AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, as described by Gonzalgo et al. (1997) Cancer Research 57: 594-599.

The term “MethyLight™” refers to the art-recognized fluorescence-based real-time PCR technique described by Eads et al. (1999) Cancer Res. 59: 2302-2306.

The term “HeavyMethyl™” refers to an assay wherein methylation specific blocking probes (also referred to herein as blockers) covering CpG positions between, or covered by, the amplification primers enable methylation-specific selective amplification of a nucleic acid sample.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones (1997) Nucleic Acids Res. 25: 2529-2531.

The term “MSP” (Methylation-specific PCR) refers to the art-recognized methylation assay described by Herman et al. (1996) Proc. Natl. Acad. Sci. USA 93: 9821-9826, and by U.S. Pat. No. 5,786,146.

The term “COBRA” (Combined Bisulfite Restriction Analysis) refers to the art-recognized methylation assay described by Xiong & Laird (1997) Nucleic Acids Res. 25: 2532-2534.

The term “MCA” (Methylated CpG Island Amplification) refers to the methylation assay described by Toyota et al. (1999) Cancer Res. 59: 2307-12, and in WO 00/26401A1.

As used herein, a “selected nucleotide” refers to one nucleotide of the four typically occurring nucleotides in a nucleic acid molecule (C, G, T, and A for DNA and C, G, U, and A for RNA), and can include methylated derivatives of the typically occurring nucleotides (e.g., when C is the selected nucleotide, both methylated and unmethylated C are included within the meaning of a selected nucleotide), whereas a methylated selected nucleotide refers specifically to a methylated typically occurring nucleotide and an unmethylated selected nucleotides refers specifically to an unmethylated typically occurring nucleotide.

The term “methylation-specific restriction enzyme” refers to a restriction enzyme that selectively digests a nucleic acid dependent on the methylation state of its recognition site. In the case of a restriction enzyme that specifically cuts if the recognition site is not methylated or is hemi-methylated (a methylation-sensitive enzyme), the cut will not take place (or will take place with a significantly reduced efficiency) if the recognition site is methylated on one or both strands. In the case of a restriction enzyme that specifically cuts only if the recognition site is methylated (a methylation-dependent enzyme), the cut will not take place (or will take place with a significantly reduced efficiency) if the recognition site is not methylated. Preferred are methylation-specific restriction enzymes, the recognition sequence of which contains a CG dinucleotide (for instance a recognition sequence such as CGCG or CCCGGG). Further preferred for some embodiments are restriction enzymes that do not cut if the cytosine in this dinucleotide is methylated at the carbon atom C5.

As used herein, the “sensitivity” of a given marker (or set of markers used together) refers to the percentage of samples that report a DNA methylation value above a threshold value that distinguishes between neoplastic and non-neoplastic samples. In some embodiments, a positive is defined as a histology-confirmed neoplasia that reports a DNA methylation value above a threshold value (e.g., the range associated with disease), and a false negative is defined as a histology-confirmed neoplasia that reports a DNA methylation value below the threshold value (e.g., the range associated with no disease). The value of sensitivity, therefore, reflects the probability that a DNA methylation measurement for a given marker obtained from a known diseased sample will be in the range of disease-associated measurements. As defined here, the clinical relevance of the calculated sensitivity value represents an estimation of the probability that a given marker would detect the presence of a clinical condition when applied to a subject with that condition.

As used herein, the “specificity” of a given marker (or set of markers used together) refers to the percentage of non-neoplastic samples that report a DNA methylation value below a threshold value that distinguishes between neoplastic and non-neoplastic samples. In some embodiments, a negative is defined as a histology-confirmed non-neoplastic sample that reports a DNA methylation value below the threshold value (e.g., the range associated with no disease), and a false positive is defined as a histology-confirmed non-neoplastic sample that reports a DNA methylation value above the threshold value (e.g., the range associated with disease). The value of specificity, therefore, reflects the probability that a DNA methylation measurement for a given marker obtained from a known non-neoplastic sample will be in the range of non-disease associated measurements. As defined here, the clinical relevance of the calculated specificity value represents an estimation of the probability that a given marker would detect the absence of a clinical condition when applied to a patient without that condition.

The term “AUC” as used herein is an abbreviation for the “area under a curve”. In particular it refers to the area under a Receiver Operating Characteristic (ROC) curve. The ROC curve is a plot of the true positive rate against the false positive rate for the different possible cut points of a diagnostic test. It shows the trade-off between sensitivity and specificity depending on the selected cut point (any increase in sensitivity will be accompanied by a decrease in specificity). The area under an ROC curve (AUC) is a measure for the accuracy of a diagnostic test (the larger the area the better; the optimum is 1; a random test would have a ROC curve lying on the diagonal with an area of 0.5; for reference: J. P. Egan. (1975) Signal Detection Theory and ROC Analysis, Academic Press, New York).

The term “neoplasm” as used herein refers to any new and abnormal growth of tissue. Thus, a neoplasm can be a premalignant neoplasm or a malignant neoplasm.

The term “neoplasm-specific marker,” as used herein, refers to any biological material or element that can be used to indicate the presence of a neoplasm. Examples of biological materials include, without limitation, nucleic acids, polypeptides, carbohydrates, fatty acids, cellular components (e.g., cell membranes and mitochondria), and whole cells. In some instances, markers are particular nucleic acid regions (e.g., genes, intragenic regions, specific loci, etc.). Regions of nucleic acid that are markers may be referred to, e.g., as “marker genes,” “marker regions,” “marker sequences,” “marker loci,” etc.

As used herein, the term “adenoma” refers to a benign tumor of glandular origin. Although these growths are benign, over time they may progress to become malignant.

The term “pre-cancerous” or “pre-neoplastic” and equivalents thereof refer to any cellular proliferative disorder that is undergoing malignant transformation.

A “site” of a neoplasm, adenoma, cancer, etc. is the tissue, organ, cell type, anatomical area, body part, etc. in a subject's body where the neoplasm, adenoma, cancer, etc. is located.

As used herein, a “diagnostic” test application includes the detection or identification of a disease state or condition of a subject, determining the likelihood that a subject will contract a given disease or condition, determining the likelihood that a subject with a disease or condition will respond to therapy, determining the prognosis of a subject with a disease or condition (or its likely progression or regression), and determining the effect of a treatment on a subject with a disease or condition. For example, a diagnostic can be used for detecting the presence or likelihood of a subject contracting a neoplasm or the likelihood that such a subject will respond favorably to a compound (e.g., a pharmaceutical, e.g., a drug) or other treatment.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. Examples of non-isolated nucleic acids include a given DNA sequence (e.g., a gene) found on the host cell chromosome in proximity to neighboring genes; RNA sequences, such as a specific mRNA sequence encoding a specific protein, found in the cell as a mixture with numerous other mRNAs which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may be single-stranded), but may contain both the sense and anti-sense strands (i.e., the oligonucleotide may be double-stranded). An isolated nucleic acid may, after isolation from its natural or typical environment, be combined with other nucleic acids or molecules. For example, an isolated nucleic acid may be present in a host cell into which it has been placed, e.g., for heterologous expression.

The term “purified” refers to molecules, either nucleic acid or amino acid sequences that are removed from their natural environment, isolated, or separated. An “isolated nucleic acid sequence” may therefore be a purified nucleic acid sequence. “Substantially purified” molecules are at least 60% free, preferably at least 75% free, and more preferably at least 90% free from other components with which they are naturally associated. As used herein, the terms “purified” or “to purify” also refer to the removal of contaminants from a sample. The removal of contaminating proteins results in an increase in the percent of polypeptide or nucleic acid of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “composition comprising” a given polynucleotide sequence or polypeptide refers broadly to any composition containing the given polynucleotide sequence or polypeptide. The composition may comprise an aqueous solution containing salts (e.g., NaCl), detergents (e.g., SDS), and other components (e.g., Denhardt's solution, dry milk, salmon sperm DNA, etc.).

The term “sample” is used in its broadest sense. In one sense it can refer to an animal cell or tissue. In another sense, it refers to a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the various embodiments of the present disclosure.

As used herein, a “remote sample” as used in some contexts relates to a sample indirectly collected from a site that is not the cell, tissue, or organ source of the sample. For instance, when sample material originating from the pancreas is assessed in a stool sample the sample is a remote sample.

As used herein, the terms “patient” or “subject” refer to organisms to be subject to various tests described herein. The term “subject” includes animals, preferably mammals, including humans. In a preferred embodiment, the subject is a primate. In an even more preferred embodiment, the subject is a human. Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject” includes both human and animal subjects. Thus, veterinary therapeutic uses are provided herein. As such, the present disclosure provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to carnivores such as cats and dogs; swine, including pigs, hogs, and wild boars; ruminants and/or ungulates such as cattle, oxen, sheep, giraffes, deer, goats, bison, and camels; pinnipeds; and horses. Thus, also provided is the diagnosis and treatment of livestock, including, but not limited to, domesticated swine, ruminants, ungulates, horses (including racehorses), and the like. Embodiments of the present disclosure further include a system for diagnosing one or more types or subtypes of gynecological cancers in a subject. The system can be provided, for example, as a commercial kit that can be used to screen for a risk of one or more types or subtypes of gynecological cancers or diagnose one or more types or subtypes of gynecological cancers in a subject from whom a biological sample has been collected. An exemplary system provided in accordance with the various embodiments of present disclosure includes assessing the methylation state or profile of a marker, as described herein.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining to a subject (e.g., a human, plant, or animal). The term “genomic information” refers to information pertaining to a genome including, but not limited to, nucleic acid sequences, genes, percentage methylation, allele frequencies, RNA expression levels, protein expression, phenotypes correlating to genotypes, etc. “Allele frequency information” refers to facts or data pertaining to allele frequencies, including, but not limited to, allele identities, statistical correlations between the presence of an allele and a characteristic of a subject (e.g., a human subject), the presence or absence of an allele in an individual or population, the percentage likelihood of an allele being present in an individual having one or more particular characteristics, etc.

### 2. METHYLATED DNA MARKERS AND BIOMARKER PANELS

Embodiments of the present disclosure provide methods, compositions, and systems for screening multiple types of gynecological cancer from a biological sample. In accordance with these embodiments, the present disclosure includes, but is not limited to, methods and compositions for detecting the presence of multiple types or subtypes of gynecological cancer from a biological sample. In some embodiments, the biological sample is a tissue sample, a blood sample, a plasma sample, a serum sample, a whole blood sample, a secretion sample, an organ secretion sample, a cerebrospinal fluid (CSF) sample, a saliva sample, a urine sample, and/or a stool sample. In some embodiments, the tissue sample is a gynecological tissue sample comprising one or more of vaginal tissue, vaginal cells, cervical tissue, cervical cells, endometrial tissue, endometrial cells, ovarian tissue, and ovarian cells. In some embodiments, the tissue sample is an ovarian tissue sample, an endometrial tissue sample, or a cervical tissue sample. In some embodiments, the secretion sample is a secretion or discharge from any gynecological organ or tissue, including but not limited to, vaginal tissue, cervical tissue, uterine tissue, endometrial tissue, and ovarian tissue. In some embodiments, the subject is a human.

As described further herein, embodiments of the present disclosure include novel differentially methylated regions (DMRs), each individually capable of distinguishing a specific type of gynecological cancer (i.e., endometrial cancer (EC), or ovarian cancer (OC), or cervical cancer (CC)) from a benign gynecological tissue sample. In accordance with these embodiments, these novel DMRs comprise one or more CpG sites in ADAM8, ADHFE1, AES, AGBL2, AIM1, AK5, ALKBH3, ARAP1, ARHGAP20, ASCL2, BCAT1, BEGAIN, BEND4_3696, BMP6, C12orf68, C13orf18, C14orf169_7694, C14orf169_8382, C18orf18, C1orf61, C20orf195, C4orf31, C5orf52, C6orf147, C7orf51, CD14, CELF2, CHCHD5, CHMP2A, CHST10, CLIC6, CLIP4, COL13A1, COL19A1, COL6A2, COPZ2, CREB3L1, CXCL2, CXXC5, CYTH2, DAB2IP, DGKZ, DLGAP3, DNASE2, DSCAML1, EBF1, EDARADD, EGR2, EIF5A2, ELMO1, ELMOD1, ELOVL4, EME2, EML6, EPSTI1, FADS2, FAM109B, FAM126A, FAM174B, FGF18, FKBP11, FLI1, FLOT1, FOXD3, FYN, GAL3ST2, GALR3, GAS7, GATA2_5878, GLT25D2, GNB2, HDAC7, HIC1, HLA-F, HNRNPF, HPDL, HS3ST4, HSPA1A, IDUA, IGSF9B, IL12RB2, IRAK3, IRF7, IRF8, ITPKA, KCNA2, KCNC3_6487, KCNC3_7105, KCNC4, KCNH8, KDM2B, LBX2, LCMT2, LOC100129726, LOC100287216, LOC255130, LOC339290, LOC729678, LPPR3, LRRC41, LRRC8D_8856, LTBP2, LYPLAL1, MAST4, MAX.chr1.2152, HIVEP3, GRAMD1B, MAX.chr11.0394, MAX.chr11.3750, FAT3, SLC16A7, MTUS2, LINC02323, MAX.chr14.7696, MCTP2, LOC107984974, TRIM80P, MAX.chr19.5552, ZNF433-AS1, ZNF254, MAX.chr19.0548, B3GALT1, MAX.chr2.8918, MAX.chr2.4778, MAX.chr20.3853, MAX.chr20.2903, MAX.chr21.5011, DSCR9, MAX.chr22.5665, MAX.chr3.6408, LINC02028, LINC02084, MAX.chr5.3588, CTD-2532K18.1, HS3 ST5, ARHGAP18, GRM4, LINC01004, MAX.chr8.5938, MAX.chr9.4007, MAX.chr9.2025, TRPM3, MED12L, MIAT, MLH1_4513, MLH1_5193, MMP16, MRPS21, MSI1, MT1E, MX1, MYC, MYH10, MYO15B, N4BP2L1, NBR1, NDRG2, NEGR1, NEU1, NOL3, NR3C1_2223, NR3C1_4614, NRP2, NTN1, NTNG1, PAPL, PAQR9, PDE10A, PDE3B, PDE4A, PDXK, PER1, PISD, PLEC, PLIN2, PLXND1, PPM1E, PPP1R9A, PPP2R5C, PRDM5, PTP4A3, PYCARD, RAB3C, RAI1, RARG, RASA3, RPRM, RREB1, S100A6, SAMD5, SBNO2, SDC2, SDK2, SELM, SERP2, SFMBT2_2029, SHF, SHE, SLC16A11, SLC16A5, SLC25A22, SLCO3A1, SMTN, SPDYA, SPINK2, SPOCK2, SPON1, SQSTM1_4156, ST8SIA1, TAF4B, TAF7, TEAD3, TERC, TIAM1, TLE4, TMEM101, TMEM106A, TRIM9, TRPC3, TSC22D4, TSPAN2, TSPAN5, TTC14, UBB_4001, UBB_4646, UST, VAMP5, VIM, VSTM2B, ZBTB7B, ZEB2, ZFP3, ZFP36L2, ZIC2, ZMIZ1, ZNF14, ZNF211, ZNF280B, ZNF302, ZNF382, ZNF480, ZNF483, ZNF491, ZNF569, ZNF610, ZNF702P, ZNF709, ZNF773, ZNF845, ZNF91, CDH4, LRRC34, MAX.chr10.4460, NBPF24, OBSCN, SEPT9, ZNF323, ZNF506, and/or ZNF90 (Table 1), including any combinations thereof. In some embodiments, the novel DMR(s) is from any gene or region selected from Table 1, including any combinations thereof. Each novel DMR alone is capable of distinguishing one or more gynaecological cancers from a control sample, and combining two or more of the novel DMRs can provide increased sensitivity. Therefore, combinations of two or more novel DMRs selected from Table 1 are provided.

Embodiments of the present disclosure also include novel differentially methylated regions (DMRs), each individually capable of distinguishing gynecological cancer from a benign gynecological tissue sample; these DMRs are universally present in all three types of gynecological cancer (i.e., endometrial cancer (EC), ovarian cancer (OC), and cervical cancer (CC)). In accordance with these embodiments, these novel DMRs comprise one or more CpG sites in ACSF2, AJAP1, ARL10, ARL5C, ASCL4, ATP6V1B1, BARHL1, BEND4_2963, C17orf64, C1QL3, C2orf55, C4orf48, CA3, CDO1, CELF2, CLEC14A, CSDAP1, CYTH2_4197, DLGAP1, DSCR6, EPS8L1_2819, EPS8L1_8496, FAIM2, FGF12, GATA2, HIST1H2BE, IRF4, IRX4, ITGA5, KCNA1, LECT1, LHX1, LOC440925, LPHN1, LINC02767, MAX.chr1.2533, SOX1-OT, MAX.chr13.3357, MAX.chr14.2093, MAX.chr17.2455, MAX.chr18.4390, MAX.chr19.2732, MAX.chr19.4467, PANTR1, MAX.chr2.0490, MAX.chr2.8148, MAX.chr2.3137, RIPOR3, SCRG1, MAX.chr4.4210, HMX1, CTC-359M8.1, MAX.chr5.0931, MAX.chr5.9924, LIN28B, MAX.chr6.9522, TTLL2, RNA5SP243, DLGAP2, MEX3B, MNX1, NEFL, NETO1, PAX2, PDX1, psiTPTE22, RASGEF1A, SALL3_9136, SALL3_0615, SEZ6L2, SHANK2, SHANK3, SKI, SLC35D3, SORCS3_0305, SORCS3_1038, SOX1, SQSTM1, TBXT, TCERG1L, TERT, TNFSF11, TUBB6, ULBP1, VAC14, VWC2, WDR69, ZBTB16, ZNF132, ZSCAN12, ZSCAN23, KRT86, CYP26C1, GYPC, DIDO1, EEF1A2, EMX2OS, GDF7, JSRP1, SMPD5, MDFI, MPZ, and/or VILL (Table 2), including any combinations thereof. In some embodiments, the novel DMR(s) is from any gene or region selected from Table 2, including any combinations thereof. Each novel DMR alone is capable of distinguishing one or more gynaecological cancers from a control sample, and combining two or more of the novel DMRs can provide increased sensitivity. Therefore, combinations of two or more novel DMRs selected from Table 2 are provided.

Embodiments of the present disclosure also include novel differentially methylated regions (DMRs), each individually capable of distinguishing a specific subtype of a gynecological cancer (i.e., serous ovarian cancer, clear cell ovarian cancer, endometrioid ovarian cancer, mucinous ovarian cancer, adenocarcinoma cervical cancer, squamous cervical cancer, or endometrioid endometrial cancer) from a benign gynecological tissue sample. In accordance with these embodiments, these novel DMRs comprise one or more CpG sites in AIM1, AK5, c18orf18, CDO1, DLGAP1, ELMOD1, FKBP11, FLOT1, GAL3ST2, LRRC41, LYPLAL1, MAX.chr11.3750, MLH1_4513, NR3C1_2223, PISD. RABC3, RAI1, TERC, TRPC3, ZIC2, ZMIZ1, ZNF480, ZNF491, ZNF610, and/or ZNF91 (Table 3), including any combinations thereof. In some embodiments, the novel DMR(s) is from any gene or region selected from Table 3, including any combinations thereof. Each novel DMR alone is capable of distinguishing one or more gynaecological cancers from a control sample, and combining two or more of the novel DMRs can provide increased sensitivity. Therefore, combinations of two or more novel DMRs selected from Table 3 are provided.

Embodiments of the present disclosure also include novel differentially methylated regions (DMRs), each individually capable of distinguishing a specific subtype of a gynecological cancer (i.e., serous ovarian cancer, clear cell ovarian cancer, endometrioid ovarian cancer, mucinous ovarian cancer, adenocarcinoma cervical cancer, squamous cervical cancer, or endometrioid endometrial cancer) from a benign gynecological tissue sample. In accordance with these embodiments, these novel DMRs comprise one or more CpG sites in LBX2, SPDYA, TERC, ZSCAN12, CYP26C1, and/or GYPC (Table 4), including any combinations thereof. In some embodiments, the novel DMR(s) is from any gene or region selected from Table 4, including any combinations thereof. Each novel DMR alone is capable of distinguishing one or more gynaecological cancers from a control sample, and combining two or more of the novel DMRs can provide increased sensitivity. Therefore, combinations of two or more novel DMRs selected from Table 4 are provided.

Embodiments of the present disclosure also include novel differentially methylated regions (DMRs), each individually capable of distinguishing a specific subtype of a gynecological cancer (i.e., serous ovarian cancer, clear cell ovarian cancer, endometrioid ovarian cancer, mucinous ovarian cancer, adenocarcinoma cervical cancer, squamous cervical cancer, or endometrioid endometrial cancer) from a benign gynecological tissue sample. In accordance with these embodiments, these novel DMRs comprise one or more CpG sites in KRT86, CDH4, c17orf64, EMX2OS, NBPF24, SFMBT2_0970, JSRP1, DIDO1, MAX.chr10.4460, MPZ, ZNF506, GATA2_6370, VILL, LINC02323, CYTH2_4043, LRRC8D_8831, LYPLAL1, SMPD5, SQSTM1_3864, ZNF323, OBSCN, ZNF90, LRRC34, GDF7, MDFI, EEF1A2, LRRC41, and/or SEPT9 (Table 8), including any combinations thereof. In some embodiments, the novel DMR(s) is from any gene or region selected from Table 8, including any combinations thereof. Each novel DMR alone is capable of distinguishing one or more gynaecological cancers from a control sample, and combining two or more of the novel DMRs can provide increased sensitivity. Therefore, combinations of two or more novel DMRs selected from Table 8 are provided.

As described in the forgoing Examples, experiments were conducted to identify DMRs, also referred to herein as methylated DNA markers (MDMs), capable of distinguishing types and subtypes of gynecological cancer from controls (e.g., healthy or benign samples). These experiments involved a validation study of the utility and performance of a panel of methylated DNA markers and proteins for detecting one more types or subtypes of gynecological cancer by testing an independent set of case/control samples with a refined panel of markers. Such experiments resulted in the identification of MDMs useful for simultaneously detecting the presence of multiple types of gynecological cancer (i.e., endometrial cancer (EC), or ovarian cancer (OC), or cervical cancer (CC)) from a benign gynecological tissue sample (e.g., stool sample, tissue sample, organ sample, secretion sample (e.g., vaginal secretion sample), CSF sample, saliva sample, blood sample, plasma sample or urine sample).

In some embodiments, the present disclosure provides compositions and methods for identifying, determining, and/or classifying multiple types or subtypes of gynecological cancer from a biological sample (e.g., stool sample, tissue sample, organ sample, secretion sample, CSF sample, saliva sample, blood sample, plasma sample or urine sample). The methods generally comprise determining the methylation profile of at least one methylation marker in a biological sample isolated from a subject. In some embodiments, a change in the methylation state or profile of the marker is indicative of the presence, class, or site of a specific type of gynecological cancer. Generally, such methods include, but are not limited to, detecting the presence or absence of specific types or subtypes of gynecological cancer. In some embodiments, the types and subtypes of cancer include, but are not limited to, endometrial cancer, ovarian cancer, cervical cancer, serous ovarian cancer, clear cell ovarian cancer, endometrioid ovarian cancer, mucinous ovarian cancer, adenocarcinoma cervical cancer, squamous cervical cancer, and endometrioid endometrial cancer.

In some embodiments, methods are provided that comprise contacting a nucleic acid (e.g., genomic DNA) in a biological sample obtained from a subject with at least one reagent or series of reagents that distinguishes between methylated and non-methylated nucleotides (e.g., CpG dinucleotides) within at least one methylation marker; and detecting for the presence or absence of one or more types or subtypes of gynecological cancer (e.g., afforded with a sensitivity of greater than or equal to 80% and a specificity of greater than or equal to 80%).

In some embodiments, methods are provided that comprise measuring one or both of a methylation level for one or more genes or methylated DNA markers in a biological sample from a human individual through treating genomic DNA in the biological sample with a reagent that modifies DNA in a methylation-specific manner; amplifying the treated genomic DNA using a set of primers for the selected one or more genes or methylation markers; and determining the methylation level of the one or more genes or methylation markers.

In some embodiments, methods are provided that comprise measuring an amount of one or more methylated DNA markers or genes in DNA from a biological sample; measuring an amount of at least one reference marker in the DNA; calculating a value for the amount of the at least one methylated marker gene measured in the DNA as a percentage of the amount of the reference marker gene measured in the DNA, wherein the value indicates the amount of the at least one methylated marker DNA measured in the biological sample.

In some embodiments, methods are provided that comprise measuring a methylation level of a CpG site for one or more genes in a biological sample of a human individual through treating genomic DNA in the biological sample with bisulfite a reagent capable of modifying DNA in a methylation-specific manner; amplifying the modified genomic DNA using a set of primers for the selected one or more genes; determining the methylation level of the CpG site for the selected one or more genes.

In some embodiments, the present disclosure provides methods for characterizing a biological sample comprising measuring one or both of a methylation level of a CpG site for one or more genes in a biological sample of a human individual through treating genomic DNA in the biological sample with bisulfite; amplifying the bisulfite-treated genomic DNA using a set of primers for the selected one or more genes; and determining the methylation level of the CpG site. In some embodiments, the method comprises comparing one or both of the methylation level of a methylation marker to a methylation level of a corresponding set of genes in control samples without a specific type of cancer; and/or determining that a subject has one or more types or subtypes of gynecological cancer when one or both of the methylation level measured in the one or more genes is higher than the methylation level measured in the respective control samples.

In some embodiments, the present disclosure provides methods comprising one or both of measuring in a biological sample a methylation level of one or more genes or markers through treating genomic DNA in the biological sample with bisulfite; amplifying the bisulfite-treated genomic DNA using a set of primers for the selected one or more genes; and determining the methylation level of the one or more genes or markers.

In some embodiments, the present disclosure provides methods of screening for one or more types or subtypes of gynecological cancer in a sample obtained from a subject. In accordance with these embodiments, the method includes one or both of assaying a methylation state or profile of one or more methylated DNA markers; and identifying the subject as having one or more types or subtypes of gynecological cancer when the methylation state or profile of the marker is different than a methylation state or profile of the marker assayed in a subject that does not have the one or more types of cancer.

In some embodiments, the present disclosure provides methods that comprise measuring a methylation level for one or more genes or markers in a biological sample of a human individual through treating genomic DNA in the biological sample with a reagent that modifies DNA in a methylation-specific manner; amplifying the treated genomic DNA using a set of primers for the selected one or more genes or markers; and determining the methylation level of the one or more genes or markers.

In some embodiments, the present disclosure provides methods for characterizing a biological sample comprising measuring an amount of at least one methylated DNA marker in DNA extracted from the biological sample; treating genomic DNA in the biological sample with bisulfite; and amplifying the bisulfite-treated genomic DNA using primers specific for a CpG site for each marker. In some embodiments, primers specific for each marker are capable of binding an amplicon bound by a primer sequence for the marker recited in Tables 1 or 2, wherein the amplicon bound by the primer sequence for the marker is at least a portion of a genetic region for a methylated marker recited in Tables 1 or 2; determining the methylation level of the CpG site for one or more genes.

In some embodiments, the present disclosure provides methods comprising measuring the methylation level of one or more methylated DNA markers in DNA extracted from a biological sample through extracting genomic DNA from a biological sample of a human individual suspected of having or having one or more types or subtypes of gynecological cancer; treating the extracted genomic DNA with bisulfite; and amplifying the bisulfite-treated genomic DNA with primers specific for the one or more markers. In some embodiments, primers specific for the one or more markers are capable of binding at least a portion of the bisulfite-treated genomic DNA for a chromosomal region for the marker (e.g., one or more markers recited in Tables 1 or 2), and measuring the methylation level of one or more methylated markers.

In some embodiments, the present disclosure provides methods comprising measuring the methylation level of one or more methylated DNA markers in DNA extracted from a biological sample through extracting genomic DNA from a biological sample of a human individual suspected of having or having one or more types or subtypes of gynecological cancer; treating the extracted genomic DNA with bisulfite, amplifying the bisulfite-treated genomic DNA with primers specific for the one or more markers, wherein the primers specific for the one or more markers are capable of binding at least a portion of the bisulfite-treated genomic DNA for a chromosomal region for the marker recited in Table 2; and measuring the methylation level of one or more methylated markers.

In some embodiments, the present disclosure provides methods comprising measuring the methylation level of one or more methylated DNA markers in DNA extracted from a biological sample through extracting genomic DNA from a biological sample of a human individual suspected of having or having one or more types or subtypes of gynecological cancer; treating the extracted genomic DNA with bisulfite, amplifying the bisulfite-treated genomic DNA with primers specific for the one or more markers, wherein the primers specific for the one or more markers are capable of binding at least a portion of the bisulfite-treated genomic DNA for a chromosomal region for the marker recited in Table 3; and measuring the methylation level of one or more methylated markers.

In some embodiments, the present disclosure provides methods comprising measuring the methylation level of one or more methylated DNA markers in DNA extracted from a biological sample through extracting genomic DNA from a biological sample of a human individual suspected of having or having one or more types or subtypes of gynecological cancer; treating the extracted genomic DNA with bisulfite, amplifying the bisulfite-treated genomic DNA with primers specific for the one or more markers, wherein the primers specific for the one or more markers are capable of binding at least a portion of the bisulfite-treated genomic DNA for a chromosomal region for the marker recited in Table 4; and measuring the methylation level of one or more methylated markers.

In some embodiments, the present disclosure provides methods comprising measuring the methylation level of one or more methylated DNA markers in DNA extracted from a biological sample through extracting genomic DNA from a biological sample of a human individual suspected of having or having one or more types or subtypes of gynecological cancer; treating the extracted genomic DNA with bisulfite, amplifying the bisulfite-treated genomic DNA with primers specific for the one or more markers, wherein the primers specific for the one or more markers are capable of binding at least a portion of the bisulfite-treated genomic DNA for a chromosomal region for the marker recited in Table 8; and measuring the methylation level of one or more methylated markers.

In some embodiments, the present disclosure provides methods comprising extracting genomic DNA from a biological sample of a human individual suspected of having or having cancer, treating the extracted genomic DNA with bisulfite, amplifying the bisulfite-treated genomic DNA using separate primers specific for CpG sites for one or more of the methylated DNA markers, and measuring a methylation level of the CpG site for each of the one or more markers.

In some embodiments, the present disclosure provides methods for preparing a DNA fraction from a biological sample of a human individual useful for analyzing one or more genetic loci involved in one or more chromosomal aberrations. In accordance with these embodiments, the method comprises extracting genomic DNA from a biological sample of a human individual; producing a fraction of the extracted genomic DNA by treating the extracted genomic DNA with a reagent that modifies DNA in a methylation-specific manner; amplifying the bisulfite-treated genomic DNA using separate primers specific for one or more methylated DNA markers; analyzing one or more genetic loci in the produced fraction of the extracted genomic DNA by measuring a methylation level of the CpG site for each of the one or more markers.

In some embodiments, the present disclosure provides methods for preparing a DNA fraction from a biological sample of a human individual useful for analyzing one or more DNA fragments involved in one or more chromosomal aberrations. In accordance with these embodiments, the method comprises extracting genomic DNA from a biological sample of a human individual; producing a fraction of the extracted genomic DNA by treating the extracted genomic DNA with a reagent that modifies DNA in a methylation-specific manner; amplifying the bisulfite-treated genomic DNA using separate primers specific for one or more methylated DNA markers; and analyzing one or more DNA fragments in the produced fraction of the extracted genomic DNA by measuring a methylation level of the CpG site for each of the one or more markers.

As would be appreciated by one of ordinary skill in the art based on the present disclosure, the various methods described herein are not limited to the use of any one specific methylated DNA markers, methylated marker genes, methylated genes, and/or DMRs. That is, one or more of the methylated DNA markers, methylated marker genes, methylated genes, and/or DMRs of the present disclosure can be used to distinguish and/or identify one or more types or subtypes of a gynecological cancer, including any combinations thereof. Additionally, the methylated DNA markers, methylated marker genes, methylated genes, and/or DMRs of the present disclosure can comprise a region or subregion (e.g., a gene on a chromosome, a single nucleotide, a CpG island, etc.) of any of the markers described herein.

In some embodiments, at least one DMR comprises one or more CpG sites in AIM1, FLOT1, GAL3ST2, LRRC41, LYPLAL1, MAX.chr11.3750, PISD, RAI1, ZIC2, ZMIZ1, CDH4, ZNF506, ZNF323, OBSCN, ZNF90, and/or SEPT9; and the subject has or is suspected of having ovarian cancer (OC). In some embodiments, at least one DMR comprises one or more CpG sites in AIM1, FLOT1, GAL3ST2, LYPLAL1, and/or OBSCN; and the subject has or is suspected of having serous OC. In some embodiments, at least one DMR comprises one or more CpG sites in LRRC41, PISD, ZIC2, OBSCN, and/or SEPT9; and the subject has or is suspected of having clear cell OC. In some embodiments, at least one DMR comprises one or more CpG sites in MAX.chr11.3750; and the subject has or is suspected of having endometroid OC. In some embodiments, the at least one DMR comprises one or more CpG sites in RAI1 and/or ZMIZ1; and the subject has or is suspected of having mucinous OC. In some embodiments, determining the methylation profile of the one or more CpG sites in AIM1, FLOT1, GAL3ST2, LRRC41, LYPLAL1, MAX.chr11.3750, PISD, RAI1, ZIC2, ZMIZ1, CDH4, ZNF506, ZNF323, OBSCN, ZNF90, and/or SEPT9 comprises comparing the methylation profile to a corresponding region from a control DNA sample obtained from a subject that does not have OC.

In some embodiments, at least one DMR comprises one or more CpG sites in AK5, ELMOD1, RABC3, TRPC3, ZNF480, ZNF491, ZNF610, ZNF91, and/or NBPF24; and the subject has or is suspected of having cervical cancer (CC). In some embodiments, at least one DMR comprises one or more CpG sites in AK5, ELMOD1, TRPC3, and/or ZNF480; and the subject has or is suspected of having adenocarcinoma CC. In some embodiments, at least one DMR comprises one or more CpG sites in ZNF491, ZNF610, ZNF91, and/or NBPF24; and the subject has or is suspected of having squamous cell CC. In some embodiments, determining the methylation profile of one or more CpG sites in AK5, ELMOD1, RABC3, TRPC3, ZNF480, ZNF491, ZNF610, and/or ZNF91 comprises comparing the methylation profile to a corresponding region from a control DNA sample obtained from a subject that does not have CC.

In some embodiments, at least one DMR comprises one or more CpG sites in c18orf18, FKBP11, MLH1, NR3C1, and/or TERC; and the subject has or is suspected of having endometrial cancer (EC). In some embodiments, at least one DMR comprises one or more CpG sites in MLH1 and/or SEPT9; and the subject has or is suspected of having clear cell EC. In some embodiments, at least one DMR comprises one or more CpG sites in NR3C1; and the subject has or is suspected of having endometrioid EC. In some embodiments, determining the methylation profile of one or more CpG sites in c18orf18, FKBP11, MLH1, NR3C1, and/or TERC comprises comparing the methylation profile to a corresponding region from a control DNA sample obtained from a subject that does not have EC.

In some embodiments, at least one DMR comprises one or more CpG sites in CDO1 and/or DLGAP1; and wherein the subject has or is suspected of having CC, OC, or EC. In some embodiments, determining the methylation profile of one or more CpG sites CDO1 and/or DLGAP1 comprises comparing the methylation profile to a corresponding region from a control DNA sample obtained from a subject that does not have CC, OC, or EC.

In some embodiments, the methods of the present disclosure comprise determining the methylation profile of one or more CpG sites in AIM1, FLOT1, GAL3ST2, LRRC41, LYPLAL1, MAX.chr11.3750, PISD, RAI1, ZIC2, and/or ZMIZ1. In some embodiments, the method comprises determining the methylation profile of one or more CpG sites in AK5, ELMOD1, RABC3, TRPC3, ZNF480, ZNF491, ZNF610, and/or ZNF91. In some embodiments, the method comprises determining the methylation profile of one or more CpG sites in c18orf18, FKBP11, MLH1, NR3C1, and/or TERC.

In some embodiments, the at least one DMR comprises NBPF24, and wherein the subject has or is suspected of having CC. In some embodiments, determining the methylation profile of NBPF24 comprises comparing the methylation profile to a corresponding region from a control DNA sample obtained from a subject that does not have CC.

In some embodiments, the at least one DMR comprises one or more CpG sites in CDH4, NBPF24, MAX.chr10.4460, ZNF506, ZNF323, OBSCN, ZNF90, LRRC34, SFMBT2, LINC02323, CYTH2, LRRC8D, LYPLAL1, LRRC41, and/or SEPT9, and wherein the subject has or is suspected of having EC. In some embodiments, determining the methylation profile of one or more CpG sites in CDH4, NBPF24, MAX.chr10.4460, ZNF506, ZNF323, OBSCN, ZNF90, LRRC34, SFMBT2, LINC02323, CYTH2, LRRC8D, LYPLAL1, LRRC41, and/or SEPT9 comprises comparing the methylation profile to a corresponding region from a control DNA sample obtained from a subject that does not have EC.

In some embodiments, the at least one DMR comprises one or more CpG sites in CDH4, ZNF506, ZNF323, OBSCN, ZNF90, SFMBT2, LINC02323, CYTH2, LRRC8D, LYPLAL1, LRRC41, and/or SEPT9, and wherein the subject has or is suspected of having OC. In some embodiments, determining the methylation profile of one or more CpG sites in CDH4, ZNF506, ZNF323, OBSCN, ZNF90, SFMBT2, LINC02323, CYTH2, LRRC8D, LYPLAL1, LRRC41, and/or SEPT9 comprises comparing the methylation profile to a corresponding region from a control DNA sample obtained from a subject that does not have OC. 101711 In some embodiments, the at least one DMR comprises one or more CpG sites in KRT86, EMX2OS, JSRP1, DIDO1, MPZ, VILL, SMPD5, GDF7, MDFI, c17orf64, GATA2, SQSTM1, and/or EEF1A2; and wherein the subject has or is suspected of having CC, OC, or EC. In some embodiments, determining the methylation profile of one or more CpG sites in KRT86, EMX2OS, JSRP1, DIDO1, MPZ, VILL, SMPD5, GDF7, MDFI, c17orf64, GATA2, SQSTM1, and/or EEF1A2 comprises comparing the methylation profile to a corresponding region from a control DNA sample obtained from a subject that does not have CC, OC, or EC.

As one of ordinary skill in the art would understand based on the present disclosure, one or more types or subtypes of gynecological cancers can be predicted by various combinations of markers (e.g., as identified by statistical techniques related to specificity and sensitivity of prediction). Embodiments of the present disclosure provide methods for identifying predictive combinations and validated predictive combinations for one or more types or subtypes of gynecological cancers.

Such methods are not limited to a subject type. In some embodiments, the subject is a mammal. In some embodiments, the subject is a human. Such methods are not limited to a particular manner or technique for measuring protein expression and/or activity. Techniques for measuring protein expression and/or activity levels are known in the art. Indeed, any known technique for measuring protein expression and/or activity levels are contemplated and herein incorporated.

Such methods are not limited to a particular manner or technique for determining characterizing, measuring, or assaying methylation for one or more methylated markers, methylated marker genes, genes, DMRs, and/or DNA methylated markers. In some embodiments, such techniques are based upon an analysis of the methylation status (e.g., CpG methylation status) of at least one marker, region of a marker, or base of a marker comprising a DMR.

In some embodiments, measuring the methylation state or profile of a methylated DNA marker in a sample comprises determining the methylation state of one nucleotide base. In some embodiments, measuring the methylation state of a methylated DNA marker in the sample comprises determining the extent of methylation at a plurality of nucleotide bases. Moreover, in some embodiments, the methylation state or profile of a methylated DNA marker comprises an increase in methylation of the marker relative to a normal methylation state or profile of the marker. In some embodiments, the methylation state or profile of the marker comprises decreased methylation of the marker relative to a normal methylation state of the marker. In some embodiments the methylation state or profile of the marker comprises a different pattern of methylation of the marker relative to a normal methylation state or profile of the marker.

Furthermore, in some embodiments the marker is a region of 100 or fewer nucleotide bases. In some embodiments, the marker is a region of 500 or fewer nucleotide bases. In some embodiments, the marker is a region of 1000 or fewer nucleotide bases. In some embodiments, the marker is a region of 5000 or fewer nucleotide bases. In some embodiments, the marker is one nucleotide base. In some embodiments, the marker is in a high CpG density promoter region.

In certain embodiments, methods for analyzing a nucleic acid for the presence of 5-methylcytosine involves treatment of DNA with a reagent that modifies DNA in a methylation-specific manner. Examples of such reagents include, but are not limited to, a methylation-sensitive restriction enzyme, a methylation-dependent restriction enzyme, a bisulfate reagent, a TET enzyme, and a borane reducing agent.

A frequently used method for analyzing a nucleic acid for the presence of 5-methylcytosine is based upon the bisulfite method described by Frommer, et al. for the detection of 5-methylcytosines in DNA (Frommer et al. (1992) Proc. Natl. Acad. Sci. USA 89: 1827-31 explicitly incorporated herein by reference in its entirety for all purposes) or variations thereof. The bisulfite method of mapping 5-methylcytosines is based on the observation that cytosine, but not 5-methylcytosine, reacts with hydrogen sulfite ion (also known as bisulfite). The reaction is usually performed according to the following steps: first, cytosine reacts with hydrogen sulfite to form a sulfonated cytosine. Next, spontaneous deamination of the sulfonated reaction intermediate results in a sulfonated uracil. Finally, the sulfonated uracil is desulfonated under alkaline conditions to form uracil. Detection is possible because uracil base pairs with adenine (thus behaving like thymine), whereas 5-methylcytosine base pairs with guanine (thus behaving like cytosine). This makes the discrimination of methylated cytosines from non-methylated cytosines possible by, e.g., bisulfite genomic sequencing (Grigg G, & Clark S, Bioessays (1994) 16: 431-36; Grigg G, DNA Seq. (1996) 6: 189-98), methylation-specific PCR (MSP) as is disclosed, e.g., in U.S. Pat. No. 5,786,146, or using an assay comprising sequence-specific probe cleavage, e.g., a QuARTS flap endonuclease assay (see, e.g., Zou et al. (2010) “Sensitive quantification of methylated markers with a novel methylation specific technology” Clin Chem 56: A199; and in U.S. Pat. Nos. 8,361,720; 8,715,937; 8,916,344; and 9,212,392.

In some embodiments, conventional techniques include methods comprising enclosing the DNA to be analyzed in an agarose matrix, thereby preventing the diffusion and renaturation of the DNA (bisulfite only reacts with single-stranded DNA), and replacing precipitation and purification steps with a fast dialysis (Olek A, et al. (1996) “A modified and improved method for bisulfite based cytosine methylation analysis” Nucleic Acids Res. 24: 5064-6). It is thus possible to analyze individual cells for methylation status, illustrating the utility and sensitivity of the method. An overview of conventional methods for detecting 5-methylcytosine is provided by Rein, T., et al. (1998) Nucleic Acids Res. 26: 2255.

The bisulfite technique typically involves amplifying short, specific fragments of a known nucleic acid subsequent to a bisulfite treatment, then assaying the product by sequencing (Olek & Walter (1997) Nat. Genet. 17: 275-6) or using a primer extension reaction (Gonzalgo & Jones (1997) Nucleic Acids Res. 25: 2529-31; WO 95/00669; U.S. Pat. No. 6,251,594) to analyze individual cytosine positions. Some methods use enzymatic digestion (Xiong & Laird (1997) Nucleic Acids Res. 25: 2532-4). Detection by hybridization has also been described in the art (Olek et al., WO 99/28498). Additionally, use of the bisulfite technique for methylation detection with respect to individual genes has been described (Grigg & Clark (1994) Bioessays 16: 431-6; Zeschnigk et al. (1997) Hum Mol Genet. 6: 387-95; Feil et al. (1994) Nucleic Acids Res. 22: 695; Martin et al. (1995) Gene 157: 261-4; WO 9746705; WO 9515373).

Various methylation assay procedures can be used in conjunction with bisulfite treatment according to embodiments of the present disclosure. These assays allow for determination of the methylation state of one or a plurality of CpG dinucleotides (e.g., CpG islands) within a nucleic acid sequence. Such assays involve, among other techniques, sequencing of bisulfite-treated nucleic acid, PCR (for sequence-specific amplification), Southern blot analysis, and use of methylation-specific restriction enzymes, e.g., methylation-sensitive or methylation-dependent enzymes.

For example, genomic sequencing has been simplified for analysis of methylation patterns and 5-methylcytosine distributions by using bisulfite treatment (Frommer et al. (1992) Proc. Natl. Acad. Sci. USA 89: 1827-1831). Additionally, restriction enzyme digestion of PCR products amplified from bisulfite-converted DNA finds use in assessing methylation state, e.g., as described by Sadri & Hornsby (1997) Nucl. Acids Res. 24: 5058-5059 or as embodied in the method known as COBRA (Combined Bisulfite Restriction Analysis) (Xiong & Laird (1997) Nucleic Acids Res. 25: 2532-2534).

COBRA™ analysis is a quantitative methylation assay useful for determining DNA methylation levels at specific loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the CpG islands of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples.

Typical reagents (e.g., as might be found in a typical COBRA™-based kit) for COBRA™ analysis may include, but are not limited to: PCR primers for specific loci (e.g., specific genes, markers, DMR, regions of genes, regions of markers, bisulfite treated DNA sequence, CpG island, etc.); restriction enzyme and appropriate buffer; gene-hybridization oligonucleotide; control hybridization oligonucleotide; kinase labeling kit for oligonucleotide probe; and labeled nucleotides. Additionally, bisulfite conversion reagents may include DNA denaturation buffer; sulfonation buffer; DNA recovery reagents or kits (e.g., precipitation, ultrafiltration, affinity column); desulfonation buffer; and DNA recovery components.

Assays such as “MethyLight™” (a fluorescence-based real-time PCR technique) (Eads et al., Cancer Res. 59:2302-2306, 1999), Ms-SNuPE™ (Methylation-sensitive Single Nucleotide Primer Extension) reactions (Gonzalgo & Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), and methylated CpG island amplification (“MCA”; Toyota et al., Cancer Res. 59:2307-12, 1999) are used alone or in combination with one or more of these methods.

The “HeavyMethyl™” assay, technique is a quantitative method for assessing methylation differences based on methylation-specific amplification of bi sulfite-treated DNA. Methylation-specific blocking probes (“blockers”) covering CpG positions between, or covered by, the amplification primers enable methylation-specific selective amplification of a nucleic acid sample.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers. The HeavyMethyl™ assay may also be used in combination with methylation specific amplification primers.

Typical reagents (e.g., as might be found in a typical MethyLight™-based kit) for HeavyMethyl™ analysis may include, but are not limited to: PCR primers for specific loci (e.g., specific genes, markers, regions of genes, regions of markers, bisulfite treated DNA sequence, CpG island, or bisulfite treated DNA sequence or CpG island, etc.); blocking oligonucleotides; optimized PCR buffers and deoxynucleotides; and Taq polymerase.

MSP (methylation-specific PCR) allows for assessing the methylation status of virtually any group of CpG sites within a CpG island, independent of the use of methylation-sensitive restriction enzymes (Herman et al. Proc. Natl. Acad. Sci. USA 93:9821-9826, 1996; U.S. Pat. No. 5,786,146). Briefly, DNA is modified by sodium bisulfite, which converts unmethylated, but not methylated cytosines, to uracil, and the products are subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to methylated and unmethylated PCR primers for specific loci (e.g., specific genes, markers, regions of genes, regions of markers, bisulfite treated DNA sequence, CpG island, etc.); optimized PCR buffers and deoxynucleotides, and specific probes.

The MethyLight™ assay is a high-throughput quantitative methylation assay that utilizes fluorescence-based real-time PCR (e.g., TaqMang) that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed in a “biased” reaction, e.g., with PCR primers that overlap known CpG dinucleotides. Sequence discrimination occurs both at the level of the amplification process and at the level of the fluorescence detection process.

The MethyLight™ assay is used as a quantitative test for methylation patterns in a nucleic acid, e.g., a genomic DNA sample, wherein sequence discrimination occurs at the level of probe hybridization. In a quantitative version, the PCR reaction provides for a methylation specific amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (e.g., a fluorescence-based version of the HeavyMethyl™ and MSP techniques) or with oligonucleotides covering potential methylation sites.

The MethyLight™ process is used with any suitable probe (e.g., a “TaqMang” probe, a Lightcycler® probe, etc.) For example, in some applications double-stranded genomic DNA is treated with sodium bisulfite and subjected to one of two sets of PCR reactions using TaqMang probes, e.g., with MSP primers and/or HeavyMethyl blocker oligonucleotides and a TaqMang probe. The TaqMang probe is dual-labeled with fluorescent “reporter” and “quencher” molecules and is designed to be specific for a relatively high GC content region so that it melts at about a 10° C. higher temperature in the PCR cycle than the forward or reverse primers. This allows the TaqMang probe to remain fully hybridized during the PCR annealing/extension step. As the Taq polymerase enzymatically synthesizes a new strand during PCR, it will eventually reach the annealed TaqMang probe. The Taq polymerase 5′ to 3′ endonuclease activity will then displace the TaqMang probe by digesting it to release the fluorescent reporter molecule for quantitative detection of its now unquenched signal using a real-time fluorescent detection system.

Typical reagents (e.g., as might be found in a typical MethyLight™-based kit) for MethyLight™ analysis may include, but are not limited to: PCR primers for specific loci (e.g., specific genes, markers, regions of genes, regions of markers, bisulfite treated DNA sequence, CpG island, etc.); TaqMang or Lightcycler® probes; optimized PCR buffers and deoxynucleotides; and Taq polymerase.

The QM™ (quantitative methylation) assay is an alternative quantitative test for methylation patterns in genomic DNA samples, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (a fluorescence-based version of the HeavyMethyl™ and MSP techniques) or with oligonucleotides covering potential methylation sites.

The QM™ process can be used with any suitable probe, e.g., “TaqMang” probes, Lightcycler® probes, in the amplification process. For example, double-stranded genomic DNA is treated with sodium bisulfite and subjected to unbiased primers and the TaqMang probe. The TaqMang probe is dual-labeled with fluorescent “reporter” and “quencher” molecules, and is designed to be specific for a relatively high GC content region so that it melts out at about a 10° C. higher temperature in the PCR cycle than the forward or reverse primers. This allows the TaqMang probe to remain fully hybridized during the PCR annealing/extension step. As the Taq polymerase enzymatically synthesizes a new strand during PCR, it will eventually reach the annealed TaqMang probe. The Taq polymerase 5′ to 3′ endonuclease activity will then displace the TaqMang probe by digesting it to release the fluorescent reporter molecule for quantitative detection of its now unquenched signal using a real-time fluorescent detection system. Typical reagents (e.g., as might be found in a typical QM™-based kit) for QM™ analysis may include, but are not limited to: PCR primers for specific loci (e.g., specific genes, markers, regions of genes, regions of markers, bisulfite treated DNA sequence, CpG island, etc.); TaqMang or Lightcycler® probes; optimized PCR buffers and deoxynucleotides; and Taq polymerase.

The Ms-SNuPE™ technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site of interest. Small amounts of DNA can be analyzed (e.g., microdissected pathology sections) and it avoids utilization of restriction enzymes for determining the methylation status at CpG sites.

Typical reagents (e.g., as might be found in a typical Ms-SNuPE™-based kit) for Ms-SNuPE™ analysis may include, but are not limited to: PCR primers for specific loci (e.g., specific genes, markers, regions of genes, regions of markers, bisulfite treated DNA sequence, CpG island, etc.); optimized PCR buffers and deoxynucleotides; gel extraction kit; positive control primers; Ms-SNuPE™ primers for specific loci; reaction buffer (for the Ms-SNuPE reaction); and labeled nucleotides. Additionally, bisulfite conversion reagents may include DNA denaturation buffer; sulfonation buffer; DNA recovery reagents or kit (e.g., precipitation, ultrafiltration, affinity column); desulfonation buffer; and DNA recovery components.

Reduced Representation Bisulfite Sequencing (RRBS) begins with bisulfite treatment of nucleic acid to convert all unmethylated cytosines to uracil, followed by restriction enzyme digestion (e.g., by an enzyme that recognizes a site including a CG sequence such as MspI) and complete sequencing of fragments after coupling to an adapter ligand. The choice of restriction enzyme enriches the fragments for CpG dense regions, reducing the number of redundant sequences that may map to multiple gene positions during analysis. As such, RRBS reduces the complexity of the nucleic acid sample by selecting a subset (e.g., by size selection using preparative gel electrophoresis) of restriction fragments for sequencing. As opposed to whole-genome bisulfite sequencing, every fragment produced by the restriction enzyme digestion contains DNA methylation information for at least one CpG dinucleotide. As such, RRBS enriches the sample for promoters, CpG islands, and other genomic features with a high frequency of restriction enzyme cut sites in these regions and thus provides an assay to assess the methylation state of one or more genomic loci.

A typical protocol for RRBS comprises the steps of digesting a nucleic acid sample with a restriction enzyme such as MspI, filling in overhangs and A-tailing, ligating adaptors, bisulfite conversion, and PCR. See, e.g., et al. (2005) “Genome-scale DNA methylation mapping of clinical samples at single-nucleotide resolution” Nat Methods 7: 133-6; Meissner et al. (2005) “Reduced representation bisulfite sequencing for comparative high-resolution DNA methylation analysis” Nucleic Acids Res. 33: 5868-77.

In some embodiments, a quantitative allele-specific real-time target and signal amplification (QuARTS) assay is used to evaluate methylation state. Three reactions sequentially occur in each QuARTS assay, including amplification (reaction 1) and target probe cleavage (reaction 2) in the primary reaction; and FRET cleavage and fluorescent signal generation (reaction 3) in the secondary reaction. When target nucleic acid is amplified with specific primers, a specific detection probe with a flap sequence loosely binds to the amplicon. The presence of the specific invasive oligonucleotide at the target binding site causes a 5′ nuclease, e.g., a FEN-1 endonuclease, to release the flap sequence by cutting between the detection probe and the flap sequence. The flap sequence is complementary to a non-hairpin portion of a corresponding FRET cassette. Accordingly, the flap sequence functions as an invasive oligonucleotide on the FRET cassette and effects a cleavage between the FRET cassette fluorophore and a quencher, which produces a fluorescent signal. The cleavage reaction can cut multiple probes per target and thus release multiple fluorophores per flap, providing exponential signal amplification. QuARTS can detect multiple targets in a single reaction well by using FRET cassettes with different dyes. See, e.g., in Zou et al. (2010) “Sensitive quantification of methylated markers with a novel methylation specific technology” Clin Chem 56: A199), and U.S. Pat. Nos. 8,361,720; 8,715,937; 8,916,344; and 9,212,392, each of which is incorporated herein by reference for all purposes.

The term “bisulfite reagent” refers to a reagent comprising bisulfite, disulfite, hydrogen sulfite, or combinations thereof, useful as disclosed herein to distinguish between methylated and unmethylated CpG dinucleotide sequences. Methods of said treatment are known in the art (e.g., PCT/EP2004/011715 and WO 2013/116375, each of which is incorporated by reference in its entirety). In some embodiments, bisulfite treatment is conducted in the presence of denaturing solvents such as but not limited to n-alkyleneglycol or diethylene glycol dimethyl ether (DME), or in the presence of dioxane or dioxane derivatives. In some embodiments the denaturing solvents are used in concentrations between 1% and 35% (v/v). In some embodiments, the bisulfite reaction is carried out in the presence of scavengers such as but not limited to chromane derivatives, e.g., 6-hydroxy-2,5,7,8,-tetramethylchromane 2-carboxylic acid or trihydroxybenzone acid and derivates thereof, e.g., Gallic acid (see: PCT/EP2004/011715, which is incorporated by reference in its entirety). In certain preferred embodiments, the bisulfite reaction comprises treatment with ammonium hydrogen sulfite, e.g., as described in WO 2013/116375.

In some embodiments, fragments of the treated DNA are amplified using sets of primer oligonucleotides and an amplification enzyme, according to the method and compositions described herein. The amplification of several DNA segments can be carried out simultaneously in one and the same reaction vessel. Typically, the amplification is carried out using a polymerase chain reaction (PCR). Amplicons are typically 100 to 2000 base pairs in length.

In some embodiments of the method, the methylation status or profile of CpG positions within or near a differentially methylated region (e.g., Tables 1 and 2) may be detected by use of methylation-specific primer oligonucleotides. This technique (MSP) has been described in U.S. Pat. No. 6,265,171 to Herman. The use of methylation status specific primers for the amplification of bisulfite treated DNA allows the differentiation between methylated and unmethylated nucleic acids. MSP primer pairs contain at least one primer that hybridizes to a bisulfite treated CpG dinucleotide. Therefore, the sequence of said primers comprises at least one CpG dinucleotide. MSP primers specific for non-methylated DNA contain a “T” at the position of the C position in the CpG.

Such methods are not limited to a specific type or kind of primer or primer pair related to the one or more methylated markers, methylated marker genes, genes, DMRs, and/or methylated DNA markers. In some embodiments, the primer or primer pair specific for each methylated marker gene are capable of binding an amplicon bound by a primer sequence for the marker gene recited in Tables 1 or 2, wherein the amplicon bound by the primer sequence for the marker gene is at least a portion of a genetic region for the methylated marker gene recited in Tables 1 or 2.

In another embodiment, the present disclosure provides a method for converting an oxidized 5-methylcytosine residue in cell-free DNA to a dihydrouracil residue (see, Liu et al., 2019, Nat Biotechnol. 37, pp. 424-429; U.S. Patent Application Publication No. 202000370114). The method involves reaction of an oxidized 5mC residue selected from 5-formylcytosine (5fC), 5-carboxymethylcytosine (5caC), and combinations thereof, with a borane reducing agent. The oxidized 5mC residue may be naturally occurring or, more typically, the result of a prior oxidation of a 5mC or 5hmC residue, e.g., oxidation of 5mC or 5hmC with a TET family enzyme (e.g., TET1, TET2, or TET3), or chemical oxidation of 5mC or 5hmC, e.g., with potassium perruthenate (KRuO₄) or an inorganic peroxo compound or composition such as peroxotungstate (see, e.g., Okamoto et al. (2011) Chem. Commun. 47:11231-33) and a copper (II) perchlorate/2,2,6,6-tetramethylpiperidine-1-oxyl (TEMPO) combination (see Matsushita et al. (2017) Chem. Commun. 53:5756-59).

The borane reducing agent may be characterized as a complex of borane and a nitrogen-containing compound selected from nitrogen heterocycles and tertiary amines. The nitrogen heterocycle may be monocyclic, bicyclic, or polycyclic, but is typically monocyclic, in the form of a 5- or 6-membered ring that contains a nitrogen heteroatom and optionally one or more additional heteroatoms selected from N, O, and S. The nitrogen heterocycle may be aromatic or alicyclic. Preferred nitrogen heterocycles herein include 2-pyrroline, 2H-pyrrole, 1H-pyrrole, pyrazolidine, imidazolidine, 2-pyrazoline, 2-imidazoline, pyrazole, imidazole, 1,2,4-triazole, 1,2,4-triazole, pyridazine, pyrimidine, pyrazine, 1,2,4-triazine, and 1,3,5-triazine, any of which may be unsubstituted or substituted with one or more non-hydrogen substituents. Typical non-hydrogen substituents are alkyl groups, particularly lower alkyl groups, such as methyl, ethyl, n-propyl, isopropyl, n-butyl, isobutyl, t-butyl, and the like. Exemplary compounds include, but are not limited to, borane, pyridine borane, 2-methylpyridine borane (also referred to as 2-picoline borane or pic-BH3), 5-ethyl-2-pyridine, sodium borohydride, sodium cyanoborohydride, sodium triacetoxyborohydride, diborane, decaborane, borane tetrahydrofuran, borane-dimethyl sulfide, borane-N,N-diisopropylethylamine, borane-2-chloropyridine, borane-aniline, N,N-dimethylamine borane, tert-butylamine borane sodium triacetoxyborohydride, boron hydride, hydrazine or dibutylamine borane, morpholine borane, borane-ammonia complex (BH3NH3), dicyclohexylamine borane, morpholine borane, 4-methylmorpholine borane, alkali and tetramethylamine boranes (e.g. NaBH4) and other —BH3 containing complexes and/or derivatives. In some embodiments, the reducing agent is pyridine borane and/or pic-BH3.

The reaction of the borane reducing agent with the oxidized 5mC residue in cell-free DNA is advantageous insofar as non-toxic reagents and mild reaction conditions can be employed; there is no need for any bisulfate, nor for any other potentially DNA-degrading reagents. Furthermore, conversion of an oxidized 5mC residue to dihydrouracil with the borane reducing agent can be carried out without need for isolation of any intermediates, in a “one-pot” or “one-tube” reaction. This is quite significant, since the conversion involves multiple steps, i.e., (1) reduction of the alkene bond linking C-4 and C-5 in the oxidized 5mC, (2) deamination, and (3) either decarboxylation, if the oxidized 5mC is 5caC, or deformylation, if the oxidized 5mC is 5fC.

In addition to a method for converting an oxidized 5-methylcytosine residue in cell-free DNA to a dihydrouracil residue, the present disclosure also provides a reaction mixture related to the aforementioned method. The reaction mixture comprises a sample of cell-free DNA containing at least one oxidized 5-methylcytosine residue selected from 5caC, 5fC, and combinations thereof, and a borane reducing agent effective to effective to reduce, deaminate, and either decarboxylate or deformylate the at least one oxidized 5-methylcytosine residue. The borane reducing agent is a complex of borane and a nitrogen-containing compound selected from nitrogen heterocycles and tertiary amines, as explained above. In a preferred embodiment, the reaction mixture is substantially free of bisulfite, meaning substantially free of bisulfite ion and bisulfite salts. Ideally, the reaction mixture contains no bisulfite.

In a related aspect of the present disclosure, a kit is provided for converting 5mC residues in cell-free DNA to dihydrouracil residues, where the kit includes a reagent for blocking 5hmC residues, a reagent for oxidizing 5mC residues beyond hydroxymethylation to provide oxidized 5mC residues, and a borane reducing agent effective to reduce, deaminate, and either decarboxylate or deformylate the oxidized 5mC residues. The kit may also include instructions for using the components to carry out the above-described method.

In another embodiment, a method is provided that makes use of the above-described oxidation reaction. The method enables detecting the presence and location of 5-methylcytosine residues in cell-free DNA, and comprises the following steps: (a) modifying 5hmC residues in fragmented, adapter-ligated cell-free DNA to provide an affinity tag thereon, wherein the affinity tag enables removal of modified 5hmC-containing DNA from the cell-free DNA; (b) removing the modified 5hmC-containing DNA from the cell-free DNA, leaving DNA containing unmodified 5mC residues; (c) oxidizing the unmodified 5mC residues to give DNA containing oxidized 5mC residues selected from 5caC, 5fC, and combinations thereof; (d) contacting the DNA containing oxidized 5mC residues with a borane reducing agent effective to reduce, deaminate, and either decarboxylate or deformylate the oxidized 5mC residues, thereby providing DNA containing dihydrouracil residues in place of the oxidized 5mC residues; (e) amplifying and sequencing the DNA containing dihydrouracil residues; (f) determining a 5-methylation pattern from the sequencing results in (e).

In some embodiments, the present disclosure provides a method for identifying 5-methylcytosine (5mC) or 5-hydroxymethylcytosine (5hmC) in a target nucleic acid. In some embodiments, the method comprises providing a biological sample comprising the target nucleic acid, modifying the target nucleic acid by converting the 5mC and 5hmC in the nucleic acid sample to 5-carboxylcytosine (5caC) and/or 5-formylcytosine (5fC) by contacting the nucleic acid sample with a TET enzyme so that one or more 5caC or 5fC residues are generated, and converting the 5caC and/or 5fC to dihydrouracil (DHU) by treating the target nucleic acid with a borane reducing agent to provide a modified nucleic acid sample comprising a modified target nucleic acid, and detecting the sequence of the modified target nucleic acid; wherein a cytosine (C) to thymine (T) transition or a cytosine (C) to DHU transition in the sequence of the modified target nucleic acid compared to the target nucleic acid provides the location of either a 5mC or 5hmC in the target nucleic acid. In some embodiments, the borane reducing agent is 2-picoline borane.

In some embodiments, detecting the sequence of the modified target nucleic acid comprises one or more of chain termination sequencing, microarray, high-throughput sequencing, and restriction enzyme analysis. In some embodiments, the TET enzyme is selected from the group consisting of human TET1, TET2, and TET3; murine Tet1, Tet2, and Tet3; Naegleria TET (NgTET); and Coprinopsis cinerea (CcTET). In some embodiments, the method further comprises a step of blocking one or more modified cytosines. In some embodiments, the step of blocking comprises adding a sugar to a 5hmC. In some embodiments, the method further comprises a step of amplifying the copy number of one or more nucleic acid sequences. In some embodiments, the oxidizing agent is potassium perruthenate or Cu(II)/TEMPO (2,2,6,6-tetramethylpiperidine-1-oxyl.)

The cell-free DNA is typically extracted from a biological sample from a subject, where the sample can be whole blood, plasma, urine, saliva, mucosal excretions, organ secretions, sputum, stool, or tears. In some embodiments, the cell-free DNA is derived from a tumor (e.g., a gynecological tumor). In other embodiments, the cell-free DNA is from a patient with a disease or other pathogenic condition. The cell-free DNA may or may not be derived from a tumor. In some embodiments, the cell-free DNA in which 5hmC residues are to be modified is in purified, fragmented form, and adapter-ligated. DNA purification in this context can be carried out using any suitable method known to those of ordinary skill in the art and/or described in the pertinent literature, and, while cell-free DNA can itself be highly fragmented, further fragmentation may occasionally be desirable, as described, for example, in U.S. Patent Publication No. 2017/0253924. The cell-free DNA fragments are generally in the size range of about 20 nucleotides to about 500 nucleotides, more typically in the range of about 20 nucleotides to about 250 nucleotides. The purified cell-free DNA fragments that are modified in step (a) have been end-repaired using conventional means (e.g., a restriction enzyme) so that the fragments have a blunt end at each 3′ and 5′ terminus. In a preferred method, as described in WO 2017/176630, the blunted fragments have also been provided with a 3′ overhang comprising a single adenine residue using a polymerase such as Taq polymerase. This facilitates subsequent ligation of a selected universal adapter, i.e., an adapter such as a Y-adapter or a hairpin adapter that ligates to both ends of the cell-free DNA fragments and contains at least one molecular barcode. Use of adapters also enables selective PCR enrichment of adapter-ligated DNA fragments.

In some embodiments, the “purified, fragmented cell-free DNA” comprises adapter-ligated DNA fragments. Modification of 5hmC residues in these cell-free DNA fragments with an affinity tag is done so as to enable subsequent removal of the modified 5hmC-containing DNA from the cell-free DNA. In one embodiment, the affinity tag comprises a biotin moiety, such as biotin, desthiobiotin, oxybiotin, 2-iminobiotin, diaminobiotin, biotin sulfoxide, biocytin, or the like. Use of a biotin moiety as the affinity tag allows for facile removal with streptavidin, e.g., streptavidin beads, magnetic streptavidin beads, etc.

Tagging 5hmC residues with a biotin moiety or other affinity tag is accomplished by covalent attachment of a chemoselective group to 5hmC residues in the DNA fragments, where the chemoselective group is capable of undergoing reaction with a functionalized affinity tag so as to link the affinity tag to the 5hmC residues. In one embodiment, the chemoselective group is UDP glucose-6-azide, which undergoes a spontaneous 1,3-cycloaddition reaction with an alkyne-functionalized biotin moiety, as described in Robertson et al. (2011) Biochem. Biophys. Res. Comm. 411(1):40-3, U.S. Pat. No. 8,741,567, and WO 2017/176630. Addition of an alkyne-functionalized biotin-moiety thus results in covalent attachment of the biotin moiety to each 5hmC residue.

The affinity-tagged DNA fragments can then be pulled down using, in one embodiment, streptavidin, in the form of streptavidin beads, magnetic streptavidin beads, or the like, and set aside for later analysis, if so desired. The supernatant remaining after removal of the affinity-tagged fragments contains DNA with unmodified 5mC residues and no 5hmC residues.

In some embodiments, the unmodified 5mC residues are oxidized to provide 5caC residues and/or 5fC residues, using any suitable means. The oxidizing agent is selected to oxidize 5mC residues beyond hydroxymethylation, i.e., to provide 5caC and/or 5fC residues. Oxidation may be carried out enzymatically, using a catalytically active TET family enzyme. A “TET family enzyme” or a “TET enzyme” as those terms are used herein refer to a catalytically active “TET family protein” or a “TET catalytically active fragment” as defined in U.S. Pat. No. 9,115,386, the disclosure of which is incorporated by reference herein. A preferred TET enzyme in this context is TET2; see Ito et al. (2011) Science 333(6047):1300-1303. Oxidation may also be carried out chemically, as described in the preceding section, using a chemical oxidizing agent. Examples of suitable oxidizing agent include, without limitation: a perruthenate anion in the form of an inorganic or organic perruthenate salt, including metal perruthenates such as potassium perruthenate (KRuO₄), tetraalkylammonium perruthenates such as tetrapropylammonium perruthenate (TPAP) and tetrabutylammonium perruthenate (TBAP), and polymer supported perruthenate (PSP); and inorganic peroxo compounds and compositions such as peroxotungstate or a copper (II) perchlorate/TEMPO combination. It is unnecessary at this point to separate 5fC-containing fragments from 5caC-containing fragments, insofar as in the next step of the process, converts both 5fC residues and 5caC residues to dihydrouracil (DHU).

In some embodiments, 5-hydroxymethylcytosine residues are blocked with β-glucosyltransferase (β3GT), while 5-methylcytosine residues are oxidized with a TET enzyme effective to provide a mixture of 5-formylcytosine and 5-carboxymethylcytosine. The mixture containing both of these oxidized species can be reacted with 2-picoline borane or another borane reducing agent to give dihydrouracil. In a variation on this embodiment, 5hmC-containing fragments are not removed. Rather, “TET-Assisted Picoline Borane Sequencing (TAPS),” 5mC-containing fragments and 5hmC-containing fragments are together enzymatically oxidized to provide 5fC- and 5caC-containing fragments. Reaction with 2-picoline borane results in DHU residues wherever 5mC and 5hmC residues were originally present. “Chemical Assisted Picoline Borane Sequencing (CAPS),” involves selective oxidation of 5hmC-containing fragments with potassium perruthenate, leaving 5mC residues unchanged.

As disclosed in International PCT Appln. PCT/US2019/012627, incorporated herein by reference in its entirety, TAPS comprises the use of mild enzymatic and chemical reactions to detect 5mC and 5hmC directly and quantitatively at base-resolution without affecting unmodified cytosines. In a related embodiment, the above method further includes identifying a hydroxymethylation pattern in the 5hmC-containing DNA removed from the cell-free DNA. This can be carried out using the techniques described in detail in WO 2017/176630. The process can be carried out without removal or isolation of intermediates in a one-tube method. For example, initially, cell-free DNA fragments, preferably adapter-ligated DNA fragments, are subjected to functionalization with βGT-catalyzed uridine diphosphoglucose 6-azide, followed by biotinylation via the chemoselective azide groups. This procedure results in covalently attached biotin at each 5hmC site. In a next step, the biotinylated strands and strands containing unmodified (native) 5mC are pulled down simultaneously for further processing. The native 5mC-containing strands are pulled down using an anti-5mC antibody or a methyl-CpG-binding domain (MBD) protein, as is known in the art. Then, with the 5hmC residues blocked, the unmodified 5mC residues are selectively oxidized using any suitable technique for converting 5mC to 5fC and/or 5caC, as described elsewhere herein.

The fragments obtained by means of the amplification can carry a directly or indirectly detectable label. In some embodiments, the labels are fluorescent labels, radionuclides, or detachable molecule fragments having a typical mass that can be detected in a mass spectrometer. Where said labels are mass labels, some embodiments provide that the labeled amplicons have a single positive or negative net charge, allowing for better delectability in the mass spectrometer. The detection may be carried out and visualized by means of, e.g., matrix assisted laser desorption/ionization mass spectrometry (MALDI) or using electron spray mass spectrometry (ESI).

Methods for isolating DNA suitable for these assay technologies are known in the art. In particular, some embodiments comprise isolation of nucleic acids as described in U.S. patent application Ser. No. 13/470,251 (“Isolation of Nucleic Acids”), incorporated herein by reference in its entirety.

In some embodiments, the markers described herein find use in QUARTS assays performed on stool samples. In some embodiments, methods for producing DNA samples and, in particular, to methods for producing DNA samples that comprise highly purified, low-abundance nucleic acids in a small volume (e.g., less than 100, less than 60 microliters) and that are substantially and/or effectively free of substances that inhibit assays used to test the DNA samples (e.g., PCR, INVADER, QuARTS assays, etc.) are provided. Such DNA samples find use in diagnostic assays that qualitatively detect the presence of, or quantitatively measure the activity, expression, or amount of, a gene, a gene variant (e.g., an allele), or a gene modification (e.g., methylation) present in a sample taken from a patient. For example, some cancers are correlated with the presence of particular mutant alleles or particular methylation states, and thus detecting and/or quantifying such mutant alleles or methylation states has predictive value in the diagnosis and treatment of cancer.

Many valuable genetic markers are present in extremely low amounts in samples and many of the events that produce such markers are rare. Consequently, even sensitive detection methods such as PCR require a large amount of DNA to provide enough of a low-abundance target to meet or supersede the detection threshold of the assay. Moreover, the presence of even low amounts of inhibitory substances can compromise the accuracy and precision of these assays directed to detecting such low amounts of a target. Accordingly, provided herein are methods providing the requisite management of volume and concentration to produce such DNA samples.

In some embodiments, the sample comprises stool, tissue sample, an organ secretion, CSF, saliva, blood, or urine. In some embodiments, the subject is human. Such samples can be obtained by any number of means known in the art, such as will be apparent to the skilled person. Cell free or substantially cell free samples can be obtained by subjecting the sample to various techniques known to those of skill in the art which include, but are not limited to, centrifugation and filtration. Although it is generally preferred that no invasive techniques are used to obtain the sample, it still may be preferable to obtain samples such as tissue homogenates, tissue sections, and biopsy specimens. The technology is not limited in the methods used to prepare the samples and provide a nucleic acid for testing. For example, in some embodiments, a DNA is isolated from a sample (e.g., stool sample, tissue sample, organ secretion sample, CSF sample, saliva sample, blood sample, plasma sample or urine sample) using direct gene capture, e.g., as detailed in U.S. Pat. Nos. 8,808,990 and 9,169,511, and in WO 2012/155072, or by a related method.

The analysis of markers can be carried out separately or simultaneously with additional markers within one test sample. For example, several markers can be combined into one test for efficient processing of multiple samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states over time. Changes in methylation state, as well as the absence of change in methylation state, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events.

The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

Genomic DNA may be isolated by any means, including the use of commercially available kits. Briefly, wherein the DNA of interest is encapsulated by a cellular membrane the biological sample must be disrupted and lysed by enzymatic, chemical or mechanical means. The DNA solution may then be cleared of proteins and other contaminants, e.g., by digestion with proteinase K. The genomic DNA is then recovered from the solution. This may be carried out by means of a variety of methods including salting out, organic extraction, or binding of the DNA to a solid phase support. The choice of method will be affected by several factors including time, expense, and required quantity of DNA. All clinical sample types comprising neoplastic matter or pre-neoplastic matter are suitable for use in the present method, e.g., cell lines, histological slides, biopsies, paraffin-embedded tissue, body fluids, stool, tissue, colonic effluent, urine, blood plasma, blood serum, whole blood, isolated blood cells, cells isolated from the blood, and combinations thereof.

The technology is not limited in the methods used to prepare the samples and provide a nucleic acid for testing. For example, in some embodiments, a DNA is isolated from a stool sample or from blood or from a plasma sample using direct gene capture, e.g., as detailed in U.S. Pat. Appl. Ser. No. 61/485,386 or by a related method.

The genomic DNA sample is then treated with at least one reagent, or series of reagents, which distinguishes between methylated and non-methylated CpG dinucleotides within at least one marker comprising a DMR (e.g., DMRs Tables 1 or 2).

In some embodiments, the reagent converts cytosine bases which are unmethylated at the 5′-position to uracil, thymine, or another base which is dissimilar to cytosine in terms of hybridization behavior. However, in some embodiments, the reagent may be a methylation sensitive restriction enzyme.

In some embodiments, the genomic DNA sample is treated in such a manner that cytosine bases that are unmethylated at the 5′ position are converted to uracil, thymine, or another base that is dissimilar to cytosine in terms of hybridization behavior. In some embodiments, this treatment is carried out with bisulfite (hydrogen sulfite, disulfite) followed by alkaline hydrolysis.

The treated nucleic acid is then analyzed to determine the methylation state of the target gene sequences (at least one gene, genomic sequence, or nucleotide from a marker comprising a DMR, e.g., at least one DMR chosen from the DMRs in Tables 1 or 2). The method of analysis may be selected from those known in the art, including those listed herein (e.g., QuARTS and MSP as described herein).

Such samples can be obtained by any number of means known in the art, such as will be apparent to the skilled person. For instance, urine and fecal samples are easily attainable, while blood, ascites, serum, or pancreatic fluid samples can be obtained parenterally by using a needle and syringe, for instance. Cell free or substantially cell free samples can be obtained by subjecting the sample to various techniques known to those of skill in the art which include, but are not limited to, centrifugation and filtration. Although it is generally preferred that no invasive techniques are used to obtain the sample, it still may be preferable to obtain samples such as tissue homogenates, tissue sections, and biopsy specimens.

Embodiments of the present disclosure further provide compositions. In some embodiments, the present disclosure provides composition comprising a nucleic acid comprising a DMR and a bisulfite reagent. In some embodiments, composition comprising a nucleic acid comprising a DMR and one or more primers are provided (e.g., primers capable of binding at least a portion of a region of a DMR recited in Tables 1 or 2, or primers capable of binding an amplicon bound by a primer capable of binding of at least a portion of a region of a DMR recited in Tables 1 or 2). In certain embodiments, compositions comprising a nucleic acid comprising a DMR and a methylation-sensitive restriction enzyme are provided. In certain embodiments, compositions comprising a nucleic acid comprising a DMR and a polymerase are provided.

### 3. METHODS OF TREATMENT

In some embodiments, the present disclosure provides methods for treating a subject (e.g., a patient having or suspected of having one or more types or subtypes of gynecological cancer). In accordance with these embodiments, the method includes determining a methylation state or profile of one or more methylated DNA markers provided herein, and/or measuring the expression and/or activity level of one or more protein markers, and administering a treatment to the patient based on the results of determining the methylation state and/or protein marker expression and/or activity level. The treatment may be administration of a pharmaceutical compound, a vaccine, performing a surgery, imaging the patient, performing another test. In some embodiments, treating a subject includes a method of clinical screening, a method of prognosis assessment, a method of monitoring the results of therapy, a method to identify patients most likely to respond to a particular therapeutic treatment, a method of imaging a patient or subject, and a method for drug screening and development.

In some embodiments, a method for diagnosing a specific type of cancer in a subject is provided. The terms “diagnosing” and “diagnosis” as used herein refer to methods by which the skilled artisan can estimate and even determine whether or not a subject is suffering from a given disease or condition or may develop a given disease or condition in the future. The skilled artisan often makes a diagnosis on the basis of one or more diagnostic indicators, such as for example one or more biomarkers (e.g., one or more methylated markers, methylated marker genes, genes, DMRs, and/or DNA methylated markers as disclosed herein), the methylation state of which is indicative of the presence, severity, or absence of the condition, and/or the expression and/or activity level of one or more protein markers.

Along with diagnosis, clinical cancer prognosis relates to determining the aggressiveness of the cancer and the likelihood of tumor recurrence to plan the most effective therapy. If a more accurate prognosis can be made or even a potential risk for developing the cancer can be assessed, appropriate therapy, and in some instances less severe therapy for the patient can be chosen. Assessment (e.g., determining methylation state) of cancer biomarkers is useful to separate subjects with good prognosis and/or low risk of developing cancer who will need no therapy or limited therapy from those more likely to develop cancer or suffer a recurrence of cancer who might benefit from more intensive treatments.

As such, “making a diagnosis” or “diagnosing”, as used herein, is further inclusive of determining a risk of developing cancer or determining a prognosis, which can provide for predicting a clinical outcome (with or without medical treatment), selecting an appropriate treatment (or whether treatment would be effective), or monitoring a current treatment and potentially changing the treatment, based on the measure of the diagnostic biomarkers (e.g., DMR) disclosed herein. Further, in some embodiments of the presently disclosed subject matter, multiple determination of the biomarkers over time can be made to facilitate diagnosis and/or prognosis. A temporal change in the biomarker can be used to predict a clinical outcome, monitor the progression of cancer or a subtype of cancer, and/or monitor the efficacy of appropriate therapies directed against the cancer. In such an embodiment for example, one might expect to see a change in the methylation state of one or more biomarkers (e.g., DMR) disclosed herein (and potentially one or more additional biomarker(s), if monitored) and/or expression and/or activity level of a protein marker in a biological sample over time during the course of an effective therapy.

The presently disclosed subject matter further provides in some embodiments a method for determining whether to initiate or continue prophylaxis or treatment of a cancer in a subject. In some embodiments, the method comprises providing a series of biological samples over a time period from the subject; analyzing the series of biological samples to determine a methylation state or profile of at least one marker disclosed herein in each of the biological samples; and comparing any measurable change in the methylation states of one or more of the biomarkers in each of the biological samples. Any changes over the time period can be used to predict risk of developing cancer, predict clinical outcome, determine whether to initiate or continue the prophylaxis or therapy of the cancer, and whether a current therapy is effectively treating the cancer. For example, a first time point can be selected prior to initiation of a treatment and a second time point can be selected at some time after initiation of the treatment. Methylation states and protein marker expression/activity levels can be measured in each of the samples taken from different time points and qualitative and/or quantitative differences noted. A change in the methylation states of the biomarker levels and/or protein marker expression/activity levels from the different samples can be correlated with a specific cancer risk, prognosis, determining treatment efficacy, and/or progression of the cancer in the subject. In some embodiments, the methods and compositions of the present disclosure are for treatment or diagnosis of disease at an early stage, for example, before symptoms of the disease appear. In some embodiments, the methods and compositions of the present disclosure are for treatment or diagnosis of disease at a clinical stage.

In some embodiments, multiple determinations of one or more diagnostic or prognostic biomarkers can be made, and a temporal change in the marker can be used to determine a diagnosis or prognosis. For example, a diagnostic marker can be determined at an initial time, and again at a second time. In such embodiments, an increase in the marker from the initial time to the second time can be diagnostic of a particular type or severity of cancer, or a given prognosis. Likewise, a decrease in the marker from the initial time to the second time can be indicative of a particular type or severity of cancer, or a given prognosis. Furthermore, the degree of change of one or more markers can be related to the severity of the cancer and future adverse events. The skilled artisan will understand that, while in certain embodiments comparative measurements can be made of the same biomarker at multiple time points, one can also measure a given biomarker at one time point, and a second biomarker at a second time point, and a comparison of these markers can provide diagnostic information.

As used herein, the phrase “determining the prognosis” refers to methods by which the skilled artisan can predict the course or outcome of a condition in a subject. The term “prognosis” does not refer to the ability to predict the course or outcome of a condition with 100% accuracy, or even that a given course or outcome is predictably more or less likely to occur based on the methylation state of a biomarker (e.g., a DMR). Instead, the skilled artisan will understand that the term “prognosis” refers to an increased probability that a certain course or outcome will occur; that is, that a course or outcome is more likely to occur in a subject exhibiting a given condition, when compared to those individuals not exhibiting the condition. For example, in individuals not exhibiting the condition (e.g., having a normal methylation state of one or more DMR, and/or protein marker expression and/or activity levels), the chance of a given outcome (e.g., suffering from a specific type of cancer) may be very low.

In some embodiments, a statistical analysis associates a prognostic indicator with a predisposition to an adverse outcome. For example, in some embodiments, a methylation state and/or protein marker expression/activity level different from that in a normal control sample obtained from a patient who does not have a cancer can signal that a subject is more likely to suffer from a cancer than subjects with a level that is more similar to the methylation state in the control sample, as determined by a level of statistical significance. Additionally, a change in methylation state and/or protein marker expression/activity level from a baseline (e.g., “normal”) level can be reflective of subject prognosis, and the degree of change in methylation state and/or protein marker expression/activity level can be related to the severity of adverse events. Statistical significance is often determined by comparing two or more populations and determining a confidence interval and/or a p value. See, e.g., Dowdy and Wearden, Statistics for Research, John Wiley & Sons, New York, 1983, incorporated herein by reference in its entirety. Exemplary confidence intervals of the present subject matter are 90%, 95%, 97.5%, 98%, 99%, 99.5%, 99.9% and 99.99%, while exemplary p values are 0.1, 0.05, 0.025, 0.02, 0.01, 0.005, 0.001, and 0.0001.

In other embodiments, a threshold degree of change in the methylation state and/or protein marker expression/activity level of a prognostic or diagnostic biomarker disclosed herein (e.g., a DMR; protein marker) can be established, and the degree of change in the methylation state and/or protein marker expression/activity level of the biomarker in a biological sample is simply compared to the threshold degree of change in the methylation state and/or protein marker expression/activity level. A preferred threshold change in the methylation state and/or protein marker expression/activity level for biomarkers provided herein is about 5%, about 10%, about 15%, about 20%, about 25%, about 30%, about 50%, about 75%, about 100%, and about 150%. In yet other embodiments, a “nomogram” can be established, by which a methylation state and/or protein marker expression/activity level of a prognostic or diagnostic indicator (biomarker or combination of biomarkers) is directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

In some embodiments, a control sample is analyzed concurrently with the biological sample, such that the results obtained from the biological sample can be compared to the results obtained from the control sample. Additionally, it is contemplated that standard curves can be provided, with which assay results for the biological sample may be compared. Such standard curves present methylation states and/or protein marker expression/activity levels of a biomarker as a function of assay units, e.g., fluorescent signal intensity, if a fluorescent label is used. Using samples taken from multiple donors, standard curves can be provided for control methylation states of the one or more biomarkers in normal tissue, as well as for “at-risk” levels of the one or more biomarkers in plasma taken from donors with a specific type of cancer. In certain embodiments of the method, a subject is identified as having cancer upon identifying an aberrant methylation state of one or more DMR and/or protein marker expression/activity level provided herein in a biological sample obtained from the subject. In other embodiments of the method, the detection of an aberrant methylation state and/or protein marker expression/activity level of one or more of such biomarkers in a biological sample obtained from the subject results in the subject being identified as having cancer.

The analysis of markers can be carried out separately or simultaneously with additional markers within one test sample. For example, several markers can be combined into one test for efficient processing of a multiple of samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states and/or protein marker expression/activity levels over time. Changes in methylation state and/or protein marker expression/activity level, as well as the absence of change in methylation state, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events.

The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

In some embodiments, the subject is diagnosed as having a specific type of cancer if, when compared to a control methylation state and/or protein marker expression/activity level, there is a measurable difference in the methylation state and/or protein marker expression/activity level of at least one biomarker in the sample. Conversely, when no change in methylation state and/or protein marker expression/activity level is identified in the biological sample, the subject can be identified as not having a specific type of cancer, not being at risk for the cancer, or as having a low risk of the cancer. In this regard, subjects having the cancer or risk thereof can be differentiated from subjects having low to substantially no cancer or risk thereof. Those subjects having a risk of developing a specific type of cancer can be placed on a more intensive and/or regular screening schedule. On the other hand, those subjects having low to substantially no risk may avoid being subjected to additional testing for cancer risk (e.g., invasive procedure), until such time as a future screening, for example, a screening conducted in accordance with the various embodiments of the present disclosure, indicates that a risk of cancer risk has appeared in those subjects.

As mentioned above, depending on the embodiment of the method of the present disclosure, detecting a change in methylation state and/or protein marker expression/activity level of the one or more biomarkers can be a qualitative determination or it can be a quantitative determination. As such, the step of diagnosing a subject as having, or at risk of developing, a specific type of cancer indicates that certain threshold measurements are made, e.g., the methylation state and/or protein marker expression/activity level of the one or more biomarkers in the biological sample varies from a predetermined control methylation state and/or control protein marker expression/activity level. In some embodiments of the method, the control methylation state is any detectable methylation state of the biomarker. In some embodiments, the control protein marker expression/activity level is any measurable and/or protein marker expression/activity level of the protein marker. In other embodiments of the method where a control sample is tested concurrently with the biological sample, the predetermined methylation state is the methylation state in the control sample, and the predetermined protein marker expression/activity level control state is the and/or protein marker expression/activity level in the control sample. In other embodiments of the method, the predetermined methylation state and/or predetermined protein marker expression/activity level is based upon and/or identified by a standard curve. In other embodiments of the method, the predetermined methylation state and/or predetermined protein marker expression/activity level is a specifically state or range of state. As such, the predetermined methylation state and/or predetermined protein marker expression/activity level can be chosen, within acceptable limits that will be apparent to those skilled in the art, based in part on the embodiment of the method being practiced and the desired specificity, etc.

Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject” includes both human and animal subjects. Thus, veterinary therapeutic uses are provided herein. As such, embodiments of the present disclosure provide for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to carnivores such as cats and dogs; swine, including pigs, hogs, and wild boars; ruminants and/or ungulates such as cattle, oxen, sheep, giraffes, deer, goats, bison, and camels; and horses. Thus, also provided is the diagnosis and treatment of livestock, including, but not limited to, domesticated swine, ruminants, ungulates, horses (including racehorses), and the like.

### 4. SAMPLES, KITS, AND CONTROLS

Embodiments of the present disclosure provide technology for screening multiple types of gynecological cancer from a biological sample. In accordance with these embodiments, the present disclosure includes, but is not limited to, methods and compositions for detecting the presence of multiple types and/or subtypes of gynecological cancer from a biological sample. In some embodiments, the biological sample is a tissue sample, a blood sample, a plasma sample, a serum sample, a whole blood sample, a secretion sample, an organ secretion sample, a cerebrospinal fluid (CSF) sample, a saliva sample, a urine sample, and/or a stool sample. In some embodiments, the tissue sample is a gynecological tissue sample comprising one or more of vaginal tissue, vaginal cells, cervical tissue, cervical cells, endometrial tissue, endometrial cells, ovarian tissue, and ovarian cells. In some embodiments, the tissue sample is an ovarian tissue sample, an endometrial tissue sample, or a cervical tissue sample. In some embodiments, the subject is a human.

In other embodiments, “sample,” “test sample,” and “biological sample” refer to fluid sample containing or suspected of containing a methylated DNA marker of the present disclosure. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis. In a particular example, the source is a mammalian (e.g., human) bodily substance (e.g., bodily fluid, blood such as whole blood, serum, plasma, urine, saliva, sweat, sputum, semen, mucus, lacrimal fluid, lymph fluid, amniotic fluid, interstitial fluid, cerebrospinal fluid, feces, tissue, organ, one or more dried blood spots, or the like). Tissues may include, but are not limited to gynecological tissue, oropharyngeal tissue, nasopharyngeal tissue, skeletal muscle tissue, liver tissue, lung tissue, kidney tissue, myocardial tissue, brain tissue, bone marrow, cervix tissue, skin, etc. The sample may be a liquid sample or a liquid extract of a solid sample. In some embodiments, the source of the sample may be an organ or tissue, such as a biopsy sample and/or a secretion sample (e.g., gynecological secretion), which may be solubilized by tissue disintegration/cell lysis. Additionally, the sample can be a nasopharyngeal or oropharyngeal sample obtained using one or more swabs that, once obtained, is placed in a sterile tube containing a virus transport media (VTM) or universal transport media (UTM), for testing.

A wide range of volumes of the fluid sample may be analyzed. In a few exemplary embodiments, the sample volume may be about 0.5 nL, about 1 nL, about 3 nL, about 0.01 μL, about 0.1 μL, about 1 μL, about 5 μL, about 10 μL, about 100 μL, about 1 mL, about 5 mL, about 10 mL, or the like. In some cases, the volume of the fluid sample is between about 0.01 μL and about 10 mL, between about 0.01 μL and about 1 mL, between about 0.01 μL and about 100 μL, or between about 0.1 μL and about 10 μL.

In some cases, the fluid sample may be diluted prior to use in an assay. For example, in embodiments where the source containing a methylated DNA marker is a human body fluid (e.g., blood, serum, secretion), the fluid may be diluted with an appropriate solvent (e.g., a buffer such as PBS buffer). A fluid sample may be diluted about 1-fold, about 2-fold, about 3-fold, about 4-fold, about 5-fold, about 6-fold, about 10-fold, about 100-fold, or greater, prior to use. In other cases, the fluid sample is not diluted prior to use in an assay.

In some cases, the sample may undergo pre-analytical processing. Pre-analytical processing may offer additional functionality such as nonspecific protein removal and/or effective yet cheaply implementable mixing functionality. General methods of pre-analytical processing may include the use of electrokinetic trapping, AC electrokinetics, surface acoustic waves, isotachophoresis, dielectrophoresis, electrophoresis, or other pre-concentration techniques known in the art. In some cases, the fluid sample may be concentrated prior to use in an assay. For example, in embodiments where the source containing a methylated DNA marker is a human body fluid (e.g., blood, serum, secretion), the fluid may be concentrated by precipitation, evaporation, filtration, centrifugation, or a combination thereof. A fluid sample may be concentrated about 1-fold, about 2-fold, about 3-fold, about 4-fold, about 5-fold, about 6-fold, about 10-fold, about 100-fold, or greater, prior to use.

It may be desirable to include a control. The control may be analyzed concurrently with the sample from the subject as described above. The results obtained from the subject sample can be compared to the results obtained from the control sample. Standard curves may be provided, with which assay results for the sample may be compared. Such standard curves present levels of one or more methylated DNA markers as a function of assay units. Using samples taken from multiple donors, standard curves can be provided for reference levels of a methylated DNA marker in normal healthy tissue, as well as for “at-risk” levels of the methylated DNA marker in tissue taken from donors, who may have one or more characteristics of a gynecological cancer.

Embodiments of the present disclosure also include a kit for performing the methods described herein. The kits comprise embodiments of the compositions, devices, apparatuses, etc. described herein, and instructions for use of the kit. Such instructions describe appropriate methods for preparing an analyte from a sample, e.g., for collecting a sample and preparing a nucleic acid from the sample. Individual components of the kit are packaged in appropriate containers and packaging (e.g., vials, boxes, blister packs, ampules, jars, bottles, tubes, and the like) and the components are packaged together in an appropriate container (e.g., a box or boxes) for convenient storage, shipping, and/or use by the user of the kit. It is understood that liquid components (e.g., a buffer) may be provided in a lyophilized form to be reconstituted by the user. Kits may include a control or reference for assessing, validating, and/or assuring the performance of the kit. For example, a kit for assaying the amount of a nucleic acid present in a sample may include a control comprising a known concentration of the same or another nucleic acid for comparison and, in some embodiments, a detection reagent (e.g., a primer) specific for the control nucleic acid. The kits are appropriate for use in a clinical setting and, in some embodiments, for use in a user's home. The components of a kit, in some embodiments, provide the functionalities of a system for preparing a nucleic acid solution from a sample. In some embodiments, certain components of the system are provided by the user.

In some embodiments, the present disclosure provides compositions (e.g., reaction mixtures). In some embodiments, the present disclosure provides a composition comprising a nucleic acid comprising a DMR and a reagent capable of modifying DNA in a methylation-specific manner (e.g., a methylation-sensitive restriction enzyme, a methylation-dependent restriction enzyme, and a bisulfite reagent) (e.g., a methylation-sensitive restriction enzyme, a methylation-dependent restriction enzyme, Ten Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), borane reducing agent). Some embodiments provide a composition comprising a nucleic acid comprising a DMR and an oligonucleotide as described herein. Some embodiments provide a composition comprising a nucleic acid comprising a DMR and a methylation-sensitive restriction enzyme. Some embodiments provide a composition comprising a nucleic acid comprising a DMR and a polymerase.

In some embodiments, the technology described herein is associated with a programmable machine designed to perform a sequence of arithmetic or logical operations as provided by the methods described herein. For example, some embodiments of the technology are associated with (e.g., implemented in) computer software and/or computer hardware. In one aspect, the technology relates to a computer comprising a form of memory, an element for performing arithmetic and logical operations, and a processing element (e.g., a microprocessor) for executing a series of instructions (e.g., a method as provided herein) to read, manipulate, and store data. In some embodiments, a microprocessor is part of a system for determining a methylation state (e.g., of one or more DMRs in Tables 1 or 2); comparing methylation states; generating standard curves; determining a Ct value; calculating a fraction, frequency, or percentage of methylation; identifying a CpG island; determining a specificity and/or sensitivity of an assay or marker; calculating an ROC curve and an associated AUC; sequence analysis; all as described herein or is known in the art. In some embodiments, a microprocessor is part of a system for determining a level of protein expression and/or activity (e.g., one or more protein markers described herein); comparing level of protein marker expression or activity in comparison to a standard non-cancerous level; all as described herein or is known in the art. In some embodiments, a microprocessor is part of a system for determining a methylation state (e.g., of one or more DMRs in Tables 1 or 2); comparing methylation states; generating standard curves; determining a Ct value; calculating a fraction, frequency, or percentage of methylation; identifying a CpG island; determining a specificity and/or sensitivity of an assay or marker; calculating an ROC curve and an associated AUC; sequence analysis; all as described herein or is known in the art; and/or determining a level of protein expression and/or activity (e.g., one or more protein markers described herein); comparing level of protein marker expression or activity in comparison to a standard non-cancerous level; all as described herein or is known in the art.

In some embodiments, a software or hardware component receives the results of multiple assays and determines a single value result to report to a user that indicates a cancer risk based on the results of the multiple assays (e.g., determining the methylation state of one or more DMRs in Tables 1 or 2, and determining protein marker expression and/or activity levels). Related embodiments calculate a risk factor based on a mathematical combination (e.g., a weighted combination, a linear combination) of the results from the multiple assays (e.g., determining the methylation state of one or more DMRs in Tables 1 or 2, and determining protein marker expression and/or activity levels). In some embodiments, the methylation state of a DMR defines a dimension and may have values in a multidimensional space and the coordinate defined by the methylation states of multiple DMR is a result, e.g., to report to a user, e.g., related to a cancer risk.

In some embodiments, the various embodiments of the present disclosure are associated with a plurality of programmable devices that operate in concert to perform a method as described herein. For example, in some embodiments, a plurality of computers (e.g., connected by a network) may work in parallel to collect and process data, e.g., in an implementation of cluster computing or grid computing or some other distributed computer architecture that relies on complete computers (with onboard CPUs, storage, power supplies, network interfaces, etc.) connected to a network (private, public, or the internet) by a conventional network interface, such as Ethernet, fiber optic, or by a wireless network technology.

For example, some embodiments provide a computer that includes a computer-readable medium. The embodiment includes a random access memory (RAM) coupled to a processor. The processor executes computer-executable program instructions stored in memory. Such processors may include a microprocessor, an ASIC, a state machine, or other processor, and can be any of a number of computer processors, such as processors from Intel Corporation of Santa Clara, California and Motorola Corporation of Schaumburg, Illinois. Such processors include, or may be in communication with, media, for example computer-readable media, which stores instructions that, when executed by the processor, cause the processor to perform the steps described herein.

Computers are connected in some embodiments to a network. Computers may also include a number of external or internal devices such as a mouse, a CD-ROM, DVD, a keyboard, a display, or other input or output devices. Examples of computers are personal computers, digital assistants, personal digital assistants, cellular phones, mobile phones, smart phones, pagers, digital tablets, laptop computers, internet appliances, and other processor-based devices. In general, the computers related to aspects of the technology provided herein may be any type of processor-based platform that operates on any operating system, such as Microsoft Windows, Linux, UNIX, Mac OS X, etc., capable of supporting one or more programs comprising the technology provided herein. Some embodiments comprise a personal computer executing other application programs (e.g., applications). The applications can be contained in memory and can include, for example, a word processing application, a spreadsheet application, an email application, an instant messenger application, a presentation application, an Internet browser application, a calendar/organizer application, and any other application capable of being executed by a client device. All such components, computers, and systems described herein as associated with the technology may be logical or virtual.

In some embodiments, the present disclosure provides systems for screening for one or more types or subtypes of gynecological cancer in a sample obtained from a subject. Exemplary embodiments of systems include, e.g., a system for screening for multiple types or subtypes of gynecological cancer in a sample obtained from a subject (e.g., a stool sample, a tissue sample, an organ secretion sample, a CSF sample, a saliva sample, a blood sample, a plasma sample, or a urine sample). In some embodiments, the system comprises an analysis component configured to one or both of determining the methylation state of one or more methylated markers in a sample and determining the expression and/or activity level of one or more protein markers in the sample, a software component configured to compare the methylation state of the one or more methylated markers in the sample and/or expression and/or activity level of the one or more protein markers in the sample with a control sample or a reference sample recorded in a database, and an alert component configured to alert a user of a cancer associated state.

In some embodiments, an alert is determined by a software component that receives the results from multiple assays (e.g., determining the methylation states of the one or more methylated markers) (e.g., determining the expression and/or activity level of the one or more protein markers) and calculating a value or result to report based on the multiple results.

Some embodiments provide a database of weighted parameters associated with each methylated marker and/or protein marker expression and/or activity level provided herein for use in calculating a value or result and/or an alert to report to a user (e.g., such as a physician, nurse, clinician, etc.). In some embodiments all results from multiple assays are reported. In some embodiments, one or more results are used to provide a score, value, or result based on a composite of one or more results from multiple assays that is indicative of a cancer risk in a subject. Such methods are not limited to particular methylation markers. In such methods and systems, the one or more methylation markers comprise a base in a DMR selected from the DMRs in Tables 1 or 2.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

The various components of the kit optionally are provided in suitable containers as necessary. The kit can further include containers for holding or storing a sample (e.g., a container or cartridge for a urine, whole blood, plasma, serum sample, tissue, or bodily secretion sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like. In some embodiments, the instrument is a collection device that includes, but is not limited to, a tampon, a lavage that releases liquid into the vagina and re-collects fluid, a cervical brush, a Fournier cervical self-sampling device, and a swab. In some embodiments, the biological sample is obtained from the subject, and the method further comprises extracting the DNA sample from the biological sample. In some embodiments, the biological sample is collected with a collection device having an absorbing member capable of collecting the biological sample upon contact. In some embodiments, the absorbing member is a sponge configured for insertion into an orifice.

### 5. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Experiments were conducted to assess the feasibility of a panel of methylated DNA markers (MDMs) for detecting non-specific gynecological cancer, site specific gynecological cancer (e.g., cervical cancer, ovarian cancer, endometrial cancer), and specific subtypes of gynecological cancer.

A proprietary methodology of sample preparation, sequencing, analyses pipelines, and filters were utilized to identify and narrow differentially methylated regions (DMRs) to those which would pinpoint specific types of gynecological cancer and excel in a clinical testing environment. From the cancer-to-cancer analysis of the RRBS data, 249 hypermethylated gynecological cancer specific (either endometrial cancer (EC), ovarian cancer (OC), or cervical cancer (CC)) DMRs were identified (Table 1). They include specific hypermethylated regions for one or two (at most) of the gynecological cancers as well as subtype specific regions. Such experiments also uncovered 89 regions universally hypermethylated in all three gynecological cancers (Table 2). The characteristics of the markers include extremely low background noise in leukocytes (≤0.01), which mitigates inflammatory signals and potentially allows for plasma-based testing. The signal in BCV tissues is also low for the markers (≤0.05), which would be the dominant cell type from a tampon device. AUCs for all MDMs were in excess of 0.90 in separating the cancers from leukocytes and ≥0.85 in distinguishing one cancer from another.

TABLE 1
Methylated regions distinguishing specific types of gynecological
cancer (either endometrial cancer (EC), ovarian cancer (OC), or
cervical cancer (CC)) from benign tissue (genomic coordinates can
be obtained using the Human Feb. 2009 (GRCh37/hg19) Assembly).

| | | | Accession No. |
|---|---|---|---|
| DMR | Gene Annotation | Chromosome No. | or SEQ ID NO |
| 1 | ADAM8 | 10 | NM_001109 |
| | | | NM_001164490 |
| | | | NM_001164489 |
| 2 | ADHFE1 | 8 | NM_144650 |
| 3 | AES | 19 | NM_198970 |
| | | | NM_198969 |
| | | | NM_001130 |
| 4 | AGBL2 | 11 | NM_024783 |
| 5 | AIM1 | 6 | NM_001624 |
| 6 | AK5 | 1 | NM_174858 |
| | | | NM_012093 |
| 7 | ALKBH3 | 11 | NM_139178 |
| 8 | ARAP1 | 11 | NM_015242 |
| | | | NM_001135190 |
| | | | NM_001040118 |
| 9 | ARHGAP20 | 11 | NM_020809 |
| 10 | ASCL2 | 11 | NM_005170 |
| 11 | BCAT1 | 12 | NM_001178092 |
| | | | NM_005504 |
| | | | NM_001178091 |
| 12 | BEGAIN | 14 | NM_001159531 |
| | | | NM_020836 |
| 13 | BEND4_3696 | 4 | NM_001159547 |
| | | | NM_207406 |
| 14 | BMP6 | 6 | NM_001718 |
| 15 | C12orf68 | 12 | NM_001013635 |
| 16 | C13orf18 | 13 | NM_025113 |
| 17 | C14orf169_7694 | 14 | NM_024644 |
| 18 | C14orf169_8382 | 14 | NM_024644 |
| 19 | C18orf18 | 18 | NR_026849 |
| 20 | C1orf61 | 1 | NM_006365 |
| 21 | C20orf195 | 20 | NM_024059 |
| 22 | C4orf31 | 4 | NM_024574 |
| 23 | C5orf52 | 5 | NM_001145132 |
| 24 | C6orf147 | 6 | NR_027005 |
| 25 | C7orf51 | 7 | NM_173564 |
| 26 | CD14 | 5 | NM_001174105 |
| | | | NM_001040021 |
| | | | NM_000591 |
| | | | NM_001174104 |
| 27 | CELF2 | 10 | NM_001025077 |
| | | | NM_006561 |
| 28 | CHCHD5 | 2 | NM_032309 |
| 29 | CHMP2A | 19 | NM_198426 |
| | | | NM_014453 |
| 30 | CHST10 | 2 | NM_004854 |
| 31 | CLIC6 | 21 | NM_053277 |
| 32 | CLIP4 | 2 | NM_024692 |
| 33 | COL13A1 | 10 | NM_080802 |
| | | | NM_080800 |
| | | | NM_080798 |
| | | | NM_080805 |
| | | | NM_001130103 |
| | | | NM_080801 |
| 34 | COL19A1 | 6 | NM_001858 |
| 35 | COL6A2 | 21 | NM_058175 |
| | | | NM_058174 |
| | | | NM_001849 |
| 36 | COPZ2 | 17 | NM_016429 |
| 37 | CREB3L1 | 11 | NM_052854 |
| 38 | CXCL2 | 4 | NM_002089 |
| 39 | CXXC5 | 5 | NM_016463 |
| 40 | DAB2IP | 9 | NM_032552 |
| 41 | DGKZ | 11 | NM_201532 |
| 42 | DLGAP3 | 1 | NM_001080418 |
| 43 | DNASE2 | 19 | NM_001375 |
| 44 | DSCAML1 | 11 | NM_020693 |
| 45 | EBF1 | 5 | NM_024007 |
| 46 | EDARADD | 1 | NM_145861 |
| | | | NM_080738 |
| 47 | EGR2 | 10 | NM_001136177 |
| | | | NM_000399 |
| | | | NM_001136179 |
| | | | NM_001136178 |
| 48 | EIF5A2 | 3 | NM_020390 |
| 49 | ELMO1 | 7 | NM_014800 |
| 50 | ELMOD1 | 11 | NM_018712 |
| | | | NM_001130037 |
| 51 | ELOVL4 | 6 | NM_022726 |
| 52 | EME2 | 16 | NM_001010865 |
| 53 | EML6 | 2 | NM_001039753 |
| 54 | EPSTI1 | 13 | NM_033255 |
| | | | NM_001002264 |
| 55 | FADS2 | 11 | NM_004265 |
| 56 | FAM109B | 22 | NM_001002034 |
| 57 | FAM126A | 7 | NM_032581 |
| 58 | FAM174B | 15 | NM_207446 |
| 59 | FGF18 | 5 | NM_003862 |
| 60 | FKBP11 | 12 | NM_001143782 |
| | | | NM_001143781 |
| | | | NM_016594 |
| 61 | FLI1 | 11 | NM_002017 |
| | | | NM_001167681 |
| 62 | FLOT1 | 6 | NM_005803 |
| 63 | FOXD3 | 1 | NM_012183 |
| 64 | FYN | 6 | NM_002037 |
| 65 | GAL3ST2 | 2 | NM_022134 |
| 66 | GALR3 | 22 | NM_003614 |
| 67 | GAS7 | 17 | NM_201433 |
| 68 | GATA2_5878 | 3 | NM_001145662 |
| | | | NM_032638 |
| | | | NM_001145661 |
| 69 | GLT25D2 | 1 | NM_015101 |
| 70 | GNB2 | 7 | NM_005273 |
| 71 | HDAC7 | 12 | NM_001098416 |
| | | | NM_015401 |
| 72 | HIC1 | 17 | NM_001098202 |
| | | | NM_006497 |
| 73 | HLA-F | 6 | NM_001098478 |
| | | | NM_018950 |
| | | | NM_001098479 |
| 74 | HNRNPF | 10 | NM_001098205 |
| | | | NM_001098207 |
| | | | NM_001098206 |
| | | | NM_004966 |
| | | | NM_001098208 |
| | | | NM_001098204 |
| 75 | HPDL | 1 | NM_032756 |
| 76 | HS3ST4 | 16 | NM_006040 |
| 77 | HSPA1A | 6 | NM_005345 |
| 78 | IDUA | 4 | NM_000203 |
| 79 | IGSF9B | 11 | NM_014987 |
| 80 | IL12RB2 | 1 | NM_001559 |
| 81 | IRAK3 | 12 | NM_007199 |
| | | | NM_001142523 |
| 82 | IRF7 | 11 | NM_004031 |
| | | | NM_004029 |
| | | | NM_001572 |
| 83 | IRF8 | 16 | NM_002163 |
| 84 | ITPKA | 15 | NM_002220 |
| 85 | KCNA2 | 1 | NM_004974 |
| 86 | KCNC3_6487 | 19 | NM_004977 |
| 87 | KCNC3_7105 | 19 | NM_004977 |
| 88 | KCNC4 | 1 | NR_036437 |
| | | | NM_001039574 |
| | | | NM_004978 |
| 89 | KCNH8 | 3 | NM_144633 |
| 90 | KDM2B | 12 | NM_001005366 |
| | | | NM_032590 |
| 91 | LBX2 | 2 | NM_001009812 |
| 92 | LCMT2 | 15 | NM_014793 |
| 93 | LOC100129726 | 2 | NR_027251 |
| 94 | LOC100287216 | 2 | NR_029193 |
| 95 | LOC255130 | 4 | NR_034081 |
| 96 | LOC339290 | 18 | NR_015389 |
| 97 | LOC729678 | 5 | NR_027183 |
| 98 | LPPR3 | 19 | NM_024888 |
| 99 | LRRC41 | 1 | NM_006369 |
| 100 | LRRC8D_8856 | 1 | NM_018103 |
| | | | NM_001134479 |
| 101 | LTBP2 | 14 | NM_000428 |
| 102 | LYPLAL1 | 1 | NM_138794 |
| 103 | MAST4 | 5 | NM_001164664 |
| | | | NM_198828 |
| 104 | MAX.chr1.2152 | 1 | SEQ ID NO: 2 |
| 105 | HIVEP3 | 1 | NR_038261 |
| 106 | GRAMD1B | 11 | NM_001367418 |
| 107 | MAX.chr11.0394 | 11 | SEQ ID NO: 3 |
| 108 | MAX.chr11.3750 | 11 | SEQ ID NO: 4 |
| 109 | FAT3 | 11 | NM_001378141 |
| 110 | SLC16A7 | 12 | NM_001270622 |
| 111 | MTUS2 | 13 | NM_001384605 |
| 112 | LINC02323 | 14 | NR_146561 |
| 113 | MAX.chr14.7696 | 14 | SEQ ID NO: 5 |
| 114 | MCTP2 | 15 | NM_001385011 |
| 115 | LOC107984974 | 17 | NR_171380 |
| 116 | TRIM80P | 17 | ENSG00000232724 |
| 117 | MAX.chr19.5552 | 19 | SEQ ID NO: 6 |
| 118 | ZNF433-AS1 | 19 | NR_134930 |
| 119 | ZNF254 | 19 | NM_001278663 |
| 120 | MAX.chr19.0548 | 19 | SEQ ID NO: 7 |
| 121 | B3GALT1 | 2 | NM_020981 |
| 122 | MAX.chr2.8918 | 2 | SEQ ID NO: 8 |
| 123 | MAX.chr2.4778 | 2 | SEQ ID NO: 9 |
| 124 | MAX.chr20.3853 | 20 | SEQ ID NO: 10 |
| 125 | MAX.chr20.2903 | 20 | SEQ ID NO: 11 |
| 126 | MAX.chr21.5011 | 21 | SEQ ID NO: 12 |
| 127 | DSCR9 | 21 | NR_026719 |
| 128 | MAX.chr22.5665 | 22 | SEQ ID NO: 13 |
| 129 | MAX.chr3.6408 | 3 | SEQ ID NO: 14 |
| 130 | LINC02028 | 3 | NR_136179 |
| 131 | LINC02084 | 3 | ENSG00000272282 |
| 132 | MAX.chr5.3588 | 5 | SEQ ID NO: 15 |
| 133 | CTD-2532K18.1 | 5 | ENSG00000251670 |
| 134 | HS3ST5 | 6 | NM_001387047 |
| 135 | ARHGAP18 | 6 | NM_033515 |
| 136 | GRM4 | 6 | NM_000841 |
| 137 | LINC01004 | 7 | NR_039981 |
| 138 | MAX.chr8.5938 | 8 | SEQ ID NO: 16 |
| 139 | MAX.chr9.4007 | 9 | SEQ ID NO: 17 |
| 140 | MAX.chr9.2025 | 9 | SEQ ID NO: 18 |
| 141 | TRPM3 | 9 | NM_206948 |
| 142 | MED12L | 3 | NM_053002 |
| 143 | MIAT | 22 | NR_003491 |
| | | | NR_033321 |
| | | | NR_033320 |
| | | | NR_033319 |
| 144 | MLH1_4513 | 3 | NM_001167619 |
| | | | NM_001167618 |
| | | | NM_001167617 |
| | | | NM_000249 |
| 145 | MLH1_5193 | 3 | NM_001167619 |
| | | | NM_001167618 |
| | | | NM_001167617 |
| | | | NM_000249 |
| 146 | MMP16 | 8 | NM_005941 |
| 147 | MRPS21 | 1 | NM_018997 |
| | | | NM_031901 |
| 148 | MSI1 | 12 | NM_002442 |
| 149 | MT1E | 16 | NM_175617 |
| 150 | MX1 | 21 | NM_001178046 |
| | | | NM_002462 |
| | | | NM_001144925 |
| 151 | MYC | 8 | NM_002467 |
| 152 | MYH10 | 17 | NM_005964 |
| 153 | MYO15B | 17 | NR_003587 |
| 154 | N4BP2L1 | 13 | NM_052818 |
| | | | NM_001079691 |
| 155 | NBR1 | 17 | NM_031858 |
| | | | NM_031862 |
| | | | NM_005899 |
| 156 | NDRG2 | 14 | NM_201535 |
| | | | NM_201537 |
| | | | NM_201541 |
| | | | NM_201539 |
| | | | NM_016250 |
| | | | NM_201538 |
| | | | NM_201536 |
| | | | NM_201540 |
| 157 | NEGR1 | 1 | NM_173808 |
| 158 | NEU1 | 6 | NM_000434 |
| 159 | NOL3 | 16 | NM_001185057 |
| | | | NM_001185058 |
| | | | NM_003946 |
| 160 | NR3C1_2223 | 5 | NM_000176 |
| | | | NM_001018076 |
| | | | NM_001024094 |
| | | | NM_001018074 |
| | | | NM_001018075 |
| | | | NM_001018077 |
| | | | NM_001020825 |
| 161 | NR3C1_4614 | 5 | NM_000176 |
| | | | NM_001018076 |
| | | | NM_001024094 |
| | | | NM_001018074 |
| | | | NM_001018075 |
| | | | NM_001018077 |
| | | | NM_001020825 |
| 162 | NRP2 | 2 | NM_018534 |
| | | | NM_201266 |
| | | | NM_201264 |
| | | | NM_201267 |
| | | | NM_201279 |
| | | | NM_003872 |
| 163 | NTN1 | 17 | NM_004822 |
| 164 | NTNG1 | 1 | NM_014917 |
| | | | NM_001113226 |
| | | | NM_001113228 |
| 165 | PAPL | 19 | NM_001004318 |
| 166 | PAQR9 | 3 | NM_198504 |
| 167 | PDE10A | 6 | NM_001130690 |
| | | | NM_006661 |
| 168 | PDE3B | 11 | NM_000922 |
| 169 | PDE4A | 19 | NM_001111307 |
| 170 | PDXK | 21 | NM_003681 |
| 171 | PER1 | 17 | NM_002616 |
| 172 | PISD | 22 | NM_014338 |
| 173 | PLEC | 8 | NM_201381 |
| | | | NM_201383 |
| | | | NM_000445 |
| | | | NM_201378 |
| | | | NM_201380 |
| | | | NM_201384 |
| | | | NM_201382 |
| | | | NM_201379 |
| 174 | PLIN2 | 9 | NM_001122 |
| 175 | PLXND1 | 3 | NM_015103 |
| 176 | PPM1E | 17 | NM_014906 |
| 177 | PPP1R9A | 7 | NM_001166161 |
| | | | NM_017650 |
| | | | NM_001166162 |
| | | | NM_001166160 |
| | | | NM_001166163 |
| 178 | PPP2R5C | 14 | NM_001161726 |
| | | | NM_001161725 |
| 179 | PRDM5 | 4 | NM_018699 |
| 180 | PTP4A3 | 8 | NM_032611 |
| | | | NM_007079 |
| 181 | PYCARD | 16 | NM_145182 |
| | | | NM_013258 |
| 182 | RAB3C | 5 | NM_138453 |
| 183 | RAI1 | 17 | NM_030665 |
| 184 | RARG | 12 | NM_000966 |
| | | | NM_001042728 |
| 185 | RASA3 | 13 | NM_007368 |
| 186 | RPRM | 2 | NM_019845 |
| 187 | RREB1 | 6 | NM_001003699 |
| | | | NM_001003698 |
| | | | NM_001168344 |
| | | | NM_001003700 |
| 188 | S100A6 | 1 | NM_014624 |
| 189 | SAMD5 | 6 | NM_001030060 |
| 190 | SBNO2 | 19 | NM_001100122 |
| | | | NM_014963 |
| 191 | SDC2 | 8 | NM_002998 |
| 192 | SDK2 | 17 | NM_001144952 |
| 193 | SELM | 22 | NM_080430 |
| 194 | SERP2 | 13 | NM_001010897 |
| 195 | SFMBT2_2029 | 10 | NM_001029880 |
| | | | NM_001018039 |
| 196 | SHF | 15 | NM_138356 |
| 197 | SHH | 7 | NM_000193 |
| 198 | SLC16A11 | 17 | NM_153357 |
| 199 | SLC16A5 | 17 | NM_004695 |
| 200 | SLC25A22 | 11 | NM_001191061 |
| | | | NM_001191060 |
| | | | NM_024698 |
| 201 | SLCO3A1 | 15 | NM_013272 |
| | | | NM_001145044 |
| 202 | SMTN | 22 | NM_134269 |
| | | | NM_006932 |
| | | | NM_134270 |
| 203 | SPDYA | 2 | NM_182756 |
| | | | NM_001142634 |
| 204 | SPINK2 | 4 | NM_021114 |
| 205 | SPOCK2 | 10 | NM_014767 |
| | | | NM_001134434 |
| 206 | SPON1 | 11 | NM_006108 |
| 207 | SQSTM1_4156 | 5 | NM_003900 |
| | | | NM_001142299 |
| | | | NM_001142298 |
| 208 | ST8SIA1 | 12 | NM_003034 |
| 209 | TAF4B | 18 | NM_005640 |
| 210 | TAF7 | 5 | NM_005642 |
| 211 | TEAD3 | 6 | NM_003214 |
| 212 | TERC | 3 | NR_001566 |
| 213 | TIAM1 | 21 | NM_003253 |
| 214 | TLE4 | 9 | NM_007005 |
| 215 | TMEM101 | 17 | NM_032376 |
| 216 | TMEM106A | 17 | NM_145041 |
| 217 | TRIM9 | 14 | NM_015163 |
| | | | NM_052978 |
| 218 | TRPC3 | 4 | NM_001130698 |
| 219 | TSC22D4 | 7 | NM_030935 |
| 220 | TSPAN2 | 1 | NM_005725 |
| 221 | TSPAN5 | 4 | NM_005723 |
| 222 | TTC14 | 3 | NM_133462 |
| | | | NM_001042601 |
| 223 | UBB_4001 | 17 | NM_018955 |
| 224 | UBB_4646 | 17 | NM_018955 |
| 225 | UST | 6 | NM_005715 |
| 226 | VAMP5 | 2 | NM_006634 |
| 227 | VIM | 10 | NM_003380 |
| 228 | VSTM2B | 19 | NM_001146339 |
| 229 | ZBTB7B | 1 | NM_015872 |
| 230 | ZEB2 | 2 | NR_033258 |
| | | | NM_014795 |
| | | | NM_001171653 |
| 231 | ZFP3 | 17 | NM_153018 |
| 232 | ZFP36L2 | 2 | NM_006887 |
| 233 | ZIC2 | 13 | NM_007129 |
| 234 | ZMIZ1 | 10 | NM_020338 |
| 235 | ZNF14 | 19 | NM_021030 |
| 236 | ZNF211 | 19 | NM_198855 |
| | | | NM_006385 |
| 237 | ZNF280B | 22 | NM_080764 |
| 238 | ZNF302 | 19 | NM_018443 |
| | | | NM_001012320 |
| 239 | ZNF382 | 19 | NM_032825 |
| 240 | ZNF480 | 19 | NM_144684 |
| 241 | ZNF483 | 9 | NM_133464 |
| | | | NM_001007169 |
| 242 | ZNF491 | 19 | NM_152356 |
| 243 | ZNF569 | 19 | NM_152484 |
| 244 | ZNF610 | 19 | NM_001161426 |
| | | | NM_001161427 |
| | | | NM_001161425 |
| 245 | ZNF702P | 19 | NR_003578 |
| 246 | ZNF709 | 19 | NM_152601 |
| 247 | ZNF773 | 19 | NM_198542 |
| 248 | ZNF845 | 19 | NM_138374 |
| 249 | ZNF91 | 19 | NM_003430 |
| 339 | CDH4 | 20 | NM_001794 |
| 340 | LRRC34 | 3 | NM_001172780 |
| 341 | MAX.chr10.4460 | 10 | SEQ ID NO: 1 |
| 342 | NBPF24 | 1 | NM_001037501 |
| 343 | OBSCN | 1 | NM_001271223 |
| 344 | SEPT9 | 17 | NM_001293695 |
| 345 | ZNF323 | 6 | NM_001243242 |
| 346 | ZNF506 | 19 | NR_171023 |
| 347 | ZNF90 | 19 | NM_007138 |
| 348 | SFMBT2_0970 | 10 | NM_001029880 |
| | | | NM_001018039 |
| 348 | CYTH2_4043 | 19 | NM_004228 |
| | | | NM_017457 |
| 350 | LRRC8D_8831 | 1 | NM_018103 |
| | | | NM_001134479 |

### Example 2

In accordance with the experiments described in Example 1, a novel set of differentially methylated regions (DMRs) discriminating multiple types of gynecological cancers from non-neoplastic control DNA were identified, as shown in Table 2.

TABLE 2
Universally methylated regions present in all three gynecological
cancers assayed (e.g., endometrial cancer (EC), ovarian
cancer (OC), and cervical cancer (CC)) from benign gynecological
tissue (genomic coordinates can be obtained using the
Human Feb. 2009 (GRCh37/hg19) Assembly).

| | | | Accession No. |
|---|---|---|---|
| DMR | Gene Annotation | Chromosome No. | or SEQ ID NO |
| 250 | ACSF2 | 17 | NM_025149 |
| 251 | AJAP1 | 1 | NM_018836 |
| | | | NM_001042478 |
| 252 | ARL10 | 5 | NM_001079685 |
| | | | NM_001079684 |
| | | | NM_020444 |
| 253 | ARL5C | 17 | NM_001143968 |
| 254 | ASCL4 | 12 | NM_203436 |
| 255 | ATP6V1B1 | 2 | NM_001692 |
| 256 | BARHL1 | 9 | NM_020064 |
| 257 | BEND4_2963 | 4 | NM_001159547 |
| | | | NM_207406 |
| 258 | C17orf64 | 17 | NM_181707 |
| 259 | C1QL3 | 10 | NM_001010908 |
| 260 | C2orf55 | 2 | NM_207362 |
| 261 | C4orf48 | 4 | NM_001168243 |
| | | | NM_001141936 |
| 262 | CA3 | 8 | NM_005181 |
| 263 | CDO1 | 5 | NM_001801 |
| 264 | CELF2 | 10 | NM_001025076 |
| | | | NM_001083591 |
| | | | NM_001025077 |
| | | | NM_006561 |
| 265 | CLEC14A | 14 | NM_175060 |
| 266 | CSDAP1 | 16 | NR_027011 |
| 267 | CYTH2_4197 | 19 | NM_004228 |
| | | | NM_017457 |
| 268 | DLGAP1 | 18 | NM_001003809 |
| | | | NM_004746 |
| 269 | DSCR6 | 21 | NM_018962 |
| 270 | EPS8L1_2819 | 19 | NM_017729 |
| | | | NM_133180 |
| 271 | EPS8L1_8496 | 19 | NM_017729 |
| | | | NM_133180 |
| 272 | FAIM2 | 12 | NM_012306 |
| 273 | FGF12 | 3 | NM_004113 |
| | | | NM_021032 |
| 274 | HIST1H2BE | 6 | NM_003523 |
| 275 | IRF4 | 6 | NM_001195286 |
| | | | NR_036585 |
| | | | NM_002460 |
| 276 | IRX4 | 5 | NM_016358 |
| 277 | ITGA5 | 12 | NM_002205 |
| 278 | KCNA1 | 12 | NM_000217 |
| 279 | LECT1 | 13 | NM_007015 |
| | | | NM_001011705 |
| 280 | LHX1 | 17 | NM_005568 |
| 281 | LOC440925 | 2 | NR_027433 |
| 282 | LPHN1 | 19 | NM_001008701 |
| | | | NM_014921 |
| 283 | LINC02767 | 1 | NR_167982 |
| 284 | MAX.chr1.2533 | 1 | SEQ ID NO: 19 |
| 285 | SOX1-OT | 13 | NR_120392 |
| 286 | MAX.chr13.3357 | 13 | SEQ ID NO: 20 |
| 287 | MAX.chr14.2093 | 14 | SEQ ID NO: 21 |
| 288 | MAX.chr17.2455 | 17 | SEQ ID NO: 22 |
| 289 | MAX.chr18.4390 | 18 | SEQ ID NO: 23 |
| 290 | MAX.chr19.2732 | 19 | SEQ ID NO: 24 |
| 291 | MAX.chr19.4467 | 19 | SEQ ID NO: 25 |
| 292 | PANTR1 | 2 | NR_037883 |
| 293 | MAX.chr2.0490 | 2 | SEQ ID NO: 26 |
| 294 | MAX.chr2.8148 | 2 | SEQ ID NO: 27 |
| 295 | MAX.chr2.3137 | 2 | SEQ ID NO: 28 |
| 296 | RIPOR3 | 20 | NR_110890 |
| 297 | SCRG1 | 4 | NM_001329597 |
| 298 | MAX.chr4.4210 | 4 | SEQ ID NO: 29 |
| 299 | HMX1 | 4 | NM_001306142 |
| 300 | CTC-359M8.1 | 5 | ENSG00000250025 |
| 301 | MAX.chr5.0931 | 5 | SEQ ID NO: 30 |
| 302 | MAX.chr5.9924 | 5 | SEQ ID NO: 31 |
| 303 | LIN28B | 6 | ENSG00000187772 |
| 304 | MAX.chr6.9522 | 6 | SEQ ID NO: 32 |
| 305 | TTLL2 | 6 | ENSG00000120440 |
| 306 | RNA5SP243 | 7 | ENSG00000252866 |
| 307 | DLGAP2 | 8 | NM_001346810 |
| 308 | MEX3B | 15 | NM_032246 |
| 309 | MNX1 | 7 | NM_005515 |
| | | | NM_001165255 |
| 310 | NEFL | 8 | NM_006158 |
| 311 | NETO1 | 18 | NM_153181 |
| | | | NM_138966 |
| | | | NM_ 138999 |
| 312 | PAX2 | 10 | NM_003989 |
| | | | NM_000278 |
| | | | NM_003988 |
| | | | NM_003990 |
| | | | NM_003987 |
| 313 | PDX1 | 13 | NM_000209 |
| 314 | psiTPTE22 | 22 | NR_001591 |
| 315 | RASGEF1A | 10 | NM_145313 |
| 316 | SALL3_9136 | 18 | NM_171999 |
| 317 | SALL3_0615 | 18 | NM_171999 |
| 318 | SEZ6L2 | 16 | NM_001114099 |
| | | | NM_012410 |
| | | | NM_201575 |
| | | | NM_001114100 |
| 319 | SHANK2 | 11 | NM_133266 |
| | | | NM_012309 |
| 320 | SHANK3 | 22 | NM_001080420 |
| 321 | SKI | 1 | NM_003036 |
| 322 | SLC35D3 | 6 | NM_001008783 |
| 323 | SORCS3_0305 | 10 | NM_014978 |
| 324 | SORCS3_1038 | 10 | NM_014978 |
| 325 | SOX1 | 13 | NM_005986 |
| 326 | TBXT | 6 | NM_003181 |
| 327 | TCERG1L | 10 | NM_174937 |
| 328 | TERT | 5 | NM_001193376 |
| | | | NM_198253 |
| 329 | TNFSF11 | 13 | NM_003701 |
| | | | NM_033012 |
| 330 | TUBB6 | 18 | NM_032525 |
| 331 | ULBP1 | 6 | NM_025218 |
| 332 | VAC14 | 16 | NM_018052 |
| 333 | VWC2 | 7 | NM_198570 |
| 334 | WDR69 | 2 | NM_178821 |
| 335 | ZBTB16 | 11 | NM_001018011 |
| | | | NM_006006 |
| 336 | ZNF132 | 19 | NM_003433 |
| 337 | ZSCAN12 | 6 | NR_028077 |
| | | | NM_001163391 |
| 338 | ZSCAN23 | 6 | NM_001012455 |
| 351 | KRT86 | 12 | ENSG00000170442 |
| 352 | CYP26C1 | 10 | NM_183374 |
| 353 | GYPC | 2 | NM_016815 |
| | | | NM_002101 |
| 354 | DIDO1 | 20 | NM_033081 |
| 355 | EEF1A2 | 20 | NM_001958 |
| 356 | EMX2OS | 10 | NR_002791 |
| 357 | GDF7 | 2 | NM_182828 |
| 358 | JSRP1 | 19 | NM_144616 |
| 359 | SMPD5 | 8 | ENSG00000204791 |
| 360 | MDFI | 6 | NM_001300804 |
| 361 | MPZ | 1 | NM_001315491 |
| 362 | VILL | 3 | NM_001385039 |
| 363 | GATA2_6370 | 3 | NM_001145662 |
| | | | NM_032638 |
| | | | NM_001145661 |
| 364 | SQSTM1_3864 | 5 | NM_003900 |
| | | | NM_001142299 |
| | | | NM_001142298 |

### Example 3

From the two groups of markers in Examples 1 and 2, 25 candidate markers were chosen for a validation study with independent cases and controls (Table 3). Methylation-specific PCR assays were developed from the DMR sequences and tested on tissue samples. Short amplicon primers (<150 bp) were designed to target the most discriminant CpGs within a DMR and tested on analytical controls to ensure that fully methylated fragments amplified robustly and in a linear fashion; that unmethylated and/or unconverted fragments did not amplify. Tissue samples for 82 EC (16 serous, 18 carcinosarcoma, 7 clear cell, 17 endometrioid grade 1/2, 24 endometrioid grade 3), 82° C. (36 serous, 21 clear cell, 4 mucinous, 21 endometrioid), and 64 CC (36 squamous cell, 28 adenocarcinoma) were compared to controls of benign epithelium (29 cervicovaginal, 29 fallopian tube, 14 benign endometrial tissues). As shown in Table 3, while CDO1 and DLGAP1 discriminated any cancer type from benign control tissue, gynecological cancer specificity was evident for most MDMs.

TABLE 3
Candidate markers chosen for validation (OC: ovarian cancer; Ser
OC: serous ovarian cancer; clear cell OC: clear cell ovarian
cancer; endo OC: endometrioid ovarian cancer; muc OC: mucinous
ovarian cancer; CC: cervical cancer; Ad CC: adenocarcinoma cervical
cancer; sq CC: squamous cervical cancer; EC: endometrial cancer;
endo EC: endometrioid endometrial cancer; pan gyne: non-specific
gynecological cancer (e.g., OC, EC, and CC)).

| DMR | | |
|---|---|---|
| No. | Candidate Marker | Determined Specificity |
| 5 | AIM1 | Ser OC |
| 6 | AK5 | Ad CC |
| 19 | c18orf18 | EC |
| 263 | CDO1 | pan gyne |
| 268 | DLGAP1 | pan gyne |
| 50 | ELMOD1 | Ad CC |
| 11 | FKBP11 | EC |
| 62 | FLOT1 | Ser OC |
| 65 | GAL3ST2 | Ser OC |
| 99 | LRRC41 | Clear cell EC, Clear cell OC |
| 102 | LYPLAL1 | EC, OC |
| 108 | MAX.chr11.3750 | Endo OC |
| 144 | MLH1_4513 | Clear cell EC |
| 160 | NR3C1_2223 | Endo EC |
| 172 | PISD | Clear cell OC |
| 182 | RABC3 | CC |
| 183 | RAI1 | Muc OC |
| 212 | TERC | EC |
| 218 | TRPC3 | Ad CC |
| 233 | ZIC2 | Clear cell OC |
| 234 | ZMIZ1 | Muc OC |
| 240 | ZNF480 | Ad CC |
| 242 | ZNF491 | Sq CC |
| 244 | ZNF610 | Sq CC |
| 249 | ZNF91 | Sq CC |

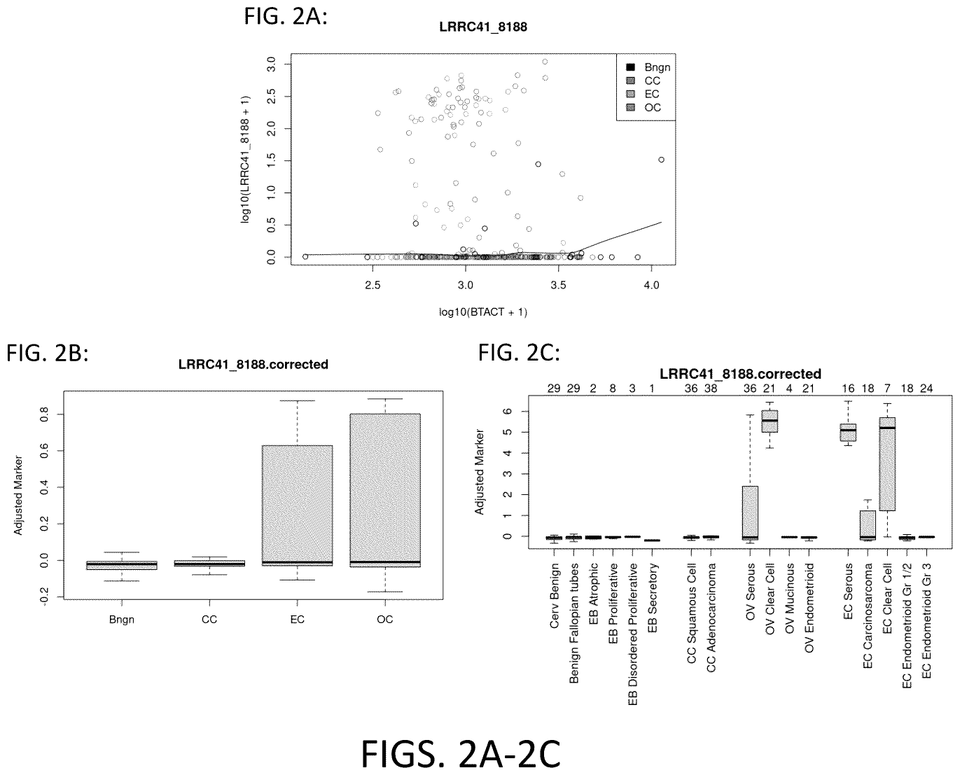

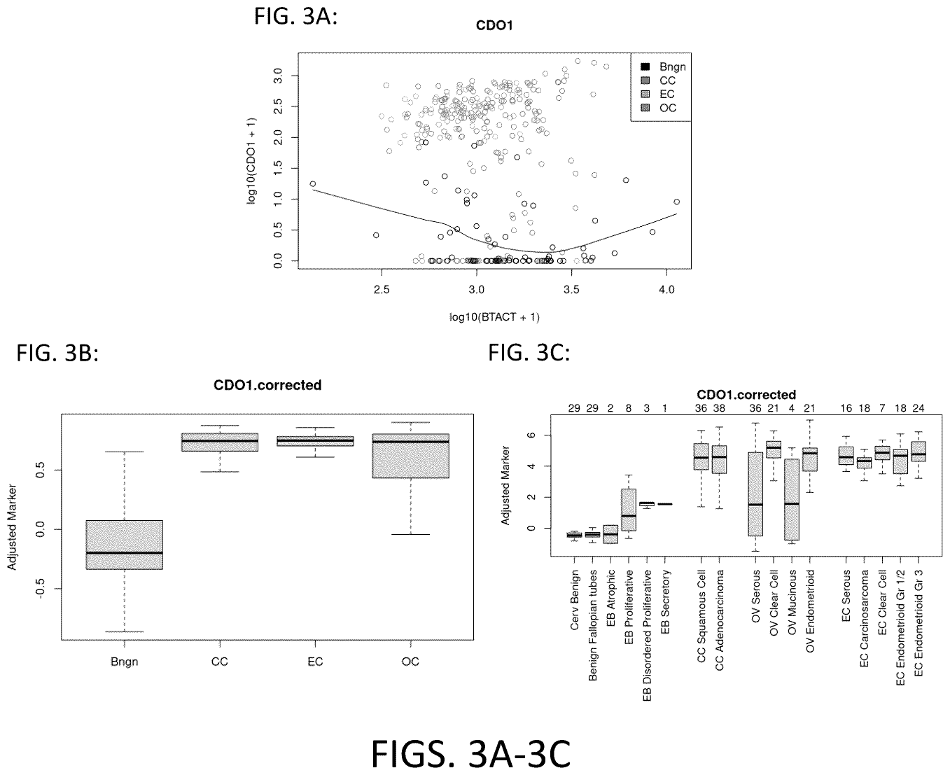

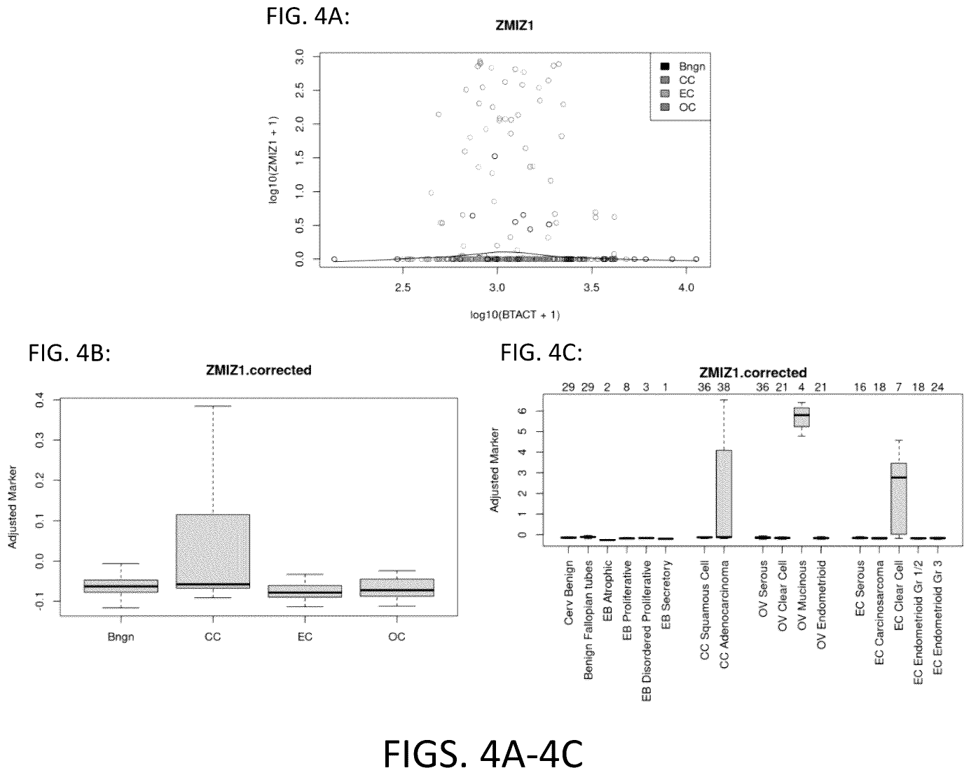

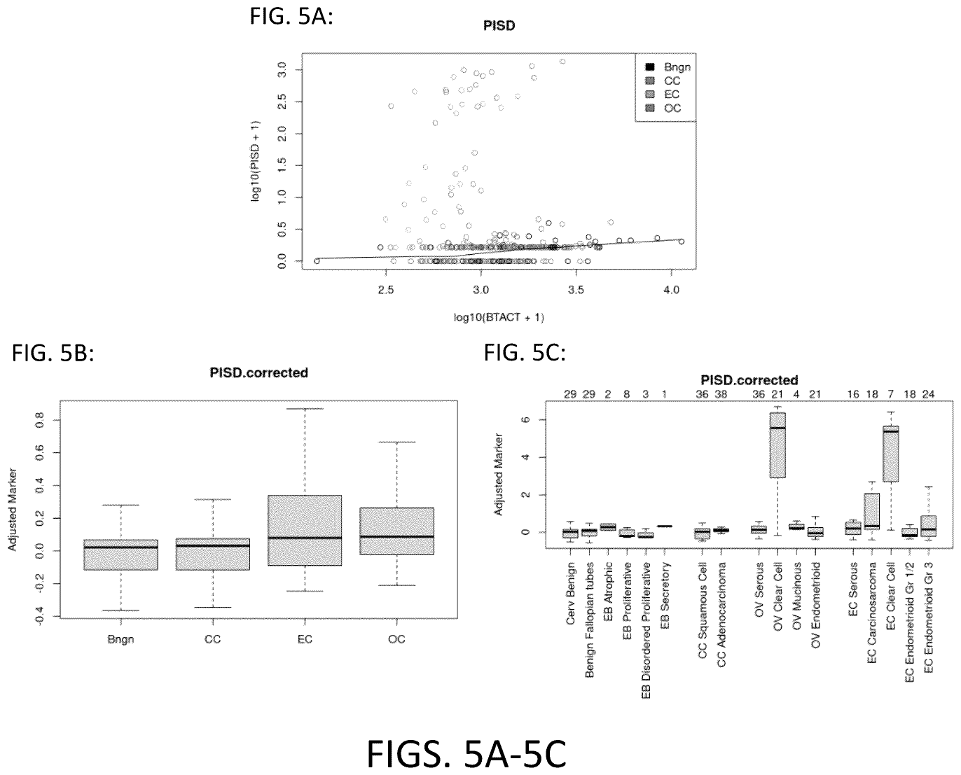

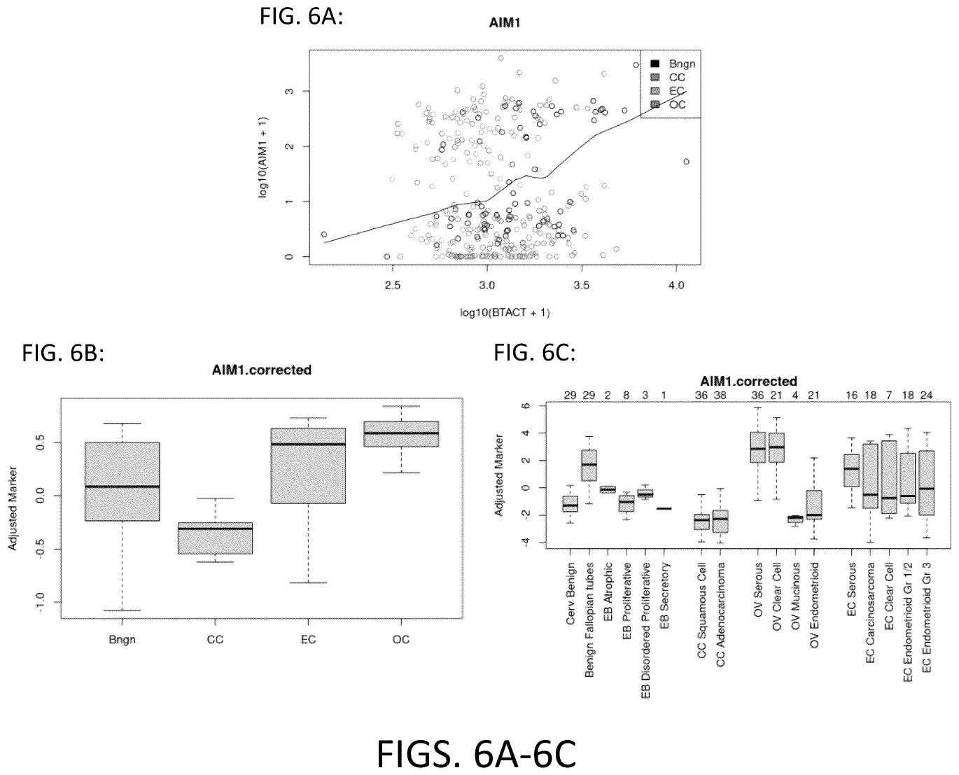

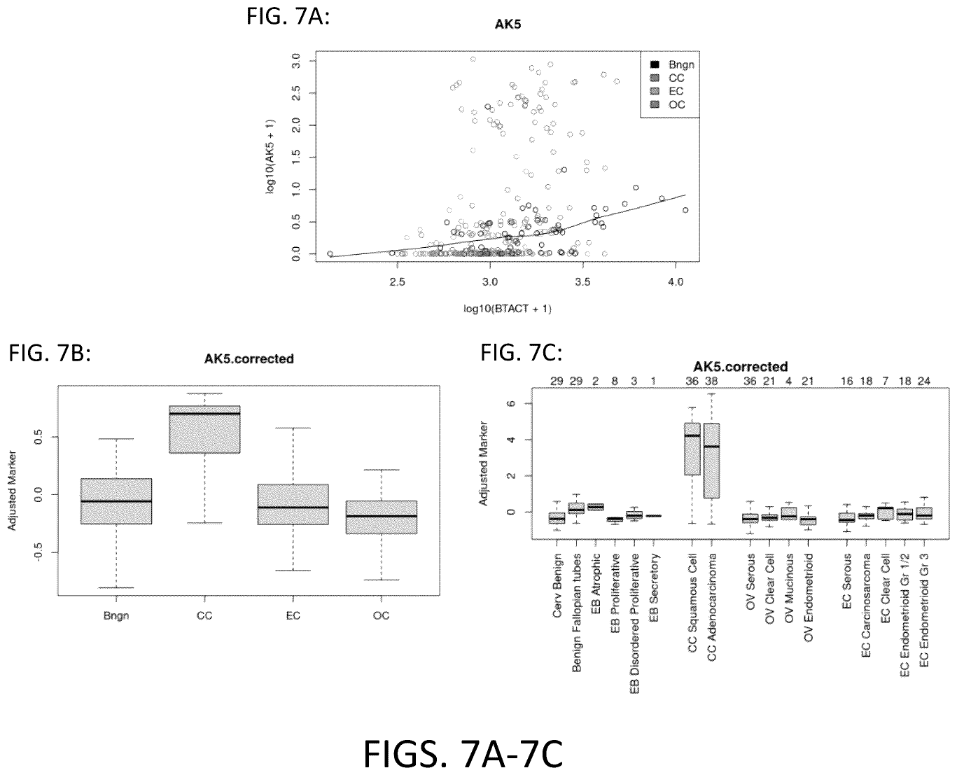

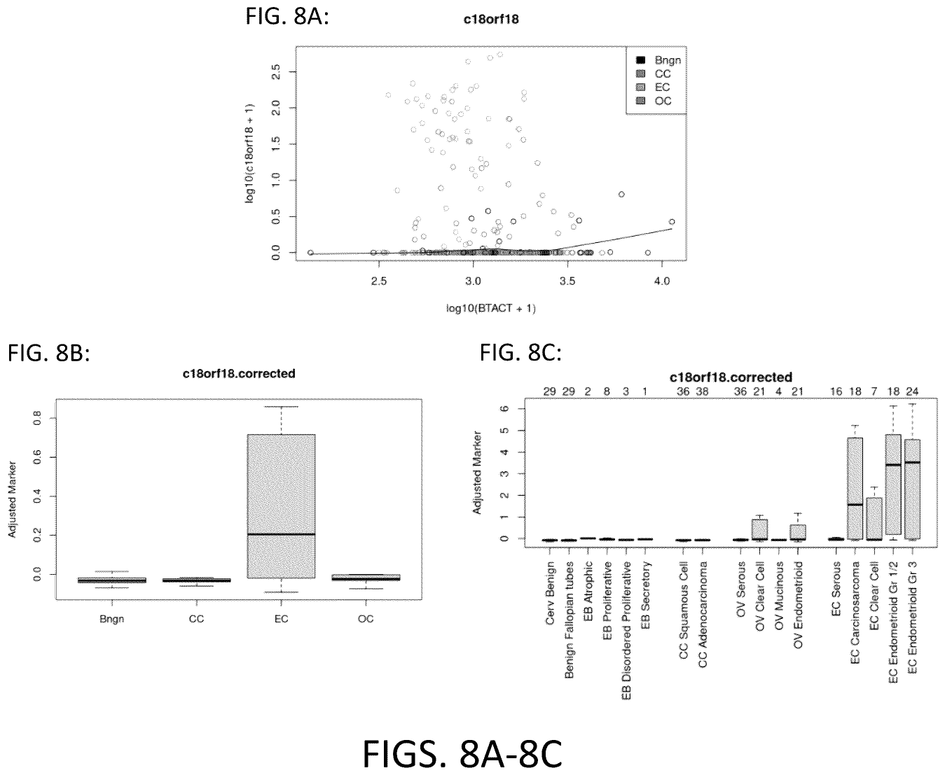

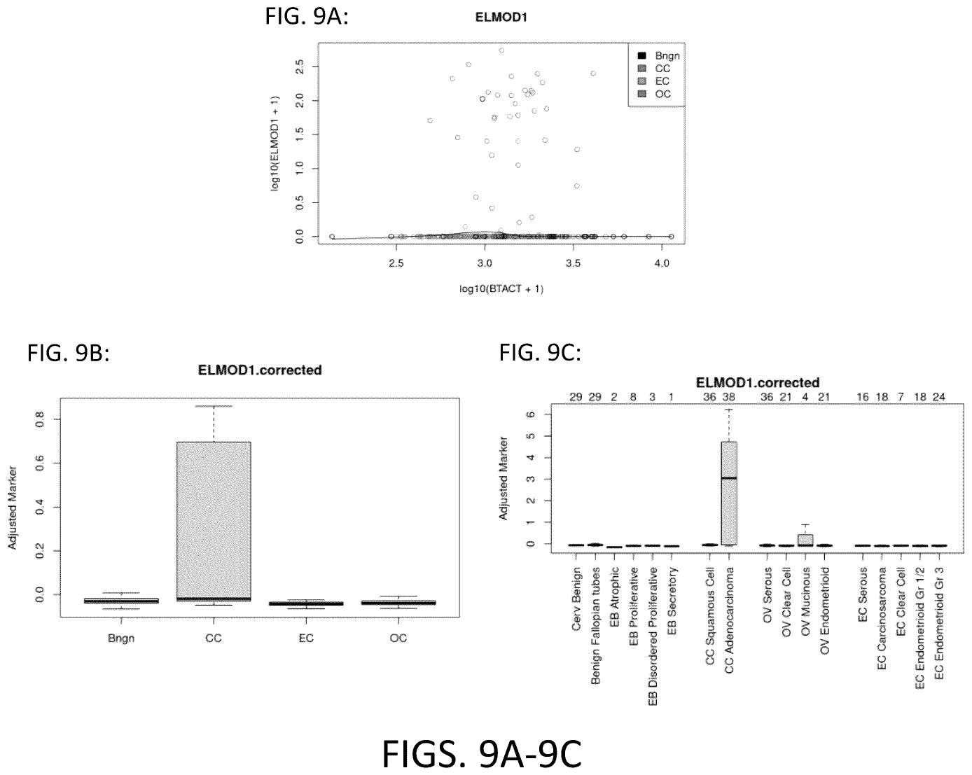

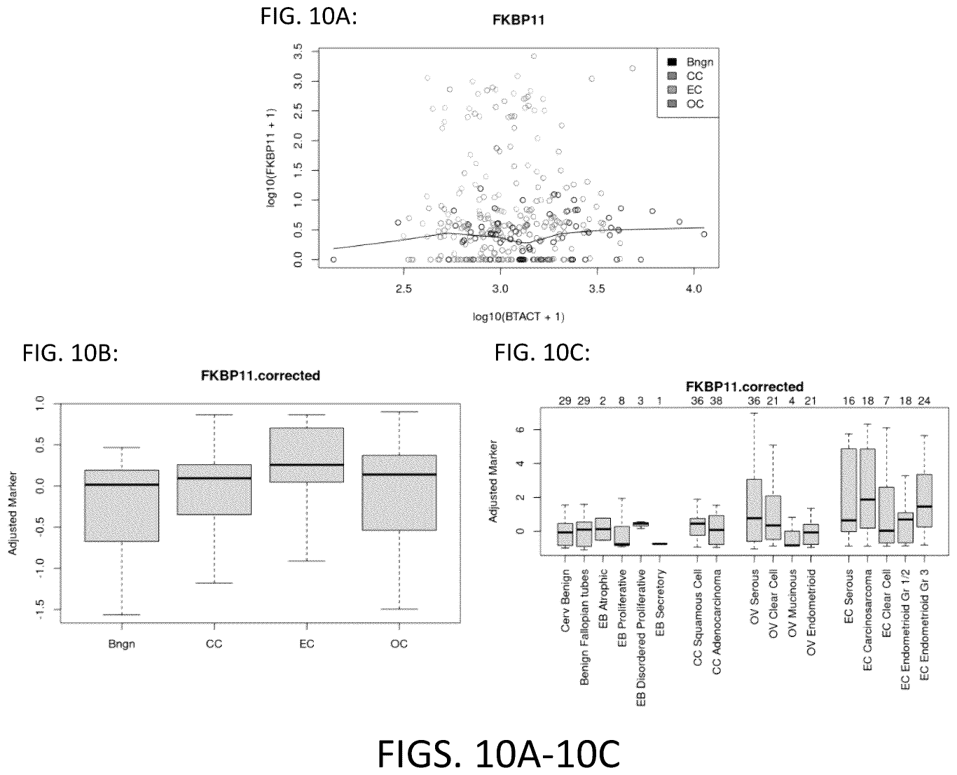

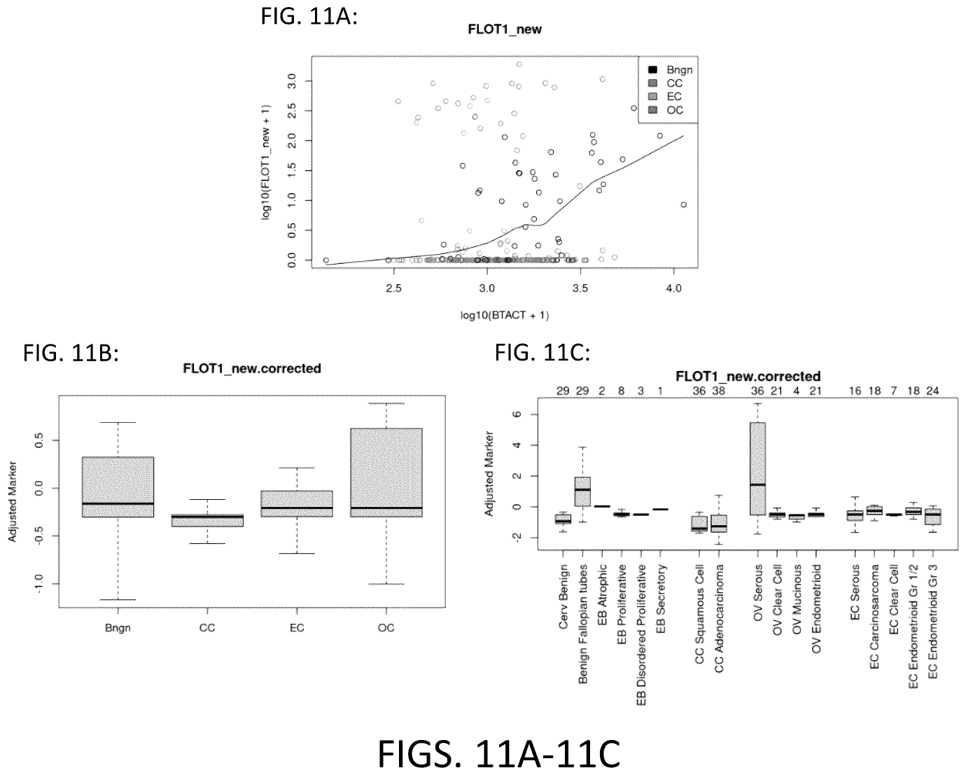

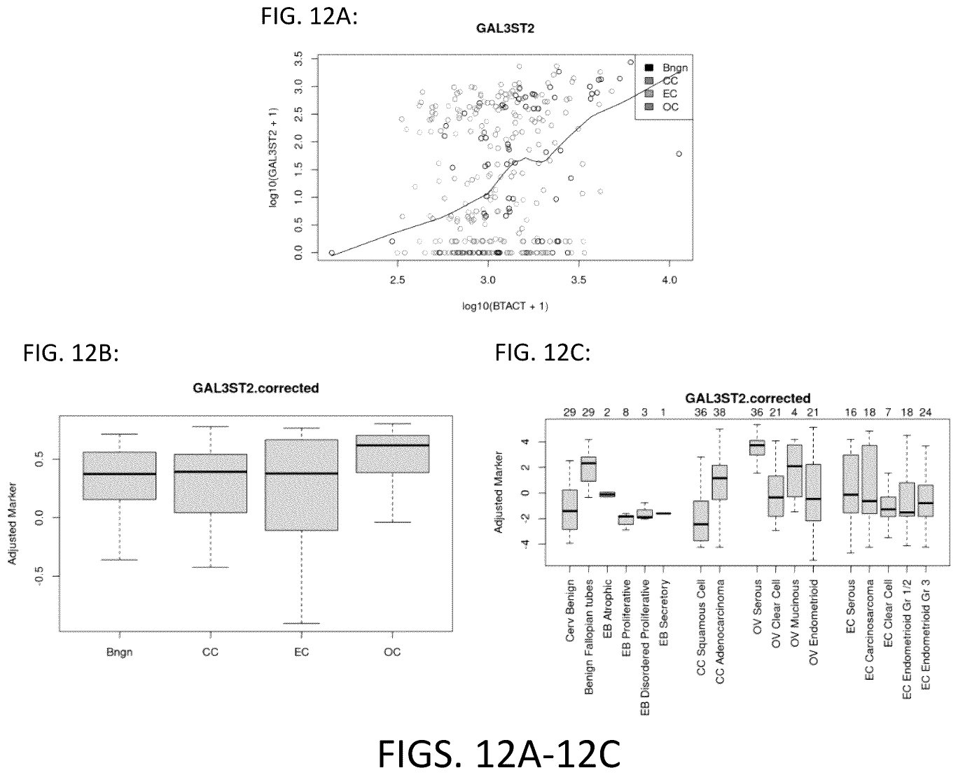

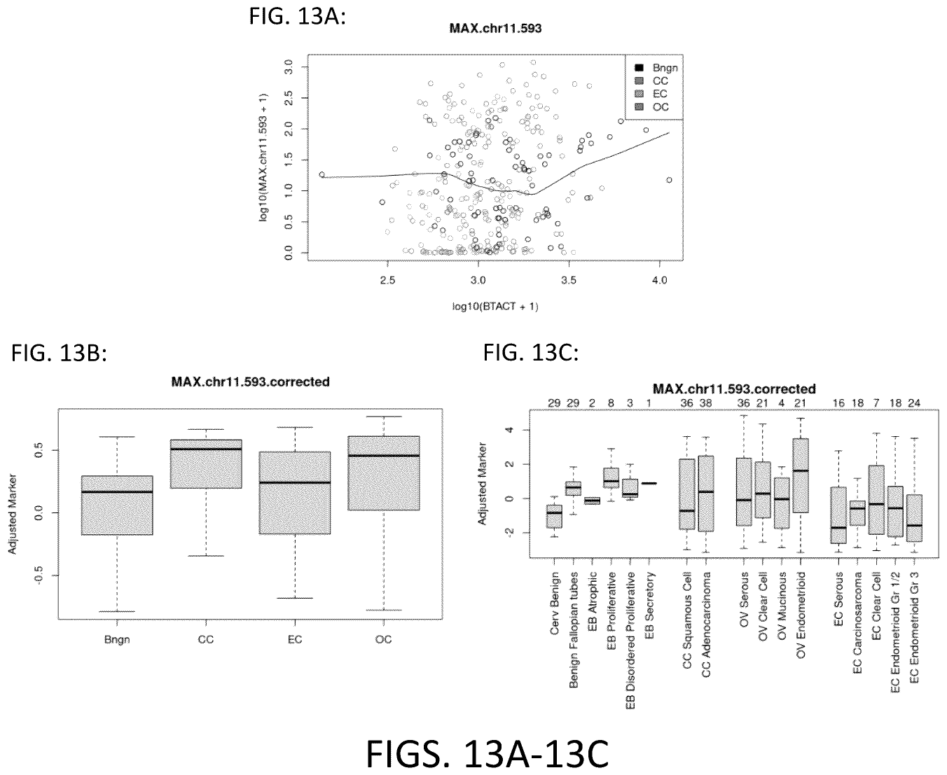

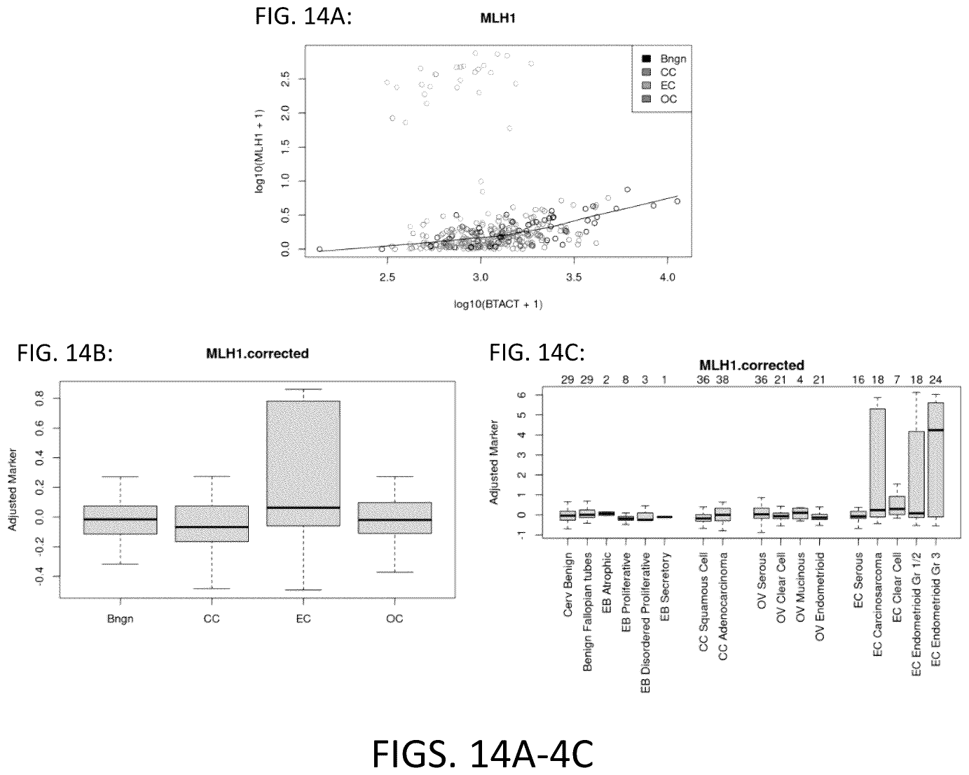

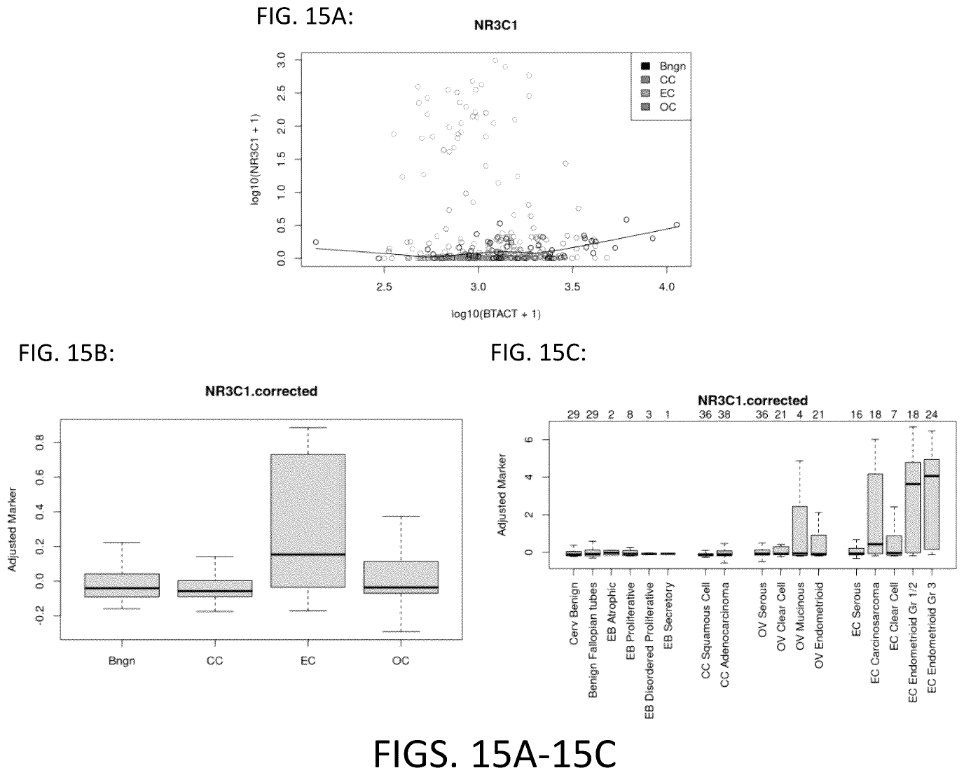

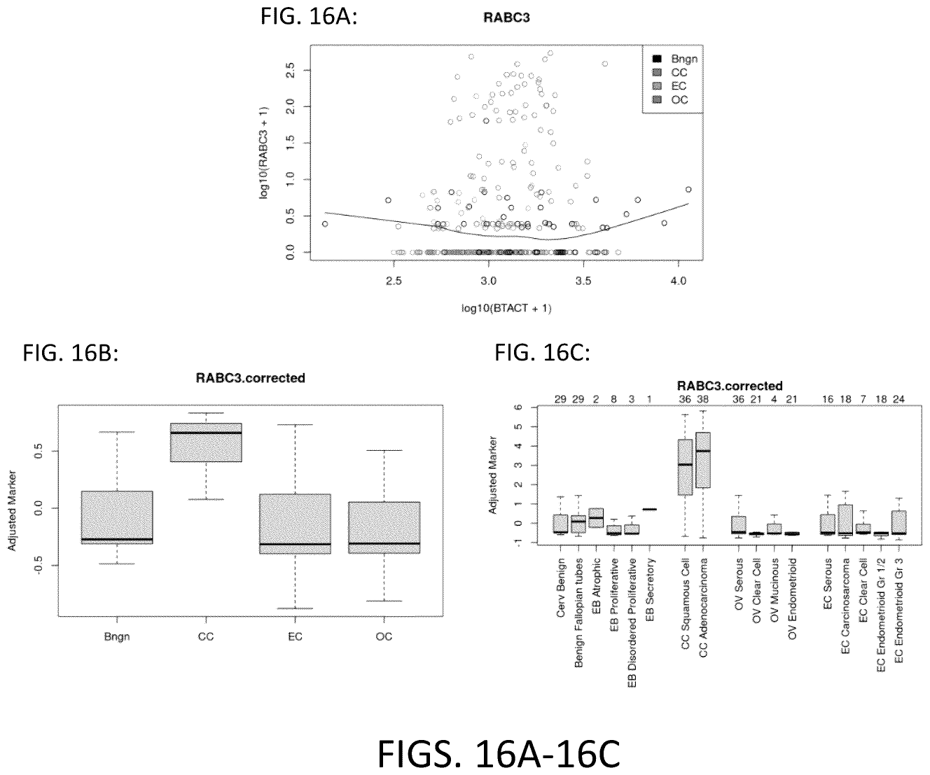

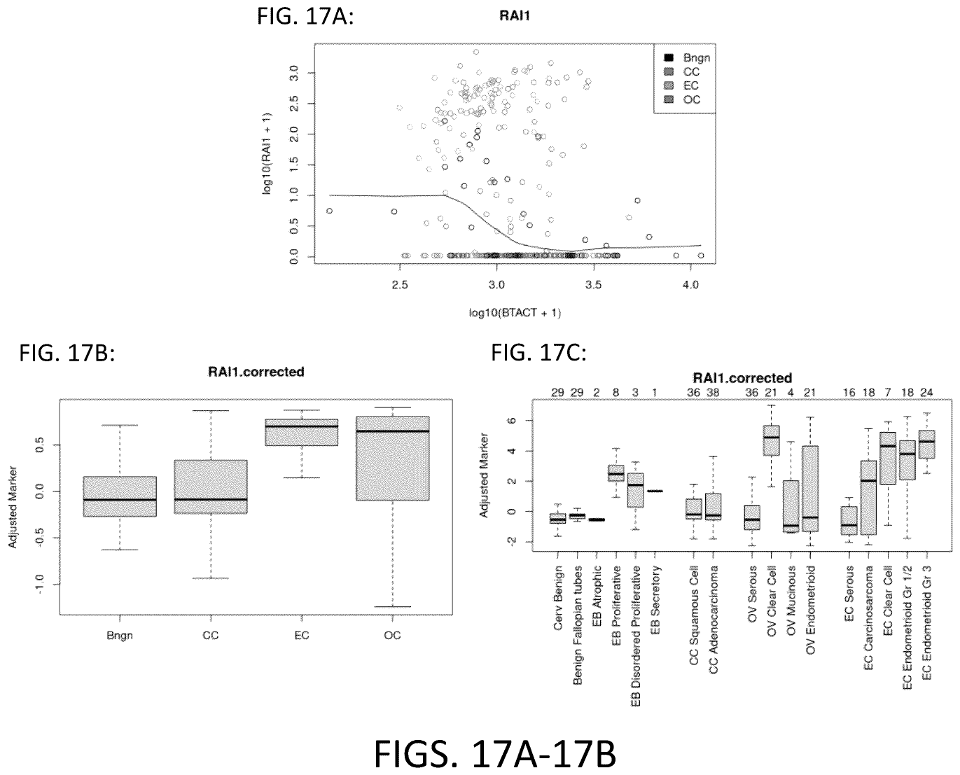

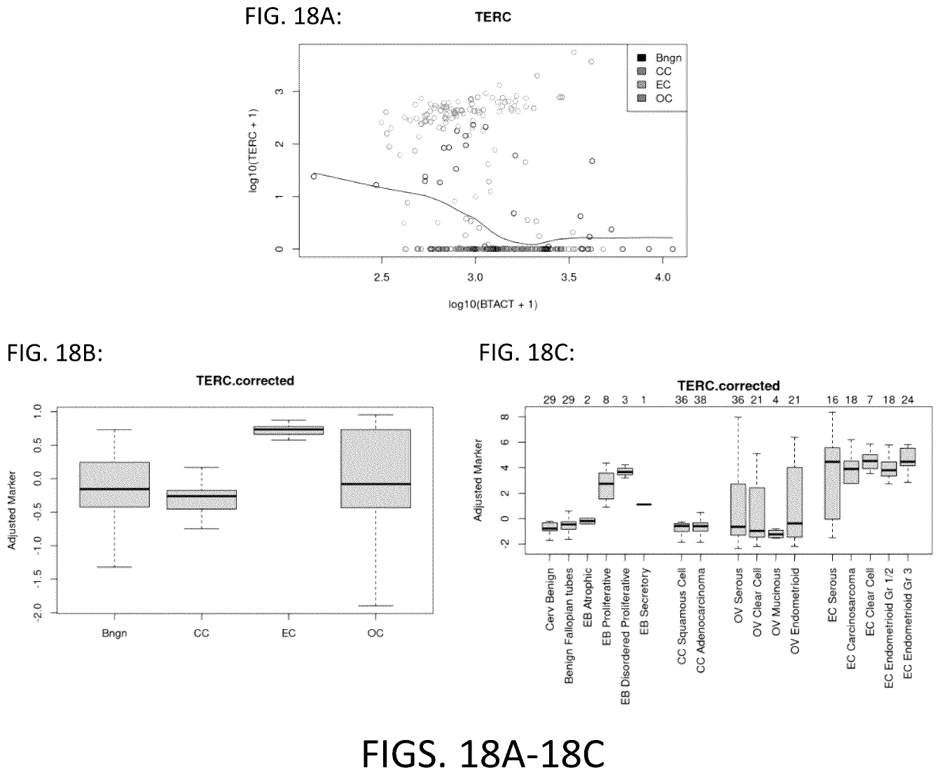

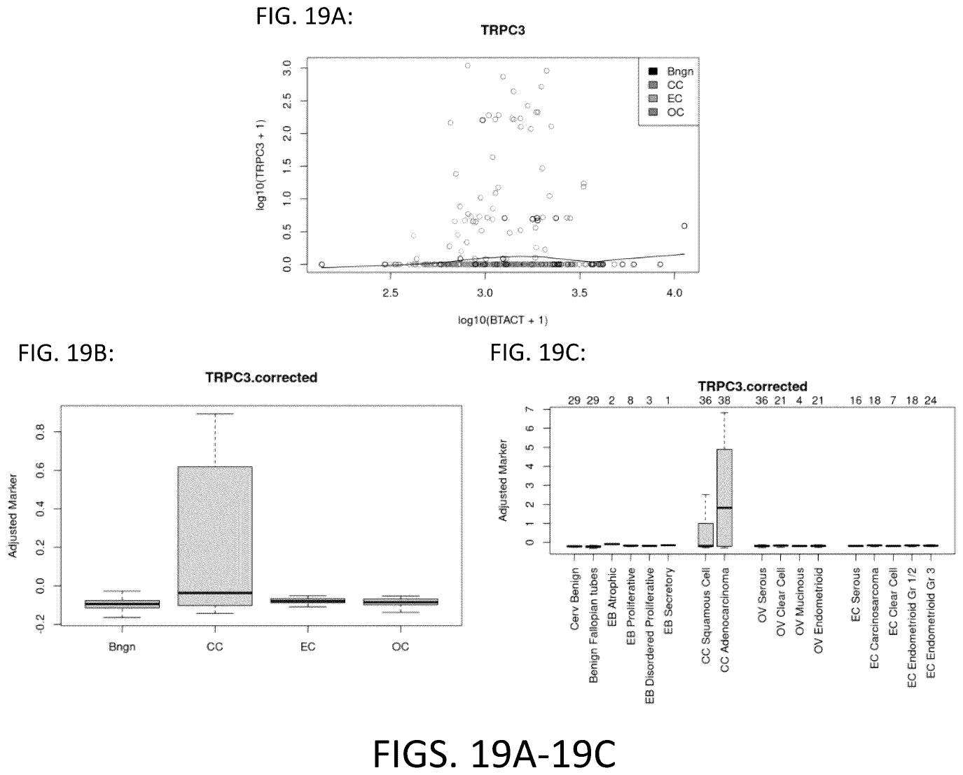

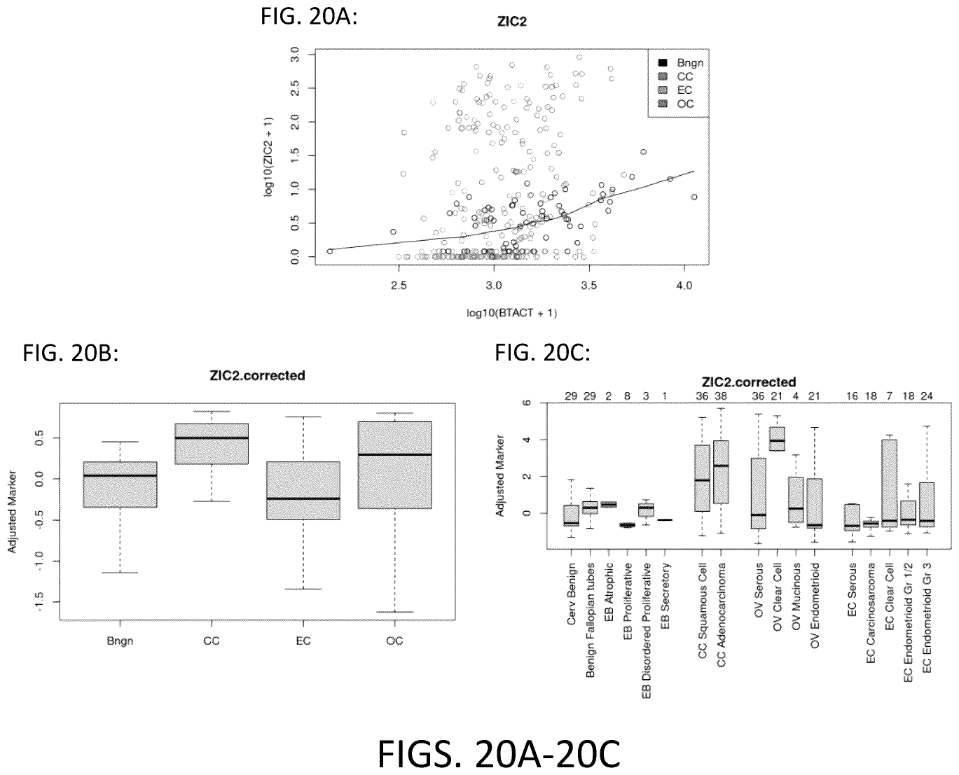

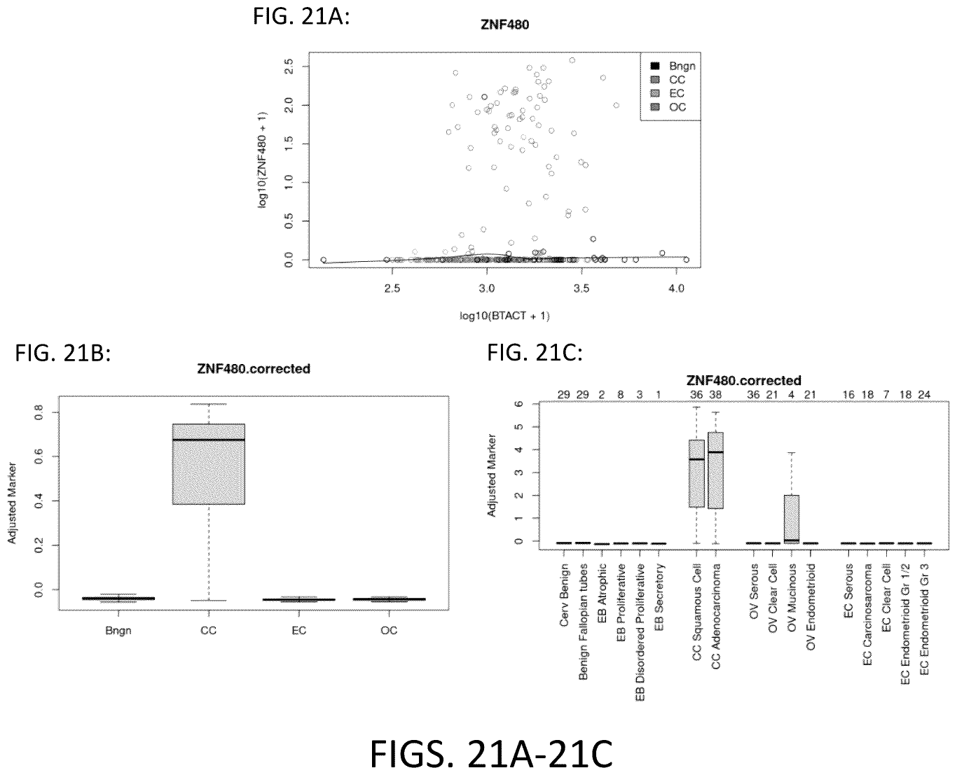

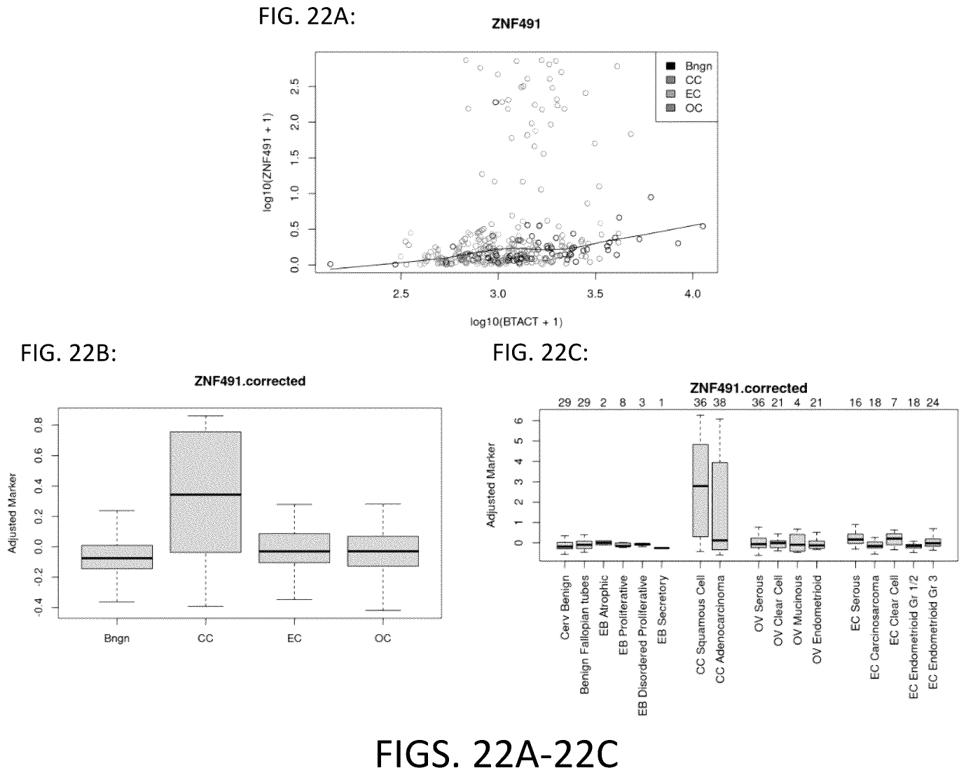

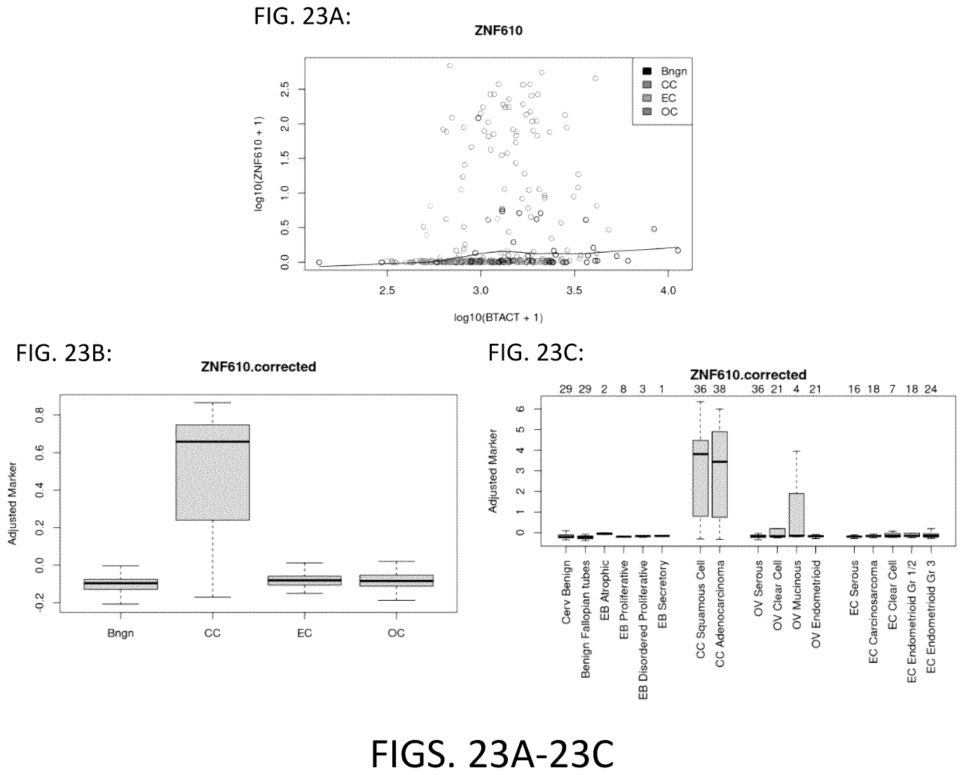

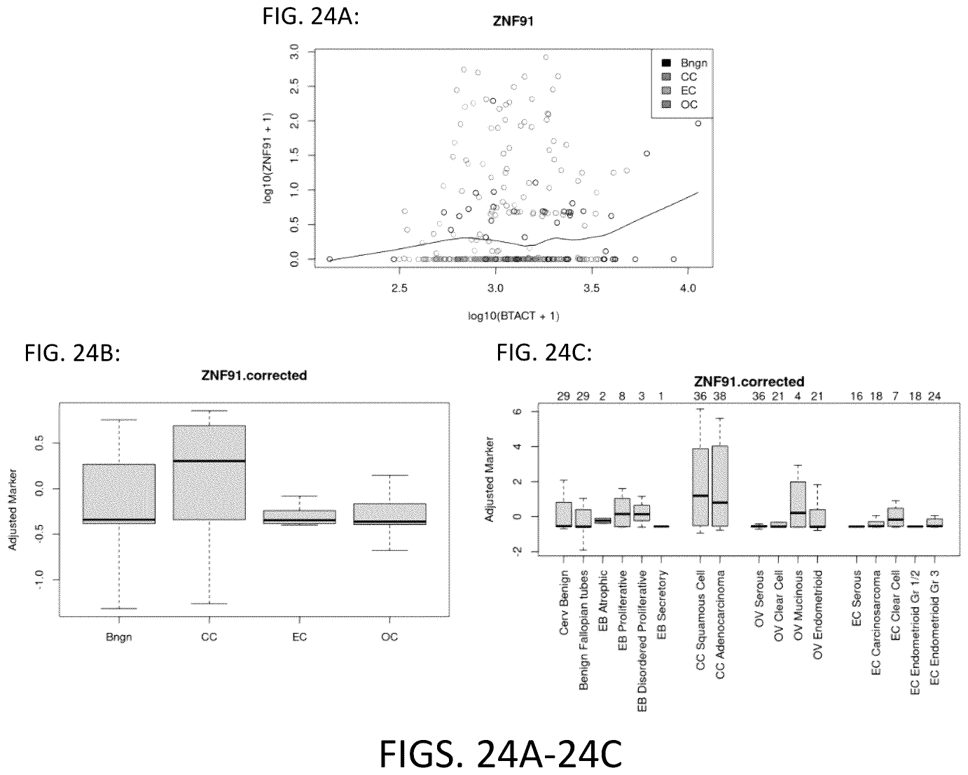

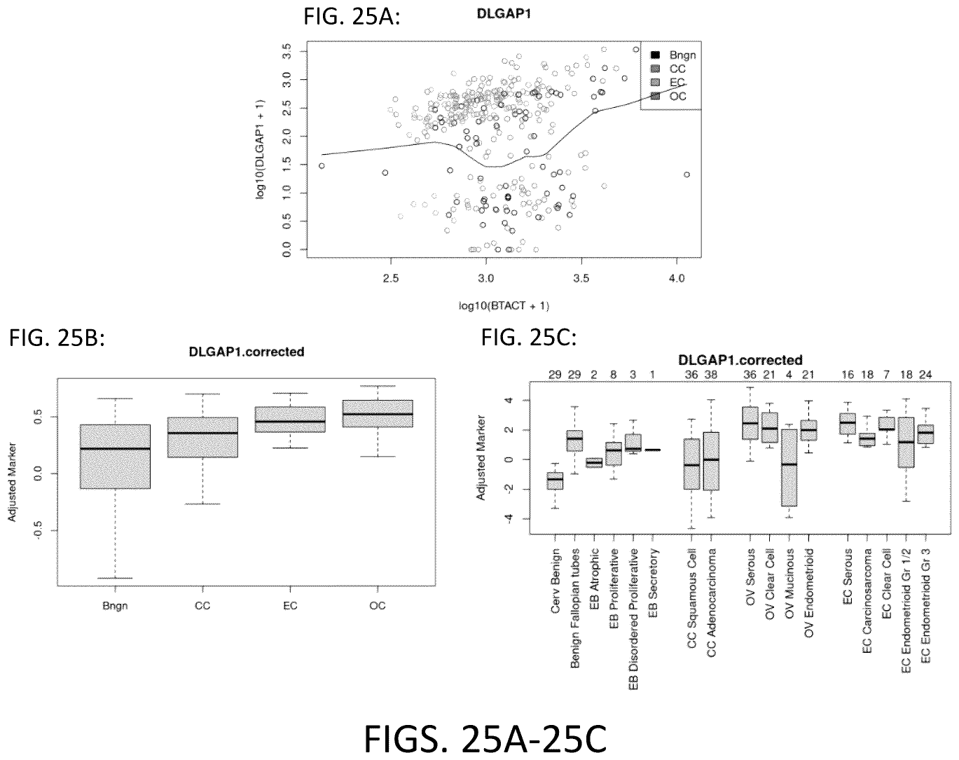

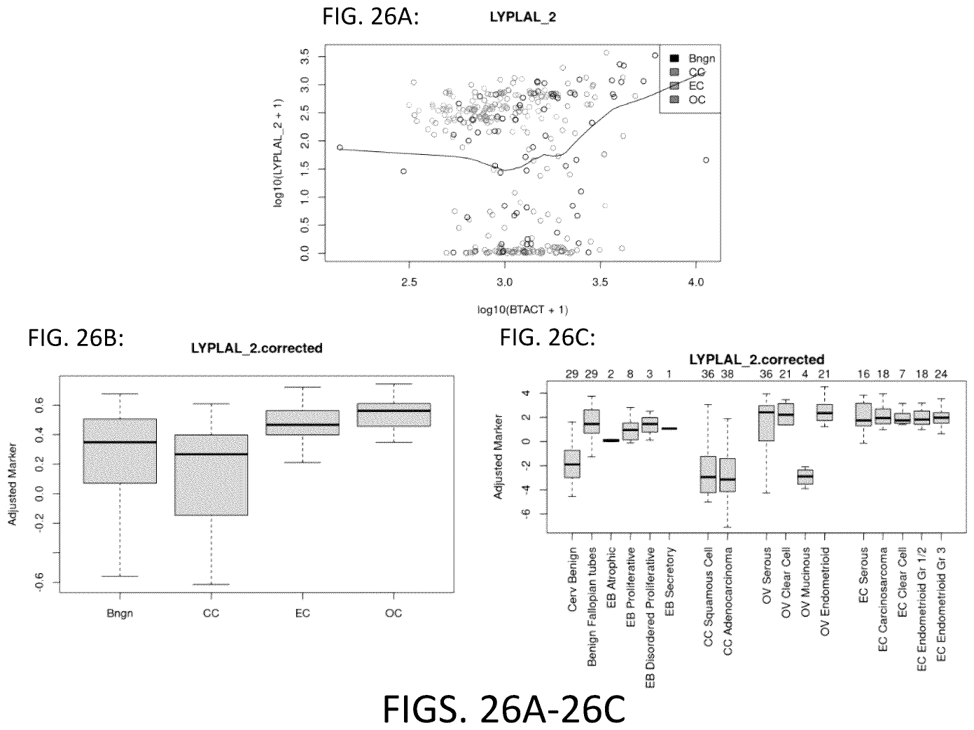

Additionally, as shown in FIGS. 2-26, representative data of calibration plots, adjusted boxplots, and adjusted boxplots by subtype are provided for each of the 25 candidate MDMs identified. Taken together, whole methylome sequencing, stringent filtering criteria, and biological validation of gynecological cancers yielded candidate MDMs for site specific and universal detection of gynecological cancers.

### Example 4

DNA methylation is an early event in endometrial cancer (EC) development and may have utility in EC detection. One of the most promising sample types for a clinical test is vaginal fluid from a tampon or similar collection device. A whole methylome NGS study was previously performed, followed by validation on independent tissues to identify discriminant EC-associated methylated DNA marker (MDM) candidates, which were subsequently tested in self-collected tampon samples from women with and without EC. In this example, an additional round of testing was conducted on a tampon sample subset with several new MDMs and a panel of novel epithelial reference assays which provide a measure of total epithelial exfoliation.

Briefly, an earlier reduced representation bisulfite sequencing (RRBS) study, which included DNA from frozen EC, benign endometrium (BE), benign cervicovaginal (BCV) tissues, and benign buffy coat samples, was reanalyzed to identify epithelial reference genes and several new EC MDMs. Candidate reference markers were selected based on receiver operating characteristic (ROC) discrimination, methylation level fold-change, methylation differentials, and p-values of all three epithelial tissue types vs the buffy coat (leukocyte) samples. EC MDMs were selected by the same criteria but comparing EC tissues to the other three sample types. Several other previously identified EC MDMs were also selected for vaginal fluid testing. Quantitative methylation specific PCR (qMSP) assays were developed and tested on 50 women ≥45 years of age with abnormal uterine bleeding (AUB) or postmenopausal bleeding (PMB) or any age with biopsy-proven EC self-collected vaginal fluid using a tampon prior to clinically indicated endometrial sampling or hysterectomy. Cases included 25 women with biopsy proven EC and 25 controls with benign biopsy.

Four candidate epithelial reference markers were selected from the earlier tissue RRBS data, FNBP1, NCOR2, and two regions associated with S1PR4. Methylation in EC, EB, and BCV tissues was consistent, concordant (all CpGs) and robust (>50%). Leukocyte methylation, conversely, was <1%. Two new EC MDMs, GYPC and CYP26C1, met the cancer-specific criteria in the RRBS data (AUC >0.85; absolute average CpG methylation >20% in ECs; methylation fold-change ratio (cases/controls)>10; p-value <0.001). These markers, along with LBX2, SPDYA, ZSCAN12, and TERC (forward and reverse strands) from Examples 1 and 2, were tested in the 50-sample tampon pilot. The four reference gene markers were strongly positive in all 25 cases and 25 controls, with the forward-strand S1PR4 assay demonstrating the most robust and consistent methylation between cases and controls. For the EC specific MDMs, TERC (forward strand) had the highest performance (AUC=0.88) and SPDYA the lowest (AUC=0.60).

Reanalysis of endometrial/cervical RRBS discovery data for reference epithelial markers yielded MDM candidates for vaginal fluid samples, and most of the tested EC-associated MDMs performed with promisingly high performance in tampon-collected vaginal fluid (Table 4).

TABLE 4
Methylated regions capable of distinguishing endometrial cancer
(EC) from benign tissue (genomic coordinates can be obtained
using the Human Feb. 2009 (GRCh37/hg19) Assembly).

| | | | |
|---|---|---|---|
| | Gene | Chromosome | |
| DMR | Annotation | No. | Accession No. |

EC DMRs:

| | | | |
|---|---|---|---|
| 91 | LBX2 | 2 | NM_001009812 |
| 203 | SPDYA | 2 | NM_182756; |
| | | | NM_001142634 |
| 212 | TERC | 3 | NR_001566 |
| 337 | ZSCAN12 | 6 | NR_028077; |
| | | | NM_001163391 |
| 341 | CYP26C1 | 10 | NM_183374 |
| 342 | GYPC | 2 | NM_016815; |
| | | | NM_002101 |

Reference DMRs:

| | | | |
|---|---|---|---|
| 343 | FNBP1 | 9 | NM_015033 |
| 344 | NCOR2 | 12 | NM_00107726; |
| | | | NM_006312 |
| 345 | S1PR4_8378 | 19 | NM_003775 |
| 346 | S1PR4_9843 | 19 | NM_003775 |

### Example 5

Early detection and treatment of endometrial cancer (EC) portends an excellent prognosis with surgery alone often being curative, especially in the setting of stage IA disease. However, presentation of EC at advanced stages most often requires multimodal therapy and oncologic outcomes are less favorable. Accordingly, investigations were conducted to broaden the repertoire of current candidate methylated DNA markers (MDMs) for EC using methylome sequencing discovery and independent sample validation experiments, which included both the more common endometrioid histology and the less common, more aggressive EC histologies. The performance of these novel EC MDMs were tested in vaginal fluid obtained via patient self-collected tampons from women presenting with perimenopausal AUB, PMB, or a new diagnosis of biopsy-proven EC.

This study was performed in three phases. First, tissue-based discovery of methylated DNA markers (MDMs) was performed using reduced representation bisulfite sequencing (RRBS) on DNA extracted from frozen EC and benign tissues. Second, biological validation of EC-specific MDMs was performed using quantitative methylation specific PCR (qMSP) on DNA extracted from an independent group of formalin fixed paraffin embedded (FFPE) EC and benign endometrium (BE). The third phase involved clinical translation of MDM detection via qMSP in DNA extracted from vaginal fluid samples, obtained from women with EC, atypical endometrial hyperplasia (AEH), endometrial hyperplasia without atypia, or benign endometrium (BE) collected via patient self-collected intravaginal tampon.

Primary fresh frozen EC tissues were identified from a prospectively maintained EC biorepository of >1,500 frozen samples collected from consenting patients at the time of hysterectomy for EC or AEH. ECs included in the discovery phase represented the five most common EC histologies (grade 1/2 endometrioid, grade 3 endometrioid, serous, and clear cell carcinomas, and uterine carcinosarcoma). Frozen tissue blocks were required to have at least 70% tumor purity for inclusion. Benign endometrium (BE) tissue was collected from consenting patients Pipelle or EndoSampler as an additional sample for research immediately following a clinically indicated office endometrial biopsy in women ≥45 years of age presenting for a workup of AUB or PMB. EC histologies and BE menstrual phases or atrophic endometrium were confirmed by a gynecologic pathologist. Benign cervicovaginal (BCV) squamous tissue was collected from both premenopausal and postmenopausal women undergoing hysterectomy for benign indications. BE and BCV tissues were fresh-frozen until DNA extraction. Buffy coats were collected from healthy control female donors without cancer who were current on cervical cancer screening and mammography. Women diagnosed with other cancers or who had received chemotherapy class drugs within the previous 5 years, had prior pelvic radiation, a synchronous cancer diagnosed at the time of EC, or a prior solid organ or bone marrow transplant were excluded. Clinical variables were abstracted from the electronic medical record for all included subjects.

An independent set of women with a new diagnosis of EC who underwent hysterectomy for initial treatment was identified for the biological validation cohort. Formalin-fixed paraffin embedded (FFPE) EC tissues representing the same histologies as the discovery cohort were included. Additionally, FFPE BE tissues from women who underwent hysterectomy for benign indications, frequency-matched by age, and FFPE endometrial hyperplasia without atypia and AEH tissues from women who underwent hysterectomy were obtained. All histologies were confirmed by a gynecologic pathologist (MES) who also selected the tissue block site for macro-dissection. Eligibility criteria were the same as in the discovery set.

Vaginal fluid was collected from two groups of women via self-placed tampon (tampon pilot). One group included women ≥45 years of age presenting to the Mayo Clinic Division of Gynecology for workup of AUB or PMB or without bleeding but postmenopausal and referred for evaluation of a thickened endometrial stripe (ES) on pelvic ultrasound. Women were excluded if they did not undergo a clinical endometrial sampling or if they had undergone endometrial sampling within the prior 3 months. Final clinical pathology diagnosis was utilized. EC on endometrial sampling or on hysterectomy pathology were included as EC cases. All final clinical diagnoses of AEH or endometrial hyperplasia without atypia were included for exploratory analyses, and any with benign endometrial sampling were eligible as BE controls. The other group was comprised of women ≥18 years of age with biopsy-proven EC or AEH presenting to the Mayo Clinic Division of Gynecologic Oncology Surgery for clinically indicated hysterectomy. Eligibility criteria were the same as in the discovery and biological validation cohorts.

Following verification of diagnosis and tissue block selection by one of the study gynecologic pathologists (JKS, SEK), frozen EC and BCV tissue embedded in optimal cutting temperature (OCT) compound underwent microtome cutting to provide ten 10-micron scrolls. BE whole frozen tissue samples, collected by 1 or 2 passes with an office biopsy Pipelle or EndoSampler, were used. Genomic DNA was purified from tissue and buffy coat specimens with the DNeasy Blood and Tissue protocol and QIAamp DNA blood protocol (Qiagen, Valencia, CA), respectively. DNA was re-purified with AMPure XP beads (Beckman-Coulter, Brea CA) and quantified by PicoGreen (Thermo-Fisher, Waltham MA). DNA quality was assessed using real time quantitative PCR. RRBS libraries were prepared. Briefly, 300 ng of DNA was 1) digested with MspI, 2) ligated to methylated sequencing adaptors, 3) treated with sodium bisulfite (Epitect Bisulfite protocol, Qiagen), 4) amplification enriched with adapter specific primers, and 5) size selected (160-280 bp) using AMPure beads to remove primer-dimers and larger CpG sparse regions. Libraries were sequenced using the Illumina HiSeq 2500 instrument (Illumina, San Diego CA) at the Mayo Clinic Medical Genomics Facility. Candidate genomic differentially methylated regions (DMRs) were selected as described below.

Quantitative methylation specific PCR assays were developed from the CpG methylation signatures of selected DMRs. Primers were designed using MethPrimer to target the bisulfite-modified sequences for each gene identified, as well as a CpG-free reference region within the β-actin gene. Primers were quality control checked on 20 ng (6250 genome equivalents) of positive and negative methylation controls. DNA was bisulfite converted using the EZ-96 DNA Methylation kit (Zymo Research, Irvine CA) and amplified using SYBR Green detection on Roche 480 LightCyclers (Roche, Basel Switzerland). Serially diluted universally methylated DNA samples were utilized as positive control standards, and negative controls included bisulfite converted and unconverted leukocyte-derived genomic DNA and converted whole genome amplified (unmethylated) DNA. MDM results were normalized to β-actin. Assay performance was verified using the discovery cohort samples. Markers that performed sub-optimally compared to the RRBS results and cut-offs (described below) were not considered further.

MDMs were then tested using qMSP on DNA extracted from independent FFPE EC and BE tissues. These MDMs were also tested in AEH and endometrial hyperplasia without atypia tissues. Following histologic verification and selection of macrodissection sites most representative of the diagnosis by a study gynecologic pathologist (MES), tissue blocks underwent macrodissection using a 1 mm or 2 mm core punch. DNA was purified using the Qiagen QIAmp FFPE DNA Tissue Kit (part #56404) and bisulfite converted as described above. Samples were blinded, randomized, and assayed by qMSP, as above.

Consented women in both groups within the tampon pilot self-placed a regular sized, unscented Playtex® tampon to collect vaginal fluid. Those enrolled in the group presenting for workup of AUB, PMB or thickened ES placed the tampon in the clinic prior to their gynecology consult and removed the tampon before clinically indicated pelvic examination and endometrial sampling. Those in the group that presented with a biopsy-proven EC or AEH placed the tampon in the preoperative area on the day of their hysterectomy and the tampon was removed in the operating room. Intravaginal tampon dwell time was recorded for both groups.

After removal, each tampon was placed in a 50 mL conical tube containing sterile PBS buffer, centrifuged through a mesh filter, and separated into pellet and supernatant portions which were stored at −80 C until DNA extraction. Approximately half-way through prospective enrollment to the tampon pilot, 50 mM EDTA was added to the PBS buffer to enhance DNA recovery and reduce nuclease degradation. Tampon pellet DNA was extracted using the High Pure Viral Nucleic Acid Kit (Roche, Basel Switzerland) and quantified using a Qubit Fluorometer (Invitrogen, Walther MA). DNA was bisulfite converted and assayed by qMSP on selected MDMs as described above with β-actin as the reference gene.

For discovery, a previously published approach was used. Briefly, Streamlined Analysis and Annotation Pipeline for RRBS (SAAP-RRBS), a Mayo Clinic in-house analysis software package, was used for quality scoring, sequence alignment, annotation to a University of California Santa Cruz reference genome, and differential analysis of DMRs. Candidate CpGs were excluded if the coverage of data within each sample group was <50%. CpG islands are typically biochemically defined by an observed to expected CpG ratio >0.6. However, for this model, DMRs were created based on the distance between CpG site locations for each chromosome with regions containing five or fewer CpGs excluded. DMRs were then selected for a background methylation ratio in the benign controls (BE, BCV, and buffy coat) of <2% and then ranked by AUCs for EC histologies referent to benign controls. Statistical significance was determined by over-dispersed logistic regression of the methylation percentage per candidate DMR, based on read counts. To account for varying read depths across individual subjects, an over-dispersed logistic regression model was used, where the dispersion parameter was estimated using a Pearson Chi-square statistic of the residuals from fitted model. Candidate genomic DMRs were ranked and selected for further testing according to their significance level, AUC, and fold-change difference between ECs and benign controls (BE, BCV, and buffy coat). Sample size estimates for the discovery are based on methods previously described.

A secondary DMR analysis was undertaken to identify endometrium specific MDMs methylated in both EC and BE and unmethylated in BCV and buffy coat.

For independent tissue validation, sample sizes were chosen to increase precision (minimizing the widths of 95% confidence intervals (95% CIs)) of sensitivity and specificity. With an assumed specificity of 95%, a control set of 29 would provide a 95% CI no wider than ±10%. To achieve a 95% CI that was no wider than ±7% for a target sensitivity of 90%, a minimum of 84 samples was required. Distributions of individual markers were examined using boxplots and marker intensity maps. AUC values were generated for each marker to assess accuracy. Random forest (rForest) models were used to generate the predicted probability of a sample representing an EC case. Random forest uses 500 randomized unique training and test sets from a bootstrap selection (approximately 2/3 for the training set and 1/3 for the test set) to perform cross-validation of randomized marker sets to generate 500 models. Accuracy error based on out-of-bag samples is then averaged over the 500 models. Marker selection was performed using the VSURF package in R, which uses random forests in three steps to: 1) eliminate predictors with least importance, 2) select all predictors that relate to the response variable, and 3) reduce redundancy in final marker selection.

For the tampon pilot, sample size estimates were defined to detect an AUC of 0.70 (AUC=0.50 represents chance). With 100 EC and 92 BE, there was greater than 90% power to detect this difference using a one-sided test at a 5% significance level. ECs were frequency matched to control BEs by subject menopausal status and tampon collection date.

All women in the clinic group that underwent evaluation for AUB, PMB or thickened ES and diagnosed with AEH or endometrial hyperplasia without atypia after tampon collection were included for exploratory analyses. Additionally, the following tampon pilot subanalyses were performed: 1) MDM sensitivity and specificity for EC when limited to vaginal fluid samples collected prior to endometrial sampling, a setting of presumed spontaneous endometrial DNA shedding, and 2) MDM performance specifically in PBS/EDTA buffered vaginal fluid samples.

RRBS was performed on 69 ECs (16 grade 1/2 endometrioid, 16 grade 3 endometrioid, 11 serous, and 11 clear cell carcinomas, and 15 uterine carcinosarcomas), 44 BE (14 proliferative, 18 disordered proliferative, and 12 atrophic), 18 BCV, and 18 buffy coat samples from healthy donor women. Clinicopathologic characteristics for discovery phase EC cases and BE controls are detailed in Table 5.

TABLE 5
Clinicopathologic characteristics of discovery
cohort EC cases and BE controls.

| | Endometrial Cancer | | Benign Endometrium | |
|---|---|---|---|---|
| | (EC) (N = 69) | | (BE) (N = 44) | |
| Age, years; median [IQR] | 68 | [60, 73] | 51.5 | [48, 57] |
| BMI, kg/m2; median [IQR] | 30.1 | [26.5, 36.9] | 26.1 | [22.2, 33.6] |
| Pregnancies; median [IQR] | 2 | [2, 4] | 2 | [1, 3] |
| Live Births; median [IQR] | 2 | [1.8, 4] | 2 | [1, 3] |
| Race; N (%) | | | | |
| White | 65 | (94.2%) | 43 | (97.7%) |
| Non-white | 0 | (0.0%) | 1 | (2.3%) |
| Unknown | 4 | (5.8%) | 0 | (0.0%) |
| Tobacco use; N (%) | | | | |
| Current | 8 | (11.8%) | 7 | (15.9%) |
| Previous | 14 | (20.6%) | 15 | (34.1%) |
| Never | 46 | (67.6%) | 21 | (47.7%) |
| Unknown | 0 | (0.0%) | 1 | (2.3%) |
| Menopausal Status; N (%) | | | | |
| Postmenopausal | 62 | (91.2%) | 19 | (43.2%) |
| Perimenopausal | 1 | (1.5%) | 9 | (20.5%) |
| Premenopausal | 5 | (7.4%) | 16 | (36.4%) |
| Unknown | 0 | (0.0%) | 0 | (0.0%) |
| Diabetes mellitus; N (%) | 11 | (15.9%) | 5 | (11.4%) |
| Hypertension; N (%) | 36 | (52.2%) | 9 | (20.5%) |
| Hyperlipidemia; N (%) | 25 | (36.2%) | 0 | (0%) |

| BE Histology | | | |
|---|---|---|---|
| Atrophic | — | 12 | (27.3%) |
| Proliferative | — | 14 | (31.8%) |
| Disordered Proliferative | — | 18 | (40.9%) |

| EC Histology; N (%) | | | |
|---|---|---|---|
| Grade 1/2 endometrioid | 16 | (23.1%) | — |
| Grade 3 endometrioid | 16 | (23.2%) | — |
| Serous | 11 | (15.9%) | — |
| Clear Cell | 11 | (15.9%) | — |
| Uterine carcinosarcoma | 15 | (21.7%) | — |
| EC Stage; N (%) | | | |
| I | 34 | (49.3%) | — |
| II | 5 | (7.2%) | — |
| III | 24 | (34.8%) | — |
| IV | 6 | (8.7%) | — |

Sequencing coverage depth across all the samples was approximately in the 40-50× range. On average, filtered Cs in the CpG context with at least 10× coverage (our minimum requirement for inclusion) averaged −1.7 million/sample. The DMR calling algorithm applied to multiple comparisons (all EC vs all controls, histologic EC subtype vs BE, histologic EC subtype vs BCV, etc.) yielded a total of 323 statistically significant DMRs. Imposing performance cut-offs (AUC >0.85; absolute average CpG methylation >20% in ECs; methylation fold-change ratio (cases/controls)>10; p-value <0.001) reduced the number of DMRs to 54. Targeted qMSP assays were constructed and tested for the 54 selected DMRs. Twenty-one targeted qMSP assays were subsequently discarded due to QC failures or inferior performance relative to the respective sequencing data and/or the cut-offs indicated above.

Independent tissue testing was performed on the remaining 33 MDMs. Samples included 141 ECs (34 grade 1/2 endometrioid, 31 grade 3 endometrioid, 27 serous, 19 clear cell, and 30 uterine carcinosarcomas), 112 BEs (35 secretory, 30 proliferative, 19 disordered proliferative, and 28 atrophic), 35 AEHs, and 24 endometrial hyperplasias without atypia. See Table 6 for clinicopathologic characteristics.

TABLE 6
Clinicopathologic characteristics of biological validation cohort EC cases, benign endometrium (BE)
controls, atypical endometrial hyperplasia (AEH), and endometrial hyperplasias without atypia.

| | | | Benign | | Endometrial | | | |
|---|---|---|---|---|---|---|---|---|
| | Endometrial | | Endometrium | | Hyperplasia | | | |
| | Cancer (EC) | | (BE) | | w/o Atypia | | AEH | |
| | (N = 141) | | (N = 112) | | (N = 24) | | (N = 35) | |
| Age, years; median [IQR] | 69 | [61, 77] | 47 | [42.8, 54] | 53.5 | [47.8, 58.5] | 57 | [46.5, 65.5] |
| BMI, kg/m2; median [IQR] | 31.2 | [26.5, 36.4] | 28.3 | [24.3, 33.2] | 34.1 | [26.2, 42.7] | 40.2 | [32.8, 48] |
| Pregnancies; median [IQR] | 3 | [2, 4] | 3 | [2, 4] | 1 | [0, 3] | 2 | [1.3, 3] |
| Live births; median [IQR] | 3 | [2, 4] | 2 | [2, 3] | 1 | [0, 3] | 2 | [1, 2] |
| Race; N (%) | | | | | | | | |
| White | 136 | (96.5%) | 108 | (96.4%) | 22 | (91.7%) | 35 | (100.0%) |
| Non-white | 1 | (0.7%) | 3 | (2.7%) | 1 | (4.2%) | 0 | (0.0%) |
| Unknown | 4 | (2.8%) | 1 | (0.9%) | 1 | (4.2%) | 0 | (0.0%) |
| Tobacco use; N (%) | | | | | | | | |
| Current | 10 | (7.1%) | 17 | (15.3%) | 3 | (12.5%) | 3 | (8.6%) |
| Previous | 32 | (22.7%) | 24 | (21.6%) | 4 | (16.7%) | 6 | (17.1%) |
| Never | 96 | (68.1%) | 70 | (63.1%) | 16 | (66.7%) | 25 | (71.4%) |
| Unknown | 3 | (2.1%) | 0 | (0.0%) | 1 | (4.2%) | 1 | (2.9%) |
| Menopausal status; N (%) | | | | | | | | |
| Postmenopausal | 134 | (95.7%) | 29 | (25.9%) | 13 | (54.2%) | 15 | (42.9%) |
| Perimenopausal | 3 | (2.1%) | 9 | (8.0%) | 3 | (12.5%) | 8 | (22.9%) |
| Premenopausal | 1 | (0.7%) | 72 | (64.3%) | 8 | (33.3%) | 10 | (28.6%) |
| Unknown | 2 | (1.4%) | 2 | (1.8%) | 0 | (0.0%) | 2 | (5.7%) |
| Diabetes mellitus; N (%) | 32 | (22.7%) | 0 | (0.0%) | 0 | (0.0%) | 0 | (0.0%) |
| Hypertension; N (%) | 78 | (55.3%) | 0 | (0.0%) | 1 | (100.0%) | 2 | (40.0%) |
| Hyperlipidemia; N (%) | 66 | (46.8%) | 0 | (0.0%) | 0 | (0.0%) | 1 | (20.0%) |

| BE Histology; N (%) | | | | | |
|---|---|---|---|---|---|
| Atrophic | — | 28 | (25.0%) | — | — |
| Secretory | — | 35 | (31.2%) | — | — |
| Proliferative | — | 30 | (26.8%) | — | — |
| Disordered Proliferative | — | 19 | (17.0%) | — | — |

| EC Histology; N (%) | | | | | |
|---|---|---|---|---|---|
| Grade 1/2 endometrioid | 34 | (24.1%) | — | — | — |
| Grade 3 endometrioid | 31 | (22.0%) | — | — | — |
| Serous | 27 | (19.1%) | — | — | — |
| Clear Cell | 19 | (13.5%) | — | — | — |
| Uterine carcinosarcoma | 30 | (21.3%) | — | — | — |
| EC Stage; N (%) | | | | | |
| I | 95 | (67.4%) | — | — | — |
| II | 3 | (2.1%) | — | — | — |
| III | 30 | (21.3%) | — | — | — |
| IV | 13 | (9.2%) | — | — | — |

Several MDMs (EMX2OS, CYTH2_4043, MPZ, NBPF24) demonstrated >0.85 AUCs in discriminating between EC (all histologic types combined) and BE, most were uniquely discriminatory between a specific EC histologic subtype and BE. For example, EEF1A2, which has an AUC of 0.59 when comparing all EC histologic subtypes to BE, discriminated clear cell EC from BE with an AUC of 0.91 (0.80-1). For analyses of clear cell EC versus BE, 14 of 33 MDMs had an AUC≥0.85. In contrast, only 5 of 33 MDMs distinguished carcinosarcoma versus BE with an AUC≥0.85. When assessing both EC histology-combined and histology-specific MDMs, only 10 MDMs fell below an AUC of 0.85 in all comparisons. As such, 23 MDMs had an AUC≥0.85 on either histology-combined or histology-specific analyses.

For the tampon pilot experiments, four MDM assays (CDH4, LYPLAL1, c17orf64, and KRT86) were developed for endometrium-specific DMRs (i.e., methylated in both EC and BE, but unmethylated in BCV tissues). In addition to these four, 24 MDMs were brought forward from biological validation for a total of 28 MDMs tested in the tampon pilot: CDH4, c17orf64, CYTH2_4043, DIDO1, EEF1A2, EMX2OS, GATA2_3670, GDF7, JSRP1, LRRC8D_8831, LRRC34, LRRC41, LYPLAL1, SMPD5, MAX.chr10.4460, MAX.chr12. 52652239-52652424, LINC2323, MDFI, MPZ, NBPF24, OBSCN, SEPT9, SFMBT2_0970, SQSTM1_3864, VILL, ZNF90, ZNF323, and ZNF506.

There were 100 women with EC, 92 with BE, 11 with AEH, and 25 with endometrial hyperplasia without atypia included in the tampon pilot. EC cases included 31 women who presented for AUB or PMB clinical evaluation whose EC was diagnosed after tampon collection and 69 women with biopsy-proven EC known prior to tampon collection. All women with BE, all but 3 with AEH, and all but 1 with endometrial hyperplasia without atypia were from the group of women with perimenopausal AUB or PMB and those subjects' tampons were collected prior to endometrial sampling. EC cases included 49 grade 1/2 endometrioid, 9 grade 3 endometrioid, 24 serous, 4 clear cell carcinomas, 9 uterine carcinosarcomas, and 5 mixed EC histologies. Clinicopathologic characteristics of the EC case, BE control, and AEH and endometrial hyperplasia without atypia groups are detailed in Table 7.

TABLE 7
Clinicopathologic characteristics of tampon pilot cohort EC cases, benign endometrium (BE) controls,
endometrial hyperplasia without atypia, and atypical endometrial hyperplasia (AEH).

| | | | | | | | Atypical | |
|---|---|---|---|---|---|---|---|---|
| | | | Benign | | Endometrial | | endometrial | |
| | Endometrial | | endometrium | | hyperplasia | | hyperplasia | |
| | cancer (EC) | | (BE) | | w/o atypia | | (AEH) | |
| | N = 100 | | N = 92 | | N = 25 | | N = 11 | |
| Age, years; median [IQR] | 64 | [58-69] | 63 | [55-68] | 54 | [52-60] | 62 | [57-65] |
| BMI, kg/m2; median [IQR] | 33.8 | (29.1, 38.9) | 29.9 | [24.8-37.7] | 31.6 | [25.8-38.8] | 41.8 | [37.2-46.3] |
| Pregnancies; median [IQR] | 3 | [2, 3] | 3 | [2, 4] | 2 | [2, 3] | 2 | [1.5, 3] |
| Live births; median [IQR] | 2 | [1, 3] | 2 | [1, 3] | 2 | [1.8, 2] | 2 | [2, 2.5] |
| Race; N (%) | | | | | | | | |
| White | 91 | (91%) | 88 | (95.7%) | 23 | (92%) | 11 | (100%) |
| Non-White | 3 | (3%) | 3 | (3.3%) | 1 | (4%) | 0 | (0%) |
| Unknown | 6 | (6%) | 1 | (1.1%) | 1 | (4%) | 0 | (0%) |
| Tobacco Use; N (%) | | | | | | | | |
| Current | 3 | (3%) | 1 | (1.1%) | 1 | (4%) | 0 | (0%) |
| Previous | 18 | (18%) | 27 | (29.3) | 6 | (24%) | 1 | (9.1%) |
| Never | 78 | (78%) | 63 | (68.5%) | 18 | (72%) | 10 | (90.9%) |
| Unknown | 1 | (1%) | 1 | (1.1%) | 0 | (0%) | 0 | (0%) |
| Menopausal status; N | | | | | | | | |
| Postmenopausal | 82 | (82%) | 70 | (76.1%) | 14 | (56%) | 9 | (81.8%) |
| Perimenopausal | 3 | (3%) | 3 | (3.3%) | 5 | (20%) | 1 | (9.1%) |
| Premenopausal | 13 | (13%) | 16 | (17.4%) | 3 | (12%) | 1 | (9.1%) |
| Unknown | 2 | (2%) | 3 | (3.3%) | 3 | (12%) | 0 | (0%) |
| Diabetes mellitus; N (%) | 13 | (13%) | 5 | (5.4%) | 1 | (4%) | 3 | (27.3%) |
| Hypertension; N (%) | 51 | (51%) | 35 | (38%) | 8 | (32%) | 8 | (72.7%) |
| Hyperlipidemia; N (%) | 35 | (35%) | 38 | (41.3%) | 3 | (12%) | 5 | (45.5%) |
| EC Histology; N (%) | | | | | | | | |
| Grade 1/2 endometrioid | 49 | (49%) | — | | — | | — | |
| Grade 3 endometrioid | 9 | (9%) | — | | — | | — | |
| Serous | 24 | (24%) | — | | — | | — | |
| Clear cell | 4 | (4%) | — | | — | | — | |
| Uterine carcinosarcoma | 9 | (9%) | — | | — | | — | |
| Mixed | 5 | (5%) | | | | | | |
| EC Stage; N (%) | | | — | | — | | — | |
| I | 73 | (73%) | — | | — | | — | |
| II | 1 | (1%) | — | | — | | — | |
| III | 15 | (15%) | — | | — | | — | |
| IV | 8 | (8%) | — | | — | | — | |
| Unknown | 3 | (3%) | — | | — | | — | |
| Tampon intravaginal | 92.5 | [54.5-115.8]* | 44 | [35.3-61.5] | 45 | [30-58] | 40 | [30-62] |
| dwell time, minutes; | | | | | | | | |
| median [IQR] | | | | | | | | |

When comparing the combined EC cases from both the AUB/PMB and biopsy-proven EC groups to BE controls, the 28 MDMs individually had marked methylation fold changes compared to controls. Table 8 lists the AUCs in discriminating between EC and BE for each of the 28 MDMs tested in the tampon pilot. The 28-MDM panel discriminated between EC and BE at a set 96% (95% CI 89-99%) specificity with 76% (66-84%) sensitivity (AUC 0.88 [0.82-0.93]). When reducing the number of MDMs in a post-hoc analysis to a 3-MDM panel, the combination of SFMBT2_0970, NBPF24, and MAX.chr10.4460 yielded the same AUC as the 28-MDM panel. When considering age ≥64 years v. <64 (median age in the tampon pilot) and BMI≥30 v. <30 kg/m2, neither covariate was statistically significantly different when comparing stratified AUCs. When limiting the analysis to tampon samples collected prior to endometrial sampling, the 28-MDM panel distinguished between EC (n=31) and BE at a set 96% (95% CI 89-99%) specificity with similar sensitivity of 74% (95% CI 55-88%) (AUC 0.87 [0.77-0.98]).

Exploration of the performance of the 28 MDMs in tampon specimens from women subsequently diagnosed with AEH or endometrial hyperplasia without atypia revealed lower methylation intensities compared to EC and higher intensities compared to BE.

As previously noted, approximately half-way through the prospective vaginal fluid collection study period, 50 mM EDTA was added to the PBS tampon buffer with the goal of improved DNA stabilization. Among the total EC cases and BE controls in the tampon pilot, tampons were collected into PBS/EDTA buffer for 57 ECs and 52 BEs. The AUCs for each of the 28 individual MDMs in discriminating between EC and BE based on tampons collected into PBS/EDTA buffer are listed in Table 8. The combined 28-MDM panel demonstrated improved sensitivity when tested on tampon specimens collected into PBS/EDTA buffer (set 96% (95% CI 87-99%) specificity; 82% (70-91%) sensitivity (AUC 0.91 [0.85-0.97]) compared to the full tampon pilot including both PBS alone and PBS/EDTA buffered vaginal fluid (Table 8). Additionally, in the PBS/EDTA buffer subanalysis with a set 95% specificity, the 28-MDM panel correctly identified: 17 (85%) of the 20 endometrioid ECs, 18 (78%) of the 23 serous ECs, all (100%) of the 9 uterine carcinosarcomas, 2 (67%) of the 3 clear cell ECs, and 1 (50%) of the 2 mixed EC histologies.

TABLE 8
AUCs for 28 DMRs included in the panel tested in the tampon pilot. Analysis performed
on all samples (100 ECs, 92 BE), including tampons collected into PBS alone +
tampons collected into PBS/EDTA. The subanalysis on tampons collected into PBS/EDTA
included 57 ECs and 52 BE. AUCs are listed in descending rank based on analysis
of PBS alone + PBS/EDTA. Cancer specificity is also provided (CC: cervical
cancer; OC: ovarian cancer; Ser OC: serous ovarian cancer; clear cell OC: clear
cell ovarian cancer; EC: endometrial cancer; clear cell EC: clear cell endometrial
cancer; pan gyne: non-specific gynecological cancer (e.g., OC, EC, and CC)).

| | PBS alone + PBS/EDTA | | PBS/EDTA | | |
|---|---|---|---|---|---|
| Gene Annotation | AUC (95% CI) | | AUC (95% CI) | | Specificity |
| KRT86 | 0.87 | (0.82-0.92) | 0.92 | (0.86-0.97) | pan gyne |
| CDH4 | 0.86 | (0.81-0.92) | 0.89 | (0.82-0.95) | EC, OC |
| c17orf64 | 0.86 | (0.81-0.92) | 0.85 | (0.78-0.93) | pan gyne |
| EMX2OS | 0.86 | (0.8-0.91) | 0.91 | (0.85-0.97) | pan gyne |
| NBPF24 | 0.86 | (0.8-0.91) | 0.89 | (0.83-0.96) | CC, EC |
| SFMBT2_0970 | 0.85 | (0.8-0.91) | 0.84 | (0.77-0.92) | EC, OC |
| JSRP1 | 0.83 | (0.77-0.89) | 0.87 | (0.8-0.94) | pan gyne |
| DIDO1 | 0.82 | (0.76-0.88) | 0.92 | (0.86-0.97) | pan gyne |
| MAX.chr10.4460 | 0.81 | (0.75-0.87) | 0.78 | (0.69-0.87) | EC |
| MPZ | 0.79 | (0.73-0.86) | 0.79 | (0.7-0.88) | pan gyne |
| ZNF506 | 0.79 | (0.72-0.86) | 0.76 | (0.66-0.85) | EC, OC |
| GATA2_6370 | 0.79 | (0.72-0.85) | 0.8 | (0.72-0.88) | pan gyne |
| VILL | 0.78 | (0.72-0.85) | 0.82 | (0.74-0.91) | pan gyne |
| LINC02323 | 0.78 | (0.71-0.85) | 0.82 | (0.73-0.9) | EC, Clear cell OC |
| CYTH2_4043 | 0.76 | (0.7-0.83) | 0.85 | (0.78-0.92) | EC, OC− |
| LRRC8D_8831 | 0.76 | (0.69-0.83) | 0.85 | (0.77-0.93) | EC (OC cross-reactivity) |
| LYPLAL1 | 0.75 | (0.68-0.82) | 0.8 | (0.72-0.89) | EC, OC− |
| SMPD5 | 0.74 | (0.67-0.81) | 0.8 | (0.71-0.89) | pan gyne |
| SQSTM1_3864 | 0.71 | (0.64-0.79) | 0.81 | (0.73-0.89) | pan gyne |
| ZNF323 | 0.71 | (0.64-0.79) | 0.78 | (0.69-0.86) | EC, OC |
| OBSCN | 0.69 | (0.61-0.77) | 0.79 | (0.7-0.88) | EC, Clear cell OC, Ser OC |
| ZNF90 | 0.65 | (0.57-0.73) | 0.75 | (0.66-0.84) | EC, OC |
| LRRC34 | 0.64 | (0.59-0.68) | 0.68 | (0.62-0.75) | EC |
| GDF7 | 0.63 | (0.55-0.71) | 0.71 | (0.61-0.82) | pan gyne |
| MDFI | 0.63 | (0.55-0.71) | 0.62 | (0.51-0.73) | pan gyne |
| EEF1A2 | 0.62 | (0.54-0.7) | 0.72 | (0.63-0.82) | pan gyne |
| LRRC41 | 0.61 | (0.53-0.69) | 0.75 | (0.66-0.84) | Clear cell EC, Clear cell OC |
| SEPT9 | 0.52 | (0.44-0.6) | 0.48 | (0.37-0.6) | Clear cell EC, Clear cell OC |

Through rigorous discovery and validation in tissue, unique EC MDMs were identified, which are detectable in vaginal fluid collected with tampons and demonstrate efficacy for triaging patients with perimenopausal AUB or PMB using self-collected samples. Translation to tampon-collected vaginal fluid samples indicated the 28-MDM EC panel tested in the tampon pilot had high sensitivity and specificity in discriminating between underlying EC and BE. This high sensitivity and specificity also appeared to be maintained when a smaller, 3-marker panel was evaluated. The sensitivity to detect EC in this context also remained high in subanalyses, including only vaginal fluid samples collected from women presenting with perimenopausal AUB or PMB before underlying endometrial pathology was determined via endometrial sampling. These data support the conclusion that EC-associated MDMs are spontaneously shed into the vagina.

### 6. MATERIALS AND METHODS

The following materials and methods were used to identify the various DNA methylation markers capable of distinguishing one or more types and/or subtypes of gynecological cancer in a biological sample from a subject having or suspected of having a gynecological cancer.

Samples. Tissue and blood samples were obtained from Mayo Clinic biospecimen repositories with institutional IRB oversight. Samples were chosen with strict adherence to subject research authorization and inclusion/exclusion criteria. Tissues were macro-dissected, and histology reviewed by an expert GI pathologist. Samples were age sex matched, randomized, and blinded. Cervical cancer (CC) sub-types included 1) adenocarcinomas and 2) squamous cell cancers. Controls included benign cervicovaginal (BCV) tissue and whole blood derived leukocytes. Endometrial cancers (EC) subtypes included 1) serous EC, 2) clear cell EC, 3) carcinosarcoma EC, and 4) endometrioid EC. Controls included non-neoplastic uterine tissue and whole blood derived leukocytes. Ovarian cancer (OC) subtypes included 1) serous OC, 2) clear cell OC, 3) mucinous OC, and 4) endometrioid OC. Controls included non-neoplastic fallopian tissue and whole blood derived leukocytes. DNA from 190 frozen tissues (16 grade 1/2 endometrioid (G1/2E), 16 grade 3 endometrioid (G3E), 11 serous, 11 clear cell ECs, 15 uterine carcinosarcomas, 44 benign endometrial (BE) tissues (14 proliferative, 12 atrophic, 18 disordered proliferative, 18 serous OC, 15 clear cell OC, 6 mucinous OC, 18 endometrioid OC, 6 benign fallopian tube, 14 benign fallopian tube brushings), 88 formalin fixed paraffin embedded (FFPE) cervical cancers (CC) and controls (36 squamous cell, 34 adenocarcinomas, 18 BCV), and 36 buffy coats from cancer-free females was purified using the QIAamp DNA Tissue Mini kit (frozen tissues), QIAamp DNA FFPE Tissue kit (FFPE tissues), and QIAamp DNA Blood Mini kit (buffy coat samples) (Qiagen, Valencia CA). DNA was re-purified with AMPure XP beads (Beckman-Coulter, Brea CA) and quantified by PicoGreen (Thermo-Fisher, Waltham MA). DNA integrity was assessed using qPCR.

Sequencing. Reduced representation bisulfite sequencing (RRBS) was run in two sample batches: First, endometrial and cervical samples and second, ovarian samples. A random selection of samples from batch 1 were also included in batch 2 to account for variation. Sequencing libraries were prepared following the Meissner protocol (Gu et al. Nature Protocols 2011) with modifications. Samples were combined in a 4-plex format and sequenced by the Mayo Genomics Facility on the Illumina HiSeq 2500 instrument (Illumina, San Diego CA). Reads were processed by Illumina pipeline modules for image analysis and base calling. Secondary analysis was performed using SAAP-RRBS, a Mayo developed bioinformatics suite. Briefly, reads were cleaned-up using Trim-Galore and aligned to the GRCh37/hg19 reference genome build with BSMAP. Methylation ratios were determined by calculating C/(C+T) or conversely, G/(G+A) for reads mapping to reverse strand, for CpGs with coverage ≥10× and base quality score ≥20.

Biomarker Selection. A proprietary DMR (differentially methylated region) identification pipeline and regression package was used to derive DMRs based on average methylation values of the CpG. The difference in average methylation percentage was compared between cancers and buffy coat controls; a tiled reading frame within 100 base pairs of each mapped CpG was used to identify DMRs where control methylation was <2%; DMRs were only analyzed if the total depth of coverage was 10 reads per subject on average and the variance across subgroups was >0. Assuming a biologically relevant increase in the odds ratio of >3× and a coverage depth of 10 reads, ≥18 samples per group were required to achieve 80% power with a two-sided test at a significance level of 5% and assuming binomial variance inflation factor of 1.

Following regression, DMRs were ranked by p-value, area under the receiver operating characteristic curve (AUC) and fold-change difference (FCD) between cancers and buffy coat controls. AUCs were required to be >0.90 and FCDs >20. No adjustments for false discovery were made during this phase as independent validation was planned a priori.

The three cancers were analyzed, as described above, separately and by subtype—generating individual lists of optimally performing DMRs. The merged sample and CpG level data was then appended to the three DMR lists. Each CpG had to have 80% or more representation in the samples being compared. DMRs were then ranked for each list by hypermethylation ratio, namely the number of methylated cytosines at a given locus over the total cytosine count at that site. For cancers, the ratios were required to be ≥0.20 (20%); for BCV tissue controls, ≤0.05 (5%); for buffy coat controls, ≤0.01 (1%). Regions which did not meet these criteria were discarded. In addition, the pattern of CpG methylation within a region was required to be contiguous or concordant. Subsequently, candidate DMRs (per cancer and per subtype) were analyzed logistically (using mean CpG methylation) to the other two cancers, individually. For example, the serous EC regions which met the filtering criteria were compared against the ovarian cancers (in aggregate) and then compared against the cervical cancers (in aggregate). To qualify as a site-specific DMR, in this case a serous EC DMR, the FCR between the serous EC cancer samples and either the OC or CC samples (or both) had to be 5-fold or greater. To qualify as a universal DMR, the marker needed to be represented on each of the optimal lists (above).

Biomarker Validation. A subset of site specific and universal cancer DMRs was chosen for further development. The criteria were primarily the logistic-derived area under the ROC curve metric which provides a performance assessment of the discriminant potential of the region. An AUC of 0.85 was chosen for the cancer vs cancer tissue comparison cut-off. The difference in methylation also factored prominently. Mainly there was a feasibility limit of 20-30 methylated DNA markers (MDM). This was primarily due to limiting sample DNA amounts and the degree of work it takes to develop high performing analytical assays. Quantitative methylation specific PCR (qMSP) primers were designed for candidate regions using MethPrimer (Li LC and Dahiya R. MethPrimer: designing primers for methylation PCRs. Bioinformatics 2002 November; 18(11):1427-31 PMID: 12424112) and QC checked on 20 ng (6250 equivalents) of positive and negative genomic methylation controls. Multiple annealing temperatures were tested for optimal discrimination. Validation was performed on independent tissue samples by qMSP.

These tissues were identified as before, with expert clinical and pathological review. DNA purification was performed as previously described. The EZ-96 DNA Methylation kit (Zymo Research, Irvine CA) was used for the bisulfite conversion step. 10 ng of converted DNA (per marker) was amplified using SYBR Green detection on Roche 480 LightCyclers (Roche, Basel Switzerland). Serially diluted universal methylated genomic DNA (Zymo Research) was used as a quantitation standard. A CpG agnostic ACTB (β-actin) assay was used as an input reference and normalization control. Results were expressed as methylated copies (specific marker)/copies of ACTB.

Statistics. Results were analyzed for individual MDM performance. Calibration plots were tested to confirm the suitability of the ACTB normalization. Box plots (ray and corrected) and heat matrices were created to represent the myriad of epigenetic relationships between cancers, subtypes, and controls.

Sequences. The various nucleotide sequences referenced in the present disclosure are provided below.

MAX.chr10.4460:
(SEQ ID NO: 1)
CGCCACCCCAGTTCGGCCCTGCTGGGCGCGCGAGCCAAGGCCGCG
GGGCACCGGGAGGCCATTTTGCGCGTGCGCTGCTCGCCTCGCGCC
GCCCTCGGCTCTGCGGACTCGGATCCCGCCAAATTTGAACGCGAG
ATTGTCAGGCCCTGAGGGGCTTGAGGGGCGGGGGAACGACGCCGC
TCTCCAAAGTTGGACCCCGTGGCGAGCGGCGGCGACAGCCGGGTG
CTCGCTGCCTCCCGAGGTGCTCCCTTTTCCCGCCGAAGCCCTCCA
CAGCGGCAGGCCGAGGCGCAGCGACGTGTCCCTGTACCCCGAGTT
CAGCGCGGGCGGGAAAACGACCTGCACCCGGGGAGGCAGCGGCTT
CGCGGGCAGAGCCCACGGGAGCGCGCCCTGCTAGGAGCCAGGCCG
GATAATCGCCTTTCTTTGTCCTCCTCCCTCTTCGAGTCCAATCAA
TGCCCTTTCTCCTTAATGAACGAGGTGTCCTTGGAGTTTGAGGTT
TTGTTGGATGATTTTAAATAAAATTATTAAGTTATAAAGTGGCCA
CCCTGAAGGTTCCCGAAGGCGACTTCATGTCTGTGACTGGAAAGG
CCTAGAGGAGAGGGTCCTCCCGCTGGGCTCGTTTAATAGAACGCG
CTCGAATCCCCTGGGAAAGAGCCTTGACTGGGTGACAGGGCTGAG
GAGGGGTGGCTGCGCGGCGGGAATCTCAAGATCTGGGCAAAGGCT
CGCGTCTCGGGACGCGAAGTCGACGCCAAAATGGGTCCCCGGACA
AGGCGACCCTGGGAGTGCCGGCGCCCCCGGCCGGGCAGAGGAGCG
GGTGGGCCGAGGCTGGGACATCGCCTCCGAAAGCTGCCGGGACGC
GGCGGCTTCCTGCAGAGCCTGCGCCTGCCGGATCCCCAGAACACA
GAAGCTTCTCGGACATGGGAGCTCCCCGTGCGCCCTAAAACCAGG
AGAGGAAGGGACGACTTGGGAAAAGGGACTGGGGAAACAGCGGAG
AAGTGAAAGCGGCCTAAAATGGGCGACGGCGGGCGAGTCCTCTTT
ATCAGTGCAGCAGGCTGCCGGAGCCGCCATTTGGTGGCGGATCTC
GGTAGTTCAGTAGCACGTTGTGCTGAACGTCACAACTGGCTTGTC
TACGTGGCATCGTCATTTCTTAACCGCGGTTTTACGAAATGCAAA
TTTCCCCCTGGCCTTCCTCCTCCGCGGCCGTCGACCCCCCTCGGC
GCTCCGGGTGGACGGCTCCGGGGCGCGGCTCGTCCCTCGGGTGGT
GCAGCCCCCGCGGCCCGAGACCCGGGGAGGGCCGGGGGTACTTTC
TGCGAGGCGCCTTCCCCGCGGCTTCTGCCCGCGCCAAAGCCTGGT
GGAATCCAGCGCAGACCTAAAGCACGCTTGACACCCCGATTTTTC
GAGACTAGGACGACTCTCTGAGCCAGCAGCTTTCCTCTCCCTCTC
GGGGAGAATCTCATTTCCTTGGGGTGGTGAGGGTGACGGGCACTG
TCTTTTGGCCCCGCGTGTCCGTTCCCCGGTCTCCCGCCTCACCCC
TCTGCGAGGTGAGGAGGGGAAACGGCGAGCTTAGGCCTGGCGGGA
AGGAGCCTACCCGACGAGAGGGCTCCGCGGGGAGGGTCGGTTGGA
ATCCCGCCCTAGCGCCTCCTGCTCTGCCCGGTCCCCACCGGGGAC
GGGGAATGCCAGTCATTTCTGTTGAGTGCTAGCAGGGCCGGTGTC
ACCACCTCGGGTGGCCGAGGCTTCGAGGTTTTCATGAAAAGCCCC
CGAAGCGTGAGGCGCCCGCCCAGTGGAGAACAAAGGGCCGAGGGC
CGAAGGCGAGGCGAGGCAGCGCGCGCGGCTCCCTTGGCTCGACCT
AGCTGGGAGTCGGGGGCGCGGGCAGGGCTCACTCCCGGCCTAGAA
ACTGGAGCCCGCCACCCCCGCCCCGCAGGCGACCGCAGGGATCCC
ATTCTTGGAGCCCGAGCTGCCATGTTGCCTTCGCGGAGGCCGCCA
GTCACTTGACGCTTCCGAGACAGCGAAGCCCCCAACCTGAGAGCC
CTTCGGCCGTCTTTGCCGCACAGCTGCAGTCAAGGCCCGGAGGGA
CTGCGGGACGCGGGCGGGAGCGAGAGCCCTGTGGGCTGCCAAGCC
GGCGCGGCCGCGCCGCGGCAGCCGCTTCCCTTGCCACCTTCGTTC
CAGGGGCTGCGGGGCTGCGCGCTCGGCAGAGGCTCGGTTGCCAGT
AGCAACCACACGACGGCGATTTGCAGCCAGGGCCGCCGCCGCAGC
CGCTGGTACCTCTGCCTCCTCCTACACCTCGGGCTCGAGCATTTG
AAACCCTGGGGGTTGCCTTCGGTGACATCTCCCGCCCCCACCTCC
AGTCCTCAGTCTCCAAAATCCTCAGCTCTGCTCAAAAGCCAGCGC
CCCCGGCTGGGCCCTGCCCCCACCGCAGACAATAGGAGCGGCTGG
GAGCGCACAGGGCGGCGCGCGCCGCGAGCAGCGGGCACCTGAGCC
CCCAAATCCGGGCGCGTCGCTGAGTCTCAGCCCCAGGTGCCCTCT
TCGCG.
MAX.chr1.2152:
(SEQ ID NO: 2)
CGACTCTTCGAGCGCCCCTCTGCTTCTGTAGAGGGGTCGAGCCAT
GTCAAGGTAGACCCTGTGTCGGCCCGTCTCCCTCGGATCCTCCGC
ACCAATCACTGTTGCTGAATCCGACACCCGGCGGATCCAGTGCGG
AGTCTCGAACAGCTGCGGAGCTGGGAGCTACGGGACATGAGGAGT
GCGGGGGGGAAGAGAAGACGGCGGAGGAAAATCCCCCGGCGGTGC
TCAACTGCGGCTTTCTCTCTCGGCTGTGAGCCGGCTCCGCCCTCC
GGCTTCCAGAGCAAGTGGCTTCTGCGTTCACCGCCCCCCGCCGTT
TGTGGGGCGGGGCCGATTCATAAGAATCGGTTCTCACCAATGGAG
GGCTTAGCATGTTTAACCTCAGGATCATAAACAAAAGACACTGCT
AGAACGGTCGGGAAAGTCATACGCTTTGCTTATCTTATATATAGA
TTTCTAAAATTCCAAACCGGGGACGCGTTGGTGGTGTAGTGGTGA
GCACAGCTGCCTTTCAAGCAGTTAACGCGGGTTCGATTCCCGGGT
AACGAAACGTTTTTGTCTTTCCTTCTACGAAAAACTTTTCTGAGC
CG.
MAX.chr11.0394:
(SEQ ID NO: 3)
CCCTTGAGGCCAGGAGTTCGAGACCAGCCTGTGCAACACAGAAGA
CACTATCTCTACAAAAAATTAAAAAATTAGCTAGGCATAGTGGCA
CATGCCTGTGGTCCCAGCTACTCCGGAGATTGAAGCAGGAGGATC
ACTTGAGTGAGGGAGGTGGAGGCTCCAGTGAGTCGTGATCGTGCC
ACTGCACTCCAGCCTGGACGACAGAGCGAGACCCTGCCCCCTCGC
CAAAAAAAAAATACTGGGATGCTATACACAAAATTGCCTTGAAAA
CTTGAGCACGGAACACCAAACAGCTAAGCGTGCCGGTTTGGGGAG
GGCGGGGGAGGAATAAGGAGCTGCAACGGTAAGAGGCCGCCACAC
GGTGGCGCAGTGAGGCTGGGAAACGGTGCACCCCGCGCAGGAGGG
GGCACTCCCCGTCGCGGCCACCCGGGGTGGGCAGGAGGCGGCGCG
GGCTGGCTGGTCTCTCCCGAGAAGGTTCTCTCCCGAGAAGGGTGC
GTCTCAGGGCTTGTCAGTGGACCCCTGGAACATGGGGAAGACGCA
CAGACAAGGGTTTCGCTCTTTGCTCTCCTCTCTCCTTGTCAGACC
TCTGTGACC.
MAX.chr11.3750:
(SEQ ID NO: 4)
GTCTTCCAGTTCCACTGAGGGCCGAGACTTTGTCTTTGCGGCCCC
AGTACTTGCTTAGTTCCGAGAGTGCGGTTTGCACTCAGTAAGTAG
CCACTTACTGAGTCCAATCGATTATTGGAAACCTAATTTTTCATC
ACTGCTTCTCCCACAAGAAGCTCTAGGACTGACTCCTCAAAGACC
AAAACTGGAATTAGCAATCCCGCTGTTTACCCGGAGGCCCGGTCA
AATGTCTTAAATCTGGGAGGATTCCTCCTGGGAAATTCCAGTAAG
GGCGCGGAGCAGGTCAGGAAGGAGGTTACTTTTTGGGTCTTTATC
GTCTATGATGGGAGAAAAGGAGAAATGAAGACTCGATTTTGCTGA
ACGCCTGCTCATTGTCAATTTTGCCGGTTCATCTCTCAAGAAATC
AGCAAAAAGACTCAGAATTGTAATCGCGAAGGGAAAGAATGCGGC
CACGTGGCCTATTTTCCTGTGGATAGACTAAGCAAACGCTTTTCT
TCAGGGGCCCGGATAGCTCAGTCGGTAGAGCATCAGACTTTTAAT
CTGAGGGTCCGGGGTTCAAGTCCCTGTTCGGGCGGATGCTGTTTT
AGTTTCCAATAAAATGGATTTGGGCGAGGCTGAGAGAAAGGAACG
TTATGTGAAACCCGCTTGGGGTGCCTCCTCCTTGAGGGAAACCAG
AACTTGCTAGTGGGTTCTTACCGGAAGAAGTGAAACGTGTGGAAA
ATGCCAAGAAACTTTATCTTCCAATAGCAGGCTTTTCTTTTCCAA
CCTTTATACGTTGCTTTGTCTTAGGATATTTTTTCTTTTAAATTG
TATTTTATATTCAAAACAGATCAATAAACACATCGTCAGAGTCAC
AATTAGTAATATTCTTGGCAAGAATTGTGCAGCTTTTGGCACCGA
GGAATGTTTTCAGGCACTTTTTATTAAAAGTGCGATGAGGAAACT
GAGACTCAAGAAATATTTCAGATGAAGACACGTAAAGACGCAAGA
TTCCTACATCTCCAACTGGACGCAGTCCTTCACCAGATTCTTAAT
GCTCTGGTGGGC.
MAX.chr14.7696:
(SEQ ID NO: 5)
CGGCACGTGGGTGGGCGATGACGCCATTTACTGAGATTTGATCCC
CACCACACGGCTCCGGGGTGAGAATTATGACATCTGGCTCAACAC
GGCTGCCCGGGACCCACACAGCCGAGCGGCCGAGCTCGGGCGGAG
TCCCAGGGCGCCCACAGCACCCCGCCAGCGCGCCCCGTCCAGCAG
GGCAGCTTTTGGGCGGAGGCGACCCCCACCGCAGGTCCCAGGACC
CTGCGTGCTCTTGAGCCAGGGGTGGAGAGGCCCGACCGCGGGGGG
CTGCCCCACCCCGCCGCCCTTCACCGCGAGCCGGGGCCCAGACCG
CCCAGCCACGCCCAGAGCCCGCGGGCGGAGACGCCAGGGGCGGTG
CCAGCGAGGTCCCGTCCCCGGTACCCCGTCCCGCCCCCCCACACG
CGGTGACCTGGGGACGCCCCGCGGGAGTCGTTCTGCGGCTCCCCC
TGGCGTCGGCTGGGGCCACCGCCCGGGCTCCCACCTCAACCCTGC
AATATGGGGTTGGGGCAGAGTGGTCTGCTGCCCGCTGCCCGCAGC
CGCTTCCGGTTAGGGAGGGAGCCTGGGCCTCTGGGTGCTCACGCT
GCGCTTAACGCTGGTCCCGGCAGCAGTGAGGGTGGAAGCGGCCG.
MAX.chr19.5552:
(SEQ ID NO: 6)
CGGACCGTAGCTCCTTCCACGCATGAACCCCGCACACGAGTCGGG
ATTCCCCCCATGACCCTCCCGTGGCCCCCGCACAATCTGGAGAGA
CGCGGGGCTGCGGGCGCGGAGCTGCCCAGAGAGGACTCCTGCCCG
GGCCCGCAGTCGCCGCGAAGGGACGGGACAGGACGCCCGGGGTCC
CGGCTGCCAGCCCAGCCCCACCCTGCGGCCGAGGGGACCGAGGGC
CGAGCTCCGCCAGCGGTACTCCGGTCCACAGAGCCCGGAGTCGCT
GGCTGGGAGGCCGGGGACCCGCCACGGCCAGTTCCAACCAGCCCC
TCCTCCCGTCTCGGGATCCCTGGCCCCTCACGCTCACCATTTTCC
GAATTCCTCCGTGTCCCGGGGGCCTCTCTGCGGCTCCCACGACCA
GTGCAGGTCCCTGTGTGACAGAGGCTGCCGCAGACTCTCCAGAGT
GCCTCTCAGCGACAGAGACAGGAGCCCAGCGAAGTGGCGTGTAGA
AGACGCCGCGGGCTTTTTCAATCTCGCACCCTCTTAGCTGAAGTG
CGCCTGATTGACAGTTCCCACGACCCCGCCCCACGGCCCTGATTG
GATAGTGCGACAGATCCCGCCCCCTGACGACTGAGTTACAGAAGC
GATCTCACG.
MAX.chr19.0548:
(SEQ ID NO: 7)
CGCGGGAAGAGACTGCCCAGCCCGAGGGTCGCAGGGGCAGAAAAC
CCCAGGTCCTGAACGCGCCTGGGCCCCGCGGCGAACCTGGCGTCC
CCACCAGACACCAGAATCAGATTCAGGCCAGGCCAGGAACCACCC
CAGCTTCCTGCATCCGCCTTGGTCCAGGCAGCAATGACGGCGGCG
GCCGCCAGGGGGACGGAAGCTCAAGGGCGGGGATCCAGGCCCAAG
CCTGGGCGCGCCCACCAAGGCGTGGATCCGGACGAAGGTTCCGGA
ACAGCCGGTCCCGAGCACCCACGTGCAGCTTCCGATCACAGCCCT
GGCGTGGCTTCTCTGTCCCCGGCTAAGGCCCGCGGTCGCTGCAGG
GTGCCTCGCCGAGGGTGCGGGCTCGGGGCTCACAGTGCTCCCAGC
CTCTCCCACCCCAACCCCGCCATTAAGGGGAGCCCCAGCGACGCC
CTGAACCCTGAAAGTCACACTGGGGCCGGGGCTGCACTTGGCGTC
CGCTTCCCCTCCCGTCACAGTGACCAGGGCTAGGGCCGCGGTCGG
GGCCGTCGGGGAGAGCGGAGGGCGCGTGGGAATGGGGCTCGCTGC
GCCACGGAAATCCCGCGCCGCCCTTCGAGTCCTCCCAGCTGCTCC
GCAGAGCCGGGGCCGCTGCCATCGCGTCTGCCCCGAGGGTGCGCA
GGCGGGTACCTGTCCCGACTCGGGGACAGCGGGAGCCCGGAGACG
CCCGTCGGGCTTCCCAGCCCCACCTGGGACGTCTCTAGGGGCGGG
AGGCCAGGAGAGAAGAGGGTGGGAGAGATGAGCTGCAGGGGGATA
CGGGACCCAGGGACCCGGGATCTTATAACACGCTTTCCACCCGTA
GGATTGGGGCCCACAAATGACCAGAAAGGTGGGACTCATTCGCCC
CCTTTGCAGATGGACCCATGTTGGCGCCCCTTAGATCTGCAGTGG
GGGCACCAGGACCTGCGGTGAGCGCCTCTGCGCCCCAAACGCCAG
CAGGTCCGCCCGACCGCATTCCCAGCTGGCTTGCTTTGCACAAAT
GCTGCGTCAGGGCACGCCCCACACCCACTCCTTCCCAGATCGCGA
CCCTCACGCCTTCGCAGACAGAAAGTGTCCTAAGCACAGGCAGGC
TCAGAGCCCGCCTCCGCCTCGGATTCGCTGTGTGGCCCGGAGCCA
GTATCTTGGCCTCTCTGGGTCTCAGTTTGCTCATCTCAGTGAATG
GGACACAGACACAGTCGCGCTGCGGGCTAACGCTTTATTTGCCAG
CCAAGGCCCCGGGCCCGCCTGGGCTTCTGCTCAGAAGATCCTCAC
GGAGTCCAGCTGCACGTCCCCGCCCACCTCCACCAGGCGCACGCG
CGCCAGCGGCAGGCGGTGGCGGAAGTGGTGGTACTGGGCGTCCCC
AACCACGGCCTGCAGGGGAGGGTCGGTGGTGAGGATTCCGGAGGC
CCGTGCTGGGTGGCCCTGGGGAAATCACTCATCCCCTCTGGGCCT
CAGTTTCCTCACTGGGAAAATGGGGCTATTGTTCATTCTAACTCT
TGCGTGAGGATCAAACGAGTTGACTGTGTGGCACAGTAAAAAGAG
GCTTTTTTAGTGCTGGTAATGGATATTCTCATTTCAGCGACCATT
ACCCGCTATTAAAGCGCAGAGGAGGGAGGTGAATTCGCGTAAGCT
GTGGGTGGTGGAGGATCTGCCGCCACTCCCACCCGCCAATCCTTG
CTAGGACGAGTTCCTGGGCGCTGTTTCCAACCCATCCCTCCCCAT
GCCTCAAACCCCGGACCTCAGGAAGGAATGAACTGGGAGTAGGGT
CTGGGATGGCGAGTCTGGGCCACGCCTTCCCGCTAGGACGCCCAC
CCCTTGGACACTTGGCTGGTGCTCGCCTCGTCCTGACCCTGCTGT
CTCTCTGTCCCTCGGACCCAAGTGGGAGTTGTTTAGGCGAGAGAG
AGGGTCGAAGGACACCCTTCTCCGCCTTGGCCACGACTTCCCTAC
CCCCCTCACCCCGCCCCGAACCTCCCTGCCTTCCACCAATAGCCT
GGCTTTGCCCAACCCTCTGCTCCAGGGACCTAAGTCTTGGCGTCC
ACGCCCCTGTCGCAGAGACGCACCTTGAAGCCGTCGTCTGACGCG
ATGATGAGCACCTCGAAGGGCTGCCCGCGCTGGAAAGGAACGCCC
GGCCCGCGCTCCTCGCGGCCCCAGGAGCCTTGCTCCTTGCTGTTG
AAGACCACCTCCGACGTGTCCAGCCGGGGGTTGAAATGCAGCGCG
GCATCG.
MAX.chr2.8918:
(SEQ ID NO: 8)
GAAGTCAGGGCAGTGCTGCAAAACCTCCACAGTGCGGAATTCCGG
GAAAATTCTTTACAGAGGTGTGGAGGTGGAGGAAAGCTTCCTGGG
CAGGCCTTTGGGGTCGTCCCCACGCAGGCGCTTGCAGCCACCCCA
GCTCGCGCGGGGCCGGGCTTTGGGGTGTGAGAGCTGGGACGGGAG
TCGGGTGGATGCCTGGCCGGAGCCGCCAGCTCCCCTCGTCCTCTT
TGCTTGTCCTTTAGCACAAGGGCGAGCAGCGTAGGACAAAGACTC
GGGCGGCAGCTGCCTGGTTCGGCGCGCAGGGGCGGCCTCGGCCAC
CCGGGGCGCCCGCCGCCTCCACCGCCCCGCGGGGGAGGCCCGATG
CCCGTCTTTGTCTGTGCCGCCGCCGTGGGCCGGGTCCGCAGGAAG
CGGGCGCCATCGTGCGGCCTGAGCTGGACACTGCGCCCCCGGAGG
CGCGGAGGCGCGAACCACCAAGCGTGGCTCCAAGCTCCACGGGGA
CGCTGGTGTCATCGTGGCCACGACTGCTTGTACTGTTGTGGTGCG
TTCTCTTTTGTATACTAAGTGCTGTGTGAACACAGAACCACTTCC
AGTAAATGCAACTGAGCCGTCGCCAGCAGAAAAAG.
MAX.chr2.4778:
(SEQ ID NO: 9)
AACAGTGGCGCTCAGAGAAGACAGGACAGCGGGCGAGAGCTTGGG
GGGCGATGGGAGGTGGAGAGGCACTCCAGGTCCCCAGGGGGCCAG
GCGGAGCTGCGGGACAGGGCGCAGACCCCGAGGCCCAGGGAGCAC
CGGGTGGCCGGCGGCCTGCAGGCTGGCGAGGGCGTCGGGCGGCGC
AGGGCAGGCCAGGGGGCGGGGGCGTCTGGGGCCCTGGCGTGGCGC
CCGGAACACCCCGTGCCGGAAGCTCCATGTGACCGTGACTCCGCA
GAAGCCGCGAGCGCAGCGAAACAAAGGGCGGCTCTGCGGCCGCCT
CGAGCTCAGGCTGGCACCGAGGGCCCGGACCCCCATCCCACTCCG
CACCCCCGGGCCTCCCGGCCCTTCTTGCCCTCCGACCCCGGGCTC
TGGCAGGGCCGGGAGGCGCAGGAACCCCGCGGGGGATGGGGCCGG
CGGACTGGCACTGAAGACACTGGGATGCAAGCGGGAGGCTGGGGG
GGGGGGGCTGGGGGGGGGGGGCGGGGCTGCAGGGCGTGGACGGTC
TC.
MAX.chr20.3853:
(SEQ ID NO: 10)
CGGAGCGGATATTCCCGGAGCCCCTCTGCGAGCCACGCGCCCCTC
TGGGAAGCCCGCTTCCCCCTGCAGACAGGCGCTGTGACACGCTTG
CGCCCCGGTCGAACAGGCGAAGAGGCCGAGGCCCAGAGCGGCGCA
GGGCGAGCCTGGAGGCTGCGCCCCAGACCTGGACCAGCCACGGAC
GCCGCTCCCGCCGCTCCCTCCGCTCCGCTGCGCTCCGCACGCTGG
CGCCGGCTCCCCGAGGCCCCGGGCGCCCCGGCCGCACGCCTGGGT
AAAAGGTCCCGAGGAGTCCGCAGAAGCGCGCCCACGCCCGAGACG
GCCGTTTCCGCCGGCCTGGGAAAGGGGCGGAGAAGGGGGTCGCCC
GGGCCGCAGCGTGCCGGTCCCCGCCGGCCGAGCCGTGTTTGGGGC
CAGTCCCCGCACCCCGCTTCTTCCCCACCTGGGGAGCGGGGCGCC
GCGGTAGGGGCACTGGAGCGCACGATGCACCCCGCCAACGAGTCC
TTTCTGCAGACGGGGTTCCTGTTTTCATGCAAATGCCTTTGTTAG
CGCACCGGGAACCAGGGGGACGGAACTGCAGCTGACGCGGGCTGC
GCGCCGCTTTTCCGCCTTCGCTTGATTCGGCCTCAACGACTTAAA
CGCGCCGGGAACAAAAACGCCGGCGCCGCGGAAACCCTCAGGAGC
GGCACGAGAAGCGCGGCTCCGCTGGGGTCCGCGAGAAGCGGTGCG
GGCGGCCG.
MAX.chr20.2903:
(SEQ ID NO: 11)
CGCTCGCCCCCTTCTCCGCGCGGGCCCTCAGCTCAGCTCCCTCTT
CGCTCCCCGTGTCCCCGCGAGCGGGAGGGAGGGGATGCTAGGACG
CCCTGTCGGCGTCGTCGCCGCTTTCCGCCATTGTTTAGTCGTGAT
GCTCTCATTTTCTCTGAATCAACAATTTTCTGCTCGGCTCCGCGC
CGACCGGCGAACGCGGGGCTTTTCCTCGCCCGCCTGATGACAGCA
GAGCGGCGCGGAGCAGCTGGTCCGGAAGGAAGCGCCAGGCGCCTG
CCCGGTCCCAGGCGTCCGCTGCCGCCCACCCACACCAGACCCCGC
CCCCGCGCGTCAAGCCCCGCCCATCCATACCAAGTCCCGCCCCCA
CACCCTCACCCACACACCAGGCCGTCCCCACCCCGCCCCCAGAGC
CCCGGGGCGCCCCGCCCGCTAGCCGCGCACGCGCAGTGAGCACGG
CGACCCCCGGTGGTCGGGTGTCTCCGCAGGCCGAACACGCTGCTC
GCCCAGCTGCGGATCATTACCGCCCTTTTGTTCTCCGTCGCGCGC
TCGCCCCACGCTAGGAATGCAAACTGTAGGCGCCG.
MAX.chr21.5011:
(SEQ ID NO: 12)
TGTTTTTCCAAAAGATAATAAGCGTCAACAACAACAAAAAAATAA
AAAGTCCAACTCCGCCCCAAAGCAGCATCTGGCTGGCCTGCGAGA
TGCCCACTGGGGAGGCGAGTCCGCAGCTTAGGACTCAAGCCCGGG
GTCGGAAGCTATTGCCGAAATCCGAAACGCAGCGCTCGCAGCTGC
AGTGACGCGACCTGCTCATAAGTCCCCGTGCTCACAGCATCCCGG
CAACTTACGAGCTAGTGCTTCCGGGTCACCCCGGCCCAGGAAGGC
GCACGCGCGAAGCATAGCGAGCTTCACTCCGCACTCTTAGGCTGC
GTGTGAGGCCTGCGAGTGCTCGGGAGCTGCCGCGGTCACAAGAGA
AAGCCTAGCTGTCAATGACAGCCCCAGAGCATCTGGGCGCCTTGC
GATACCCGGGTGTCTGTAGGCAGCCAGGAGACACTTCCAAGCTGA
TCTGGAATCTTTCCTCGCCCAGCTCTGTCCCTCGCAGGGATGGCA
AAGGACATTACGACCTACATCCCTTCCCGGATCTGATGGCTTCAG
ATTGGCAGATTGTGTTAAAGTGGAAGGCTCGTGGTGCCCCTTTGC
TGAGTTTTTATGGACTTAGTTTTCCCAAGTAGTTCTAATTATCG.
MAX.chr22.5665:
(SEQ ID NO: 13)
CGGCTGTCTTTGTCTCCCGCGAGGCAACTCTGACTCAGGCTCCAG
CTGCCCGTGGGAGGGAGGGGGCGCCCGGGCTCCTGAGGTCGCCAG
GGAGCGGCGGGACTGGGAGGCTCCAAAGCCCTCAGTGTACGTGCG
AATCCGGAGCGGACACCGAGACCTTAGCGCGGGAACCAAGAGAGG
ACAGAGCTCCACGGAGGCCACAGCGCGTGCACGGGGACAGGTGCG
CCCTCCCCGGCAGCCCCCCTGCTCCTCGGTCACAGTTCTGTGCGG
AGGCGTCTTGCGCCCTCCCCCCTGAGCCTCGCCCTTGAGTCGGGG
CCGTGGGCCGCATCCAGGCCCCCAGGGCTCGGGATGCGCGTGAGG
ACCCGGACTCCCGAGGGCGCAGAGGTCGGGAGCCCGAAGCAGGCG
CCCTTGGCCTTGGTCCCGCCCCTTATCCGGTCCCAAGCTTTTTCC
TCGCCCCTTGGCCTTGACTCCACCCCTTAGGCATGCCGCTGGCCC
CGCCCCTTTCCGGCCACCTTGAGGCTTGGGGGTCCCTCAGCCCCG
CCTCTCTTCTTGACCCCGCCCCTTGGCAGCACCCCCTACCCCCGC
CCCACGTCCAAATCTCCCGGGGCCGGTGGTGGCCGGGGCTGACGG
CGGAAGCCGCGCAGAGACTCGCTTGCCCCGAAGTCGCTGGATTCG
GGCCTGGATCCCAGATTATCCGCAGCCTAGGGGAGTGGAGAGATG
CCCAAGGTTCCTCTGGGTCCCGGGACCCCAGTAGCGTCCCTCCCC
CCGTCCCCCACGCCAACCACTGAGCGCCCTTCGGAGTCCCGGGAG
GAAAGCGTAGGGGGGGGAACTCTGGCATCTCTCTCCTCCCGGTTG
CTCCCCGACTCTGCCCCGCTATTCCGCTATTTGGGGCAGTCGTTT
CTACCG.
MAX.chr3.6408:
(SEQ ID NO: 14)
CGCCGACCCCAGACCCAGTCCTAGTCCGGCCAGAGGAGGCCGTTT
ACGAGCCCACACCCGTAGGTGGCGCCACAGCCGGAGAATTGGCTT
TGGTTCTGTTGGAGCCGCGCCGCCTTTAAATTAGCCCCACGCATG
CGCGACTTTTCTAGCCCGAGCCCGCCGTCTGCGCCTGCGATTTCG
CCCATACTCCCCGGTGCCCGCCTCGTGACGTGCCGCAGTGTTACG
AAGGGACACCAGGGCGCAGGCGCAGCTCGCTCCTCAGGCCTGCGA
GAGGCCCGCCGGCCCAGCAGAGGGCGCCCACCAATCTGCACGCGG
GCCCAGCGAGTTATCTTGATTTCGGCCAAGCTTTCTGACTGCTCC
AAAAAACGAAGAAAAAGATTCAGGGAGAGTAAGAGGATGAAGAGA
GCTGGTGAAGCAGCTGACCAAATGGCCCGAGGTGGTATGCAGCCG
CGGTAAAGCAGGGCCCCTCCGTGAGGCACAGCCGCCCGGGGGTTC
CCTAGGGGAAGCAGGGGCTGCGGCAGACGCCTCTCGGGCAGGTCA
GGGTATGCACCCTCCCGCAGGGGCTCCCAAGGCCGGGCGTGCGTG
AGGCCAGGTCCACGGGCACACCACTGTGAACACTGATTAAACGTG
GCCTCCACGGCTTCCAACCCCCAGGACAGGACCAACCCTCTGCCC
CCGGCTCAGGCCAGAGCTCGGCGAGACCGTC
G.
MAX.chr5.3588:
(SEQ ID NO: 15)
CGCGTCCGGAGGAAGGCTCACCCGGAGGCCGCCTGCAGGCGGCCA
GGTGCCAGCCACTGCGGGCCTCTGGGGCCGAAGCCGGCGGATGGT
GAGACGCTGGTTGGTCTGCAACACTGCCCAGACCCCGGGCACTCA
TGTCTAGAAAAAAGCTGACTCGTGACACCAAAGGAGCTCTTTCAA
GCTTCCTGCACGCTCTTAGCGCCAGAGCACCCCAGCCGTCCTGGG
AGCCCCCGAAGCCAAGCATATTCGAACTCCGAATCCGCTCGATCG
CCGGGGACCTGCCATCTGGGTTCGGTTCCCCAAGGTCGCTGCCGA
CCTTAGACCGCGGGGGTGTGGGGCGCCGGGGAAGGAGACAGAAGG
ACAGGCGCCGCCCAGGGCCGCGGGGACACTTGGGGCTGCGTCCTG
GGTGGGCGCGATGCTCCCCCAGAACGACTGGAGATGGAGAGTGTC
GGGGAGGGAAACGGGACCCACGAATTCAGGGCGCTGAGTCGGCGG
AATGCGCCCTGACTCCCCCTGGCCGAGAGCCGGCTCAGAATGAAA
GAGCGCGGAGTGGGAGGTCTGGGAAATGGCAGTATTTGTATTAGG
GAGAGAAGGAAACAGGAGTGGGAGCCGCACGGCTTGGGGAACGCG
GGAGATCGCGGATTGCGGGGATAGCGCAGCGCGGCTGCCCGGGGC
TGCTGGGAGGGGCCGGACGAGGCCAGGGCGAGCGGGGTAACTGCG
GCCGGCCGGACGGCGGCGGTAACCGGCTGCACCGAGGTGGTTCCA
CCACCGCGCTGGGCGCTTGCGGTTCGTCTGCTCCAACGAAAGCCG
CGTCCCACGCTCCCTGCCGCCGCGTGGTTTTGCCTCCTCAGAGGG
GCAGCGGCGACCCAGGGGCTGGCG.
MAX.chr8.5938:
(SEQ ID NO: 16)
CGCAGCACCGGGTGTTCCCTCCTAGCCTGGTCGCTCGGGGGGAGC
GTTGGTTGGCGGGGTGCAGGTCTGGTGCTCGCTCAGGTGGGCCAG
GCACCCGCGCGCCAGGTGAGGCGGGCGGGGGAACACACGCCCCTG
GCCCCTGCGCCGCCGTCACGGCCGCCCACCACCCGAGGGCGGGGG
TCCTGGTGGGGTGTCGATTCCGCCTCCCCGCCCACAGGCACTGGG
CCCCGGGCGGCCACCGGGGTGCGGGGCTCCCAGCGTCTCGGGCTC
CCACTGCTTCAGGCCTGTCCAGGGGGGGGGAGCGTCTCTGTGGCC
GCGGCGGGATTGCGGCGCGGTGGCCGGGCGTCCCCTGCAGGAAGC
TGTTCTCGCTCGCTGCCTCCCCCACCTGGGAGGGAAGCGCCTGGA
TTTTGGGTCCCGCCGCCCTCCGCGCCCTGGGCCTCCACCTGTGTT
CCCAAAGCCCAGCCACGAGTCTGGGGGTGCCGGGCGTGCCGTGGT
GGGCGGAGGCTTCCACAGCCCCTCCCTGCCAGGGACGGCGGGGGG
GGACATGGCGGGGCCGCACGCACCGGGTGGACGACAGGGGATGCC
GCGGGCTCGCGTCAGCCAGGGCG.
MAX.chr9.4007:
(SEQ ID NO: 17)
CGCCGTTTGCTCAATGTCCCCGCCAGCCTTGTCGGTCCTTACCGC
CGTTTGACTCCACTGTTTTTCTCGTGGTTTCTGCTGCTTCTCTAA
ATTGTCCAACGACCGTTATTCAGTAAAAATGAATGAAACGGGGCC
GTGTGATCTAGGCAGCCTGGAGATGAGATTTTGGAATCATAAGCT
ACATTCCAACGTATAAACCGATTTTACTCGTTTTGGATACTCGAT
GTACGCGGAATGGGCGCTGTAAAATGCGGCTGCCCCGCCGGAGGC
ATCTGCTTGGGACTTGCTGGCAGCCGCCGGTCCCCTCTGCTTGCG
ACCCTCGGCCCAGCCGCCGGGACCCTGGTGCACCTGTTCCTGGGC
GTCCTCTCTACTCCCCAGTGGCCGCCAGCTCCACTCCCAGCCTGT
GGCCCCGGACCCGCCGGCCTGAGCGTTCGCAGAGGGCCGGTCGTC
GCCACAGCCCCGCGTCCCGGCCCCCGCGCCCCTTGGACCTTCGCC
CCAGGCCGGCGCAGCCCAGCTTCCCGGGCAGGCTCCACGCTACCG
GGGTCCAGTGCGCGGCGACGAAGCGGAGAGCTGTGTCCAGACTCC
GGAGAGAAACTCCGGCTCCGCGGGGCGGCGCGGGGCGGCGCGGGG
CCCGGAGCTGCCCAACTCCGCCGCCTCGGGAAGGCGGCTTCGGGC
CCGCAGGGAGCCCCGGGGAGGGTTCCCGGTTCCGCCGGCAGCGGC
GTCGAGGGGTGCCTGGGCTCCTGGGGACCGCGAGAGGAAAAAGAA
CGGAAATCGCACCGGGGAGGAAGGACGCGCAGAACGCCCCCGTGA
AGCGGGGTGCTCCGGTCAGGCGTGCGCGGGAGCGCGGTCCGGGGG
AGTCCGGCGGCGCCGTCGCGCGCACTCGGCAGAGGCTTCGCGGGA
GAACGCGCAGCCCGGGGCGTGGGGCGGGGAACTGCCCGCGCGAGG
CTTTCGGCGCGTCTGGGTCTCGGCGAGAGCAAAGCGCGTCCTGGC
ACCGGGGGCGGCGGCGCAGAGGCCGGGAGGAAGAAATCCGGGCCC
TGGCCCAGGTCGGGCTTCCACCCCTGCGACCCGCGAGAGGCCCAG
GCGGGAAAGGCGGCGAGTGGCGTCAGCGGTTCCGAAAGCAAACCT
GGCCCGGTGCTACTGCCCGAGGGTCGCCGGGCGCGTTTCCTAATT
CCCCCGAGTCTGGAAAACGGAGACTTCCGTAGCGTCTTCTTCAGT
GCGTGCTGCGAGTGCTGAAGGAGGACCCGGTGCCTGGACGACCCG
GAGCAGGGGAAGCACTCGGCCGACGCTGTCGCTGTCATCGGCGTC
ATTGGCGGGCAGGACAGTGGGGGGGGTAAGGGGCCTCCCCGCGCC
TCCCGGCCCTTCGCGCTCGGCGCCAGCTCTTTGGCTCCCTTCCCT
GCGCAGCTCTAGGCTTAGCTCTCAGCCATTTCTCAAGAAGACGAT
CCCGAGGGTCGAAGGCCGCCCTTGACCCTTGACCACGGACTCTCC
GTGTAACTCGGAAGAGCCGTGATTTTAAAACCCGGCCTCGGGGTT
ACAGAAGCCCGAGATCTGGGAGGCGTCCGGGACCTCCCTCCCAGA
ACCGCAGGGACCCGGCCTGGGATCCAGGGTGTGGCCTCTCGCTCT
GCGCGGTCGGGAAGGCGGCCGGGTCCGGTCACCGCGCCAAGCACT
GCGCACCCCTGGGACGCGTCGTTGCGGGGGGCTGGGGGGCTGGGG
CGCCTCCACGACGCCTGGTCTGCCCGGCCAGTGCTTGGTGTCGTT
GGTGGGTTCGTGGCTGCGACGGGTAAACGTCCGTTCCGCGAGCCG
GGCAAGGCAACCCCTGCGGGTCGCGCCCGAAGGCCGGACCCCTCC
AAGCCGCCTGGGAGCTTCCAAACAGGTGGACCCGAAGCTCCTGTT
TGATCGGAGAATAACGTTCAATTTACTCCGCCG.
MAX.chr9.2025:
(SEQ ID NO: 18)
TGATGTACGCCCTGGTGGACAAAAGCTGCTAGTGTCAGCTTGATT
TGCAAGATCAACATTCATGAGTTTCACCGCTTAGAAAGGGGCATT
ATCTGCAAACCGGAGACTGAATGGAAGCCATAAACAAGTGATTTC
ACACTACCAAGCAGGAAGAATATTCTACCTCCTCAATTATTCTGA
ACAGCATGTTTGGCCCCTTCCAGGTTCCCACCGCTAGCGAGCCCT
CCACCCCTGGTGTGTGCAAACGGTGGACCTTTCGGCGCCTAAGAA
ACCGGGTGCTGAGCCCGGGAGCAGCGCCTGCTTTTCTTCCCAAGA
TCCACTCCGGGTTTTGCCTAGCGCTGCTCCGGGAACCCATTCCAG
ACGCAGGTAACGCCAGGCAACGTTTTCCTTCTCACCCGCCCAAGG
CCAGCCCCGAGCCGCCGGGGTTCCAGGCCCAACACAGCACAGATG
CACGTTTCAAAATGTGCTCGAATATGCAGCCTGCATCAAAGGCGT
TGGGAGGCTCTTTCATCCTCTCAGCTGCCTAAGAAGGGACATGCT
CCCAGCTACCCTCATTTGTGGCTGGGTTTACTCTGAAATGAAGAT
GTACCTCTGGATGCAAAAAGAAAGGGTGGAAGGTTTTTTTCCCCC
TAC.
MAX.chr1.2533:
(SEQ ID NO: 19)
CGGCGGGCTGGATTAGGGCGTGACGCCCCCCACCACGCACACAAA
CATACACAGCCCACTGGATGTCTGCCGGGTGGGAGCCGCAATCTC
CGCGCGGTCGATGGGGCCCTCCGCTGCGCACTCGGCCCTGCGCCG
AGCACCCTGCAGCCTCCTCCCGCGACACGGCGCTTTGAACTCGGC
GGATTGATTTTGCTTCCCTTCCCCCTTTTGTGTGTGTTTGCGTTC
AATTGGTTAGGTTTTTAAGATTTGGGAGGGCTGGTGTGAAAGAAT
TAAAATACTCTTAACTGGAGCCCCTCCGCCGAGAACTGGAGGTCC
CGCCTCCTAGTTCGGCGCTTTCAGGACCCTCTTCCCAGAGGGAAT
TTCTTTCAGAAATTCCAGGGTGGGCTTGTAAAAGACGCTTCCGCA
GAGCAGGTCCCGTCAGGGTCTTTTTCCTGTTCCTGGTGCCAGCGG
TCGGCCCGGGCGCCCCGCAGACCTCGGCGAGGTAGATGTTAAGCT
CGGAGAGTGCCCCTCCCGCAGGCGCCGTGGCGAGATCACTCTGAA
TATGTAACATATTTGTAACGTGCGCCGAGGTGTGATGTGTGTGCT
GAAATAGGGGGATGGGGGAATTCGAAGCCGGATTGGGAAGGCGGG
GGGGAGGCGCACAGAACTCACAATGTACTTCGCAATCTAACAATC
TGAACATTCATTTATTAAAAGCTGCTGCGTGACATTTACACTGAG
CCACCAGTCTCTGCCTCTAATCCGGGCGAAAACGATTGTACTGCC
GAGTTATGGCTGCAGCGTATGGGGACGCTGCTGTCCGCGGCCGGA
CAGAGCCCATCAGCTACAACGCGGAAGGCCTCTGCACCCCCTTGG
GGGCGGGAGGAAAGTACTGCCAGTCCTGCCTGGGGGCCGAGGGTA
ACAAGCACCGAGCCTCTCGCTCCACGCAGGGCCAGCTGCCCAGCT
CAGCGAAGCTCTTGTGATCTGGTGCGTGTCTCTCGCTCTTCCCTC
CCCATCAAAGAAGTAAACTTTCTACCTACTCCCCCTAATCCGATC
GTTTAGAGCTGCTGTTTTCCTTTTGTCAGATTCCTCCTCCCCGAT
CAGTCTGAGTACACGATCAGAACTGCTCAGAGAGCAGGAAGCACA
TTGATTTCAGCTTGTTCTGTCCACAGACAGGCCCTGACAAGGTTG
TTAGAACAGCCGGAGAGGTCTATACAATCACTTAATTACCAAAAC
TGTCAGTCAGGCGGGACGCGGATCCGCGTCCCGGGCTGCGCTAGG
CATTCCAGCACTGGGCCGCGCGCGTGATTGATCGGTGCTGATAGC
ACCGCAAAATAATTACGGCGAATTTTCTGATGTGTGATTTTATCC
CAAGTTCATGCTTCAGAGAGGTAATCGGAGAATGAGAAGGGTCAG
TGCCATTTCGGATTACCTGGAATCTGCGAGAAAGGGTAAAATGGG
GGAAGGAGCTCCGAGGAAAACGGGAGAGATGGGGGTGCAGAGAGA
GAGGGAAGAAGAAAGCGAGTTATGGATTGCTGGAGGGACTGCAAG
CAATTCGTCAAACTGTGCAAGTGATTTCCTTCAGAGCCAGCATAT
GGCAGATTGATTTTGTCCAACGTCGGTTTTAGCCACATTTAAAAT
GATCCAGCGGTTATTACTGCGATTGGCTTAGGAACTGACAGGCAG
TTTTAGGCGCAAGGAGTATAGATCCTGTTTACCGGAGATGTGTTC
GTAACTGCTGTCAAATACAGTTAAGTAAATATCATTAGCGAAGAG
CTCTGTTAAGAGAAATGCCAATCCAATAAATATGCTTTTCCTCCC
CGCCCTCCGCATGGCTGCCTGCGCTTCCTCCAGAGGTTCTCCTTC
CTGCTCCTTTGCTGCTTGGGTCAGACGTCCCAGGCATGGTGCTGA
CTCCCGCCACCTTGGAGCCCCGAGCTGAGCCTCGGGCAGAAGATG
ACAGGCCAGCCGTGGGGCAAGGAGGCCGCGGAAACGCGGAACGGC
TTCGGGGAGACGGAAGCGCCCAATGAGATTCACCCTGCAGCCCGG
GTCCAGCCCACCTTCCTCGGAGATTGCCGCGGCCCTCGAACCCGG
GCCTAGGTCTTCATGTCCCGGCGGCCAGAGGACGTTGCGGGGACC
ACTGGGGAGCTGCCCTCAGTCAGCTCTCTGCCCCACGCCGGAGGT
CCTGGCGCGGCTTCTTTCCCGAACTAGACTGGCGACTCTGGGCCA
GGCCCCAAGGACCGCCCCGGCCTCTCCGGCTTTGCGGGGAGAATC
TGAGGAACCGAGTCCAAGATAGCCGACCTAGGCTGTTTTCACCCA
GACCCTGCGTCCCCGACCCG.
MAX.chr13.3357:
(SEQ ID NO: 20)
AGGGTTGACCCCAGTACCTGACTTCTCCGGGAGCTGTCAGCTCTC
CTCTGTTCTTCGGGCTTGGCGCGCTCCTTTCATAATGGACAGACA
CCAGTGGCCTTCAAAAGGTCTGGGGTGGGGGAACGGAGGAAGTGG
CCTTGGGTGCAGAGGAAGAGCAGAGCTCCTGCCAAAGCTGAACGC
AGTTAGCCCTACCCAAGTGCGCGCTGGCTCGGCATATGCGCTCCA
GAGCCGGCAGGACAGCCCGGCCCTGCTCACCCCGAGGAGAAATCC
AACAGCGCAGCCTCCTGCACCTCCTTGCCCCAGAGACCGTCCGAG
CTGGAGCCACAAGCCCTCCATTCCTCTTGGAATCTTCAACCCCAA
GGTAAGGTAAGTTCACCGAGCACCGCCCAGCGATGCGCAGGATCC
GGGGGGGATCACGCGCGGCGACCCTACCGAGCGCTCCGTGCGCGC
CCCCATCTCTCGGATCGTGTTCCTGGCTCTGTCGAAGCTGCTGAG
TCCCGCGATTCGGGAAATCCGGCACTTGTTTCTCACCCTACACCA
TCACGTGGAAATCATTGAAAATGGGAACCCTGGTGGAGTATCTGG
GAGAGCACGCTTGTGCCGAGGGGCCTGAGCTATGGGACTTCCTCC
AGGTCCCTCTGTTTCCTGCCGGCGTAGGGGACTCGTAGTGTCGGA
TCGCATAGTGCCAAAAAATAGTGCATGGGAAACAAACAA.
MAX.chr14.2093:
(SEQ ID NO: 21)
GTAGAGACGGTGTTTCACCATGTTGGCCAGGATGGTCTCGAACTC
CTGACCTCGTGATCTGCCCGCCTCGGTCTCCCAAAGTGCTGGGAT
TACAGGCGTGAGCCACTGCGCCCAGCCCCAAAATTGGGAATTATT
TCAAAATAAAAAGCTGGATAAATGCATACACACAAGGCAGTATCG
CGTATTTTCCACGAGTGCCTGTGCAGGCAGGTAAGGATTTAGGAA
AGGTCTGGAAGGATGTGCAAAATGTTCCGCCTGCGAAGGTTCCGC
GGTGGCGGGGACACTGCTCCGGCTCCGCTCCCGCCCGCCCGAGCG
CTCGGATGGGGCCGCCTCTGCACTGCGTGGCCACAGGCGCGGCCC
GGCTGCCCACGGGCGCCCTTTGCAGCTGCTGCCCCCTGGCGGCCG
CGGGCGGCTACTAGCGGGAAAGCGAAACCCGCCCGGTCCATTCAA
GCCCCGCTGCCTGGCGCCCTCTAGGGTCGTTCTTGGGAACGGGCG
GACCTTTCGTCAACACTTTGCCTGCAAGATCCCCCATTGGGGGAA
CCGAGGAGGAAGTTAAAGGAAGATGTGTGTTTTTGAGCGCTGCTT
TGTGCCAGGCTCATCTTAGGTGTGGGACGTGTACTATCTGAATTA
ATACCCCACCAGGCCTGTGGGACAGTCACTGTCACCATTCGCAAA
TTATGGATGAAGAAAGGAGGTACCAAGTGGTGGTATCACCTGTCC
ATAGTGAGCTGTCCCTCAGGAGGGTGGCCGCCCCAC.
MAX.chr17.2455:
(SEQ ID NO: 22)
CGTTAACAATGTCGCGTACACGCCCGAACCGGAGGAACCCCATTC
CACGCTCCTTCTGGAACCGAATTCACCTCTGAGGCTTTGGGGCTT
CAGAGCCGGAGCCGCTTGGGCAAAACCAGCAGAACAGCGAGAGGG
AACGGGCTGGTCTAGCCCTGCCCTGAGCATTTCTACTGAGACCCC
CGGTCCTGCTTCTTCCAGCCTCTGCTGGATTTCTCTCCGACCCCT
CTGGAGCGAAGCCCTTTGGCCCTGCGTTGCATGCGGCACGGTGCG
GGTTCGGGCTCTGCGCTGGAGCCGGGATGCCCTCCGGCGGAGGGT
GCGCGTAGGCGGCGCCTGGGCGTGAGCCCCGCCTGCAAGGCTCAG
CGTCGGGGAAGCACTTTTCTCGTCGACCCGGGGTCTTTTTCCGCC
AAGGAGCTCGGGGCTCAAGAACTCGGGACTGGGCTGTGGGCGGGG
CATGGTTTTCCTCTCTGGGCGTCCTAATCTCCAATTTCAGGCAAA
TTCGCTAGGAAGAACCTTCCCGAGCGCG.
MAX.chr18.4390:
(SEQ ID NO: 23)
CGGTCACAGAGAAGACGCCCATCCCGGAACGCGAGCGGGAGCCAC
CCCCGCCCCCACACTCGGCCCTCTTTGTCCCCTGCTCAGCGGTCA
AGGACCTTGTGGTGAGCGCCTCCCCACAAACGCAGCCTCCTGCGG
AATTCAGCCCTGCACTTTTGCAGAGCTTGGAGCCAAGACAAATGA
CATTTGTGATCATGAGAAAGCCAAGAACGATGGAAACGGTAGCAT
CGAAGTTGTGCCGCTTTCTGAAACTTCTTTGAACTCGTTGTGCAG
GGCCGGGGAGCTCTACGGCCAGGAAAAGTGCGCAGGGGGCGTCCC
CGCGTCGGGCGCGCACACGGCCAGAGCACGGGGCTCCCCACGCGG
GTTTGTCTCGGACGCAGAGGGGCCGCGAGCGGAGACATGGACGCG
GCATTTCTCACGCCAGGAGCTCCCCGCGCGCGCTCCCCTTCCACA
GTCCCCGCCCCGCAGGCCGAGAGAGGACCGCGGGGACCTGCGAGG
GGCTGGGCCGTCCAGGAGGCCTCGGGTCTGCGCCCCGCTCAGCCC
CCGCGGGACGCCTTTGGCGAGAGACGCGGTTCTGAAATCAGCTGT
GGGGTTTCGCCCAGGCCCGTCCTCTGGCTGCGGCCATCCAAGTGG
CCCCCGCGTGGTGAGGCGGGGCCAGACCCGGTGACCTCCGAGGGG
TTAGAGACCTGGGCGGGGGCGGGGGCCAGTCCTCCTCCCGAGAGG
GCGCCGCGGGGACACAGCCCACCGCCGGGAGCCAGCGGGACACGG
GCCTCGGGCCTGACGCCGCCCACCCGAGGGTGCCCGAGCCCCGCT
GGGACCCGCTCAGAGCCCTGGCACCGCCCTGGGACGGGACCGACG
GGAGCGGGGGGAGCGAGGACCCGTCCTGCCGTCGGAGTGGAGCCC
GGAGCCAGGGGGTCCCCCGTCCGCCCCCAACCCTCGCGGCCTCGC
TAATGAGGAAACTTGGGGGGCGGGGTCCCCGTGCTGCCGTCCCCG
CGCCTGTGGCCACATTCTTTCCACAGTCACCTCCCCGCCCCCATT
TGGCGCGCGACGTCTGAGGTCGCGGATATGCGGTGGGAACAGCCC
GCGCCGGGGCGTGTGGAATGAGGGTGCCCGGGCGCCCCTCCCTGC
ACGTGGGGTCCCGCAGGCAGCCGCGCCTTAAGGCCAGAGTCGAAG
CCTGTGGGTGCGGACACAGGGAACGTTCGAGGAGACAGAAACTGG
GGTCCTCCCTGCGTTCCACCCGCCGCACCCTTAAGCCTCGCTCTC
CCCAAAACGCGCCCGAAACTCGGCCTCGACGGGGCCTCGGGGCCC
GGCGACCCTCGCAGCCTCCCCTGGGCAAATCCGGAGCGCCCCTGG
GACCCTTCGCACGCGCACGCGCACGCGCGCACTCGCACGGACGGG
CGCGCGGGAAAAGGCTCGTCCCCGCGCTCAAGCAGCCCGGACTGG
CGCGGGGGGGGCGGGGCGGATGAAGGGAAGCGAGGGGGCAGGAAA
TGCCGTTAATTGAGGGAAACGCGCATGCATTGCACGGGCGGCCTT
TGATGTGCGCCTCCGGGCCAGCCCGGCCCCTCCACGCCGGCGAGC
CCACCCGGCGTGCGCCCCTCTCCGCCGGCGCTCCCGGGAGCGCAG
GGCCAGCTTGAGCGCCGAGGACGCGTGGCACTTCCAACGAGCAGG
AGGCTGTGGGCTCACTCTGTCTCTAACGGGAGACAGTGCGTGGAG
CCCTTTTTGTTTCTCCCCCAACCCCTGGGCCTCCCGGGGTGGGTC
CGGAGACCGAGCGCTGCGGGGGATGACCACGCTGACCGCG.
MAX.chr19.2732:
(SEQ ID NO: 24)
CTGTTTCAAAACTGTGCCATCTGGATGTTGCAGTATACCCATTTT
GTCCTTCCCATACCTGTGCCCGGCCACCTGATGCAAGATGGGCAC
ACAGCCACTGAGGAAGCGGAGTCTGCCGCCTGCCGGCTGCAGGGT
GCCCTTAGGGGTGGCCTCGATCGCCGGTGGGGTCCGCATTTCTGG
GGGACCCGGCGCCTCGACCCGGAGCGGGGATGGTGGCTCTCTTGC
CATAACGGAGAACAGAAGCGGTAGGGTCAGCAAGAGCAGGAAAAG
AAAAATAGGGGGAGGGAGGGGGCGCCGGAGAACCCAGGGGTCGCT
CAGGCTCGGGCGCGAGGAGGCCCGGGGGTTCCCGCGGCTGGTGCC
CGCTGAGGTGAGGGGAGGGGGCCCATGACGCCGCGGCGGCGCGGG
CACTCCCTCTGCCCAACTCTCGGCTGAGCGCGGCTCCCGGCTCAG
GCCCCTCTGCCGCCGCAGCCGCGGGCCCAGTAGACACAACCCAGC
CGAGGAGCAGCAGCAGCAGCAGCGGCGCCCCGCGCTCCCTGGGGC
CCTCCAGAAAGTTTTTTTATGGATATCAGCAATCTAATTCTACAA
TTTATATGGAGAGACAAAAGACTCAGAATAACCAACACAATATTG
AAGGA.
MAX.chr19.4467:
(SEQ ID NO: 25)
CTGTTCCTCTGTGGTGGAGGAAGGGACACGCGCTTTTTTTTCCGA
CCTTAGGAAGGAACAAGGGAGCCGGGGTCCCCTCCCAGCCTGGGA
GCCCTGGGCACAGTCCCGGCTCATTTGTCAGAGCTATCGGAGCCG
TCCTCGGGCTGGTGGGAGTTCAGGGCTCTGAAAGGTTTTCTGTCA
AGGCTTGAAAGGGGGCCAGGTTTTTTTCCCCCCGGAGCCGCGCAG
TCTCGGGGCTGTTGTTCTCAGCAATCGCAGGGCCTCGTGTTAGCA
GGAAGCACAGCCAAGTAGGGTTTCCTGCGTGTTGGAGAGAGGAAG
CTCCGTAATGTTCTGGGAGGCGATGGTTAAAAATAACTCCGGTAT
ATAAAGACAGCGGAGGGTCCCCTTGTTCGCTCACTCGGGCGCCGG
CCGGCTGGACGCAGGGCCGAGCAGGTGGTTTGGGGCCTCGGGAAG
GCCAAACCCCCGCCTCTGGGCCCCTGGCTGGGGAAGACACCAGCC
AAGTTCAGAGCCCCAAGTCGGCCTCACTTCCACAACTCAGCGTCA
GGGACACCGTGGGCGTTTCTGTTTCAAAACGCTTTTCTCCAGCAA
AGAACGTAACCTCAAGCTGCTGTCAGGGTAGAGGAATCCCTGCCC
CCCGCC.
MAX.chr2.0490:
(SEQ ID NO: 26)
CGAAGGATGCGGCGCGTGGAAGGAGATGCGCTGACTTGTTCCAAC
CCATAACCTTTCGCTCGGGTCCCCATGTGCGGGCAGAAGAAGTCA
GAGCGGAACAGCCTAGTGCACTGGCAGGGCTCATTGTCTGGGAAG
ACACCGAGGTCTAGGCAGCTGGGACTGCGGAGTGGAGGCAAGGCC
GGAGGCGGCCGGCGGCTTTGTGGAAGTTTCGCGCCGCCAGGCCCT
GCGCGCCGCACGGGGCGGTGGAGTTCTTGGGCAGCCCCCGGCGCT
TGGCCCACGCCTCCGCTTCCCGCGTGTGGGAAACTCGAGCACCCT
ACAGGCACCAGGGTAAACTGCCTGTGCCTGGCCCGGTGAGGGTCG
CTCCCCCAGGCCCCGTCTCCGCCCGAGGACTGCAGGCCTAGGCCT
GCGGGGAGATCCTGAGACCGCGGTGTGCGGGCGCCGGCAGCAGGG
CAAGGCAGGGACTGTGCCCAGTCCGCCCGCCAAGGAGATCGCACG
CCGGCTTCGCTTCTGAAGCTGCAGACGGAGGCCGTGGTGAGCCTT
AGAAAGATCCCGGGACAAAGGCG.
MAX.chr2.8148:
(SEQ ID NO: 27)
CGCCGGGGCGCAAGGCCGAGTCATCCCAGGCGTCCGTGGGCCGTG
ATTCCCACTCACGCCGGGGGCCCAGGCAGGCAGAGAAGAGTTAAT
GAGCGCGCAAGTGCAGGCGGTCACTCCTGGGCCTGAAACTCCCGC
GCTGTGCATTCAGGGCCCTCGTGGCTCTCAGAGGCGCGTCCCAGG
GGCGCACACTGCACCTTGGGCTGGGCAGCTCCGCCGGGTTGTGGC
GAGCGGATGAGGGAAGGACGCAGAAACCAGGGCGGAGGAGCCGCG
AGGGGCAGGACGAGGCTGCATGGGCCAGCGAGGGGGTCGACACCG
AGCCAGAGTGAGCGCGGGGCCTGGGGCGCAGAGCCCGCCCAGGGA
GCCGGGAGACGCCGCGCAAGCTCCCCGGACAAACGCAATGACCGA
GGACGCGCGGGCGAGGCCGTCCAGGGAGCCCTGGTCCCTCAGCTG
CACCGGACTGAGCCGCGACCGCTCAGCACGCGCTGCTTATAAATC
AGGGGTGCGCTTCCCAAGCCCCGGGTGAGGTCCCCTACGTCGGCA
CAGCCTTAGGAGCTGCAAAGCAGCGCGCGCCTCCGGGGCTCCTGC
GCGCCCCTTGAACCCCGCCTCCCGCATCCTCCTGCAACAGCCTGG
AGCTCCCTGTGCAGGACGCAGCGGGGGGCGGGGGGCGGTCTTAGG
AGGCTGCGGGGCGCACTCCCACCTCCTGCCTCCCCGAGACCCCCA
GCGCCTTCTCCAGGGTTTAGAGCGGAGGTGAAGGGGCCTCGTCCT
GCACCGCCACTGGGCGCCTGGGCTGTTCATCATCGGTTACCGCCG.
MAX.chr2.3137:
(SEQ ID NO: 28)
CGTCCTGAGAACCCGAGAGAGCAGGGCCCGCTGGGACAGGCAGGG
GAAGGCCTCGGGAGGGACACGACGGTCCGGCAGCAGAGCCTGCGG
GGCTGGAGGAGGCGCCCTCCTCTCAGCTGCTCTTCCTGCCCCTTT
CGGTGGCGAAGATGGATGGGGCCCGGGGCTTTCGGCGGGGCCCGA
GGGGCCGGCGAGGCTGCGGCCCTGGAGCCCCCTGCCTGGCAGCCA
TTTGGGCCCCAGGGAAATATCGGCGCTTTGGCTAACCGAATTATT
CTTTCGGTTTGAGCCAGCTCCCCTTTTTGAGTCAGATCCGGCGGC
AGGGCCAGAAAAGCGCTTTCTGAAACCCCAGCGCGGTCCTCGGTG
GGGGTGGAATGGGGTGGGGTGGGGGGCGCGGCCGCGCCGCTGGGC
GCCCTCCCCGCCCTCCCCCCTCCCCACCCCCAGTCCTCCCTCCGC
TGCCCGCCCCCCAAGCCCGGTGTCGCCCCCTCCGCCCCCTGCCGC
ATCCCCGGAGCCAGTGCCCACAGGGGCCAGGCAGCCCGCAGGGGT
CGCTCACGGCTGGTGTAGGGGCTTGGTCCACCACGCTAGTACTTC
GGGCACCAAAATAGAAAAAGAATAACGCTTGGAAAGAATCTGATG
TTTCCG.
MAX.chr4.4210:
(SEQ ID NO: 29)
CGAAAACTACCCCGCGGAAACTAGCACAGTGTGCCTGGATGTCTG
TGTCCCGGGACCTCGGGGAAGAGGGCCCGCACCGGTCTGCGAATT
GCAAGGCCCGGCCTTCCCCAGCGACGCTCTGGTATCCGCTGTCCC
CTCCCTGTACCTCCGCGACCCAGGGGACGCCCAGTGCACCAGGCC
CTTCCCCGGGGTCAGCGGAGGCGCAGGGCGTTAGCCACATCAGAG
GTGCAAATTTACCCCGGGCCCAGGGGAAAATGGCGACAGCGTTCG
CGGCTCCACCCGGGGCGCGTGTCAGCGTTGGAGAGCCTGCCCGGC
CTGCAGAGGGCGTAACAGGCACCGCTGGGGAGAGCCAAGCACCCC
TGCGTCCAGGATCCGTAGCGCCGAGCTGCAGGCCCGACCTGCAGG
GGGCGTGCCCGGCATGGGAAGCTCAGGCTACGTCTCCGAAGCTTG
CGCTGAAAACACCAGAGGTAGGGAAAACGGGGAGAGCGTACTGTG
CTGGGCTCTACCCTGGACACCCCAGTTTCATTCTCTGCGAAGCCA
CGCGCTGGCAGGGCTCTCGGGACGGCGATACCCAGGGATGATGGT
ACCCCTGGTCTCGGCGGGACCTCCCGGGAACTTGTCCTGGGGGAG
GGAGCCCAACTGGCCACGTACTGGTAGCAGCAGTGGGTGGAGCGC
ACAAACTCCGAGGCCCGCG.
MAX.chr5.0931:
(SEQ ID NO: 30)
CGCCTCCTGCCTGAGGCGGGCTGGGGGGTCGTTGTCCTCGCAGCG
TTAAGGCGAGTCTGGGACAGGACCCCGGCACCCCCTCCGGATCTG
TGGCATCCTCCAGGACTCCGGCGCAGGACGCGCTCCAGGAGCCGC
TCCTTCAGGGCCTCCGGTGCGCGCAGTCCGGGCGCCGGACGAGCT
CCTTTATCAGAAAGGGCAGCCGCAGAGCCCGCGTGTGCGCGATGT
GGCTGCGGGTGGGGAGCGGGCGGCGGGCCCGGGACACCGCGGCCA
CTGTTCTAGCCCCGCCTGGGCCGCCTGACCGCGGCTCCGCTGCGC
CGCAGCCCCGCGCCCCTCTGGCTCCTGTTCCCGGGCGCGGGGAGA
AGGCGGCGGGGCGCGCCTGGGCCCGCGCGGGTGCGAACGCGAGGT
CTTTCCTGGGTGCTCCCAGGTCGGAGGATTCCCAGGGCGGGGGCC
ATCAGGGTGGCGAGGAACCGGCAGGGACCAGCCTCCGCTAGGACC
GCGCTCGTGGAGCG.
MAX.chr5.9924:
(SEQ ID NO: 31)
CTTTGGTTTGAAACACTGGAGGTGGCCCAGGGCCGTTTTCCTCAA
AGGACTGAGAATCTTGATTTGCCAAGTGCTTGGGGCCTCCGCCCA
AGGTGTTTGGGGGCTGCGTGGTGAGCCGAGGCAAAGCCAGGGTAC
CCCGATCGTCTTCCGGGCGCATCCACCATGCGGCACCGCCCCAGC
CACGGCGGCCGCGCGTGGAGACCCGGGGGCTTAACAAAGGGCTCC
GCGGGGGCACGGGGGGGCGCGGCCACGTGACAGGCCCGAGCGCGA
CGTCGCTGTCCAGCCGCGGGGAGGGGCGGCCAGCCCGGGGGGCCG
TGGGGCTTCTTGACATAGAACGTCCGGGCCTCGGGCTGGCCGCGC
CGGGCCGCGCTCCGCCGGGATGAGAAGTACTTGTCTGGCTCCGCG
CTGGAGAAGCCGCACCTCTCATCTCTCCGGCTCTTACTTGAAAAA
GCACTTGGAAGAAACTGTGTGTGCGCTGGGAGGGCCGCGGGGTGG
GCCGGGGCCGGCTGCGAGGCTGAGGGGGGCCGGCTGGTGGGTGGA
TGGGGAGGAGGTTGAAGAAACAGCCCCTTTCTGAGTGACAGGACC
CCTTTTCAAAGGGCAAACAGAAAAAAAAAAGAAGAAGAAGAAGAA
GA.
MAX.chr6.9522:
(SEQ ID NO: 32)
CGAGGCGAGTTAAATTCCTTTTGCCGGTGCCTGGCTGCGAGGACA
AACGTCCGTACTTTCGTTCGGGAGCCACGGGCAGTCCAGGGGCTT
GGGTTAGAAGCAACGGCTCTCTTCCAGGGGCTGTGATCCGGGTCG
GCCAGGGAGAGCGAGGCCCCGGGGTCCTCTGTGAGGTCCCCAGCG
AAGAGACGCAGCTGGGGAAGGCGCCGCCCCCGGGCCCCCTGCGCC
ACCCTAACCGGGCCTCTCCTTAGCAAAGTTGACAAATTCTTGAGA
GTGTCAGCCCAGGGCTGCGCGTGAGGGCGCTGGGACCGGGGAGGA
AAGAGCACCTGCCGCGCTCAGCCCGACTTTGAATTTGTTTGTTGT
TACCGTTTTTGTTTTTCCTCCCAGTTTCCATAAACGCTTAGTATT
TCGAGGCACTTTGCAGGTGTTGGCGCAGGTGATGATGGGCCTCGT
TGGACTCTGCCTCCCACGCATCCTTTTGTTTTCTGCGCGCCAGCC
TGTCTGACTGTGTCCTGCGGGGACCCCGAGACAGTCCGGGGTCAG
GGCGTAGAGACTCATGCTTGCCACTTGACCCATCCGCAACCCGGG
GACCCCCTAGCCCGTCGCGGAGCTGGAGTTTGGGCTTCCGGCTCC
CAGCTCTCCGCCCTGGATACAGGAAGAGGGCGGGAGAGGTCGCGC
ACCCGCGCCGCTCGGCGGGGATCGCTCACAGGGGCTCCGGGGCCA
CCGCGAGCGCGGACTGCGGCTGCTGGCGGGCTCCTTCGTCGTCCA
ACGCACCCCATCCTCTCCCGCCCCGCAGTGTCCCAGGGAAGGCTT
CACTGAAAACAGACGCTCGACGGAAAACTGACTCTGCAGGCCCGA
GCTTTCG.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in pharmacology, biochemistry, medical science, or related fields are intended to be within the scope of the following claims.

1. A method of characterizing a biological sample, the method comprising:
determining a methylation profile in at least one differentially methylated region (DMR) of a DNA sample obtained from a subject having or suspected of having a gynecological cancer by treating the sample with a reagent that modifies DNA in a methylation-specific manner.

2. The method of claim 1, wherein the methylation profile in the at least one DMR indicates the subject has or is suspected of having at least one of ovarian cancer (OC), cervical cancer (CC), and endometrial cancer (EC).

3. The method of claim 1, wherein the at least one DMR comprises one or more CpG sites in ADAM8, ADHFE1, AES, AGBL2, AIM1, AK5, ALKBH3, ARAP1, ARHGAP20, ASCL2, BCAT1, BEGAIN, BEND4, BMP6, C12orf68, C13orf18, C14orf169, C14orf169, C18orf18, C1orf61, C20orf195, C4orf31, C5orf52, C6orf147, C7orf51, CD14, CELF2, CHCHD5, CHMP2A, CHST10, CLIC6, CLIP4, COL13A1, COL19A1, COL6A2, COPZ2, CREB3L1, CXCL2, CXXC5, CYTH2, DAB2IP, DGKZ, DLGAP3, DNASE2, DSCAML1, EBF1, EDARADD, EGR2, EIF5A2, ELMO1, ELMOD1, ELOVL4, EME2, EML6, EPSTI1, FADS2, FAM109B, FAM126A, FAM174B, FGF18, FKBP11, FLI1, FLOT1, FOXD3, FYN, GAL3ST2, GALR3, GAS7, GATA2, GLT25D2, GNB2, HDAC7, HIC1, HLA-F, HNRNPF, HPDL, HS3ST4, HSPA1A, IDUA, IGSF9B, IL12RB2, IRAK3, IRF7, IRF8, ITPKA, KCNA2, KCNC3, KCNC3, KCNC4, KCNH8, KDM2B, LBX2, LCMT2, LOC100129726, LOC100287216, LOC255130, LOC339290, LOC729678, LPPR3, LRRC41, LRRC8D, LTBP2, LYPLAL1, MAST4, MAX.chr1.2152, HIVEP3, GRAMD1B, MAX.chr11.0394, MAX.chr11.3750, FAT3, SLC16A7, MTUS2, LINC02323, MAX.chr14.7696, MCTP2, LOC107984974, TRIM80P, MAX.chr19.5552, ZNF433-AS1, ZNF254, MAX.chr19.0548, B3GALT1, MAX.chr2.8918, MAX.chr2.4778, MAX.chr20.3853, MAX.chr20.2903, MAX.chr21.5011, DSCR9, MAX.chr22.5665, MAX.chr3.6408, LINC02028, LINC02084, MAX.chr5.3588, CTD-2532K18.1, HS3ST5, ARHGAP18, GRM4, LINC01004, MAX.chr8.5938, MAX.chr9.4007, MAX.chr9.2025, TRPM3, MED12L, MIAT, MLH1, MLH1, MMP16, MRPS21, MSI1, MT1E, MX1, MYC, MYH10, MY015B, N4BP2L1, NBR1, NDRG2, NEGR1, NEU1, NOL3, NR3C1, NR3C1, NRP2, NTN1, NTNG1, PAPL, PAQR9, PDE10A, PDE3B, PDE4A, PDXK, PER1, PISD, PLEC, PLIN2, PLXND1, PPM1E, PPP1R9A, PPP2R5C, PRDM5, PTP4A3, PYCARD, RAB3C, RAI1, RARG, RASA3, RPRM, RREB1, S100A6, SAMD5, SBNO2, SDC2, SDK2, SELM, SERP2, SFMBT2, SHF, SHH, SLC16A11, SLC16A5, SLC25A22, SLCO3A1, SMTN, SPDYA, SPINK2, SPOCK2, SPON1, SQSTM1, ST8SIA1, TAF4B, TAF7, TEAD3, TERC, TIAM1, TLE4, TMEM101, TMEM106A, TRIM9, TRPC3, TSC22D4, TSPAN2, TSPAN5, TTC14, UBB, UBB, UST, VAMP5, VIM, VSTM2B, ZBTB7B, ZEB2, ZFP3, ZFP36L2, ZIC2, ZMIZ1, ZNF14, ZNF211, ZNF280B, ZNF302, ZNF382, ZNF480, ZNF483, ZNF491, ZNF569, ZNF610, ZNF702P, ZNF709, ZNF773, ZNF845, ZNF91, CDH4, LRRC34, MAX.chr10.4460, NBPF24, OBSCN, SEPT9, ZNF323, ZNF506, and/or ZNF90.

4. The method of claim 1, wherein the at least one DMR comprises one or more CpG sites in ACSF2, AJAP1, ARL10, ARL5C, ASCL4, ATP6V1B1, BARHL1, BEND4, C17orf64, C1QL3, C2orf55, C4orf48, CA3, CDO1, CELF2, CLEC14A, CSDAP1, CYTH2, DLGAP1, DSCR6, EPS8L1, EPS8L1, FAIM2, FGF12, GATA2, HIST1H2BE, IRF4, IRX4, ITGA5, KCNA1, LECT1, LHX1, LOC440925, LPHN1, LINC02767, MAX.chr1.2533, SOX1-OT, MAX.chr13.3357, MAX.chr14.2093, MAX.chr17.2455, MAX.chr18.4390, MAX.chr19.2732, MAX.chr19.4467, PANTR1, MAX.chr2.0490, MAX.chr2.8148, MAX.chr2.3137, RIPOR3, SCRG1, MAX.chr4.4210, HMX1, CTC-359M8.1, MAX.chr5.0931, MAX.chr5.9924, LIN28B, MAX.chr6.9522, TTLL2, RNA5SP243, DLGAP2, MEX3B, MNX1, NEFL, NETO1, PAX2, PDX1, psiTPTE22, RASGEF1A, SALL3, SALL3, SEZ6L2, SHANK2, SHANK3, SKI, SLC35D3, SORCS3, SORCS3, SOX1, SQSTM1, TBXT, TCERG1L, TERT, TNFSF11, TUBB6, ULBP1, VAC14, VWC2, WDR69, ZBTB16, ZNF132, ZSCAN12, ZSCAN23, KRT86, CYP26C1, GYPC, DIDO1, EEF1A2, EMX2OS, GDF7, JSRP1, SMPD5, MDFI, MPZ, and/or VILL.

5. The method of claim 1, wherein the at least one DMR comprises one or more CpG sites in AIM1, FLOT1, GAL3ST2, LRRC41, LYPLAL1, MAX.chr11.3750, PISD, RAI1, ZIC2, ZMIZ1, CDH4, ZNF506, ZNF323, OBSCN, ZNF90, and/or SEPT9; and wherein the subject has or is suspected of having OC.

6. The method of claim 5, wherein:
the at least one DMR comprises one or more CpG sites in AIM1, FLOT1, GAL3ST2, LYPLAL1, and/or OBSCN; and wherein the subject has or is suspected of having serous OC;
the at least one DMR comprises one or more CpG sites in LRRC41, PISD, ZIC2, OBSCN, and/or SEPT9; and wherein the subject has or is suspected of having clear cell OC;
the at least one DMR comprises one or more CpG sites in MAX.chr11.3750; and wherein the subject has or is suspected of having endometroid OC; or
the at least one DMR comprises one or more CpG sites in RAI1 and/or ZMIZ1; and wherein the subject has or is suspected of having mucinous OC.

7-9. (canceled)

10. The method of claim 5, wherein determining the methylation profile of one or more CpG sites AIM1, FLOT1, GAL3ST2, LRRC41, LYPLAL1, MAX.chr11.3750, PISD, RAI1, ZIC2, ZMIZ1, CDH4, ZNF506, ZNF323, OBSCN, ZNF90, and/or SEPT9 comprises comparing the methylation profile to a corresponding region from a control DNA sample obtained from a subject that does not have OC.

11. The method of claim 1, wherein the at least one DMR comprises one or more CpG sites in AK5, ELMOD1, RABC3, TRPC3, ZNF480, ZNF491, ZNF610, ZNF91, and/or NBPF24; and wherein the subject has or is suspected of having CC.

12. The method of claim 11, wherein:
the at least one DMR comprises one or more CpG sites in AK5, ELMOD1, TRPC3, and/or ZNF480; and wherein the subject has or is suspected of having adenocarcinoma CC; or
the at least one DMR comprises one or more CpG sites in ZNF491, ZNF610, and/or ZNF91; and wherein the subject has or is suspected of having squamous cell CC.

13. (canceled)

14. The method of claim 11, wherein determining the methylation profile of one or more CpG sites in AK5, ELMOD1, RABC3, TRPC3, ZNF480, ZNF491, ZNF610, ZNF91, and/or NBPF24 comprises comparing the methylation profile to a corresponding region from a control DNA sample obtained from a subject that does not have CC.

15. The method of claim 1, wherein the at least one DMR comprises one or more CpG sites in c18orf18, FKBP11, MLH1, NR3C1 and/or TERC; and wherein the subject has or is suspected of having EC.

16. The method of claim 15, wherein:
the at least one DMR comprises one or more CpG sites in MLH1 and/or SEPT9; and wherein the subject has or is suspected of having clear cell EC; or
the at least one DMR comprises one or more CpG sites in NR3C1; and wherein the subject has or is suspected of having endometrioid EC.

17. (canceled)

18. The method of claim 15, wherein determining the methylation profile of one or more CpG sites in c18orf18, FKBP11, MLH1, NR3C1, and/or TERC comprises comparing the methylation profile to a corresponding region from a control DNA sample obtained from a subject that does not have EC.

19. The method of claim 1, wherein the at least one DMR comprises one or more CpG sites in CDO1 and/or DLGAP1; and wherein the subject has or is suspected of having CC, OC, or EC.

20. The method of claim 19, wherein determining the methylation profile of at least one CpG site in CD01 and/or DLGAP1 comprises comparing the methylation profile to a corresponding region from a control DNA sample obtained from a subject that does not have CC, OC, or EC.

21. The method of claim 20, wherein:
the method further comprises determining the methylation profile of one or more CpG sites in AIM1, FLOT1, GAL3ST2, LRRC41, LYPLAL1, MAX.chr11.3750, PISD, RAI1, ZIC2, and/or ZMIZ1;
the method further comprises determining the methylation profile of one or more CpG sites in AK5, ELMOD1, RABC3, TRPC3, ZNF480, ZNF491, ZNF610, and/or ZNF91, or
the method further comprises determining the methylation profile of one or more CpG sites in c18orf18, FKBP11, MLH1, NR3C1, and/or TERC.

22-23. (canceled)

24. The method of claim 1, wherein the at least one DMR comprises one or more CpG sites in NBPF24, and wherein the subject has or is suspected of having CC; and
wherein determining the methylation profile of the one or more CpG sites in NBPF24 comprises comparing the methylation profile to a corresponding region from a control DNA sample obtained from a subject that does not have CC.

25. (canceled)

26. The method of claim 1, wherein the at least one DMR comprises one or more CpG sites in CDH4, NBPF24, MAX.chr10.4460, ZNF506, ZNF323, OBSCN, ZNF90, LRRC34, SFMBT2, LINC02323, CYTH2, LRRC8D, LYPLAL1, LRRC41, and/or SEPT9, and wherein the subject has or is suspected of having EC; and
wherein determining the methylation profile of the one or more CpG sites in CDH4, NBPF24, MAX.chr10.4460, ZNF506, ZNF323, OBSCN, ZNF90, LRRC34, SFMBT2, LINC02323, CYTH2, LRRC8D, LYPLAL1, LRRC41, and/or SEPT9 comprises comparing the methylation profile to a corresponding region from a control DNA sample obtained from a subject that does not have EC.

27. (canceled)

28. The method of claim 1, wherein the at least one DMR comprises one or more CpG sites in CDH4, ZNF506, ZNF323, OBSCN, ZNF90, SFMBT2, LINC02323, CYTH2, LRRC8D, LYPLAL1, LRRC41, and/or SEPT9, and wherein the subject has or is suspected of having OC; and
wherein determining the methylation profile of the one or more CpG sites in CDH4, ZNF506, ZNF323, OBSCN, ZNF90, SFMBT2, LINC02323, CYTH2, LRRC8D, LYPLAL1, LRRC41, and/or SEPT9 comprises comparing the methylation profile to a corresponding region from a control DNA sample obtained from a subject that does not have OC.

29. (canceled)

30. The method of claim 1, wherein the at least one DMR comprises one or more CpG sites in KRT86, EMX2OS, JSRP1, DIDO1, MPZ, VILL, SMPD5, GDF7, MDFI, c17orf64, GATA2, SQSTM1, and/or EEF1A2; and wherein the subject has or is suspected of having CC, OC, or EC; and
wherein determining the methylation profile of the one or more CpG sites in KRT86, EMX2OS, JSRP1, DIDO1, MPZ, VILL, SMPD5, GDF7, MDFI, c17orf64, GATA2, SQSTM1, and/or EEF1A2 comprises comparing the methylation profile to a corresponding region from a control DNA sample obtained from a subject that does not have CC, OC, or EC.

31. (canceled)

32. The method of claim 1, wherein the at least one DMR is associated with an area under a ROC curve (AUC) greater than or equal to 0.8, and wherein the ROC curve discriminates between a subject having or suspected of having OC, CC, or EC and a control sample.

33. The method of claim 1, wherein the biological sample is selected from a tissue sample, a blood sample, a plasma sample, a serum sample, a whole blood sample, a secretion sample, an organ secretion sample, a cerebrospinal fluid (CSF) sample, a saliva sample, a urine sample, and a stool sample.

34. The method of claim 33, wherein the tissue sample is a gynecological tissue sample.

35. The method of claim 34, wherein the gynecological tissue sample comprises one or more of vaginal tissue, vaginal cells, cervical tissue, cervical cells, endometrial tissue, endometrial cells, ovarian tissue, and ovarian cells.

36. (canceled)

37. The method of claim 33, wherein the secretion sample is a gynecological secretion sample.

38-43. (canceled)

44. The method of claim 1, wherein the reagent that modifies DNA in a methylation-specific manner comprises one or more of a borane reducing agent, a methylation-sensitive restriction enzyme, a methylation-dependent restriction enzyme, and a bisulfate reagent.


SEQUENCE LISTING (32 sequences)

SEQ ID NO: 1 - DNA, 2525 bp, Homo sapiens
  source 1..2525: mol_type = genomic DNA
cgccaccccagttcggccctgctgggcgcgcgagccaaggccgcggggcaccgggaggccattttgcgcgtgcgctgctcgcctcgcgccgccctcggctctgcggactcggatcccgccaaatttgaacgcgagattgtcaggccctgaggggcttgaggggcgggggaacgacgccgctctccaaagttggaccccgtggcgagcggcggcgacagccgggtgctcgctgcctcccgaggtgctcccttttcccgccgaagccctccacagcggcaggccgaggcgcagcgacgtgtccctgtaccccgagttcagcgcgggcgggaaaacgacctgcacccggggaggcagcggcttcgcgggcagagcccacgggagcgcgccctgctaggagccaggccggataatcgcctttctttgtcctcctccctcttcgagtccaatcaatgccctttctccttaatgaacgaggtgtccttggagtttgaggttttgttggatgattttaaataaaattattaagttataaagtggccaccctgaaggttcccgaaggcgacttcatgtctgtgactggaaaggcctagaggagagggtcctcccgctgggctcgtttaatagaacgcgctcgaatcccctgggaaagagccttgactgggtgacagggctgaggaggggtggctgcgcggcgggaatctcaagatctgggcaaaggctcgcgtctcgggacgcgaagtcgacgccaaaatgggtccccggacaaggcgaccctgggagtgccggcgcccccggccgggcagaggagcgggtgggccgaggctgggacatcgcctccgaaagctgccgggacgcggcggcttcctgcagagcctgcgcctgccggatccccagaacacagaagcttctcggacatgggagctccccgtgcgccctaaaaccaggagaggaagggacgacttgggaaaagggactggggaaacagcggagaagtgaaagcggcctaaaatgggcgacggcgggcgagtcctctttatcagtgcagcaggctgccggagccgccatttggtggcggatctcggtagttcagtagcacgttgtgctgaacgtcacaactggcttgtctacgtggcatcgtcatttcttaaccgcggttttacgaaatgcaaatttccccctggccttcctcctccgcggccgtcgacccccctcggcgctccgggtggacggctccggggcgcggctcgtccctcgggtggtgcagcccccgcggcccgagacccggggagggccgggggtactttctgcgaggcgccttccccgcggcttctgcccgcgccaaagcctggtggaatccagcgcagacctaaagcacgcttgacaccccgatttttcgagactaggacgactctctgagccagcagctttcctctccctctcggggagaatctcatttccttggggtggtgagggtgacgggcactgtcttttggccccgcgtgtccgttccccggtctcccgcctcacccctctgcgaggtgaggaggggaaacggcgagcttaggcctggcgggaaggagcctacccgacgagagggctccgcggggagggtcggttggaatcccgccctagcgcctcctgctctgcccggtccccaccggggacggggaatgccagtcatttctgttgagtgctagcagggccggtgtcaccacctcgggtggccgaggcttcgaggttttcatgaaaagcccccgaagcgtgaggcgcccgcccagtggagaacaaagggccgagggccgaaggcgaggcgaggcagcgcgcgcggctcccttggctcgacctagctgggagtcgggggcgcgggcagggctcactcccggcctagaaactggagcccgccacccccgccccgcaggcgaccgcagggatcccattcttggagcccgagctgccatgttgccttcgcggaggccgccagtcacttgacgcttccgagacagcgaagcccccaacctgagagcccttcggccgtctttgccgcacagctgcagtcaaggcccggagggactgcgggacgcgggcgggagcgagagccctgtgggctgccaagccggcgcggccgcgccgcggcagccgcttcccttgccaccttcgttccaggggctgcggggctgcgcgctcggcagaggctcggttgccagtagcaaccacacgacggcgatttgcagccagggccgccgccgcagccgctggtacctctgcctcctcctacacctcgggctcgagcatttgaaaccctgggggttgccttcggtgacatctcccgcccccacctccagtcctcagtctccaaaatcctcagctctgctcaaaagccagcgcccccggctgggccctgcccccaccgcagacaataggagcggctgggagcgcacagggcggcgcgcgccgcgagcagcgggcacctgagcccccaaatccgggcgcgtcgctgagtctcagccccaggtgccctcttcgcg

SEQ ID NO: 2 - DNA, 587 bp, Homo sapiens
  source 1..587: mol_type = genomic DNA
cgactcttcgagcgcccctctgcttctgtagaggggtcgagccatgtcaaggtagaccctgtgtcggcccgtctccctcggatcctccgcaccaatcactgttgctgaatccgacacccggcggatccagtgcggagtctcgaacagctgcggagctgggagctacgggacatgaggagtgcgggggggaagagaagacggcggaggaaaatcccccggcggtgctcaactgcggctttctctctcggctgtgagccggctccgccctccggcttccagagcaagtggcttctgcgttcaccgccccccgccgtttgtggggcggggccgattcataagaatcggttctcaccaatggagggcttagcatgtttaacctcaggatcataaacaaaagacactgctagaacggtcgggaaagtcatacgctttgcttatcttatatatagatttctaaaattccaaaccggggacgcgttggtggtgtagtggtgagcacagctgcctttcaagcagttaacgcgggttcgattcccgggtaacgaaacgtttttgtctttccttctacgaaaaacttttctgagccg

SEQ ID NO: 3 - DNA, 594 bp, Homo sapiens
  source 1..594: mol_type = genomic DNA
cccttgaggccaggagttcgagaccagcctgtgcaacacagaagacactatctctacaaaaaattaaaaaattagctaggcatagtggcacatgcctgtggtcccagctactccggagattgaagcaggaggatcacttgagtgagggaggtggaggctccagtgagtcgtgatcgtgccactgcactccagcctggacgacagagcgagaccctgccccctcgccaaaaaaaaaatactgggatgctatacacaaaattgccttgaaaacttgagcacggaacaccaaacagctaagcgtgccggtttggggagggcgggggaggaataaggagctgcaacggtaagaggccgccacacggtggcgcagtgaggctgggaaacggtgcaccccgcgcaggagggggcactccccgtcgcggccacccggggtgggcaggaggcggcgcgggctggctggtctctcccgagaaggttctctcccgagaagggtgcgtctcagggcttgtcagtggacccctggaacatggggaagacgcacagacaagggtttcgctctttgctctcctctctccttgtcagacctctgtgacc

SEQ ID NO: 4 - DNA, 1047 bp, Homo sapiens
  source 1..1047: mol_type = genomic DNA
gtcttccagttccactgagggccgagactttgtctttgcggccccagtacttgcttagttccgagagtgcggtttgcactcagtaagtagccacttactgagtccaatcgattattggaaacctaatttttcatcactgcttctcccacaagaagctctaggactgactcctcaaagaccaaaactggaattagcaatcccgctgtttacccggaggcccggtcaaatgtcttaaatctgggaggattcctcctgggaaattccagtaagggcgcggagcaggtcaggaaggaggttactttttgggtctttatcgtctatgatgggagaaaaggagaaatgaagactcgattttgctgaacgcctgctcattgtcaattttgccggttcatctctcaagaaatcagcaaaaagactcagaattgtaatcgcgaagggaaagaatgcggccacgtggcctattttcctgtggatagactaagcaaacgcttttcttcaggggcccggatagctcagtcggtagagcatcagacttttaatctgagggtccggggttcaagtccctgttcgggcggatgctgttttagtttccaataaaatggatttgggcgaggctgagagaaaggaacgttatgtgaaacccgcttggggtgcctcctccttgagggaaaccagaacttgctagtgggttcttaccggaagaagtgaaacgtgtggaaaatgccaagaaactttatcttccaatagcaggcttttcttttccaacctttatacgttgctttgtcttaggatattttttcttttaaattgtattttatattcaaaacagatcaataaacacatcgtcagagtcacaattagtaatattcttggcaagaattgtgcagcttttggcaccgaggaatgttttcaggcactttttattaaaagtgcgatgaggaaactgagactcaagaaatatttcagatgaagacacgtaaagacgcaagattcctacatctccaactggacgcagtccttcaccagattcttaatgctctggtgggc

SEQ ID NO: 5 - DNA, 629 bp, Homo sapiens
  source 1..629: mol_type = genomic DNA
cggcacgtgggtgggcgatgacgccatttactgagatttgatccccaccacacggctccggggtgagaattatgacatctggctcaacacggctgcccgggacccacacagccgagcggccgagctcgggcggagtcccagggcgcccacagcaccccgccagcgcgccccgtccagcagggcagcttttgggcggaggcgacccccaccgcaggtcccaggaccctgcgtgctcttgagccaggggtggagaggcccgaccgcggggggctgccccaccccgccgcccttcaccgcgagccggggcccagaccgcccagccacgcccagagcccgcgggcggagacgccaggggcggtgccagcgaggtcccgtccccggtaccccgtcccgcccccccacacgcggtgacctggggacgccccgcgggagtcgttctgcggctccccctggcgtcggctggggccaccgcccgggctcccacctcaaccctgcaatatggggttggggcagagtggtctgctgcccgctgcccgcagccgcttccggttagggagggagcctgggcctctgggtgctcacgctgcgcttaacgctggtcccggcagcagtgagggtggaagcggccg

SEQ ID NO: 6 - DNA, 639 bp, Homo sapiens
  source 1..639: mol_type = genomic DNA
cggaccgtagctccttccacgcatgaaccccgcacacgagtcgggattccccccatgaccctcccgtggcccccgcacaatctggagagacgcggggctgcgggcgcggagctgcccagagaggactcctgcccgggcccgcagtcgccgcgaagggacgggacaggacgcccggggtcccggctgccagcccagccccaccctgcggccgaggggaccgagggccgagctccgccagcggtactccggtccacagagcccggagtcgctggctgggaggccggggacccgccacggccagttccaaccagcccctcctcccgtctcgggatccctggcccctcacgctcaccattttccgaattcctccgtgtcccgggggcctctctgcggctcccacgaccagtgcaggtccctgtgtgacagaggctgccgcagactctccagagtgcctctcagcgacagagacaggagcccagcgaagtggcgtgtagaagacgccgcgggctttttcaatctcgcaccctcttagctgaagtgcgcctgattgacagttcccacgaccccgccccacggccctgattggatagtgcgacagatcccgccccctgacgactgagttacagaagcgatctcacg

SEQ ID NO: 7 - DNA, 2256 bp, Homo sapiens
  source 1..2256: mol_type = genomic DNA
cgcgggaagagactgcccagcccgagggtcgcaggggcagaaaaccccaggtcctgaacgcgcctgggccccgcggcgaacctggcgtccccaccagacaccagaatcagattcaggccaggccaggaaccaccccagcttcctgcatccgccttggtccaggcagcaatgacggcggcggccgccagggggacggaagctcaagggcggggatccaggcccaagcctgggcgcgcccaccaaggcgtggatccggacgaaggttccggaacagccggtcccgagcacccacgtgcagcttccgatcacagccctggcgtggcttctctgtccccggctaaggcccgcggtcgctgcagggtgcctcgccgagggtgcgggctcggggctcacagtgctcccagcctctcccaccccaaccccgccattaaggggagccccagcgacgccctgaaccctgaaagtcacactggggccggggctgcacttggcgtccgcttcccctcccgtcacagtgaccagggctagggccgcggtcggggccgtcggggagagcggagggcgcgtgggaatggggctcgctgcgccacggaaatcccgcgccgcccttcgagtcctcccagctgctccgcagagccggggccgctgccatcgcgtctgccccgagggtgcgcaggcgggtacctgtcccgactcggggacagcgggagcccggagacgcccgtcgggcttcccagccccacctgggacgtctctaggggcgggaggccaggagagaagagggtgggagagatgagctgcagggggatacgggacccagggacccgggatcttataacacgctttccacccgtaggattggggcccacaaatgaccagaaaggtgggactcattcgccccctttgcagatggacccatgttggcgccccttagatctgcagtgggggcaccaggacctgcggtgagcgcctctgcgccccaaacgccagcaggtccgcccgaccgcattcccagctggcttgctttgcacaaatgctgcgtcagggcacgccccacacccactccttcccagatcgcgaccctcacgccttcgcagacagaaagtgtcctaagcacaggcaggctcagagcccgcctccgcctcggattcgctgtgtggcccggagccagtatcttggcctctctgggtctcagtttgctcatctcagtgaatgggacacagacacagtcgcgctgcgggctaacgctttatttgccagccaaggccccgggcccgcctgggcttctgctcagaagatcctcacggagtccagctgcacgtccccgcccacctccaccaggcgcacgcgcgccagcggcaggcggtggcggaagtggtggtactgggcgtccccaaccacggcctgcaggggagggtcggtggtgaggattccggaggcccgtgctgggtggccctggggaaatcactcatcccctctgggcctcagtttcctcactgggaaaatggggctattgttcattctaactcttgcgtgaggatcaaacgagttgactgtgtggcacagtaaaaagaggcttttttagtgctggtaatggatattctcatttcagcgaccattacccgctattaaagcgcagaggagggaggtgaattcgcgtaagctgtgggtggtggaggatctgccgccactcccacccgccaatccttgctaggacgagttcctgggcgctgtttccaacccatccctccccatgcctcaaaccccggacctcaggaaggaatgaactgggagtagggtctgggatggcgagtctgggccacgccttcccgctaggacgcccaccccttggacacttggctggtgctcgcctcgtcctgaccctgctgtctctctgtccctcggacccaagtgggagttgtttaggcgagagagagggtcgaaggacacccttctccgccttggccacgacttccctacccccctcaccccgccccgaacctccctgccttccaccaatagcctggctttgcccaaccctctgctccagggacctaagtcttggcgtccacgcccctgtcgcagagacgcaccttgaagccgtcgtctgacgcgatgatgagcacctcgaagggctgcccgcgctggaaaggaacgcccggcccgcgctcctcgcggccccaggagccttgctccttgctgttgaagaccacctccgacgtgtccagccgggggttgaaatgcagcgcggcatcg

SEQ ID NO: 8 - DNA, 620 bp, Homo sapiens
  source 1..620: mol_type = genomic DNA
gaagtcagggcagtgctgcaaaacctccacagtgcggaattccgggaaaattctttacagaggtgtggaggtggaggaaagcttcctgggcaggcctttggggtcgtccccacgcaggcgcttgcagccaccccagctcgcgcggggccgggctttggggtgtgagagctgggacgggagtcgggtggatgcctggccggagccgccagctcccctcgtcctctttgcttgtcctttagcacaagggcgagcagcgtaggacaaagactcgggcggcagctgcctggttcggcgcgcaggggcggcctcggccacccggggcgcccgccgcctccaccgccccgcgggggaggcccgatgcccgtctttgtctgtgccgccgccgtgggccgggtccgcaggaagcgggcgccatcgtgcggcctgagctggacactgcgcccccggaggcgcggaggcgcgaaccaccaagcgtggctccaagctccacggggacgctggtgtcatcgtggccacgactgcttgtactgttgtggtgcgttctcttttgtatactaagtgctgtgtgaacacagaaccacttccagtaaatgcaactgagccgtcgccagcagaaaaag

SEQ ID NO: 9 - DNA, 542 bp, Homo sapiens
  source 1..542: mol_type = genomic DNA
aacagtggcgctcagagaagacaggacagcgggcgagagcttggggggcgatgggaggtggagaggcactccaggtccccagggggccaggcggagctgcgggacagggcgcagaccccgaggcccagggagcaccgggtggccggcggcctgcaggctggcgagggcgtcgggcggcgcagggcaggccagggggcgggggcgtctggggccctggcgtggcgcccggaacaccccgtgccggaagctccatgtgaccgtgactccgcagaagccgcgagcgcagcgaaacaaagggcggctctgcggccgcctcgagctcaggctggcaccgagggcccggacccccatcccactccgcacccccgggcctcccggcccttcttgccctccgaccccgggctctggcagggccgggaggcgcaggaaccccgcgggggatggggccggcggactggcactgaagacactgggatgcaagcgggaggctgggggcggggggctgggggcggggggcggggctgcagggcgtggacggtctc

SEQ ID NO: 10 - DNA, 728 bp, Homo sapiens
  source 1..728: mol_type = genomic DNA
cggagcggatattcccggagcccctctgcgagccacgcgcccctctgggaagcccgcttccccctgcagacaggcgctgtgacacgcttgcgccccggtcgaacaggcgaagaggccgaggcccagagcggcgcagggcgagcctggaggctgcgccccagacctggaccagccacggacgccgctcccgccgctccctccgctccgctgcgctccgcacgctggcgccggctccccgaggccccgggcgccccggccgcacgcctgggtaaaaggtcccgaggagtccgcagaagcgcgcccacgcccgagacggccgtttccgccggcctgggaaaggggcggagaagggggtcgcccgggccgcagcgtgccggtccccgccggccgagccgtgtttggggccagtccccgcaccccgcttcttccccacctggggagcggggcgccgcggtaggggcactggagcgcacgatgcaccccgccaacgagtcctttctgcagacggggttcctgttttcatgcaaatgcctttgttagcgcaccgggaaccagggggacggaactgcagctgacgcgggctgcgcgccgcttttccgccttcgcttgattcggcctcaacgacttaaacgcgccgggaacaaaaacgccggcgccgcggaaaccctcaggagcggcacgagaagcgcggctccgctggggtccgcgagaagcggtgcgggcggccg

SEQ ID NO: 11 - DNA, 575 bp, Homo sapiens
  source 1..575: mol_type = genomic DNA
cgctcgcccccttctccgcgcgggccctcagctcagctccctcttcgctccccgtgtccccgcgagcgggagggaggggatgctaggacgccctgtcggcgtcgtcgccgctttccgccattgtttagtcgtgatgctctcattttctctgaatcaacaattttctgctcggctccgcgccgaccggcgaacgcggggcttttcctcgcccgcctgatgacagcagagcggcgcggagcagctggtccggaaggaagcgccaggcgcctgcccggtcccaggcgtccgctgccgcccacccacaccagaccccgcccccgcgcgtcaagccccgcccatccataccaagtcccgcccccacaccctcacccacacaccaggccgtccccaccccgcccccagagccccggggcgccccgcccgctagccgcgcacgcgcagtgagcacggcgacccccggtggtcgggtgtctccgcaggccgaacacgctgctcgcccagctgcggatcattaccgcccttttgttctccgtcgcgcgctcgccccacgctaggaatgcaaactgtaggcgccg

SEQ ID NO: 12 - DNA, 629 bp, Homo sapiens
  source 1..629: mol_type = genomic DNA
tgtttttccaaaagataataagcgtcaacaacaacaaaaaaataaaaagtccaactccgccccaaagcagcatctggctggcctgcgagatgcccactggggaggcgagtccgcagcttaggactcaagcccggggtcggaagctattgccgaaatccgaaacgcagcgctcgcagctgcagtgacgcgacctgctcataagtccccgtgctcacagcatcccggcaacttacgagctagtgcttccgggtcaccccggcccaggaaggcgcacgcgcgaagcatagcgagcttcactccgcactcttaggctgcgtgtgaggcctgcgagtgctcgggagctgccgcggtcacaagagaaagcctagctgtcaatgacagccccagagcatctgggcgccttgcgatacccgggtgtctgtaggcagccaggagacacttccaagctgatctggaatctttcctcgcccagctctgtccctcgcagggatggcaaaggacattacgacctacatcccttcccggatctgatggcttcagattggcagattgtgttaaagtggaaggctcgtggtgcccctttgctgagtttttatggacttagttttcccaagtagttctaattatcg

SEQ ID NO: 13 - DNA, 907 bp, Homo sapiens
  source 1..907: mol_type = genomic DNA
cggctgtctttgtctcccgcgaggcaactctgactcaggctccagctgcccgtgggagggagggggcgcccgggctcctgaggtcgccagggagcggcgggactgggaggctccaaagccctcagtgtacgtgcgaatccggagcggacaccgagaccttagcgcgggaaccaagagaggacagagctccacggaggccacagcgcgtgcacggggacaggtgcgccctccccggcagcccccctgctcctcggtcacagttctgtgcggaggcgtcttgcgccctcccccctgagcctcgcccttgagtcggggccgtgggccgcatccaggcccccagggctcgggatgcgcgtgaggacccggactcccgagggcgcagaggtcgggagcccgaagcaggcgcccttggccttggtcccgccccttatccggtcccaagctttttcctcgccccttggccttgactccaccccttaggcatgccgctggccccgcccctttccggccaccttgaggcttgggggtccctcagccccgcctctcttcttgaccccgccccttggcagcaccccctacccccgccccacgtccaaatctcccggggccggtggtggccggggctgacggcggaagccgcgcagagactcgcttgccccgaagtcgctggattcgggcctggatcccagattatccgcagcctaggggagtggagagatgcccaaggttcctctgggtcccgggaccccagtagcgtccctccccccgtcccccacgccaaccactgagcgcccttcggagtcccgggaggaaagcgtaggggcggggaactctggcatctctctcctcccggttgctccccgactctgccccgctattccgctatttggggcagtcgtttctaccg

SEQ ID NO: 14 - DNA, 707 bp, Homo sapiens
  source 1..707: mol_type = genomic DNA
cgccgaccccagacccagtcctagtccggccagaggaggccgtttacgagcccacacccgtaggtggcgccacagccggagaattggctttggttctgttggagccgcgccgcctttaaattagccccacgcatgcgcgacttttctagcccgagcccgccgtctgcgcctgcgatttcgcccatactccccggtgcccgcctcgtgacgtgccgcagtgttacgaagggacaccagggcgcaggcgcagctcgctcctcaggcctgcgagaggcccgccggcccagcagagggcgcccaccaatctgcacgcgggcccagcgagttatcttgatttcggccaagctttctgactgctccaaaaaacgaagaaaaagattcagggagagtaagaggatgaagagagctggtgaagcagctgaccaaatggcccgaggtggtatgcagccgcggtaaagcagggcccctccgtgaggcacagccgcccgggggttccctaggggaagcaggggctgcggcagacgcctctcgggcaggtcagggtatgcaccctcccgcaggggctcccaaggccgggcgtgcgtgaggccaggtccacgggcacaccactgtgaacactgattaaacgtggcctccacggcttccaacccccaggacaggaccaaccctctgcccccggctcaggccagagctcggcgagaccgtcg

SEQ ID NO: 15 - DNA, 879 bp, Homo sapiens
  source 1..879: mol_type = genomic DNA
cgcgtccggaggaaggctcacccggaggccgcctgcaggcggccaggtgccagccactgcgggcctctggggccgaagccggcggatggtgagacgctggttggtctgcaacactgcccagaccccgggcactcatgtctagaaaaaagctgactcgtgacaccaaaggagctctttcaagcttcctgcacgctcttagcgccagagcaccccagccgtcctgggagcccccgaagccaagcatattcgaactccgaatccgctcgatcgccggggacctgccatctgggttcggttccccaaggtcgctgccgaccttagaccgcgggggtgtggggcgccggggaaggagacagaaggacaggcgccgcccagggccgcggggacacttggggctgcgtcctgggtgggcgcgatgctcccccagaacgactggagatggagagtgtcggggagggaaacgggacccacgaattcagggcgctgagtcggcggaatgcgccctgactccccctggccgagagccggctcagaatgaaagagcgcggagtgggaggtctgggaaatggcagtatttgtattagggagagaaggaaacaggagtgggagccgcacggcttggggaacgcgggagatcgcggattgcggggatagcgcagcgcggctgcccggggctgctgggaggggccggacgaggccagggcgagcggggtaactgcggccggccggacggcggcggtaaccggctgcaccgaggtggttccaccaccgcgctgggcgcttgcggttcgtctgctccaacgaaagccgcgtcccacgctccctgccgccgcgtggttttgcctcctcagaggggcagcggcgacccaggggctggcg

SEQ ID NO: 16 - DNA, 608 bp, Homo sapiens
  source 1..608: mol_type = genomic DNA
cgcagcaccgggtgttccctcctagcctggtcgctcggggggagcgttggttggcggggtgcaggtctggtgctcgctcaggtgggccaggcacccgcgcgccaggtgaggcgggcgggggaacacacgcccctggcccctgcgccgccgtcacggccgcccaccacccgagggcgggggtcctggtggggtgtcgattccgcctccccgcccacaggcactgggccccgggcggccaccggggtgcggggctcccagcgtctcgggctcccactgcttcaggcctgtccaggggcggggagcgtctctgtggccgcggcgggattgcggcgcggtggccgggcgtcccctgcaggaagctgttctcgctcgctgcctcccccacctgggagggaagcgcctggattttgggtcccgccgccctccgcgccctgggcctccacctgtgttcccaaagcccagccacgagtctgggggtgccgggcgtgccgtggtgggcggaggcttccacagcccctccctgccagggacggcggggcgggacatggcggggccgcacgcaccgggtggacgacaggggatgccgcgggctcgcgtcagccagggcg

SEQ ID NO: 17 - DNA, 1923 bp, Homo sapiens
  source 1..1923: mol_type = genomic DNA
cgccgtttgctcaatgtccccgccagccttgtcggtccttaccgccgtttgactccactgtttttctcgtggtttctgctgcttctctaaattgtccaacgaccgttattcagtaaaaatgaatgaaacggggccgtgtgatctaggcagcctggagatgagattttggaatcataagctacattccaacgtataaaccgattttactcgttttggatactcgatgtacgcggaatgggcgctgtaaaatgcggctgccccgccggaggcatctgcttgggacttgctggcagccgccggtcccctctgcttgcgaccctcggcccagccgccgggaccctggtgcacctgttcctgggcgtcctctctactccccagtggccgccagctccactcccagcctgtggccccggacccgccggcctgagcgttcgcagagggccggtcgtcgccacagccccgcgtcccggcccccgcgccccttggaccttcgccccaggccggcgcagcccagcttcccgggcaggctccacgctaccggggtccagtgcgcggcgacgaagcggagagctgtgtccagactccggagagaaactccggctccgcggggcggcgcggggcggcgcggggcccggagctgcccaactccgccgcctcgggaaggcggcttcgggcccgcagggagccccggggagggttcccggttccgccggcagcggcgtcgaggggtgcctgggctcctggggaccgcgagaggaaaaagaacggaaatcgcaccggggaggaaggacgcgcagaacgcccccgtgaagcggggtgctccggtcaggcgtgcgcgggagcgcggtccgggggagtccggcggcgccgtcgcgcgcactcggcagaggcttcgcgggagaacgcgcagcccggggcgtggggcggggaactgcccgcgcgaggctttcggcgcgtctgggtctcggcgagagcaaagcgcgtcctggcaccgggggcggcggcgcagaggccgggaggaagaaatccgggccctggcccaggtcgggcttccacccctgcgacccgcgagaggcccaggcgggaaaggcggcgagtggcgtcagcggttccgaaagcaaacctggcccggtgctactgcccgagggtcgccgggcgcgtttcctaattcccccgagtctggaaaacggagacttccgtagcgtcttcttcagtgcgtgctgcgagtgctgaaggaggacccggtgcctggacgacccggagcaggggaagcactcggccgacgctgtcgctgtcatcggcgtcattggcgggcaggacagtggggcgggtaaggggcctccccgcgcctcccggcccttcgcgctcggcgccagctctttggctcccttccctgcgcagctctaggcttagctctcagccatttctcaagaagacgatcccgagggtcgaaggccgcccttgacccttgaccacggactctccgtgtaactcggaagagccgtgattttaaaacccggcctcggggttacagaagcccgagatctgggaggcgtccgggacctccctcccagaaccgcagggacccggcctgggatccagggtgtggcctctcgctctgcgcggtcgggaaggcggccgggtccggtcaccgcgccaagcactgcgcacccctgggacgcgtcgttgcggggggctggggggctggggcgcctccacgacgcctggtctgcccggccagtgcttggtgtcgttggtgggttcgtggctgcgacgggtaaacgtccgttccgcgagccgggcaaggcaacccctgcgggtcgcgcccgaaggccggacccctccaagccgcctgggagcttccaaacaggtggacccgaagctcctgtttgatcggagaataacgttcaatttactccgccg

SEQ ID NO: 18 - DNA, 633 bp, Homo sapiens
  source 1..633: mol_type = genomic DNA
tgatgtacgccctggtggacaaaagctgctagtgtcagcttgatttgcaagatcaacattcatgagtttcaccgcttagaaaggggcattatctgcaaaccggagactgaatggaagccataaacaagtgatttcacactaccaagcaggaagaatattctacctcctcaattattctgaacagcatgtttggccccttccaggttcccaccgctagcgagccctccacccctggtgtgtgcaaacggtggacctttcggcgcctaagaaaccgggtgctgagcccgggagcagcgcctgcttttcttcccaagatccactccgggttttgcctagcgctgctccgggaacccattccagacgcaggtaacgccaggcaacgttttccttctcacccgcccaaggccagccccgagccgccggggttccaggcccaacacagcacagatgcacgtttcaaaatgtgctcgaatatgcagcctgcatcaaaggcgttgggaggctctttcatcctctcagctgcctaagaagggacatgctcccagctaccctcatttgtggctgggtttactctgaaatgaagatgtacctctggatgcaaaaagaaagggtggaaggtttttttccccctac

SEQ ID NO: 19 - DNA, 2315 bp, Homo sapiens
  source 1..2315: mol_type = genomic DNA
cggcgggctggattagggcgtgacgccccccaccacgcacacaaacatacacagcccactggatgtctgccgggtgggagccgcaatctccgcgcggtcgatggggccctccgctgcgcactcggccctgcgccgagcaccctgcagcctcctcccgcgacacggcgctttgaactcggcggattgattttgcttcccttcccccttttgtgtgtgtttgcgttcaattggttaggtttttaagatttgggagggctggtgtgaaagaattaaaatactcttaactggagcccctccgccgagaactggaggtcccgcctcctagttcggcgctttcaggaccctcttcccagagggaatttctttcagaaattccagggtgggcttgtaaaagacgcttccgcagagcaggtcccgtcagggtctttttcctgttcctggtgccagcggtcggcccgggcgccccgcagacctcggcgaggtagatgttaagctcggagagtgcccctcccgcaggcgccgtggcgagatcactctgaatatgtaacatatttgtaacgtgcgccgaggtgtgatgtgtgtgctgaaatagggggatgggggaattcgaagccggattgggaaggcgggggggaggcgcacagaactcacaatgtacttcgcaatctaacaatctgaacattcatttattaaaagctgctgcgtgacatttacactgagccaccagtctctgcctctaatccgggcgaaaacgattgtactgccgagttatggctgcagcgtatggggacgctgctgtccgcggccggacagagcccatcagctacaacgcggaaggcctctgcacccccttgggggcgggaggaaagtactgccagtcctgcctgggggccgagggtaacaagcaccgagcctctcgctccacgcagggccagctgcccagctcagcgaagctcttgtgatctggtgcgtgtctctcgctcttccctccccatcaaagaagtaaactttctacctactccccctaatccgatcgtttagagctgctgttttccttttgtcagattcctcctccccgatcagtctgagtacacgatcagaactgctcagagagcaggaagcacattgatttcagcttgttctgtccacagacaggccctgacaaggttgttagaacagccggagaggtctatacaatcacttaattaccaaaactgtcagtcaggcgggacgcggatccgcgtcccgggctgcgctaggcattccagcactgggccgcgcgcgtgattgatcggtgctgatagcaccgcaaaataattacggcgaattttctgatgtgtgattttatcccaagttcatgcttcagagaggtaatcggagaatgagaagggtcagtgccatttcggattacctggaatctgcgagaaagggtaaaatgggggaaggagctccgaggaaaacgggagagatgggggtgcagagagagagggaagaagaaagcgagttatggattgctggagggactgcaagcaattcgtcaaactgtgcaagtgatttccttcagagccagcatatggcagattgattttgtccaacgtcggttttagccacatttaaaatgatccagcggttattactgcgattggcttaggaactgacaggcagttttaggcgcaaggagtatagatcctgtttaccggagatgtgttcgtaactgctgtcaaatacagttaagtaaatatcattagcgaagagctctgttaagagaaatgccaatccaataaatatgcttttcctccccgccctccgcatggctgcctgcgcttcctccagaggttctccttcctgctcctttgctgcttgggtcagacgtcccaggcatggtgctgactcccgccaccttggagccccgagctgagcctcgggcagaagatgacaggccagccgtggggcaaggaggccgcggaaacgcggaacggcttcggggagacggaagcgcccaatgagattcaccctgcagcccgggtccagcccaccttcctcggagattgccgcggccctcgaacccgggcctaggtcttcatgtcccggcggccagaggacgttgcggggaccactggggagctgccctcagtcagctctctgccccacgccggaggtcctggcgcggcttctttcccgaactagactggcgactctgggccaggccccaaggaccgccccggcctctccggctttgcggggagaatctgaggaaccgagtccaagatagccgacctaggctgttttcacccagaccctgcgtccccgacccg

SEQ ID NO: 20 - DNA, 714 bp, Homo sapiens
  source 1..714: mol_type = genomic DNA
agggttgaccccagtacctgacttctccgggagctgtcagctctcctctgttcttcgggcttggcgcgctcctttcataatggacagacaccagtggccttcaaaaggtctggggtgggggaacggaggaagtggccttgggtgcagaggaagagcagagctcctgccaaagctgaacgcagttagccctacccaagtgcgcgctggctcggcatatgcgctccagagccggcaggacagcccggccctgctcaccccgaggagaaatccaacagcgcagcctcctgcacctccttgccccagagaccgtccgagctggagccacaagccctccattcctcttggaatcttcaaccccaaggtaaggtaagttcaccgagcaccgcccagcgatgcgcaggatccgggggggatcacgcgcggcgaccctaccgagcgctccgtgcgcgcccccatctctcggatcgtgttcctggctctgtcgaagctgctgagtcccgcgattcgggaaatccggcacttgtttctcaccctacaccatcacgtggaaatcattgaaaatgggaaccctggtggagtatctgggagagcacgcttgtgccgaggggcctgagctatgggacttcctccaggtccctctgtttcctgccggcgtaggggactcgtagtgtcggatcgcatagtgccaaaaaatagtgcatgggaaacaaacaa

SEQ ID NO: 21 - DNA, 756 bp, Homo sapiens
  source 1..756: mol_type = genomic DNA
gtagagacggtgtttcaccatgttggccaggatggtctcgaactcctgacctcgtgatctgcccgcctcggtctcccaaagtgctgggattacaggcgtgagccactgcgcccagccccaaaattgggaattatttcaaaataaaaagctggataaatgcatacacacaaggcagtatcgcgtattttccacgagtgcctgtgcaggcaggtaaggatttaggaaaggtctggaaggatgtgcaaaatgttccgcctgcgaaggttccgcggtggcggggacactgctccggctccgctcccgcccgcccgagcgctcggatggggccgcctctgcactgcgtggccacaggcgcggcccggctgcccacgggcgccctttgcagctgctgccccctggcggccgcgggcggctactagcgggaaagcgaaacccgcccggtccattcaagccccgctgcctggcgccctctagggtcgttcttgggaacgggcggacctttcgtcaacactttgcctgcaagatcccccattgggggaaccgaggaggaagttaaaggaagatgtgtgtttttgagcgctgctttgtgccaggctcatcttaggtgtgggacgtgtactatctgaattaataccccaccaggcctgtgggacagtcactgtcaccattcgcaaattatggatgaagaaaggaggtaccaagtggtggtatcacctgtccatagtgagctgtccctcaggagggtggccgccccac

SEQ ID NO: 22 - DNA, 523 bp, Homo sapiens
  source 1..523: mol_type = genomic DNA
cgttaacaatgtcgcgtacacgcccgaaccggaggaaccccattccacgctccttctggaaccgaattcacctctgaggctttggggcttcagagccggagccgcttgggcaaaaccagcagaacagcgagagggaacgggctggtctagccctgccctgagcatttctactgagacccccggtcctgcttcttccagcctctgctggatttctctccgacccctctggagcgaagccctttggccctgcgttgcatgcggcacggtgcgggttcgggctctgcgctggagccgggatgccctccggcggagggtgcgcgtaggcggcgcctgggcgtgagccccgcctgcaaggctcagcgtcggggaagcacttttctcgtcgacccggggtctttttccgccaaggagctcggggctcaagaactcgggactgggctgtgggcggggcatggttttcctctctgggcgtcctaatctccaatttcaggcaaattcgctaggaagaaccttcccgagcgcg

SEQ ID NO: 23 - DNA, 1795 bp, Homo sapiens
  source 1..1795: mol_type = genomic DNA
cggtcacagagaagacgcccatcccggaacgcgagcgggagccacccccgcccccacactcggccctctttgtcccctgctcagcggtcaaggaccttgtggtgagcgcctccccacaaacgcagcctcctgcggaattcagccctgcacttttgcagagcttggagccaagacaaatgacatttgtgatcatgagaaagccaagaacgatggaaacggtagcatcgaagttgtgccgctttctgaaacttctttgaactcgttgtgcagggccggggagctctacggccaggaaaagtgcgcagggggcgtccccgcgtcgggcgcgcacacggccagagcacggggctccccacgcgggtttgtctcggacgcagaggggccgcgagcggagacatggacgcggcatttctcacgccaggagctccccgcgcgcgctccccttccacagtccccgccccgcaggccgagagaggaccgcggggacctgcgaggggctgggccgtccaggaggcctcgggtctgcgccccgctcagcccccgcgggacgcctttggcgagagacgcggttctgaaatcagctgtggggtttcgcccaggcccgtcctctggctgcggccatccaagtggcccccgcgtggtgaggcggggccagacccggtgacctccgaggggttagagacctgggcgggggcgggggccagtcctcctcccgagagggcgccgcggggacacagcccaccgccgggagccagcgggacacgggcctcgggcctgacgccgcccacccgagggtgcccgagccccgctgggacccgctcagagccctggcaccgccctgggacgggaccgacgggagcggggggagcgaggacccgtcctgccgtcggagtggagcccggagccagggggtcccccgtccgcccccaaccctcgcggcctcgctaatgaggaaacttggggggcggggtccccgtgctgccgtccccgcgcctgtggccacattctttccacagtcacctccccgcccccatttggcgcgcgacgtctgaggtcgcggatatgcggtgggaacagcccgcgccggggcgtgtggaatgagggtgcccgggcgcccctccctgcacgtggggtcccgcaggcagccgcgccttaaggccagagtcgaagcctgtgggtgcggacacagggaacgttcgaggagacagaaactggggtcctccctgcgttccacccgccgcacccttaagcctcgctctccccaaaacgcgcccgaaactcggcctcgacggggcctcggggcccggcgaccctcgcagcctcccctgggcaaatccggagcgcccctgggacccttcgcacgcgcacgcgcacgcgcgcactcgcacggacgggcgcgcgggaaaaggctcgtccccgcgctcaagcagcccggactggcgcggggggggcggggcggatgaagggaagcgagggggcaggaaatgccgttaattgagggaaacgcgcatgcattgcacgggcggcctttgatgtgcgcctccgggccagcccggcccctccacgccggcgagcccacccggcgtgcgcccctctccgccggcgctcccgggagcgcagggccagcttgagcgccgaggacgcgtggcacttccaacgagcaggaggctgtgggctcactctgtctctaacgggagacagtgcgtggagccctttttgtttctcccccaacccctgggcctcccggggtgggtccggagaccgagcgctgcgggggatgaccacgctgaccgcg

SEQ ID NO: 24 - DNA, 635 bp, Homo sapiens
  source 1..635: mol_type = genomic DNA
ctgtttcaaaactgtgccatctggatgttgcagtatacccattttgtccttcccatacctgtgcccggccacctgatgcaagatgggcacacagccactgaggaagcggagtctgccgcctgccggctgcagggtgcccttaggggtggcctcgatcgccggtggggtccgcatttctgggggacccggcgcctcgacccggagcggggatggtggctctcttgccataacggagaacagaagcggtagggtcagcaagagcaggaaaagaaaaatagggggagggagggggcgccggagaacccaggggtcgctcaggctcgggcgcgaggaggcccgggggttcccgcggctggtgcccgctgaggtgaggggagggggcccatgacgccgcggcggcgcgggcactccctctgcccaactctcggctgagcgcggctcccggctcaggcccctctgccgccgcagccgcgggcccagtagacacaacccagccgaggagcagcagcagcagcagcggcgccccgcgctccctggggccctccagaaagtttttttatggatatcagcaatctaattctacaatttatatggagagacaaaagactcagaataaccaacacaatattgaagga

SEQ ID NO: 25 - DNA, 636 bp, Homo sapiens
  source 1..636: mol_type = genomic DNA
ctgttcctctgtggtggaggaagggacacgcgcttttttttccgaccttaggaaggaacaagggagccggggtcccctcccagcctgggagccctgggcacagtcccggctcatttgtcagagctatcggagccgtcctcgggctggtgggagttcagggctctgaaaggttttctgtcaaggcttgaaagggggccaggtttttttccccccggagccgcgcagtctcggggctgttgttctcagcaatcgcagggcctcgtgttagcaggaagcacagccaagtagggtttcctgcgtgttggagagaggaagctccgtaatgttctgggaggcgatggttaaaaataactccggtatataaagacagcggagggtccccttgttcgctcactcgggcgccggccggctggacgcagggccgagcaggtggtttggggcctcgggaaggccaaacccccgcctctgggcccctggctggggaagacaccagccaagttcagagccccaagtcggcctcacttccacaactcagcgtcagggacaccgtgggcgtttctgtttcaaaacgcttttctccagcaaagaacgtaacctcaagctgctgtcagggtagaggaatccctgccccccgcc

SEQ ID NO: 26 - DNA, 563 bp, Homo sapiens
  source 1..563: mol_type = genomic DNA
cgaaggatgcggcgcgtggaaggagatgcgctgacttgttccaacccataacctttcgctcgggtccccatgtgcgggcagaagaagtcagagcggaacagcctagtgcactggcagggctcattgtctgggaagacaccgaggtctaggcagctgggactgcggagtggaggcaaggccggaggcggccggcggctttgtggaagtttcgcgccgccaggccctgcgcgccgcacggggcggtggagttcttgggcagcccccggcgcttggcccacgcctccgcttcccgcgtgtgggaaactcgagcaccctacaggcaccagggtaaactgcctgtgcctggcccggtgagggtcgctcccccaggccccgtctccgcccgaggactgcaggcctaggcctgcggggagatcctgagaccgcggtgtgcgggcgccggcagcagggcaaggcagggactgtgcccagtccgcccgccaaggagatcgcacgccggcttcgcttctgaagctgcagacggaggccgtggtgagccttagaaagatcccgggacaaaggcg

SEQ ID NO: 27 - DNA, 810 bp, Homo sapiens
  source 1..810: mol_type = genomic DNA
cgccggggcgcaaggccgagtcatcccaggcgtccgtgggccgtgattcccactcacgccgggggcccaggcaggcagagaagagttaatgagcgcgcaagtgcaggcggtcactcctgggcctgaaactcccgcgctgtgcattcagggccctcgtggctctcagaggcgcgtcccaggggcgcacactgcaccttgggctgggcagctccgccgggttgtggcgagcggatgagggaaggacgcagaaaccagggcggaggagccgcgaggggcaggacgaggctgcatgggccagcgagggggtcgacaccgagccagagtgagcgcggggcctggggcgcagagcccgcccagggagccgggagacgccgcgcaagctccccggacaaacgcaatgaccgaggacgcgcgggcgaggccgtccagggagccctggtccctcagctgcaccggactgagccgcgaccgctcagcacgcgctgcttataaatcaggggtgcgcttcccaagccccgggtgaggtcccctacgtcggcacagccttaggagctgcaaagcagcgcgcgcctccggggctcctgcgcgccccttgaaccccgcctcccgcatcctcctgcaacagcctggagctccctgtgcaggacgcagcggggggcggggggcggtcttaggaggctgcggggcgcactcccacctcctgcctccccgagacccccagcgccttctccagggtttagagcggaggtgaaggggcctcgtcctgcaccgccactgggcgcctgggctgttcatcatcggttaccgccg

SEQ ID NO: 28 - DNA, 636 bp, Homo sapiens
  source 1..636: mol_type = genomic DNA
cgtcctgagaacccgagagagcagggcccgctgggacaggcaggggaaggcctcgggagggacacgacggtccggcagcagagcctgcggggctggaggaggcgccctcctctcagctgctcttcctgcccctttcggtggcgaagatggatggggcccggggctttcggcggggcccgaggggccggcgaggctgcggccctggagccccctgcctggcagccatttgggccccagggaaatatcggcgctttggctaaccgaattattctttcggtttgagccagctcccctttttgagtcagatccggcggcagggccagaaaagcgctttctgaaaccccagcgcggtcctcggtgggggtggaatggggtggggtggggggcgcggccgcgccgctgggcgccctccccgccctcccccctccccacccccagtcctccctccgctgcccgccccccaagcccggtgtcgccccctccgccccctgccgcatccccggagccagtgcccacaggggccaggcagcccgcaggggtcgctcacggctggtgtaggggcttggtccaccacgctagtacttcgggcaccaaaatagaaaaagaataacgcttggaaagaatctgatgtttccg

SEQ ID NO: 29 - DNA, 694 bp, Homo sapiens
  source 1..694: mol_type = genomic DNA
cgaaaactaccccgcggaaactagcacagtgtgcctggatgtctgtgtcccgggacctcggggaagagggcccgcaccggtctgcgaattgcaaggcccggccttccccagcgacgctctggtatccgctgtcccctccctgtacctccgcgacccaggggacgcccagtgcaccaggcccttccccggggtcagcggaggcgcagggcgttagccacatcagaggtgcaaatttaccccgggcccaggggaaaatggcgacagcgttcgcggctccacccggggcgcgtgtcagcgttggagagcctgcccggcctgcagagggcgtaacaggcaccgctggggagagccaagcacccctgcgtccaggatccgtagcgccgagctgcaggcccgacctgcagggggcgtgcccggcatgggaagctcaggctacgtctccgaagcttgcgctgaaaacaccagaggtagggaaaacggggagagcgtactgtgctgggctctaccctggacaccccagtttcattctctgcgaagccacgcgctggcagggctctcgggacggcgatacccagggatgatggtacccctggtctcggcgggacctcccgggaacttgtcctgggggagggagcccaactggccacgtactggtagcagcagtgggtggagcgcacaaactccgaggcccgcg

SEQ ID NO: 30 - DNA, 509 bp, Homo sapiens
  source 1..509: mol_type = genomic DNA
cgcctcctgcctgaggcgggctggggggtcgttgtcctcgcagcgttaaggcgagtctgggacaggaccccggcaccccctccggatctgtggcatcctccaggactccggcgcaggacgcgctccaggagccgctccttcagggcctccggtgcgcgcagtccgggcgccggacgagctcctttatcagaaagggcagccgcagagcccgcgtgtgcgcgatgtggctgcgggtggggagcgggcggcgggcccgggacaccgcggccactgttctagccccgcctgggccgcctgaccgcggctccgctgcgccgcagccccgcgcccctctggctcctgttcccgggcgcggggagaaggcggcggggcgcgcctgggcccgcgcgggtgcgaacgcgaggtctttcctgggtgctcccaggtcggaggattcccagggcgggggccatcagggtggcgaggaaccggcagggaccagcctccgctaggaccgcgctcgtggagcg

SEQ ID NO: 31 - DNA, 632 bp, Homo sapiens
  source 1..632: mol_type = genomic DNA
ctttggtttgaaacactggaggtggcccagggccgttttcctcaaaggactgagaatcttgatttgccaagtgcttggggcctccgcccaaggtgtttgggggctgcgtggtgagccgaggcaaagccagggtaccccgatcgtcttccgggcgcatccaccatgcggcaccgccccagccacggcggccgcgcgtggagacccgggggcttaacaaagggctccgcgggggcacgggggggcgcggccacgtgacaggcccgagcgcgacgtcgctgtccagccgcggggaggggcggccagcccggggggccgtggggcttcttgacatagaacgtccgggcctcgggctggccgcgccgggccgcgctccgccgggatgagaagtacttgtctggctccgcgctggagaagccgcacctctcatctctccggctcttacttgaaaaagcacttggaagaaactgtgtgtgcgctgggagggccgcggggtgggccggggccggctgcgaggctgaggggggccggctggtgggtggatggggaggaggttgaagaaacagcccctttctgagtgacaggaccccttttcaaagggcaaacagaaaaaaaaaagaagaagaagaagaaga

SEQ ID NO: 32 - DNA, 861 bp, Homo sapiens
  source 1..861: mol_type = genomic DNA
cgaggcgagttaaattccttttgccggtgcctggctgcgaggacaaacgtccgtactttcgttcgggagccacgggcagtccaggggcttgggttagaagcaacggctctcttccaggggctgtgatccgggtcggccagggagagcgaggccccggggtcctctgtgaggtccccagcgaagagacgcagctggggaaggcgccgcccccgggccccctgcgccaccctaaccgggcctctccttagcaaagttgacaaattcttgagagtgtcagcccagggctgcgcgtgagggcgctgggaccggggaggaaagagcacctgccgcgctcagcccgactttgaatttgtttgttgttaccgtttttgtttttcctcccagtttccataaacgcttagtatttcgaggcactttgcaggtgttggcgcaggtgatgatgggcctcgttggactctgcctcccacgcatccttttgttttctgcgcgccagcctgtctgactgtgtcctgcggggaccccgagacagtccggggtcagggcgtagagactcatgcttgccacttgacccatccgcaacccggggaccccctagcccgtcgcggagctggagtttgggcttccggctcccagctctccgccctggatacaggaagagggcgggagaggtcgcgcacccgcgccgctcggcggggatcgctcacaggggctccggggccaccgcgagcgcggactgcggctgctggcgggctccttcgtcgtccaacgcaccccatcctctcccgccccgcagtgtcccagggaaggcttcactgaaaacagacgctcgacggaaaactgactctgcaggcccgagctttc